(19) United States
(12) Patent Application Publication
(10) Pub. No.: US 2026/0263000 A1
(43) Pub. Date: Sep. 10, 2026

(54) AUTOMATIC DETECTION-THERAPY SYSTEMS FOR CARDIAC ARRHYTHMIAS AND CARDIOVASCULAR MONITORING SYSTEM

(71) Applicant: David C. Shaw, Torrance, CA (US)

(72) Inventor: David C. Shaw, Torrance, CA (US)

(21) Appl. No.: 19/660,889

(22) Filed: Apr. 28, 2026

Related U.S. Application Data
(63) Continuation-in-part of application No. 19/637,692, filed on Apr. 2, 2026.
Application No. 19/637,692 is a continuation-in-part of application No. 19/191,555, filed on Apr. 28, 2025, now Pat. No. 12,642,486.
(63) Continuation-in-part of application No. 19/332,747, filed on Sep. 18, 2025.
(60) Provisional application No. 63/732,819, filed on Sep. 25, 2024.
(60) Provisional application No. 63/732,838, filed on Sep. 27, 2024.
(60) Provisional application No. 63/732,962, filed on Oct. 12, 2024.
(60) Provisional application No. 63/833,068, filed on Oct. 21, 2024.
(60) Provisional application No. 63/833,151, filed on Oct. 26, 2024.
(60) Provisional application No. 64/129,242, filed on Mar. 9, 2026.

(51) Int. Cl. A61B 5/00; A61B 5/0205; A61B 5/021; A61B 5/026; A61B 5/0295; A61B 5/282; A61B 5/291; A61B 5/333; A61B 5/352; A61B 5/361; A61B 5/363; A61B 5/374; A61B 5/384; A61N 1/04; A61N 1/36

(52) U.S. Cl. CPC A61B 5/4836; A61B 5/0006; A61B 5/0205; A61B 5/02108; A61B 5/02141; A61B 5/0261; A61B 5/0295; A61B 5/282; A61B 5/291; A61B 5/333; A61B 5/352; A61B 5/361; A61B 5/363; A61B 5/374; A61B 5/384; A61B 5/681; A61B 5/7405; A61B 5/742; A61B 5/7455; A61B 5/746; A61B 5/7475; A61N 1/0456; A61N 1/36025; A61N 1/36031; A61N 1/36034; A61B 2560/0468; A61B 2562/164

(57) ABSTRACT

An auricular electrocardiogram (ECG) monitoring system, a Speckle Contrast Optical Spectroscope (SCOS), a speckle-plethysmography (SPG) and an auricular EEG are integrated to detect and guide therapy of serious cardiac arrhythmia and impending serious cardiac arrhythmia. An automatic detection-therapy system comprises the auricular ECG, SCOS, SPG (PPG), EEG and neuromodulation units. When ECG signals and cerebral blood flow hemodynamic data suggestive of serious cardiac arrhythmia is detected, the neuromodulation units are configured to automatically start neuromodulating electric stimulation immediately to at least one of: auricular branch of vagus nerve, auriculotemporal nerve, supraorbital nerve, infraorbital nerve, occipital nerve, greater auricular nerve and median nerve, using closed-loop control and R-wave synchronization to enhance effectiveness and safety. A cardiovascular monitoring system, comprising auricular ECG, EEG, SCOS and SPG can monitor cardiac arrhythmia, hemodynamic data, cerebral blood flow-perfusion. These earbud-shaped systems are wearable, user-installable, user-removable and ambulatory.

### CROSS REFERENCE TO RELATED APPLICATION

This application is a continuation-in-part application of U.S. Non-Provisional application Ser. No. 19/637,692, filed on Apr. 2, 2026, entitled “Automatic Detection-Therapy Systems for Neuropsychiatric Disorders” which is a continuation-in-part application of U.S. Non-Provisional application Ser. No. 19/191,555, filed on Apr. 28, 2025, entitled “Auricular Electroencephalogram (EEG) and Automatic Remedy Systems for Neuropsychiatric Disorders”, that claims priority to and the benefit of the filing date of: U.S. Provisional Application No. 63/732,819 filed on Sep. 25, 2024, entitled “Automatic auricular anti-seizure device”; U.S. Provisional Application No. 63/732,962, filed on Oct. 12, 2024, entitled “Automatic auricular detection-remedy system”; and U.S. Provisional Application No. 63/833,151, filed on Oct. 26, 2024, entitled “Automatic auricular detection-remedy system for neuropsychiatric disorders”, which are all hereby incorporated by reference in their entirety. This application also claims priority to and the benefit of the filing date of U.S. Provisional Application No. 64/129,242 filed on Mar. 9, 2026, entitled “Electrocardiogram and Blood Flow Monitoring System and Automatic Anti-Arrhythmia System” which is hereby incorporated by reference in its entity. This application also is a continuation-in-part application of U.S. Non-Provisional application Ser. No. 19/332,747, filed on Sep. 18, 2025, entitled “Wearable ECG Systems and Automatic Anti-Arrhythmia Systems” that claims priority to and the benefit of the filing date of: U.S. Provisional Application No. 63/732,838, filed on Sep. 27, 2024, entitled “Automatic Anti-arrhythmia Device”; and U.S. Provisional Application No. 63/833,068, filed on Oct. 21, 2024, entitled “Auricular Electrocardiogramavice and Automatic Anti-arrhythmia System”, which are all hereby incorporated by reference in their entirety.

### FIELD OF THE INVENTION

This patent specification relates to the field of electrocardiogram (ECG or EKG), cardiac arrhythmia, speckle contrast optical spectroscope (SCOS), speckle-plethysmography (SPG), and electroencephalogram (EEG). More specifically, the present invention relates to the field of cardiovascular monitoring and automatic neuromodulation for cardiac arrhythmia.

### BACKGROUND

There are significant advances in electrocardiography (ECG) with development of dry electrodes and even smaller devices and electronics to be included in smart watches and health trackers (or fitness trackers). Because of the small size, these smart watches or health trackers usually only have two electrodes to form a single lead for ECG. The small size and being wearable on the wrist provide major advantages. However, the currently available smart watches have a disadvantage that they cannot automatically record ECG. They usually require action from the wearer (to use a finger from the opposite hand to touch Digital Crown for Apple Watch or Home Button for Samsung Galaxy Watch) in order to start the ECG recording. They can record ECG only for a short duration.

Sophisticated automatic interpretation algorithms have also been developed for most modern ECG machines. These algorithms can analyze the ECG data and calculate ECG features such as PR interval, QT interval and QRS duration etc. They can also detect various cardiac arrhythmias, including atrial fibrillation (AFib or AF), atrial flutter (AFL), supraventricular tachycardia (SVT), ventricular tachycardia (VT), ventricular fibrillation (VF) and Wolff-Parkinson-White (WPW) syndrome etc. Most smart watches with ECG capability also have automatic ECG interpretation algorithms. With technological advancement, dry electrodes, wireless dry electrodes, and miniaturization trend, very tiny electrodes (sensor electrodes) accompanied with sophisticated algorithms have been developed in recent years. These miniature sensor electrodes are used in various smartwatches and health trackers. There are some smart watches or wearable health trackers that can do ECG.

Conventional ECGs are recorded for a very short time (e.g. 1-2 minutes). Continuous ECG monitoring, like Holter monitor and Zio Patch monitor, can monitor the heart significantly longer. Most Holter monitors employ between 3-8 electrodes, separated from each other. A newer technology, like Zio Patch (or Zio XT Patch), uses a single patch which contains two separated electrodes, forming a single bipolar lead. Both Holter monitor and Zio Patch monitor use special glue or attachment material to attach the electrodes to the chest skin. It is difficult to keep the electrodes attached for a long time. Patients often have skin reactions to the attachment material. Holter monitor is usually used for 24-48 hours. Zio Patch can be used for up to 14 days. None of them can be used for extended monitoring for months or even years.

Cardiac arrhythmia is a very common medical problem in the USA and in the world. There are several types of cardiac arrhythmia. Some of the arrhythmias could be quite serious, including atrial fibrillation (AFib or AF), atrial flutter (AFL), supraventricular tachycardia (SVT), ventricular tachycardia (VT), ventricular fibrillation (VF) and Wolff-Parkinson-White (WPW) syndrome. Ventricular fibrillation is one of the major causes of sudden cardiac death. AFib is also very prevalent and affects millions of people in the USA. AFib is one of the major causes of stroke. Prolonged AFib could lead to heart failure and other cardiac complications. Symptoms of AFib could be subtle and some patients might not be aware of it. The first time they know about the presence of AFib might be after being sent to an emergency room with a stroke and then found to have AFib. AFib could be permanent or paroxysmal. The paroxysmal AFib could start anytime of the day and could start during sleep. Ideally, therapeutic intervention for AFib, AFL, SVT, VT, VF and WPW should be started as soon as possible. However, many patients with these cardiac arrhythmias often have significant delay before they receive therapeutic intervention.

Sometimes healthcare providers might teach some patients to use self-administered maneuvers to stimulate the vagus nerve for AFib or SVT. However, there are many disadvantages with this approach. These self-administered non-electrical vagus nerve stimulations are not very effective. Many patients are unable to do these maneuvers correctly. Sometimes the patients might misjudge the situation and incorrectly use the maneuvers for the wrong situation. Not infrequently, the patients may not be aware of the presence of AFib or SVT and fail to use these maneuvers.

There are implantable chest-based ECG monitors which require a surgery for placement. For example, Abbott developed Assert-IQ ICM which is placed subcutaneously in the left anterior chest. They use enhanced algorithms to reduce false detection of AFib by 98.7% while maintaining 97.7% accuracy. It can monitor cardiac arrhythmia on a long-term basis (3 years or 6 years). It can transfer the ECG data to a monitoring center. When the presence of AFib is detected, it can notify healthcare provider. It would inevitably take some time for the healthcare provider to contact the patient and to arrange for therapeutic intervention. It requires a surgery to insert the device under the skin of the chest.

Vagus nerve stimulation (VNS) therapy has been found by various studies to be effective in treating several types of cardiac arrhythmia, hypertension and heart failure. The placement of a traditional VNS device requires a surgery. The VNS device is usually placed under the skin in the upper chest with a wire going to the neck to wrap around or attach to the left vagus nerve in the neck. Various settings of stimulating strength, pattern, duration, frequency, timing and intervals have been studied to find the most effective stimulating parameters. Similar to VNS, transcutaneous auricular vagus nerve stimulation (taVNS or tVNS) has been studied for cardiac arrhythmia and was also found to be effective in treating cardiac arrhythmia. As compared with VNS, taVNS has the advantage that it does not require a surgery for placement. For most taVNS, its stimulating electrode is usually attached to the tragus or concha portion of the external ear and the stimulating electric current is administered transcutaneously in a way similar to transcutaneous electrical nerve stimulation (TENS). The skin of tragus, concha and the external ear canal is innervated by the auricular branch of vagus nerve. Liu C. et al reported in 2020 that taVNS stimulation is effective for cardiac arrhythmia, similar to VNS. Various stimulation parameters for taVNS have been studied. The key parameters of taVNS include: duration, intensity of the electric stimuli, pulse width, pulse frequency and duty cycles and timing etc. In 2021 Kathrin Machetanz et al reported using taVNS for the heart. They applied taVNS to six different targets of the external ear, including cymba conchae, cavum conchae, outer tragus, inner tragus, crus helicis, and triangular fossa. They found that stimulation to cymba conchae, triangular fossa or inner tragus has strong effects. They applied 24 combinations of stimulus parameters, including 8 electrical charges per phase, 3 pulse durations and 8 charge-balanced current intensities, i.e., 100 microsecond (0.25-2 mA in steps of 0.25 mA), 260 microseconds (0.096-0.769 mA in steps of 0.096 mA) and 500 microseconds (0.050-0.400 mA in steps of 0.050 mA). Thirty bursts at each parameter combination were applied with a periodicity of 1 Hz. Each burst consisted of 5 pulses applied at 25 Hz. Left and right taVNS were studied. They found that 100 microsecond pulse duration and 2 mA current intensity are comfortable and effective.

The tragus and concha portions of the external ear have a unique physiological function because they have rich innervation of the autonomic nerve through the auricular branch of the vagus nerve. The auricular branch of the vagus nerve is an optimal location to place a vagus nerve stimulator. For traditional taVNS, a stimulating electrode is attached to the tragus, cymba concha or cavum concha. The stimulating electrode is connected with a long wire to a stimulating unit, similar to a TENS unit. The presence of a long wire makes it inconvenient. Some taVNS use hand-held or portable devices.

In 1997 the US Food and Drug Administration (FDA) approved cervical vagus nerve stimulation (with VNS) only for the left vagus nerve when stimulated at the neck region, due to concern of possible bradycardia when the right vagus nerve at neck region is stimulated. Traditionally, VNS devices at neck area (vagal stimulation at neck area) are typically implanted to stimulate the left vagus nerve due to concern of possibility for the right vagus nerve stimulation to have a potential adverse effect of bradycardia. However, many studies with taVNS have shown that bilateral auricular vagus nerve stimulation is safe. This is because the right cervical vagus nerve innervates the sinoatrial node, which regulates heart rhythm. The auricula branch of vagus nerve is a sensory nerve and does not have the right vagus nerve's cardiac motor efferent function. The practice of using transcutaneous auricular vagus nerve stimulation (taVNS) was reviewed by Yu Wang et al in 2020. They reviewed other studies and found that bilateral auricular vagus nerve stimulation is safe with no increase of side effects as compared with left-sided stimulation. They also stated that bilateral stimulation could be more effective. From review of many studies, they found that the stimulation parameters have a large range of variation. The most common stimulation frequency is 20 Hz or 25 Hz (range 0.5-120 Hz). The common stimulation pulse width is 1 ms or 0.25 ms (range 0.02-1 ms). They did find that pulse width of 500 microsecond is the most biological active.

In 2024, Ashraf Gerges et al published a review article that also showed safety of bilateral auricular vagus nerve stimulation. They found that taVNS has favorable therapeutic effects on a wide range of clinical pathologies, including paroxysmal atrial fibrillation, heart failure, myocardial infarction, and several neuropsychiatric diseases. They also described various electric stimulation parameters. The stimulation parameters include pulse frequency, pulse width, pulse-pause ratio, electrode type, device used, electric current type, electrode location etc. The Intensity ranged from 0.5 to 50 mA. The electrode size ranged from 2-200 mm. Pulse frequency of either 25 Hz or 20 Hz was used in 74% of the studies. The most common pulse width ranged from 0.05 to 1.0 ms, with either 0.20 or 0.25 ms. being the most commonly used. In their review, 62% of the studies stimulated only the left auricular branch of vagus nerve, while bilateral vagus nerve stimulation was done in 27% of the studies.

Recent advances in ECG analysis algorithms have helped detection of impending serious cardiac arrhythmia. Key predictive patterns of premature atrial contractions (PACs) have been found to help predict development of paroxysmal atrial fibrillation (AFib), including frequency of PACs (or PAC burden) (for example more than 3,000 PACs in a 24-hour period). Origin of PAC from pulmonary veins area is also a highly critical predictor. The pattern (clustering and timing) of PACs, including short rapid bursts or clusters of PACs, using clustering analysis algorithms and variable timing analysis algorithms can also help to predict paroxysmal atrial fibrillation. With artificial intelligence and machine learning, it has been shown that clustering PACs based on their P-wave morphology, timing, and coupling intervals can predict impending AFib. A high variability in the timing of PACs, especially in the coupling interval (the time between the premature beat and the preceding normal beat), has also been found to help predict impending AFib risk. Presence of these specific PACs patterns indicate atrial cardiomyopathy that make the patient vulnerable to atrial fibrillation.

Similarly, key predictive patterns of premature atrial contractions (PACs) have been found to help predict development of atrial flutter (AFL). These include frequency of PACs (PAC burden) (for example over 3,000 PACs in 24 hours). The temporal patterns of PACs are also correlated with risk of impending atrial flutter. With artificial intelligence and machine learning to analyze ECG data, distinct clusters (high-risk clusters) of PACs have been identified, including the PACs morphology, timing, and coupling interval and these have been developed into ECG analysis algorithms for prediction of impending AFL. Source of the PACs (from pulmonary vein area) also helps in prediction of atrial flutter.

Likewise, key predictive patterns of premature atrial contractions (PACs) have been found to help predict development of supraventricular tachycardia (SVT). Frequent PACs (for example: over 3,000 PACs in 24 hours) is again indicative of high risk for SVT. The PAC patterns can also help to predict SVT, including repetitive and organized PAC patterns significantly increase the risk of SVT as compared to simple, isolated PACs, including bigeminy and trigeminy. Occasional short run of non-sustained SVT can also predict impending sustained SVT. Early PACs that are blocked (not conducted to the ventricles) can also be a significant risk factor for impending more complex SVT.

There are ECG patterns that can help to predict impending ventricular tachycardia (VT). VT may occur with underlying cardiac diseases, for example Brugada syndrome with its specific ECG patterns (prominent, coved ST-segment elevation of at least 2 mm in leads V1 and V2 with an associated T-wave inversion.) or arrhythmogenic right ventricular cardiomyopathy (ECG showing T-wave inversion, Epsilon wave, prolong S-wave upstroke). Other ECG technics, like signal-averaged ECG and heart-rate variability may also help to predict impending ventricular tachycardia (VT). Frequent premature ventricular contractions (PVCs) or high burden of PVCs, especially with uniform morphology, can help to predict impending VT. Algorithms for differentiating VT from other wide-complex tachycardias are known in the art.

Similarly, there are ECG patterns and features that can help predict impending ventricular fibrillation (VF). These ECG markers may include T-wave alternans, prolonged T-peak to T-end interval, QT interval prolongation, early repolarization pattern, fragmented QRS, long QRS duration, Epsilon waves, frequent PVCs or episodes of non-sustained VT, etc. and presence of these may indicate increased risks of impending ventricular fibrillation.

Therefore, a need exists for a novel wearable convenient and non-invasive ECG monitoring system that can monitor the heart of a wearer for a long time. A need also exists for a novel automatic anti-arrhythmia system which can automatically initiate therapy for serious cardiac arrhythmia or impending serious cardiac arrhythmia by integrating a wearable ECG monitoring system and a neuromodulation unit.

ECG is essential in monitoring of cardiac arrhythmia. Besides ECG, monitoring of cerebral blood flow, blood pressure, cerebral blood perfusion and cerebral activities can also provide important supplemental information about the status of the heart and cardiac arrhythmia. Cardiac arrhythmia affects cerebral blood flow (CBF) during acute, rapid, or irregular heart rhythm disturbances, causing a reduction of roughly 20% or more, which triggers cerebral hypoperfusion. This often occurs during atrial fibrillation, premature contractions, or tachycardia, leading to reduced cerebral perfusion, potentially causing dizziness, faintness, or cognitive impairment over time. Studies have shown that during severe tachycardia from atrial fibrillation, atrial flutter or supraventricular tachycardia, there may be significant decrease of cerebral blood flow with an average of 23 to 40%. Frequent premature ventricular contractions (PVC's) have been shown to cause about 32% decrease of cerebral blood flow velocity. Persistent atrial fibrillation could lead to lower sustained cerebral perfusion as compared to normal sinus rhythm. When blood flow to the brain is suddenly reduced, there could be consequences, like cognitive decline, lightheadedness, dizziness, or fainting (syncope). Severe cardiac arrhythmias (e.g., ventricular tachycardia, severe bradycardia) can cause significant reductions in blood flow to the brain, which may manifest as sudden diffuse slowing or flattening (attenuation) of the EEG, often associated with syncope or near-syncope. Cardiac arrhythmias like supraventricular tachycardia (SVT) or severe tachycardia associated with atrial flutter (AFL) or atrial fibrillation (AFib) can affect electroencephalogram (EEG) findings. They may cause cerebral hypoperfusion (reduced blood flow), leading to EEG slowing (increased delta/theta waves). Studies have shown that AFib could be associated with unique neurophysiological changes, such as altered beta phase-amplitude coupling, which may be linked to cognitive decline in these patients.

There are a few different methods to assess intracranial and extracranial blood flow. The traditional ultrasound devices are usually not wearable. Transcranial Doppler Ultrasound (TCD) is a non-invasive technique for real-time monitoring of cerebral blood flow. Recent advancements include wearable ultrasound devices that can monitor cerebral blood flow. For instance, a lightweight system developed by researchers at Caltech and USC accurately measures blood flow to the brain, distinguishing it from scalp blood flow. The transcranial doppler ultrasound can be combined with carotid duplex ultrasound to assess the blood flow in stroke patients and carotid stenosis. An emerging flexible doppler ultrasound device uses wearable sensors for long-term continuous monitoring of cerebral blood flow.

Speckle-plethysmography (SPG) and photoplethysmography (PPG) have emerged as useful devices to assess regional blood flow. It can also provide estimated data regarding systolic blood pressure and diastolic blood pressure. The SPG (or PPG) can estimate systolic and diastolic blood pressure (BP) by analyzing waveform features like peak amplitude and arterial stiffness. When compared with traditional cuff method, the SPG and PPG have mean errors of 10-14 mmHg for systolic BP and 7-10 mmHg for diastolic BP. (mean error of 12.4 mmHg for SPG and 13.7 mmHg for PPG). The estimated blood pressure data are often very valuable.

New development in laser technology also provides convenient way to monitor cerebral and extracranial blood flow. There are a few laser speckle-based blood flow monitoring units. One example is Speckle Contrast Optical Spectroscopy (SCOS) which is a non-invasive, camera-based diffuse optical technique that can measure deep-tissue microvascular blood flow and perfusion at different depths. By analyzing the blurring of laser speckle patterns caused by moving red blood cells, SCOS provides high signal-to-noise ratio assessments of blood flow dynamics. When tissue is illuminated by a laser, the backscattered laser light creates a random interference pattern known as speckle. Moving red blood cells cause this speckle pattern to blur. The SCOS measures the contrast (ratio of standard deviation to mean intensity) of these patterns to determine blood flow speed. The SCOS uses multi-exposure with multi-depth approaches to analyze how speckle contrast blurs depending on the camera exposure time or the distance between the light source and light detector. It is used to monitor cerebral blood flow, brain function (in stoke patients or brain trauma patients), and deep tissue perfusion. A similar and related laser speckle-based blood flow monitoring unit, called Diffuse Correlation Spectroscopy (DCS), can measure deep tissue blood flow and microvascular hemodynamics in real-time. Another related laser speckle-based blood flow monitoring unit, called Laser Speckle Contrast Imaging (LSCI), works by illuminating tissue with a laser light, producing a speckle pattern that blurs where blood is moving, creating a contrast map that indicates blood flow velocity, with higher speeds of blood flow resulting in greater blurring.

The technology behind SCOS, DCS and LSCI is related and they use similar components. The core components for Speckle Contrast Optical Spectroscopy (SCOS), include a coherent light source (typically a laser diode at 785 nm or 850 nm), a high-sensitivity light detector (such as a CMOS or sCMOS camera, or SPAD array) for capturing speckle patterns, and a data processing unit to analyze speckle contrast, usually involving noise correction. The coherent light source is usually a laser diode (e.g., 785 nm or 850 nm) that can provide the necessary coherent illumination to produce speckle patterns. (Most SCOS may have one to three laser diodes). The light detector (camera) is usually a high-speed CMOS or Scientific CMOS (sCMOS) camera, or specialized SPAD array, to be used to capture speckle images, allowing for high-resolution measurement of speckle intensity fluctuations. (CMOS is a high-speed Complementary Metal-Oxide Semiconductor sensor). Other components may include fiber coupling/delivery to deliver the light via a fiber-coupled system to the tissue and scatted laser light can be collected either directly by the camera or through fiber bundles. The SCOS device may also include a modular probe or mount to be used to secure the laser and the camera on the scalp (or other tissue) to measure cerebral blood flow. The data processing unit uses software or hardware that calculates the speckle contrast and performs corrections for dark noise, read noise, and shot noise to ensure accurate flow measurements.

Neuromodulation of the auricular branch of vagus nerve, such as via taVNS, has been shown to be very helpful for various types of cardiac arrhythmia. Neuromodulation to other nerves has also been shown to be quite beneficial for cardiac arrhythmia, including supraorbital nerve, infraorbital nerve, auriculotemporal nerve, occipital nerve, greater auricular nerve and median nerve. Neuromodulation to more than one of these nerves simultaneously has been found to be more helpful due to their synergistic effects. The auricular branch of vagus nerve, the auriculotemporal nerve and the greater auricular nerve all innervate the skin of the ear and, therefore, the ear is a very unique and convenient location to have neuromodulation stimulations to all of these 3 nerves. The commonly used stimulation parameters for taVNS, supraorbital nerve, auriculotemporal nerve, occipital nerve, greater auricular nerve, infraorbital nerve and median nerve may be different. Generally, examples of common stimulation parameters (general approach): (1). Intensity: Individually fitted to create a strong, “tingling” but non-painful sensation, below the pain threshold. (2). Pulse width: 250-500. (3). Frequency: 1 Hz (often used for modulation) or 20-30 Hz for therapy. (4). Waveform: Often dense-sparse waves.

The human ear is the only location where the vagus nerve reaches the body surface (the skin), through auricular branch of vagus nerve. The cymba concha has almost exclusive vagal nerve innervation. Cavum concha (the lower, larger bowl-shaped part) is located around the opening of the external ear canal. Cavum concha also receives significant innervation from the auricular branch of vagus nerve. While cavum concha is largely innervated by the vagus, the anterior cavum concha receives mixed innervation from the auriculotemporal nerve (a branch of the mandibular division of the trigeminal nerve) and the inferior portion receives innervation from the greater auricular nerve. The facial, glossopharyngeal, and cervical nerves also innervate part of the cavum concha. The inner and posterior tragus is innervated by vagus nerve, while the anterior outer part of tragus is innervated by trigeminal nerve (auriculotemporal nerve) and some from greater auricular nerve (which is from cervical nerve roots). The posterior and inferior walls of the external ear canal receive innervation from the vagus nerve, while the anterior and superior walls of the external ear canal is innervated by the auriculotemporal nerve. The vagus nerve innervated auricular skin includes inner tragus, cymba-concha, cavum-concha, posterior inferior walls of the external ear canal and small adjacent regions of the external ear. Transcutaneous auricular vagus nerve stimulation (taVNS) can be placed on the vagus innervated auricular skin to stimulate the vagus nerve. The vagus nerve has huge influence on various human body functions, including brain, heart, breathing, emotions, blood pressure, GI system and metabolism, etc. The cavum concha is heavily innervated by the auricular branch of vagus nerve although the auriculotemporal nerve (a branch of trigeminal nerve) and greater auricular nerve also provides overlapping innervation. The inner (medial) posterior tragus is primarily innervated by the vagal nerve although the anterior outer part of tragus is innervated by the auriculotemporal nerve.

The auriculotemporal nerve (ATN) mainly supplies the anterior-superior part of the pinna (including anterior-superior helix), anterior outer tragus, part of the inner tragus (inner/medial surface of tragus, facing the external ear canal), anterior-superior region of cavum concha and anterior-superior walls of external ear canal. The ATN innervated auricular skin includes anterior outer tragus, part of the inner tragus (inner/medial surface of tragus), anterior and superior part of pinna (including anterior-superior helix), anterior and superior walls of the external ear canal and the anterior-superior region of cavum concha. The ATN is a branch of trigeminal nerve. The auriculotemporal nerve (ATN) stimulation unit is sometimes combined with auricular vagus nerve stimulation unit (taVNS) due to anatomical proximity or overlapping of the ATN and auricular branch of vagal nerve in the ear around tragus, cavum concha and external ear canal. The ATN stimulation unit and taVNS unit may share a same housing with a same stimulating electrode. However, they often use their own optimized stimulating electrodes with their own distinct stimulating parameters. The primary targets for taVNS stimulating electrode are cymba concha (100% vagal), cavum concha, inner-posterior portion of tragus (mixed vagal/trigeminal) and posterior-inferior walls of external ear canal for autonomous nerve modulation. The primary targets for auriculotemporal nerve include anterior outer part of tragus, anterior-superior helix, anterior portion of cavum concha and anterior-superior walls of external ear canal. If the anterior-superior helix is selected as the target for the auriculotemporal nerve, the stimulating electrode may be attached via a clip electrode. Both ATN stimulation and taVNS stimulation can be applied concurrently to the ear with about 30 minutes stimulation session. There are various stimulating parameters. For example, a common stimulating parameter for taVNS is 20-25 Hz frequency, 200-500 microsecond pulse width with intensity adjusted to a comfortable sensory level, usually below 5 mA. The stimulating parameters for ATN are often different, with higher frequency (around 100 Hz) to modulate different receptors. The stimulating strength for both of them can be titrated to the user's individual maximal tolerable level without pain. Combining these techniques can have synergistic effect.

Supraorbital nerve and infraorbital nerve are different branches of trigeminal nerve. The most easily targeted and commonly used trigeminal nerve branches for neurostimulation include the supraorbital nerve (V1 or ophthalmic division) and infraorbital nerve (V2 or maxillary division) because they are superficial, making them easily accessible for electrode placement. The other good choice is the auriculotemporal nerve (V3 or mandibular division). Limited studies have shown potential benefit of supraorbital nerve stimulation and infraorbital nerve stimulation for cardiac arrhythmia (and some neuropsychiatric disorders) when proper precaution is applied to avoid side effect from trigeminocardiac reflex. Examples of transcutaneous supraorbital nerve stimulators include a Cefaly device which is FDA-approved for migraine. The Cefaly is a wearable, non-invasive device. The Cefaly device is mounted on the forehead and uses pre-determined electrical impulses on the forehead for neuromodulation. Supraorbital nerve stimulation is a type of trigeminal nerves stimulation. The supraorbital nerve stimulation has been shown to help cardiac arrhythmia and refractory seizures.

The median nerve stimulator has been found to be quite helpful for cardiac arrhythmia, especially atrial fibrillation and ventricular arrhythmia. The median nerve stimulator usually gives transcutaneous stimulation to the median nerve near the wrist area. The median nerve is a mixed nerve with motor and sensory fibers. By adjusting the stimulation intensity, the median nerve stimulator may be configured to stimulate only the sensory fibers or both sensory and motor fibers. The most commonly used stimulation parameters of median nerve stimulator for cardiac arrhythmia are as follows: frequency: 5 Hz (may use up to 20 Hz), amplitude/intensity: 2.5-3.5 mA (up to 15-20 mA), pulse width: 100-300 microseconds, duty cycle: 10 seconds on/30 seconds off or 20 seconds on/40-50 seconds off.

The occipital nerve stimulator is an adjunct therapy for neuropsychiatric disorders and limited studies have shown potential benefit for cardiac arrhythmia. The occipital nerve (with 3 branches) arises from the C2 and C3 cervical spinal nerves. The occipital nerve stimulating electrode was often implanted surgically near the occipital nerves at the base of the skull although transcutaneous version had been developed recently. By impacting the convergence of cervical and trigeminal nerves, stimulation of occipital nerve can help to modulate the brain. The commonly used stimulating parameters for occipital nerve for cardiac arrhythmia are: amplitude: 0.5-2.0 mA or individually titrated to just below the paresthesia or discomfort threshold, frequency: 20-40 Hz (avoid high frequency that might induce asystole or bradycardia), pulse width 200-300 microseconds, duty cycle: one hour daily or intermittent.

The greater auricular nerve (GAN) stimulation has also shown promise for cardiac arrhythmia. Studies have shown GAN stimulation has potential to enhance neural plasticity and improve cognitive functions. It has been shown that GAN stimulation is effective in reducing postoperative atrial fibrillation. GAN stimulation is found to enhance parasympathetic activity, which can lead to improved heart rate variability and reduced stress responses. This modulation of the autonomic nervous system may contribute to its therapeutic effects across various neuropsychiatric conditions. The GAN (from C2-C3 nerve roots) provides sensory innervation to the skin of the inferior auricle, posterior auricle and the lower part of cavum concha.

There are other reported examples of stimulation parameters for trigeminal nerve stimulation (TNS) (including supraorbital nerve stimulation unit, auriculotemporal nerve stimulation unit and infraorbital nerve stimulation unit) and occipital nerve stimulation unit: a high-frequency, low-intensity pulse is usually used, for example a 100-120 Hz frequency and a 250 microseconds pulse width. The typical stimulation parameters for trigeminal nerve stimulation are: (1). Intensity/Strength: 2-4 mA (with a range of 1-10 mA, maximum of 16 mA) adjusted for comfort to a mild tingling sensation without pain. (2). Frequency: 60-120 Hz. (3). Pulse width (Duration) 200 to 250 microseconds. (4). Stimulation cycle: commonly 30 seconds on/30 seconds off or continuous for 20-60 minutes depending on condition. (5). Session duration: 7-9 hours (overnight). (6). Waveform: usually biphasic pulses.

The US Food and Drug Administration (FDA) approved vagus nerve stimulation only for left vagus nerve when stimulated at neck region, due to concern of possible bradycardia when right vagus nerve is stimulated. The practice of using transcutaneous auricular vagus nerve stimulation was reviewed by Yu Wang et al in 2020. They reviewed other studies and found that bilateral auricular vagus nerve stimulation is safe with no increase of side effects as compared with left-sided stimulation. They also stated that bilateral stimulation is more effective. From review of many studies, they found that the stimulation parameters have a large range of variation. The most commonly reported and effective taVNS stimulation parameters for cardiac arrhythmias are: a frequency of 20-25 Hz, a pulse width of 200-300 microseconds and an on-off cycle of 30 seconds with 30-60 minutes duration daily, and the most commonly used duty cycle is 30 seconds on/30 seconds off. In some studies, patients were allowed to adjust the stimulation strength to maximum tolerable strength.

### BRIEF SUMMARY OF THE INVENTION

According to one aspect consistent with the principles of the invention, an auricular electrocardiogram (ECG) monitoring system that is wearable, convenient, suitable for long-term monitoring and with much less muscle artifacts and movement artifacts is disclosed. In some embodiments, an auricular ECG monitoring system may comprise an ECG recording module. The ECG recording module may comprise at least two wired or wireless ECG electrodes (ECG sensor electrodes) in electronic communication with the ECG recording module. All of the ECG sensor electrodes (for example a first ECG sensor electrode and a second ECG sensor electrode) may be configured to be coupled to a wearer's first ear or a peri-auricular area around the wearer's first ear. When the first and second ECG sensor electrodes are coupled to the wearer's first ear or the peri-auricular area around the wearer's first ear the first and the second ECG sensor electrodes are positioned in contact with separate skin locations on at least one area of the wearer's skin, the area selected from at least one of the following: external ear of the wearer's first ear, external ear canal of the wearer's first ear, and the peri-auricular area around the wearer's first ear. (It should be noted that the first and the second ECG sensor electrodes may contact separate skin locations within a single area. Alternatively, the first ECG sensor electrode may contact a skin location within one area and the second ECG sensor electrode may contact a separate skin location within another area.) These skin locations that the auricular ECG sensor electrodes are attached to may be adequately separated (with different angles or directions) relative to the location of the heart of the wearer so that they can pick up some differences of the cardiac action potentials in order to enable the ECG recording module to perform an ECG. The ECG recording module may be configured to record ECG data of the wearer via the ECG sensor electrodes and the ECG recording module may be in electronic communication with a processing unit. With the help of ECG analysis algorithms, the processing unit may be configured to record and analyze the ECG data from the ECG recording module to assess the wearer's ECG profile. The processing unit may be configured to analyze ECG data recorded by the ECG recording module to detect presence or cessation of a serious cardiac arrhythmia of the wearer. The processing unit may be further configured to analyze the ECG data recorded by the ECG recording module to detect the presence or cessation of an impending serious cardiac arrhythmia of the wearer. The serious cardiac arrhythmia may include atrial fibrillation (AFib), atrial flutter (AFL), supraventricular tachycardia (SVT), ventricular tachycardia (VT), and ventricular fibrillation (VF).

As used herein, a peri-auricular area refers to a portion of the head around the auricle (pinna) and this portion of the head is typically hairless. The peri-auricular area includes a portion of the head in front of the auricle (pre-auricular area) and a portion of the head above and behind the auricle (post-auricular area). The pre-auricular area is small, about one inch wide and two inches long and curved along the anterior edge of the auricle. The post-auricular area is also small, about one inch wide and about three inches long and curved along the superior and posterior edges of the auricle (pinna). The post-auricular area is where a behind-the-ear hearing aid is usually located. The small pre-auricular area and post-auricular area together will be called “peri-auricle area” hereinafter. (Anterior, posterior, superior, in front of and behind etc. all refer to the directions relative to the wearer's head when the wearer is in an upright position.)

The auricular ECG may be housed in an auricular housing (for example a modified ear-bud housing) to be placed in an external ear canal or a tragus-concha bowl (concha auricularis) of a wearer. When both ECG sensor electrodes are located within an auricular housing (such as a modified earbud housing), the ECG signals are typically quite weak because the two ECG sensor electrodes are quite close to each other. Modern technology, such as advanced signal processing (for example denoising convolutional autoencoders), high-impedance amplifiers, specialized instrumentation amplifiers, and denoising algorithms etc. can improve the signal-to-noise ratio, allowing accurate ECG tracking from a single ear. Alternatively, the two ECG electrodes may be located more away from each other, for example, a first ECG electrode in one ear and a second ECG electrode in another ear (dual-ear setup), or a first ECG electrode in one ear and the second ECG electrode be placed in one wrist or one finger or one ankle or one toe, etc. The dual-ear setup and the ear-wrist setup etc. will have much stronger signal quality although would be more inconvenient for the wearer (user).

According to another aspect consistent with the principles of the invention, an automatic detection-therapy system for cardiac arrhythmia is disclosed. Preferably, the auricular automatic detection-therapy system for cardiac arrhythmia may comprise an auricular electrocardiogram (ECG) monitoring system, a neuromodulation system and a processing unit. The auricular electrocardiogram (ECG) monitoring system may include an ECG recording module that is configured to record ECG data of the wearer. The auricular ECG monitoring system is in electronic communication with the processing unit. The neuromodulation system may comprise at least one of the following components: a transcutaneous auricular vagus nerve stimulation unit (taVNS unit), a supraorbital nerve stimulation unit, an auriculotemporal nerve stimulation unit, an occipital nerve stimulation unit, a greater auricular nerve stimulation unit, a median nerve stimulation unit and an infraorbital nerve stimulation unit. Each component of the neuromodulation system is configured to send neuromodulating electric stimulation to the wearer when activated or prompted. Each neuromodulation component is in electronic communication with the processing unit. The taVNS unit may have a stimulating electrode attached to vagus innervated auricular skin of the wearer. (Vagus innervated auricular skin refers to areas of auricular skin that have rich innervation from the auricular branch of vagus nerve, such as external ear canal, tragus, cymba concha, cavum concha and small adjacent areas of the ear). The processing unit is configured to analyze the ECG data to detect presence or cessation of ECG signals suggestive of serious cardiac arrhythmia or impending serious cardiac arrhythmia. When presence of serious cardiac arrhythmia is detected by the processing unit, the processing unit is configured to send signals to the neuromodulation system to prompt the neuromodulation system to start sending pre-determined (or closed-loop controlled) neuromodulating electric stimulation to the wearer. When cessation of serious cardiac arrhythmia is detected by the processing unit, the processing unit is configured to send signals to the neuromodulation system to prompt the neuromodulation system to stop sending neuromodulating electric stimulation to the wearer. When presence of impending serious cardiac arrhythmia is detected by the processing unit, the processing unit is configured to send signals to the neuromodulation system to prompt the neuromodulation system to start sending pre-determined (or closed-loop controlled) neuromodulating electric stimulation to the wearer. When cessation of impending serious cardiac arrhythmia is detected by the processing unit, the processing unit is configured to send signals to the neuromodulation system to prompt the neuromodulation system to stop sending neuromodulating electric stimulation to the wearer.

It is well-known that during serious cardiac arrhythmia, there may be significant decrease of cerebral blood flow, significant change of blood pressure and change of the wearer's electroencephalogram (EEG). Besides monitoring ECG, monitoring cerebral blood flow or blood pressure or EEG (or cerebral blood perfusion) may provide valuable supplementary information regarding the wearer's cardiac arrhythmia. Monitoring the cerebral blood flow, the blood pressure and EEG will also provide valuable feedback guidance and safety when using closed-loop control system to deliver neuromodulating electric stimulation (such as taVNS neuromodulation or trigeminal nerve neuromodulation) to avoid sudden development of severe bradycardia or severe hypotension. (The trigeminal nerve neuromodulation includes stimulation to the supraorbital nerve, the auriculotemporal nerve and the infraorbital nerve).

In preferred embodiments, an automatic detection-therapy system for cardiac arrhythmia may comprise a cardiovascular monitoring apparatus, a neuromodulation system and a processing unit. The cardiovascular monitoring apparatus comprises an auricular ECG monitoring system and a cephalic blood flow monitoring system. The auricular ECG monitoring system is as described hereinbefore. The cephalic blood flow monitoring system comprises an auricular speckle contrast optical spectroscope unit (auricular SCOS unit). The auricular SCOS unit is housed in an auricular housing and is configured to monitor the wearer's cerebral and extracranial blood flow. In addition, the cephalic blood flow monitoring system may further comprise at least one of: a speckle-plethysmography (SPG) unit and a photoplethysmography (PPG) unit. The SPG (or PPG) unit may also be housed in the auricular housing and is configured to monitor the wear's regional blood flow and the wearer's systolic and diastolic blood pressure. The cardiovascular monitoring system may further comprise an auricular electroencephalogram (EEG) monitoring system. The auricular EEG monitoring system is located in the auricular housing and is configured to record the wearer's EEG data. The processing unit is configured to convert the raw EEG data into quantitative EEG (qEEG) data. It is known that serious cardiac arrhythmia may produce significant decrease of cerebral blood flow, significant drop of blood pressure and change of EEG and qEEG metrics (e.g. increase of slow waves and decrease of cordance z-score) (indicative of decreased cerebral blood perfusion.) Serious cardiac arrhythmia could result in significant decrease in cerebral blood flow (e.g. around 20-30%). Serious cardiac arrhythmia could also result in significant drop of systolic blood pressure (for example: a decrease by 20 mmHg or decrease of blood pressure below 90/60 mmHg.) Serious cardiac arrhythmia could also cause EEG changes including overall EEG suppression or flattening, decrease of fast waves (alpha and beta) and increase of slow waves (theta and delta waves). The severity of EEG changes correlates directly to the decrease in cerebral blood perfusion (hypoperfusion) caused by the cardiac arrhythmia. When the processing unit detects presence of serious cardiac arrhythmia, the processing unit is configured to send signals to the neuromodulation system to prompt the neuromodulation system to immediately start sending neuromodulating electric stimulation from at least one of the neuromodulation components. When the processing unit detects presence of serious cardiac arrhythmia, and if the cardiac arrhythmia co-exists with (or is accompanied by) at least one of the following: significant decrease of cerebral blood flow (e.g. a decrease of 30% or more), significant decrease of blood pressure (e.g. decrease by around 20 mmHg or more) and significant slowing of wearer's EEG (e.g. one of: an increase of relative delta power more than 30%, an increase of delta/alpha ratio more than 30% and a decrease of cordance z-score more than one standard deviation), the processing unit may be configured to send urgent signals to the neuromodulating system to immediately start more potent neuromodulating electric stimulation (for example, higher frequency, intensity or duration etc.) or to recruit more components of the neuromodulation system to give neuromodulation to the wearer. The processing unit further includes a closed-loop control system that is in electronic communication with the auricular ECG monitoring system, the auricular SCOS unit, the at least one of: the SPG unit and the PPG unit, the auricular EEG monitoring system and the neuromodulation system. Through the closed-loop control system, the processing unit is configured to receive real-time continuous input data (feedback data) from the auricular ECG monitoring system, the auricular SCOS unit, the at least one of: the SPG unit and the PPG unit and the auricular EEG monitoring system and the processing unit is further configured to analyze these input data, with controlling algorithms, to continuously adjust the actuating outputs to the neuromodulation system. The actuating outputs includes turning on or turning off at least one component of the neuromodulation system and adjusting the stimulating parameters (intensity, frequency, duration, cycle, waveform etc.) of the neuromodulating unit when a particular neuromodulating unit is turned on. The processing unit is also configured to deliver the signals to the neuromodulation system to time the neuromodulating electric stimulation with R-wave synchronization mode. The processing unit is further configured to use the real time ECG data to precisely time the delivery of stimulating electric stimulation in accordance with the R-wave synchronization mode to improve the efficacy and decrease the side effects. By integrating multiple monitoring systems and multiple neuromodulating units (or components) incorporated into the closed-loop control system, this automatic detection-therapy system for cardiac arrhythmia will have much higher effectiveness and much higher safety because this system will allow usage of higher stimulation frequency when needed and can avoid potential sudden drop of heart rate or sudden drop of blood pressure when high frequency stimulation is applied to the trigeminal nerve. When high frequency stimulation to the trigeminal nerve (including supraorbital nerve, auriculotemporal nerve and infraorbital nerve) is deemed needed, less-high frequency can be tried initially and gradually increase as needed with the guidance of constant feedback from the closed-loop control system. In rare situation, if sudden bradycardia or sudden hypotension occurs, the closed-loop control system may immediately adjust the stimulation parameters or stop the neuromodulation stimulation. In rare situations, if unwanted side effects from vagus nerve stimulation or trigeminal nerve stimulation occur, there are techniques available to temporarily block the nerve or reverse the stimulation effect. Specific stimulation parameters can be utilized to block or reverse the effects of neuromodulation on the trigeminal and vagus nerves, for example, switching from low-frequency activating protocols to high frequency, high-duty cycle or using specific bipolar polarity to hyperpolarize the nerve and cause temporary nerve block to stop signaling.

It is known that regular digital EEG recording can be used to generate data for quantitative EEG (qEEG). There are different methods to quantify EEG changes during cerebral hypoperfusion (such as during serious cardiac arrhythmia). Quantifying changes in EEG during decreased blood perfusion (cerebral hypoperfusion) involves monitoring the transition from fast-wave activity to slow-wave activity, characterized by a reduce of cerebral blood flow from normal (50-60 ml/100 g/min) down to ischemia-induced slowing (17-18 ml/100 g/min) and finally to suppression (<10 ml/100 g/min). Quantitative EEG (qEEG) metrics that can be used to assess cerebral hypoperfusion, include spectral power changes, frequency ratios, and symmetry indices calculated using Fast Fourier Transform (FFT). Spectral power and ratios can measure EEG slowing that include an increase of slow waves (delta and theta) and decrease in high frequency (alpha and beta). Relative delta power (the ratio of power in delta band, 0.5-3 Hz, to the total power, 1-30 Hz, is a very useful indicator of cerebral hypoperfusion. Delta/alpha ratio (DAR) can also be used to assess hypoperfusion (hypoperfusion will cause increase in delta power and decrease of alpha power.) (Delta+Theta)/(alpha+beta) ratio (DTABR) and alpha/delta ratio (ADR) may also be utilized. The processing unit is configured to analyze the EEG data recorded by the auricular EEG monitoring system to obtain quantitative EEG (qEEG) data and to detect presence of serious cardiac arrhythmia when the cerebral blood perfusion decreases below a certain level (or a decrease of more than a certain percentage) as indicated by an increase of relative delta power more than a predetermined percentage or an increase of delta/alpha ratio more than a predetermined percentage, suggestive of a decrease of cerebral blood perfusion below a certain level. There are many other data (for example fast Fourier transformation, density spectral array, cordance z-score, etc.) that are available from the qEEG that are useful for assessment of cerebral blood perfusion. Usage of other metrics from qEEG to assess the cerebral blood flow is within the scope of this invention.

As used herein, the term “auricular housing” refers to housing structures in or around the ear. Examples of an auricular housing include a modified earbud housing, a modified in-the-ear housing, a behind-the-ear-hearing-aid-style housing, and a tubular-shaped structure. As used herein, the term “tragus-concha bowl” (concha auricularis) refers to the area surrounding the opening of the external ear canal and this area is bowl-shaped and is formed by inner tragus and cavum concha. (cymba concha is above the cavum concha and they are separated by crus of the helix). The modified earbud housing includes a tubular-body portion and a stem portion. The stem portion is equivalent to the “stem” of an earbud that hangs down from the earbud. The tubular-body portion comprises a tubular-shaped structure and a body-structure. The tubular-shaped structure is modified by elongating the “ear-tip” and “nozzle” of an earbud and the tubular-shaped structure is configured to be inserted into a wearer's external ear canal when in use. The body-structure is equivalent to the “body” of an earbud and is configured to be placed at the immediate opening of the wearer's external ear canal and be placed inside the wearer's tragus-concha bowl when in use. The modified in-the-ear housing is modified from an in-the-ear (ITE) hearing aid. The modified in-the-ear housing also includes a tubular-shaped structure (to be inserted into the wearer's external ear canal when in use) and a body-structure (to be placed at immediate opening of the wearer's external ear canal and be placed inside the wearer's tragus-concha bowl when in use). A behind-the-ear-hearing-aid-style housing includes an in-the-ear portion and a behind-the-ear portion. The in-the-ear portion (of a behind-the-ear-hearing-aid-style structure) also includes a tubular-shaped structure (to be inserted into the wearer's external ear canal when in use) and a body-structure (to be placed at immediate opening of the wearer's external ear canal and be placed inside the wearer's tragus-concha bowl when in use.) It should be noted that, as described in detail hereinafter, the material and setups of the housing structures in this invention will enable easy self-installation and easy self-removal of various sensor electrodes and stimulating electrodes by the wearer (user) and there is no need for a certified technologist to perform that.

As used herein, the terms “self-installable” or “self-removable” or “user-installable” or “user-removable” electrode refer to an electrode (sensor electrodes for ECG and EEG and stimulating electrode of any component of the neuromodulation unit) that can be installed or removed by an ordinary adult wearer (or user) himself or herself without any help from other person or from a technologist (no need for the technologist to prepare the skin surface and no need to use special adhesive material to attach the electrodes to the skin). The terms “self-installation” or “self-removal” of an electrode refer to installing or removing the electrode by an ordinary adult wearer (or user) himself/herself without any help from a technologist or other person.

According to one aspect consistent with the principles of the invention, a cardiovascular monitoring apparatus is provided. The cardiovascular monitoring apparatus comprises an auricular ECG monitoring system and a cephalic blood flow monitoring system. The cephalic blood flow monitoring system is configured to monitor cerebral blood flow, such as a laser-speckle based blood flow monitoring unit, including a Speckle Contrast Optical Spectroscopy (SCOS) unit or an auricular SCOS unit. In some embodiments, the cephalic blood flow monitoring system may comprise at least one of: an auricular SCOS unit, an auricular EEG monitoring system and at least one of: a speckle-plethysmography (SPG) unit and a photoplethysmography (PPG) unit. Preferably, a speckle-plethysmography (SPG) unit may be utilized instead of the PPG unit. SPG is an improvement from PPG by using upgraded photodetector like CMOS camera (CMOS camera also used in SCOS) that provides higher signal-to-noise ratio than traditional PPG. SPG uses laser light while PPG usually uses LED light. Besides that, SPG, particularly with near-infrared (NIR) light, has a higher dynamic range and greater sensitivity to deep blood flow changes. It is less sensitive to melanin content in the skin. SPG can monitor the regional blood flow deeper than PPG, although not as deep as SCOS. Besides that, SPG is usually more accurate than PPG in monitoring systolic and diastolic blood pressure since SPG offers a higher signal-to-noise ratio with better reliability in varied temperatures, and reduced motion artifacts. (Another alternative is ultrasound and doppler based blood flow monitoring.) The SCOS unit may be configured to assess the cerebral and extracranial blood flow data from the wearer's (user) scalp while an auricular SCOS unit may be configured to assess the cerebral and extracranial blood flow data from the wearer's ear. The cardiovascular monitoring apparatus further comprises a processing unit and a network interface. The processing unit is in electronic communication with the network interface, the auricular ECG monitoring system, the auricular SCOS unit and at least one of: the SPG unit and the PPG unit. The processing unit may analyze the cerebral blood flow data transmitted from (or recorded by) the auricular SCOS unit to detect presence of a decrease of cerebral blood flow more than a predetermined percentage (for example a decrease of 30% or more). The processing unit may be configured to analyze the regional blood flow data and systolic and diastolic blood pressure data recorded by at least one of: the SPG unit and the PPG unit to detect presence of a decrease of regional blood flow more than a predetermined percentage (for example, a decrease of regional blood flow more than 30%) and a decrease systolic blood pressure more than a predetermined amount (e.g. a decrease of 20 mmHg or more) and a decrease of diastolic blood pressure more than a predetermined amount (e.g. a decrease of 20 mmHg or more). The processing unit may be configured to convert the raw EEG data recorded by the auricular EEG monitoring system into quantitative EEG (qEEG) data. The processing unit is further configured to analyze the EEG data and qEEG data to detect presence of at least one of the following: an increase of relative delta power more than a predetermined percentage (e.g. an increase of 30% or more), an increase of delta/alpha ratio more than a predetermined percentage (e.g. an increase of 30% or more) and a decrease of cordance z-score more than a predetermined amount (e.g. a decrease of 1 SD or more). The processing unit may be configured to analyze the ECG data (transmitted from or recorded by the auricular ECG monitoring system) to detect presence of ECG data suggestive of serious cardiac arrhythmia. The processing unit may be configured to analyze the cerebral blood flow data (recorded by the auricular SCOS unit) to detects presence or cessation of a decrease of cerebral blood flow more than the pre-determined percentage (for example, a decrease of 30% or more). The processing unit may be configured to analyze the blood pressure data (recorded by at least one of: the SPG unit and the PPG unit) to detect presence of a decrease of systolic blood pressure more than the pre-determined amount and presence of a decrease of diastolic blood pressure more than the predetermined amount. The processing unit is configured to analyze the regional blood flow data recorded by at least one of: the SPG unit and the PPG unit to detect a decrease of regional blood flow more than the predetermined percentage. In addition, the processing unit may be configured to analyze the ECG data (recorded by the auricular ECG monitoring system) to detect presence of ECG data suggestive of impending serious cardiac arrhythmia. When the processing unit detects at least one of the following: presence of ECG signals suggestive of serious cardiac arrhythmia, presence of a decrease of cerebral blood flow more than the pre-determined percentage, presence of a decrease of systolic blood pressure more than the pre-determined amount, presence of a decrease of diastolic blood pressure more than the predetermined amount, presence of a decrease of regional blood flow more than the predetermined percentage and presence of at least one of: an increase of relative delta power more than the pre-determined percentage, an increase of delta/alpha ratio more than the pre-determined percentage and a decrease of cordance z-score more than the predetermined amount, the processing unit is configured to send signals to the network interface which may be configured to generate a notification to a client device of the wearer or a client device of the wearer's healthcare provider. When the processing unit detects presence of ECG signals suggestive of impending serious cardiac arrhythmia, the processing unit is configured to send signals to the network interface which may be configured to generate a notification to the client device of the wearer or the client device of the wearer's healthcare provider. When the processing unit detects all of the following: cessation of ECG signals suggestive of serious cardiac arrhythmia, cessation of ECG data suggestive of impending serious cardiac arrhythmia, cessation of a decrease of cerebral blood flow more than the pre-determined percentage, cessation of a decrease of systolic blood pressure more than the pre-determined amount, cessation of a decrease of diastolic blood pressure more than the predetermined amount, cessation of a decrease of regional blood flow more than the predetermined percentage and presence of at least one of: an increase of relative delta power more than the pre-determined percentage, an increase of delta/alpha ratio more than the pre-determined percentage and a decrease of cordance z-score more than the predetermined amount, the processing unit may be further configured to send signals to the network interface to generate a notification to at least one of: the client device of the wearer and the client device of the wearer's healthcare provider. It is known that serious cardiac arrhythmia could result in significant decrease in cerebral blood flow (e.g. around 30% or more). Serious cardiac arrhythmia could also result in significant drop of blood pressure (for example decrease by 20 mmHg or more) or decrease of blood pressure below 90/60 mmHg level. Serious cardiac arrhythmia could also cause EEG changes including overall EEG suppression or flattening, decrease of fast waves (alpha and beta) and increase of slow waves (theta and delta waves), as shown by the relative delta power, delta/alpha ratio and cordance z-score, or other qEEG metrics.

According to an additional aspect consistent with the principles of the invention, an automatic detection-therapy system for cardiac arrhythmia is disclosed. In some embodiments, an automatic detection-therapy system for cardiac arrhythmia may comprise a first cardiovascular monitoring apparatus, a first neuromodulation system and a processing unit. The first cardiovascular monitoring apparatus may comprise an auricular ECG monitoring system and a cephalic blood flow monitoring system. The cephalic blood flow monitoring system may comprise at least one of: an ultrasound doppler-based blood flow monitoring unit (such as transcranial doppler ultrasound, TCD), a laser speckle-based blood flow monitoring unit (such as SCOS unit), a photoplethysmography (PPG) unit, a speckle-plethysmography (SPG) unit and an auricular EEG monitoring system. There are a few different (related) types of laser speckle-based blood flow monitoring units. The laser speckle blood flow systems, used for real-time, full-field mapping of tissue perfusion, are most commonly referred to as laser speckle contrast imaging (LSCI). Other frequently used devices include laser speckle imaging (LSI), laser speckle perfusion imaging (LSPI), laser speckle flowmetry (LSF) and laser speckle contrast analysis (LASCA). For simplicity purpose, a Speckle Contrast Optical Spectroscopy unit, (SCOS unit) will be discussed, although all of the other blood flow monitoring devices are within the scope of this invention. The SCOS unit may be configured to be housed in an auricular housing and will be called “auricular Speckle Contrast Optical Spectroscopy unit”, or “auricular SCOS unit” hereinafter. The processing unit is in electronic communication with the auricular ECG monitoring system, a first SCOS unit (or a first auricular SCOS unit) and the first neuromodulation system. The auricular ECG monitoring system comprises a first auricular ECG recording module configured to be linked to a wearer's first ear or a peri-auricular area around the wearer's first ear. The first auricular ECG recording module may include a plurality of (at least two) ECG sensor electrodes. These ECG sensor electrodes are configured to contact separate areas of the wearer's first ear or peri-auricular area around the wearer's first ear. The areas that the ECG sensor electrodes are configured to contact may be selected from at least one of the following: an external ear of the wearer's first ear, an external ear canal of the wearer's first ear, and a peri-auricular area around the wearer's first ear. The first auricular ECG recording module may be configured to record ECG data of the wearer. The first speckle contrast optical spectroscope unit (first SCOS unit) is configured to record the wearer's cerebral and extracranial blood flow from the wearer's scalp. The first SCOS unit may be configured to be housed in an auricular housing to be placed in the wearer's first ear (such as concha or external ear canal of the wearer's first ear) and will be called an “auricular SCOS unit”. The auricular SCOS unit may be configured to record the cerebral blood flow from the wearer's ear (such as concha or external ear canal). In some embodiments, an auricular SCOS unit may be housed in an auricular housing (housing refers to protective cover or protective structure) and the auricular ECG monitoring system may also be housed in the same auricular housing so that the auricular cardiovascular monitoring apparatus will exist as one device. The first neuromodulation system may comprise at least one of the following components: a first transcutaneous auricular vagus nerve stimulation unit (taVNS unit), a supraorbital nerve stimulation unit, a first auriculotemporal nerve stimulation unit, an occipital nerve stimulation unit, a first greater auricular nerve stimulation unit, an infraorbital nerve stimulation unit and a median nerve stimulation unit. The processing unit may be in electronic communication with the first cardiovascular monitoring apparatus (including the auricular ECG monitoring system and the first auricular SCOS unit). The processing unit may be configured to analyze the ECG data recorded by the auricular ECG monitoring system with the help of advanced ECG analysis algorithms together with machine learning, deep learning and artificial intelligence to detect the presence or cessation of ECG signals suggestive of serious cardiac arrhythmia. The processing unit may also be configured to analyze the cerebral blood flow data recorded by the first auricular SCOS unit to detect the presence or cessation of a decrease of cerebral blood flow more than a pre-determined percentage (for example a decrease of cerebral blood flow of 30% or more). In addition, the processing unit may be configured to analyze the ECG data recorded by the auricular ECG monitoring system with the help of advanced ECG analysis algorithms together with machine learning, deep learning and artificial intelligence to detect the presence or cessation of ECG signals suggestive of impending serious cardiac arrhythmia. When the processing unit detects presence of ECG signals suggestive of serious cardiac arrhythmia, the processing unit may be configured to immediately send signals to the first neuromodulation system to prompt the first neuromodulation system to automatically start sending pre-determined (or closed-loop controlled) neuromodulating electric stimulation to at least one of the following nerves of the wearer, including the auricular branch of vagus nerve on the wearer's first ear, the supraorbital nerve, the auriculotemporal nerve on the wearer's first ear, the occipital nerve, the greater auricular nerve on the wearer's first ear, the infraorbital nerve and a median nerve. When the processing unit detects presence of ECG signals suggestive of impending serious cardiac arrhythmia, the processing unit may be configured to immediately send signals to the first neuromodulation system to prompt the first neuromodulation system to automatically start sending pre-determined (or closed-loop controlled) neuromodulating electric stimulation to at least one of the following nerves of the wearer: the auricular branch of vagus nerve on the wearer's first ear, the supraorbital nerve, the auriculotemporal nerve on the wearer's first ear, the occipital nerve, the greater auricular nerve on the wearer's first ear, the infraorbital nerve and the median nerve. When the processing unit detects cessation of ECG signals suggestive of serious cardiac arrhythmia, the processing unit is configured to send signals to the first neuromodulation system to prompt it to stop the neuromodulating electric stimulation to any of the nerves.

In some embodiments, an automatic detection-therapy system for cardiac arrhythmia may include a neuromodulation system that may comprise a transcutaneous auricular vagus nerve stimulation unit (taVNS unit). The taVNS unit includes a taVNS stimulating electrode configured to contact the vagus nerve innervated skin of the wearer's ear. The vagus nerve innervated auricular skin includes the tragus, cymba concha, cavum concha, and external ear canal. The vagus nerve innervated auricular skin is anatomically adjacent to or overlapping with the auriculotemporal nerve innervated auricular skin. (For comparison: The vagus-innervated auricular skin includes: inner posterior portion of tragus, cymba concha, cavum concha, posterior and inferior walls of the external ear canal and small adjacent regions of the external ear. The auriculotemporal nerve innervated auricular skin includes: anterior outer part of tragus, the anterior-superior part of cavum concha, anterior and superior walls of the external ear canal and, anterior and superior part of pinna including anterior-superior helix.) Because of this, it is feasible for the auriculotemporal nerve stimulation unit and the taVNS unit to share a housing device or even share a stimulating electrode. However, it is usually preferred to have separate devices (could be integrated into one housing) with separate stimulating electrodes and separate stimulating parameters because their optimal stimulating parameters are often different. A processing unit is in electronic communication with the auricular ECG recording module and the taVNS unit. The processing unit is configured to analyze the ECG data recorded by the ECG monitoring system to detect the presence or cessation of ECG signals suggestive of serious cardiac arrhythmia. When the presence of ECG signals suggestive of serious cardiac arrhythmia is detected by the processing unit, the processing unit is configured to immediately send signals to the taVNS unit to prompt the taVNS unit to automatically start sending pre-determined (or closed-loop controlled) neuromodulating electric stimulation to the wearer's vagus nerve. When cessation of ECG signals suggestive of serious cardiac arrhythmia is detected by the processing unit, the processing unit is further configured to immediately send signals to the taVNS unit to prompt the taVNS unit to automatically stop sending neuromodulating electric stimulation to the wearer's auricular branch of vagus nerve. The processing unit is further configured to analyze the ECG data recorded by the ECG monitoring system to detect presence or cessation of ECG signals suggestive of impending serious cardiac arrhythmia. When the presence of ECG signals suggestive of impending serious cardiac arrhythmia is detected by the processing unit, the processing unit is configured to immediately send signals to the taVNS unit to prompt the taVNS unit to automatically start sending predetermined (or closed-loop controlled) neuromodulating electric stimulation to the wearer's auricular branch of vagus nerve. When cessation of ECG signals suggestive of impending serious cardiac arrhythmia is detected by the processing unit, the processing unit is further configured to immediately send signals to the taVNS unit to prompt it to automatically stop sending neuromodulating electric stimulation to the wearer's vagus nerve.

While there are many studies showing benefits and effects of taVNS neuromodulation for cardiac arrhythmia, electric neuromodulation of other nerves (including trigeminal nerve, occipital nerve, median nerve and greater auricular nerve) has also shown promise, including combination of neuromodulation with more than one nerve. Trigeminal nerve (including supraorbital nerve, infraorbital nerve and auriculotemporal nerve) neuromodulation has been found to have significant and rapid effect on cardiac arrhythmia. With stimulation at higher frequencies, trigeminal nerve stimulation may have more rapid and more pronounced effect on heart rate and blood pressure than vagal nerve stimulation. It is known that high frequency (e.g. 200 Hz) stimulation of the trigeminal nerve may have the risk of producing sudden bradycardia and hypotension due to the trigemino-cardiac reflex. The risk from the trigemino-cardiac reflex can be controlled if the trigeminal nerve neuromodulation is given under constant cerebral blood flow and blood pressure monitoring (such as under constant monitoring of the auricular SCOS, EEG and at least one of: SPG and PPG of the present invention) and with gradual increase of the stimulation frequency controlled by the closed-loop control system of the present invention. Another method to increase effectiveness and decrease side effect is through using R-wave synchronization. The processing unit is configured to precisely time the delivery of the neuromodulating electric stimulation according to R-wave synchronization (to be further described hereinafter).

In some embodiments, an automatic detection-therapy system for cardiac arrhythmia may include a neuromodulation system that may comprise an auriculotemporal nerve stimulation unit. The auriculotemporal nerve (ATN) is a branch of the mandibular division of the trigeminal nerve. The auriculotemporal nerve (ATN) stimulation unit includes an ATN stimulating electrode configured to contact the ATN innervated area of the wearer's ear. The ATN innervated area includes the anterior-outer part of tragus, upper anterior part of the helix, anterior portion of cavum concha, anterior and superior walls of external ear canal, outer surface of the tympanic membrane and area immediately in front of the tragus. The ATN innervated area is anatomically adjacent to or overlapping with the vagus-innervated auricular skin, as described hereinbefore. A processing unit is in electronic communication with the auricular ECG recording module and the ATN stimulation unit. The processing unit is configured to analyze the ECG data recorded by the ECG monitoring system to detect the presence or cessation of ECG signals suggestive of serious cardiac arrhythmia. When the presence of ECG signals suggestive of serious cardiac arrhythmia is detected by the processing unit, the processing unit is configured to immediately send signals to the ATN stimulation unit to prompt it to automatically start sending pre-determined (or closed-loop controlled) neuromodulating electric stimulation to the wearer's auriculotemporal nerve (ATN). When cessation of ECG signals suggestive of serious cardiac arrhythmia is detected by the processing unit, the processing unit is further configured to immediately send signals to the ATN stimulation unit to prompt it to automatically stop sending neuromodulating electric stimulation to the wearer's auriculotemporal nerve. The processing unit is further configured to analyze the ECG data recorded by the ECG monitoring system to detect presence or cessation of ECG signals suggestive of impending serious cardiac arrhythmia. When the presence of ECG signals suggestive of impending serious cardiac arrhythmia is detected by the processing unit, the processing unit is configured to immediately send signals to the ATN stimulation unit to prompt it to automatically start sending predetermined (or closed-loop controlled) neuromodulating electric stimulation to the wearer's ATN. When cessation of ECG signals suggestive of impending serious cardiac arrhythmia is detected by the processing unit, the processing unit is further configured to immediately send signals to the ATN stimulation unit to prompt it to automatically stop sending neuromodulating electric stimulation to the wearer's ATN.

In some embodiments, an automatic detection-therapy system for cardiac arrhythmia may include a neuromodulation system that may comprise a greater auricular nerve (GAN) stimulation unit. The GAN is a pure sensory nerve originated from the cervical spinal cord (C2, C3) and provides sensory innervation to the skin over the parotid gland, the mastoid process, and the lower two-thirds of the outer ear (pinna). The greater auricular nerve (GAN) innervated auricular skin includes skin on both surfaces of the lower two-thirds of pinna (including the lobule) and cavum concha (inferior/lower part of concha). The posterior branch of greater auricular nerve provides sensory innervation to the skin of the lower part of the cavum concha on both the posterior (back) and anterior (front/lateral) surfaces. The GAN stimulation unit includes a GAN stimulating electrode configured to contact the GAN innervated area of the wearer's auricular skin. A processing unit is in electronic communication with the auricular ECG recording module and the GAN stimulation unit. The processing unit is configured to analyze the ECG data recorded by the ECG monitoring system to detect the presence or cessation of ECG signals suggestive of serious cardiac arrhythmia. When the presence of ECG signals suggestive of serious cardiac arrhythmia is detected by the processing unit, the processing unit is configured to immediately send signals to the GAN stimulation unit to prompt the GAN stimulation unit to automatically start sending pre-determined (or closed-loop controlled) neuromodulating electric stimulation to the wearer's greater auricular nerve (GAN). When cessation of ECG signals suggestive of serious cardiac arrhythmia is detected by the processing unit, the processing unit is further configured to immediately send signals to the GAN stimulation unit to prompt the GAN stimulation unit to automatically stop sending neuromodulating electric stimulation to the wearer's GAN. The processing unit is further configured to analyze the ECG data recorded by the ECG monitoring system to detect presence or cessation of ECG signals suggestive of impending serious cardiac arrhythmia. When the presence of ECG signals suggestive of impending serious cardiac arrhythmia is detected by the processing unit, the processing unit is configured to immediately send signals to the GAN stimulation unit to prompt the GAN stimulation unit to automatically start sending predetermined (or closed-loop controlled) neuromodulating electric stimulation to the wearer's greater auricular nerve (GAN). When cessation of ECG signals suggestive of impending serious cardiac arrhythmia is detected by the processing unit, the processing unit is further configured to immediately send signals to the GAN stimulation unit to prompt the GAN stimulation unit to automatically stop sending neuromodulating electric stimulation to the wearer's GAN.

In some embodiments, an automatic detection-therapy system for cardiac arrhythmia may include a neuromodulation system that may comprise at least one of: a supraorbital nerve stimulation unit, an occipital nerve stimulation unit, an infraorbital nerve stimulation unit and a median nerve stimulation unit. The setups and the functions of the supraorbital nerve stimulation unit, the occipital nerve stimulation unit, the infraorbital nerve stimulation unit and the median nerve stimulation unit are essentially the same as the aforementioned description for taVNS unit (or auriculotemporal nerve stimulation unit or greater auricular nerve stimulation unit.)

In modified embodiments, an automatic detection-therapy system for cardiac arrhythmia may have a neuromodulation system that comprises more than one component. For example, the neuromodulation system may comprise a taVNS unit and an auriculotemporal nerve stimulation unit. When the processing unit detects presence of ECG signals suggestive of serious cardiac arrhythmia, the processing unit may be configured to send signals to both the taVNS unit and the auriculotemporal nerve stimulation unit to prompt the taVNS unit to send pre-determined (or preferably closed-loop controlled) electric stimulation to the wearer's vagus-innervated auricular skin and also to prompt the auriculotemporal nerve stimulation unit to send pre-determined (or preferably closed-loop controlled) electric stimulation to the wearer's auriculotemporal nerve innervated auricular skin. When the processing unit detects presence of ECG signals suggestive of impending serious cardiac arrhythmia, the processing unit may be configured to send signals to both the taVNS unit and the auriculotemporal nerve stimulation unit to prompt the taVNS unit to send pre-determined (or preferably closed-loop controlled) electric stimulation to the wearer's vagus-innervated auricular skin and also to prompt the auriculotemporal nerve stimulation unit to send pre-determined (or preferably closed-loop controlled) electric stimulation to the wearer's auriculotemporal nerve innervated auricular skin. When the processing unit detects both of the following: cessation of ECG signals suggestive of serious cardiac arrhythmia, and cessation of ECG signals suggestive of impending serious cardiac arrhythmia, the processing unit may be further configured to send signals to both the taVNS unit and the auriculotemporal nerve stimulation unit to prompt the taVNS unit to stop sending electric stimulation to the wearer's vagus-innervated auricular skin and also to prompt the auriculotemporal nerve stimulation unit to stop sending electric stimulation to the wearer's auriculotemporal nerve innervated auricular skin.

Other two components of the neuromodulation system may also be combined, similar to the aforementioned descriptions. In further modified embodiments, the neuromodulation system for the automatic detection-therapy system for cardiac arrhythmia may include a combination of 3 different neuromodulation units, for example, a combination of a taVNS unit, an auriculotemporal nerve stimulation unit and a supraorbital nerve stimulation unit, or a combination of a taVNS unit, an auriculotemporal nerve stimulation unit and an occipital nerve stimulation unit or a combination of a taVNS unit, an auriculotemporal nerve stimulation unit and a greater auricular nerve stimulation unit. All of these various combinations are within the scope of this invention.

In some embodiments, the processing unit may include a closed-loop control system (a “sensor-controller-actuator” setup) (or a feedback-controlled mechanism). The closed-loop control system (within the processing unit) may be in electronic communication with the cardiovascular monitoring apparatus (including the auricular ECG monitoring system and cephalic blood flow monitoring system that comprises at least one of: the auricular SCOS, at least one of: the SPG unit and the PPG unit, and auricular EEG monitoring system) and the neuromodulation system. The closed-loop control system receives real-time continuous input data of the wearer's ECG data from the auricular ECG monitoring system and real-time continuous input data from the cephalic blood flow monitoring system (including at least one of: the wearer's cerebral and extracranial blood flow data from the auricular SCOS, the regional blood flow data and the systolic and diastolic blood pressure data from at least one of: SPG unit and PPG unit, and qEEG data from the auricular EEG monitoring system.) The closed-loop control system and the processing unit are configured to analyze these real-time data, using controlling algorithms, to continuously adjust the actuating outputs to the neuromodulation system. The actuating outputs includes turning on or turning off at least one component of the neuromodulation system and adjusting the stimulating parameters (intensity, frequency, duration, cycle, wave form etc.) of the neuromodulating system during the time when the neuromodulating system is turned on. This will greatly enhance the effectiveness and safety of neuromodulation, especially trigeminal nerve (including supraorbital nerve, infraorbital nerve and auriculotemporal nerve) stimulation. In trigeminal nerve neuromodulation, some high-frequency (e.g., 200 Hz) stimulation could produce pronounced effects on heart rate and blood pressure compared to lower frequencies. Studies have shown that high frequency (e.g. 200 Hz) trigeminal nerve electric stimulation can produce a rapid, transient decrease in heart rate (bradycardia) and, in some cases, a sudden, brief drop in blood pressure (hypotension) due to activation of the trigemino-cardiac reflex. Both taVNS and trigeminal nerve stimulation are known to be effective for cardiac arrhythmia. Studies have shown that the effect of trigeminal nerve stimulation is frequency-dependent, where 200 Hz stimulation produces larger decreases in heart rate (10.77% reduction) compared to 2 Hz (6.56%) or 20 Hz (6.8%) in healthy subjects. High-frequency trigeminal nerve stimulation (for example at 200 Hz) has been shown to elicit more pronounced cardiovascular responses with heart rate reduction and increased P-wave alternans (baroreflex modulation) compared to lower frequencies. Other studies have also shown that high-frequency trigeminal nerve stimulation (e.g. around 100-200 Hz) is more effective than low-frequency stimulation (e.g., 2 Hz, 20 Hz) for modifying cardiovascular autonomic responses, specifically in inducing pronounced decreases in heart rate and pulse arrival time. Using controlled-loop control system under the guidance of cerebral blood flow monitoring from auricular SCOS unit and regional blood flow and blood pressure monitoring from the at least one of: SPG unit and PPG unit and, optionally, EEG monitoring with quantitative EEG from the auricular EEG monitoring system can minimize the risk when high frequency is used for trigeminal nerve stimulation. After starting neuromodulation, when any of the following: the cerebral blood flow data, the regional blood flow data, the blood pressure data and the EEG data worsen more than a predetermined percentage or amount (e.g. blood flow decreased by 30% or more, blood pressure decreased by 20 mmHg or more and qEEG data showing at least one of: relative delta power increase by 30% or more, delta/alpha ratio increase by 30% or more and cordance z-score decrease more than 1 SD), the stimulation may be immediately stopped or the stimulation parameters may be immediately altered. R-wave synchronization is known to be a very important aspect of neuromodulation techniques used to treat cardiac arrhythmias. R-wave synchronization involves timing electrical stimulations to coincide with the R wave of the QRS complex on an electrocardiogram (ECG). This is feasible due to the existence of continuous ECG monitoring with continuous input of the ECG data in this invention. The R-wave synchronization aims to restore normal heart rhythm by delivering an electric stimulation at the precise moment when the heart is most receptive to electrical stimulation. By delivering the stimulation during the refractory period of the cardiac cycle, the R-wave synchronization technique can minimize the risk of inducing further arrhythmia. R-wave synchronization or R-wave gating helps to avoid giving stimulation during T-wave because T-wave represents ventricular repolarization and is considered a “vulnerable period” with a very slight risk of inducing ventricular tachycardia of fibrillation. (This risk is very minimal and extremely rare in taVNS stimulation and trigeminal nerve stimulation.)

### BRIEF DESCRIPTION OF THE DRAWINGS

Some embodiments of the present invention are illustrated as an example and are not limited by the figures of the accompanying drawings, in which like references may indicate similar elements and in which:

FIG. 1—FIG. 1 depicts a diagram of an example of an electrocardiogram (ECG) monitoring system and an electroencephalogram (EEG) monitoring system in a modified earbud housing, according to various embodiments described herein.

FIG. 2—FIG. 2 illustrates a diagram of an example of an automatic detection-therapy system for cardiac arrhythmia in a modified earbud housing, according to various embodiments described herein.

FIG. 3—FIG. 3 shows a schematic diagram of some exemplary components of an automatic detection-therapy system for cardiac arrhythmia, comprising an auricular ECG monitoring system, having a first ECG sensor electrode located in an ear of the wearer and a second ECG sensor electrode located in a wrist band to be worn on a wrist of the wearer.

FIG. 4—FIG. 4 illustrates an elevation view of the anatomy of a human external ear, including the locations of tragus, cymba-concha, cavum-concha, triangular fossa, helix, lobule, external ear canal, etc.

FIG. 5—FIG. 5 shows a diagram of a further example of an automatic detection-therapy system for cardiac arrhythmia in a behind-the-ear-hearing-aid-style housing, according to various embodiments described herein.

FIG. 6—FIG. 6 depicts a diagram of yet a further example of an automatic detection-therapy system for cardiac arrhythmia in a modified in-the-ear housing, according to various embodiments described herein.

FIG. 7—FIG. 7 depicts a diagram of still another example of an automatic detection-therapy system for cardiac arrhythmia having a modified earbud housing engaged to an ear of a wearer according to various embodiments described herein.

FIG. 8—FIG. 8 shows a schematic diagram of some exemplary components of an automatic detection-therapy system for cardiac arrhythmia and an auricular ECG monitoring system having a first ECG sensor electrode located in an ear of a wearer and a second ECG sensor electrode located in a finger ring to be worn on a finger of the wearer.

FIG. 9—FIG. 9 shows a schematic diagram of some exemplary components of an automatic detection-therapy system for cardiac arrhythmia and an auricular ECG monitoring system having a first ECG sensor electrode located in an ear of the wearer and a second ECG sensor electrode located in one of: a toe ring to be worn on a toe of the wearer and an ankle band to be worn on an ankle of the wearer.

FIG. 10—FIG. 10 shows a schematic diagram of some exemplary components of an automatic detection-therapy system for cardiac arrhythmia and an auricular ECG monitoring system, having a first ECG sensor electrode located in a first finger ring to be worn on an X-finger of the wearer's X-hand and a second ECG sensor electrode located in a second finger ring to be worn on a Y-finger of the wearer's Y-hand.

FIG. 11—FIG. 11 shows a diagram of an example of an automatic detection-therapy system for cardiac arrhythmia having a modified earbud housing engaged or coupled to an ear of a wearer according to various embodiments described herein.

FIG. 12—FIG. 12 illustrates a diagram of an example of an automatic detection-therapy system for cardiac arrhythmia having a modified in-the-ear housing engaged or coupled to the ear of a wearer according to various embodiments described herein.

FIG. 13—FIG. 13 depicts a diagram of an example of an automatic detection-therapy system for cardiac arrhythmia having a behind-the-ear-hearing-aid-style housing engaged or coupled to the ear of a wearer according to various embodiments described herein.

FIG. 14—FIG. 14 illustrates a block diagram of some exemplary elements of an example of a processing unit for an ECG monitoring system or an automatic detection-therapy system for cardiac arrhythmia according to various embodiments described herein.

FIG. 15—FIG. 15 shows a block diagram of some exemplary elements of an example of an auricular ECG monitoring system according to various embodiments described herein.

FIG. 16—FIG. 16 depicts a block diagram illustrating some exemplary elements of an example of a transcutaneous auricular vagus nerve stimulation (taVNS) unit according to various embodiments described herein.

FIG. 17—FIG. 17 shows a block diagram illustrating some exemplary elements of an example of a client device according to various embodiments described herein.

FIG. 18—FIG. 18 illustrates a schematic diagram illustrating some exemplary components of auricular ECG monitoring system and an automatic detection-therapy system for cardiac arrhythmia according to various embodiments described herein.

FIG. 19—FIG. 19 illustrates a perspective diagram of an example of a tubular-shaped structure for housing of the EEG sensor electrodes and some exemplary locations for the EEG sensor electrodes according to various embodiments described herein.

FIG. 20—FIG. 20 illustrates a side elevation diagram of an example of a tubular-shaped structure for housing of the EEG sensor electrodes and some exemplary locations for the EEG sensor electrodes according to various embodiments described herein.

FIG. 21—FIG. 21 shows a sectional, through line 21-21 shown in FIG. 20, elevation view of an example of a tubular-shaped structure for housing of the EEG sensor electrodes and some exemplary locations for the EEG sensor electrodes according to various embodiments described herein.

FIG. 22—FIG. 22 depicts a sectional, through line 22-22 shown in FIG. 20, elevation view of an example of a tubular-shaped structure for housing of the EEG sensor electrodes and some exemplary locations for the EEG sensor electrodes according to various embodiments described herein.

FIG. 23—FIG. 23 shows a sectional, through line 23-23 shown in FIG. 20, elevation view of an example of a tubular-shaped structure for housing of the EEG sensor electrodes and some exemplary locations for the EEG sensor electrodes according to various embodiments described herein.

FIG. 24—FIG. 24 illustrates a sectional, through line 24-24 shown in FIG. 20, elevation view of an example of a tubular-shaped structure for housing of the EEG sensor electrodes and some exemplary locations for the EEG sensor electrodes according to various embodiments described herein.

FIG. 25—FIG. 25 depicts a sectional, through line 25-25 shown in FIG. 20, elevation view of an example of a tubular-shaped structure for housing of the EEG sensor electrodes and some exemplary locations for the EEG sensor electrodes according to various embodiments described herein.

FIG. 26—FIG. 26 shows a sectional, through line 26-26 shown in FIG. 20, elevation view of an example of a tubular-shaped structure for housing of the EEG sensor electrodes and some exemplary locations for the EEG sensor electrodes according to various embodiments described herein.

FIG. 27—FIG. 27 shows a diagram of a closed-loop neuromodulation apparatus placed in the tragus-concha bowl of a wearer's ear, according to various embodiments described herein.

FIG. 28—FIG. 28 depicts a diagram of still a further example of an automatic detection-therapy system for cardiac arrhythmia having a behind-the-ear-hearing-aid-style housing according to various embodiments described herein.

FIG. 29—FIG. 29 shows a diagram of a supraorbital nerve stimulation unit.

FIG. 30—FIG. 30 shows a diagram of an occipital nerve stimulation unit with a transparent view of the occipital nerves.

FIG. 31—FIG. 31 shows a block diagram illustrating some exemplary elements of an example of a trigeminal nerve stimulation unit. Examples of a trigeminal nerve stimulation unit includes a supraorbital nerve stimulation unit, an auriculotemporal nerve stimulation unit and an infraorbital nerve stimulation unit.

FIG. 32—FIG. 32 depicts a block diagram showing some exemplary elements of an example of one of: an occipital nerve stimulation unit and a median nerve stimulation unit.

FIG. 33—FIG. 33 shows a block diagram illustrating some exemplary elements of an example of a greater auricular nerve stimulation unit.

FIG. 34—FIG. 34 shows a block diagram illustrating some exemplary elements of an example of a speckle contrast optical spectroscope unit (SCOS unit).

FIG. 35—FIG. 35 shows a block diagram illustrating some exemplary elements of an example of one of: a speckle-plethysmography (SPG) unit and a photoplethysmography (PPG) unit. Please note the SPG unit usually has laser as its light source, while the PPG unit usually has a light-emitting diode (LED) as its light source.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

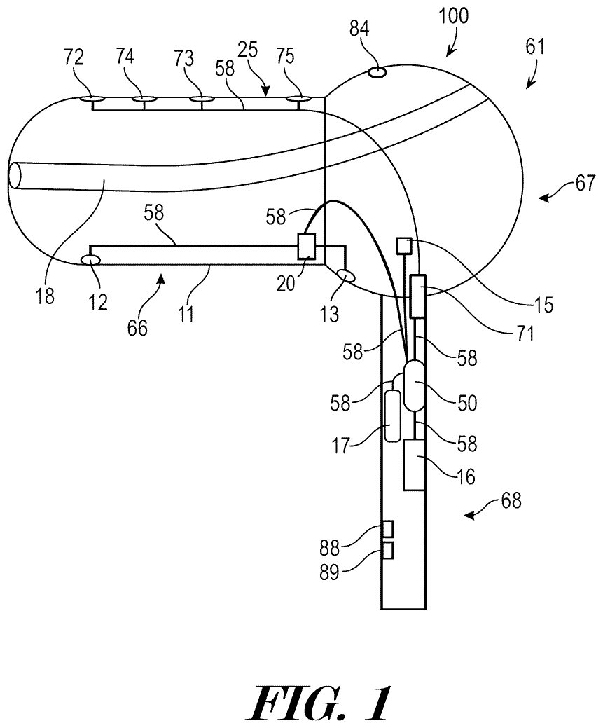

For purposes of description herein, the terms “upper”, “lower”, “left”, “right”, “rear”, “front”, “side”, “vertical”, “horizontal”, and derivatives thereof shall relate to the invention as oriented in FIG. 1. However, one will understand that the invention may assume various alternative orientations and step sequences, except where expressly specified to the contrary. Therefore, the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

The term “client device” as used herein is a type of computer or computing device comprising circuitry and configured to generally perform functions such as recording audio, photos, and videos; displaying or reproducing audio, photos, and videos; storing, retrieving, or manipulation of electronic data; providing electrical communications and network connectivity; or any other similar function. Non-limiting examples of client devices include: personal computers (PCs), workstations, servers, laptops, tablet PCs including the iPad, cell phones including iOS phones made by Apple Inc., Android OS phones, Microsoft OS phones, Blackberry phones, Apple iPads, Anoto digital pens, digital music players, or any electronic device capable of running computer software and displaying information to a user, memory cards, other memory storage devices, digital cameras, external battery packs, external charging devices, and the like. Certain types of electronic devices which are portable and easily carried by a person from one location to another may sometimes be referred to as a “portable electronic device” or “portable device”. Some non-limiting examples of portable devices include: cell phones, smartphones, tablet computers, laptop computers, tablets, digital pens, wearable computers such as Apple Watch, other smartwatches, Fitbit, other wearable fitness trackers, Google Glasses, and the like.

As used herein the term “data network” or “network” shall mean an infrastructure capable of connecting two or more computers such as client devices, using wires or wirelessly allowing them to transmit and receive data. Non-limiting examples of data networks may include the internet or wireless networks which may include BLE (Bluetooth), LoRa and LoRaWAN (and other low-power, wide-area (LPWA) networking protocols), Wi-Fi, and cellular networks. For example, a network may include a local area network (LAN), a wide area network (WAN) (e.g., the Internet), a mobile relay network, a metropolitan area network (MAN), an ad hoc network, a telephone network (e.g., a Public Switched Telephone Network (PSTN)), a cellular network, a Zigbee network, or a voice-over-IP (VOIP) network.

Although the terms “first”, “second”, “X” item, “Y” item etc. are used herein to describe various elements, these elements should not be limited by these terms. These terms are only used to distinguish one element from another element. For example, the first element may be designated as the second element, and the second element may be likewise designated as the first element without departing from the scope of the invention. As used in this application, the term “about” or “approximately” refers to a range of values within plus or minus 15% of the specified number. Additionally, as used in this application, the term “substantially” means that the actual value is within about 10% of the actual desired value, particularly within about 5% of the actual desired value and especially within about 1% of the actual desired value of any variable, element or limit set forth herein.

Novel auricular electrocardiogram (ECG) monitoring system and novel automatic detection-therapy system for cardiac arrhythmias are discussed herein. In the following description, for purposes of explanation, numerous specific details are set forth to provide a thorough understanding of the present invention. It will be evident, however, to one skilled in the art that the present invention may be practiced without these specific details. The present disclosure is to be considered as an exemplification of the invention and is not intended to limit the invention to the specific embodiments illustrated by the figures or description below.

The present invention will now be described by example and through referencing the appended figures representing preferred and alternative embodiments. According to some embodiments consistent with the principles of the present invention, an auricular electrocardiogram (ECG) monitoring system (“the auricular ECG monitoring system”) 100 is disclosed. The auricular ECG monitoring system 100 may comprise an ECG recording module 20 that may comprise at least two wired or wireless ECG electrodes 12, 13, (ECG sensor electrodes 12, 13) (for example a first ECG sensor electrode 12 and a second ECG sensor electrode 13) in electronic communication with the ECG recording module 20. The ECG sensor electrodes 12, 13, may be in electronic communication with the ECG recording module 20 via a local interface (wire) 58 or wirelessly. The ECG sensor electrodes 12, 13, may be configured to be coupled to a wearer's first ear 902 or a peri-auricular area 903 around the wearer's first ear 902. When the ECG sensor electrodes 12, 13, are coupled to the wearer's first ear 902 or the peri-auricular area 903 around the wearer's first ear 902 the ECG sensor electrodes 12, 13, are positioned in contact with separate skin locations on at least one area of the wearer 900, the area selected from at least one of the following: external ear of the wearer's first ear 902, external ear canal 904 of the wearer's first ear, and the peri-auricular area 903 around the wearer's first ear. (It should be noted that the first and the second ECG sensor electrodes 12, 13 may contact separate skin locations within a single area. Alternatively, the first ECG sensor electrode 12 may contact a skin location within one area and the second ECG sensor electrode 13 may contact a separate skin location within another area.) These skin locations that the ECG sensor electrodes 12, 13, are attached to may be adequately separated (with different angles or directions) relative to the location of the heart of the wearer 900 so that they can pick up some differences of the cardiac action potentials in order to enable the ECG recording module 20 to perform an ECG. The ECG recording module 20 may be configured to record ECG data of the wearer 90 via the ECG sensor electrodes 12, 13, and the ECG recording module 20 may be in electronic communication with a processing unit 50, 401, which may be configured to record and analyze the ECG data from the ECG recording module 20 to detect presence or cessation of a serious cardiac arrhythmia. The processing unit 50, 401, may be further configured to record and analyze the ECG data from the ECG recording module 20 to detect presence or cessation of an impending serious cardiac arrhythmia. The serious cardiac arrhythmia may include atrial fibrillation (AFib or AF), atrial flutter (AFL), supraventricular tachycardia (SVT), ventricular tachycardia (VT), and ventricular fibrillation (VF).

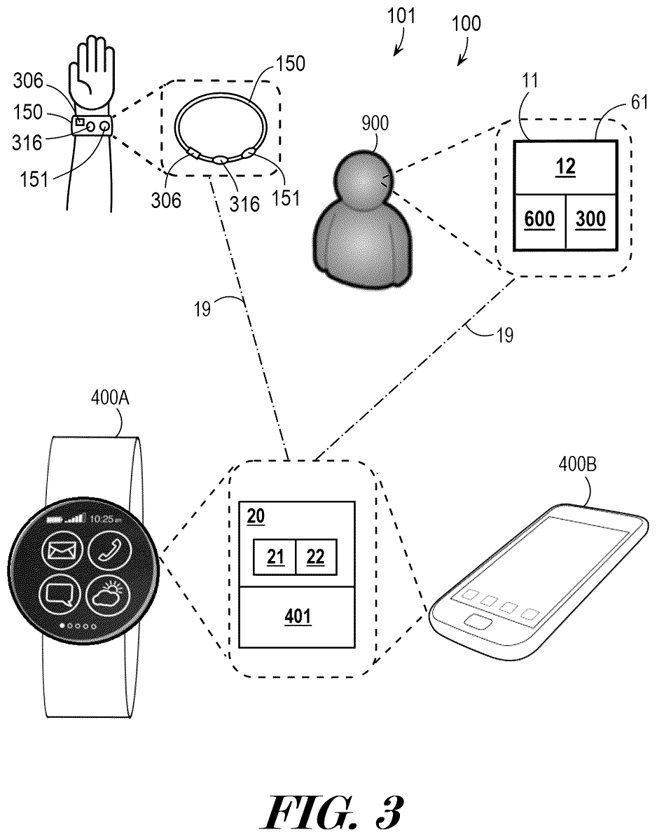

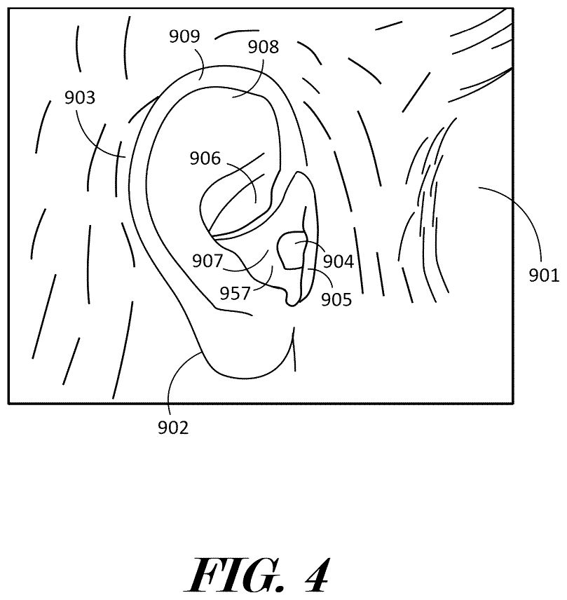

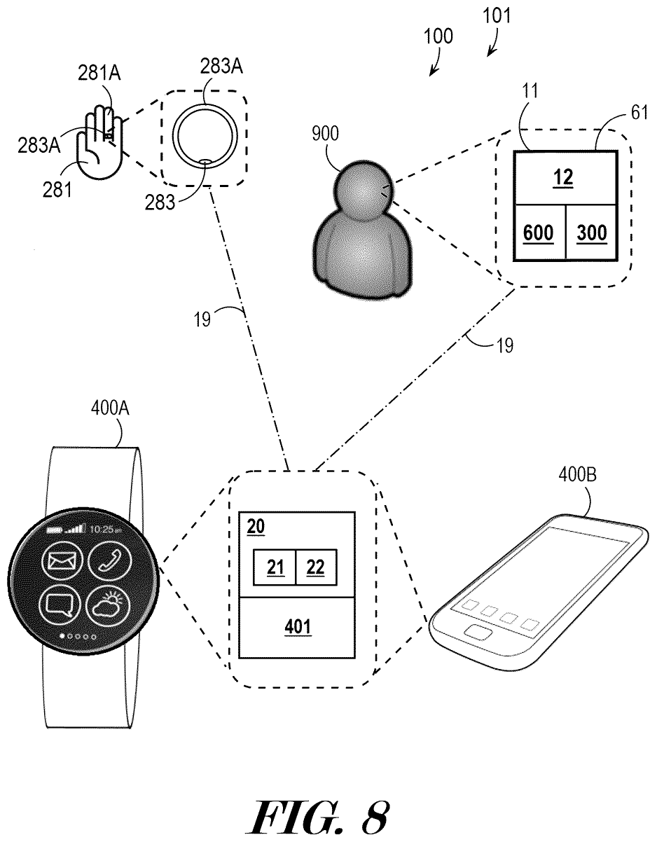

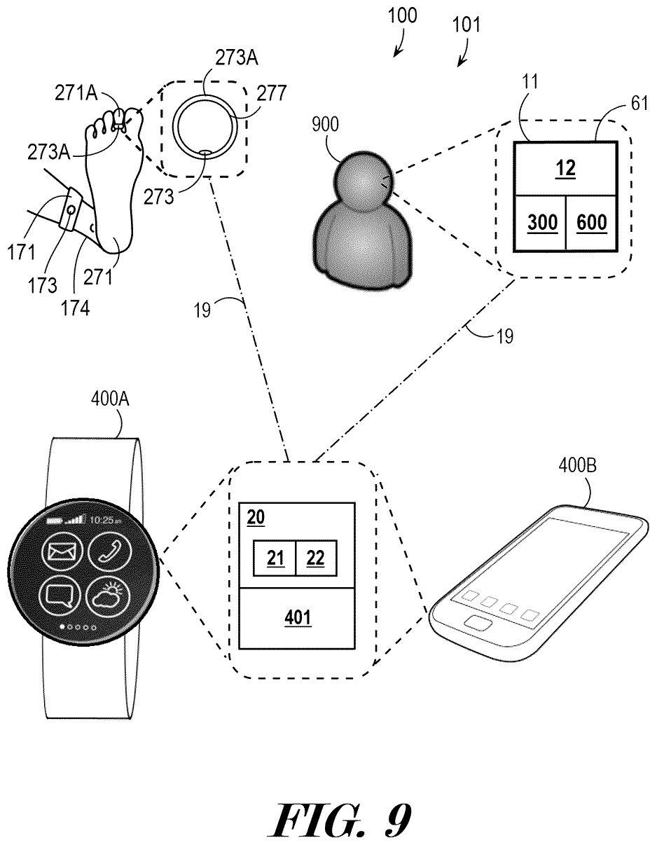

When both the first ECG sensor electrode 12 and the second ECG sensor electrode 13 are located in the first ear 902 or the peri-auricular area 903 around the wearer's first ear, it will be more convenient for the wearer although the ECG signal may be weaker. Alternatively, in some embodiments, an auricular electrocardiogram (ECG) monitoring system 100 may comprise a first ECG sensor electrode 12 located in a first ear 902 or a peri-auricular area 903 around the wearer's first ear, while the second ECG sensor electrode 13 may be configured to be located in the wearer's second ear 902 or the peri-auricular area 903 around the wearer's second ear. In some alternative embodiments, the second ECG sensor electrode 13 may be configured to be located on a wearer's 900 wrist via a wrist band 150 having a wrist electrode 151 (to serve as the second ECG sensor electrode 13) on the inner surface of the wrist band 150. Alternatively, the second ECG sensor electrode 13 may be built into a smart watch 400A (or a health tracker) with the second ECG electrode 13 on the undersurface of the smart watch (or health tracker). (FIG. 3). Alternatively, the second ECG sensor electrode 13 may be a second finger ring electrode 283 (to serve as the second ECG sensor electrode 13) located on a second finger ring 283A to be worn on a Y-finger 281A of the wearer's 900 second hand (Y-hand) 281. (FIG. 8). In another alternative design, the second ECG sensor electrode 13 may be an ankle electrode 173 (to serve as the second ECG sensor electrode 13) located on an ankle band 171 to be worn on an ankle 174 of the wearer's 900. Optionally, the second ECG sensor electrode 13 may be a toe ring electrode 273 located on an inner surface 277 of a toe ring 273A to be worn on a wearer's toe 271A. (FIG. 9). In alternative embodiments, the first ECG sensor electrode 12 may be configured as a first finger ring electrode 284 (to serve as the first ECG sensor electrode 12) located on a first finger ring 284A to be worn on an X-finger 282A of the wearer's 900 X-hand (first hand) 282, while the second ECG sensor electrode 13 may be a second finger ring electrode 283 (to serve as the second ECG sensor electrode 13) located on a second finger ring 283A to be worn on a Y-finger 281A of the wearer's 900 Y-hand (second hand) 281. The first ECG sensor electrode 12 and the second ECG sensor electrode 13 may be in wireless electronic communication with the ECG recording module 20. In these alternative setups, with the second ECG sensor electrode 13 located more separated (e.g. on a second ear, or on a wrist, a finger, a toe or an ankle) from the first ECG sensor electrode 12, the ECG signals will be stronger and easier for the ECG recording module 20 to record the wearer's ECG. The ECG recording module 20 may be in electronic communication with a processing unit 50, 401, which may be configured to record and analyze the ECG data from the ECG recording module 20 to obtain the wearer's ECG profile. The processing unit 50, 401 may be configured to analyze ECG data recorded by the ECG recording module 20 to detect presence or cessation of a serious cardiac arrhythmia of the wearer 900. The processing unit 50, 401 may be further configured to analyze the ECG data recorded by the ECG recording module 20 to detect presence or cessation of an impending serious cardiac arrhythmia of the wearer 900. The serious cardiac arrhythmia may include atrial fibrillation (AFib or AF), atrial flutter (AFL), supraventricular tachycardia (SVT), ventricular tachycardia (VT), and ventricular fibrillation (VF).

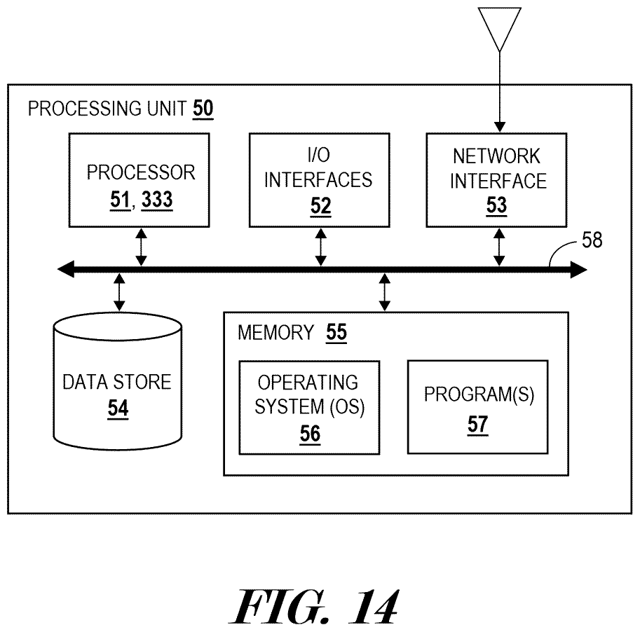

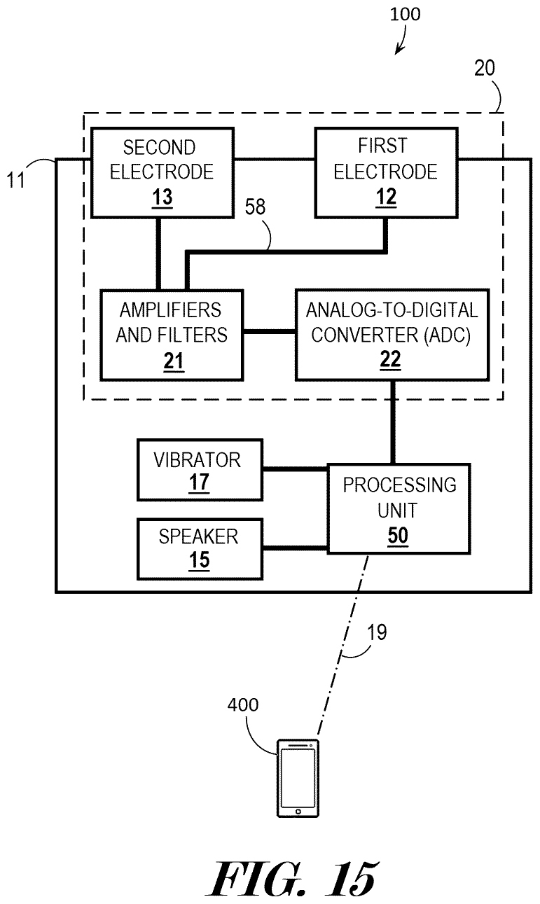

The auricular ECG monitoring system 100 may comprise an ECG recording module 20. The ECG recording module 20 may record and analyze the electrical activity of the wearer's 900 heart, such as to diagnose heart conditions and cardiac arrhythmias. Referring to FIG. 15, amplifiers and filters 21 may pick up the electrical activity of the wearer's 900 heart via the ECG sensor electrodes 12, 13. An amplifier of amplifiers and filters 21 is responsible for amplifying the weak electrical signals received from the ECG sensor electrodes 12, 13. The heart's electrical signals are typically very faint (for example, in the range of 30 microvolts to 5 millivolts, depending on chest or limb leads and depending on different waves including P, QRS or T waves, and around 5 microvolts or less for in-ear ECG). An amplifier of amplifiers and filters 21 boosts these signals to a level that can be accurately recorded and displayed. Modern ECG machines use sophisticated amplifiers that minimize noise and ensure signal clarity. Filters of amplifiers and filters 21 are used to remove unwanted noise and interference from the electrical signals. Common sources of noise include muscle contractions, electrical interference from other devices, and movement artifacts. ECG machines use various filters, such as high-pass, low-pass, and notch filters, to clean the signals, ensuring that the resulting ECG trace is clear and interpretable. An analog-to-digital converter (ADC) 22 may transform the analog electrical signals from the heart into digital data. This digital conversion is essential for processing, storing, and/or displaying the ECG data on a client device 400 or print out. The ADC 22 ensures that the data is accurately digitized, preserving the integrity of the original signal. The ADC 22 may be in communication with a processing unit 50.

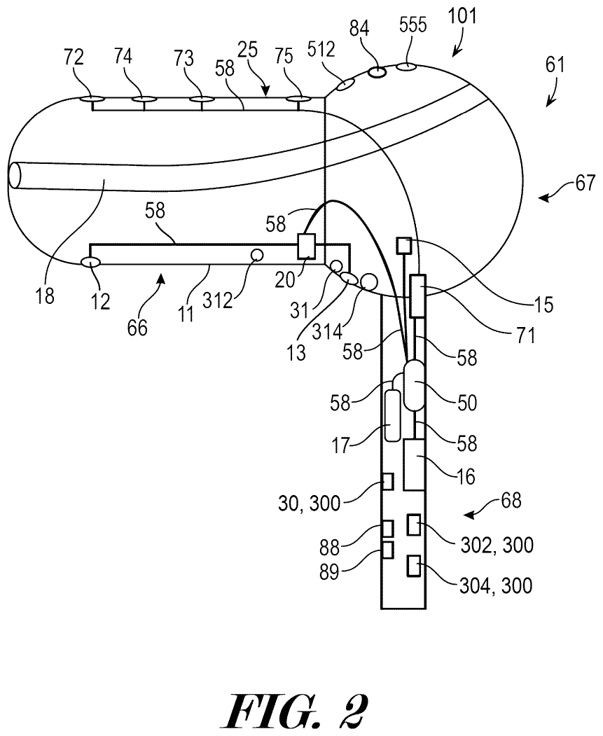

In some embodiments, an auricular ECG monitoring system 100 may comprise an ECG recording module 20 that may be contained in an auricular housing 11. In some embodiments, an auricular ECG monitoring system 100 of the present invention may comprise a first auricular housing 11 which may be configured to be coupled to a first ear 902 of a wearer 900 and/or to a peri-auricular area 903 around the first ear 902 of the wearer 900. The first auricular housing 11 may be configured to house all of the ECG sensor electrodes 12, 13. In some embodiments, when the first ECG sensor electrode 12 is located in the wearer's first ear and the second ECG sensor electrode 13 is located in the wearer's second ear, two auricular housings 11 may be utilized, with the first auricular housing 11 configured to be coupled to a first ear 902 of a wearer 900 and/or to the peri-auricular area 903 around the first ear 902 of the wearer 900 and be configured to house the first auricular ECG electrode 12, while a second auricular housing 11 may be configured to be coupled to a second ear 902 of the wearer 900 and/or to a peri-auricular area 903 around the second ear 902 of the wearer 900 and be configured to house the second ECG electrode 13. In some embodiments, the one or two auricular housing(s) 11 may house one or more components, such as a processing unit 50, an ECG recording module 20, auricular ECG electrodes 12, 13, a speaker 15, a vibrator 17, a power source 16, etc., which may be communicatively coupled via a local interface 58. The local interface 58 can be, for example but not limited to, one or more buses, circuit boards, wiring harnesses, or other wired connections or wireless connections, as is known in the art. The local interface 58 can have additional elements, which are omitted for simplicity, such as controllers, buffers (caches), drivers, repeaters, and receivers, among many others, to enable communications. Further, the local interface 58 may include address, control, and/or data connections to enable appropriate communications among the aforementioned components. In some embodiments, an auricular housing 11 may comprise as one of: a modified earbud housing 61, a modified in-the-ear housing 62, a behind-the-ear-hearing-aid-style housing 63 or a tubular-shaped structure 66. In some embodiments, an auricular housing 11, such as a modified earbud housing 61, may comprise a sound conduit 18 which may extend through the auricular housing 11 (such as shown in FIGS. 1, 2) and which may facilitate the ability of sound to enter the ear 902. The sound conduit 18 may also help to equalize the pressure inside the external ear canal 904 and the outside environment and increase the comfort of the wearer 900. Preferably, a sound conduit 18 may comprise an opening, channel, conduit, etc., which may extend through a portion of the auricular housing 11 so that sound waves may pass through the sound conduit 18 to facilitate or enable the wearer 900 to hear sounds in the environment. (As described hereinbefore, the second ECG sensor electrode 13 may be housed in one of: a wrist band 150, a finger ring 283A, an ankle band 171 and a toe-ring 273A.)

In some embodiments, an auricular ECG monitoring system 100 may comprise a processing unit 50. Referring to FIG. 14, the processing unit 50 may comprise a memory 55 that may include any of volatile memory elements (e.g., random access memory (RAM, such as DRAM, SRAM, SDRAM, etc.)), nonvolatile memory elements (e.g., ROM, hard drive, etc.), and combinations thereof. Moreover, the memory 55 may incorporate electronic, magnetic, optical, and/or other types of storage media. Note that the memory 55 may have a distributed architecture, where various components are situated remotely from one another, but can be accessed by the processor 51. Optionally, memory 55 can include one or more software programs, each of which includes an ordered listing of executable instructions for implementing logical functions. Optionally, the software in the memory system 55 includes a suitable operating system (O/S) 56 and program(s) 57. The operating system 56 essentially controls the execution of input/output interface 52 and other element functions, and provides scheduling, input-output control, file and data management, memory management, and communication control and related services. The operating system 56 may be, for example, LINUX (or another UNIX variant), Android (available from Google), Symbian OS, Microsoft Windows CE, Microsoft Windows 7 Mobile, iOS (available from Apple, Inc.), webOS (available from Hewlett Packard), Blackberry OS (Available from Research in Motion), and the like. The programs 57 may include various applications, add-ons, etc. configured to provide end user functionality of the device 100. In some embodiments, the processor 51 may comprise a closed-loop control system 333 to regulate or control the output actuation when a neuromodulation system 300 is activated.

In some embodiments, a processing unit 50 may comprise one or more I/O interfaces 52 which can be used to provide user input and display system output data, such as operational status. The I/O interfaces 52 can include, for example, buttons, knobs, switches, LED indicator lights, LED display, LCD display, a serial port, a parallel port, a small computer system interface (SCSI), an infrared (IR) interface, a radio frequency (RF) interface, a universal serial bus (USB) interface, and the like. Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequences of actions described herein can be considered to be embodied entirely within any form of computer readable storage medium having stored therein a corresponding set of computer instructions that upon execution would cause an associated processor to perform the functionality described herein. Thus, the various aspects of the invention may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

In some embodiments, an auricular ECG monitoring system 100 may comprise a network interface 53 which may be contained in the auricular housing 11 and which may enable wired and/or wireless communication between one or more components, such as ECG recording module 20, processing unit 50, etc., with one or more client devices 400. Preferably, a network interface 53 may comprise a radio that may operate via WiFi and/or Bluetooth communication standards. In further embodiments, a network interface 53 may comprise a radio that may operate on a cellular band and may communicate with or receive a Subscriber Identity Module (SIM) card or other wireless network identifier. Any number of suitable wireless data communication protocols, techniques, or methodologies can be supported by a network interface 53, including, without limitation: RF; IrDA (infrared); Bluetooth; ZigBee (and other variants of the IEEE 802.15 protocol); IEEE 802.11 (any variation); IEEE 802.16 (WiMAX or any other variation); Direct Sequence Spread Spectrum; Near-Field Communication (NFC); Frequency Hopping Spread Spectrum; Long Term Evolution (LTE); cellular/wireless/cordless telecommunication protocols (e.g. 3G/4G, etc.); wireless home network communication protocols; paging network protocols; magnetic induction; satellite data communication protocols; wireless hospital or health care facility network protocols such as those operating in the WMTS bands; GPRS; proprietary wireless data communication protocols such as variants of Wireless USB; and any other protocols for wireless communication. In further embodiments, a network interface 53 may enable wired network communication and may include an Ethernet card or adapter (e.g., 10BaseT, Fast Ethernet, Gigabit Ethernet, 10 GbE) or a wireless local area network (WLAN) card or adapter (e.g., 802.11a/b/g/n). The network interface 53 may include address, control, and data connections to enable appropriate communications on the network.

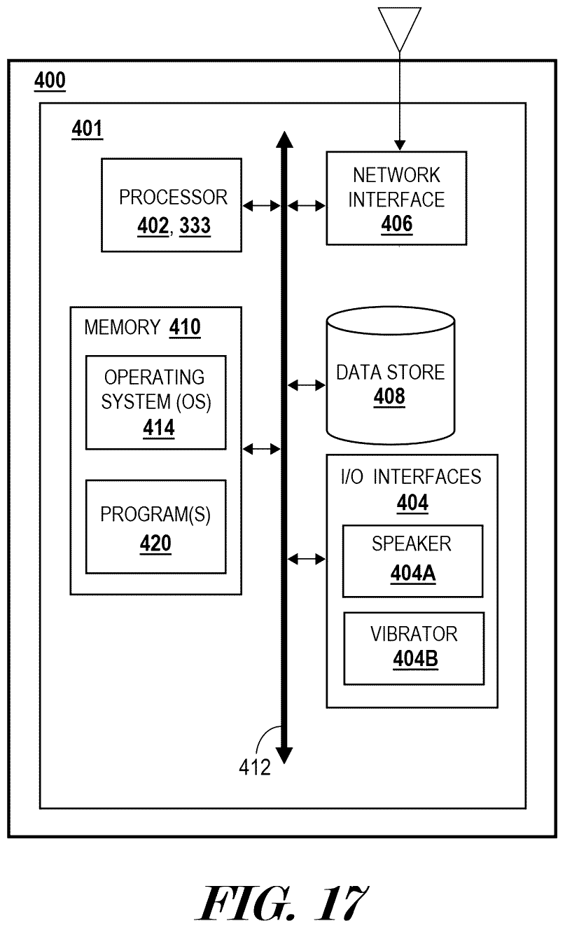

In some embodiments, the auricular ECG monitoring system 100 may be in communication with one or more client devices 400. In some embodiments, the auricular ECG monitoring system 100 may comprise one or more client devices 400. Referring to FIG. 17, in an exemplary embodiment, a block diagram illustrates a client device 400 of which may be a type of computing platform. A client device 400 can be a digital device that, in terms of hardware architecture, generally includes a processing unit 401 with a processor 402, input/output (I/O) interfaces 404, a network interface 406, a data store 408, and memory 410. It should be appreciated by those of ordinary skill in the art that FIG. 17 depicts the client device 400 in an oversimplified manner, and a practical embodiment may include additional components and suitably configured processing logic to support known or conventional operating features that are not described in detail herein. The components (402, 404, 406, 408, and 410) are communicatively coupled via a local interface 412. The local interface 412 can be, for example but not limited to, one or more buses or other wired or wireless connections, as is known in the art. The local interface 412 can have additional elements, which are omitted for simplicity, such as controllers, buffers (caches), drivers, repeaters, and receivers, etc. to enable communications.

The processor 402 is a hardware device for executing software instructions. The processor 402 can be any custom made or commercially available processor, a central processing unit (CPU), an auxiliary processor among several processors associated with the client device 400, a semiconductor-based microprocessor (in the form of a microchip or chip set), or generally any device for executing software instructions. When the client device 400 is in operation, the processor 402 is configured to execute software stored within the memory 410, to communicate data to and from the memory 410, and to generally control operations of the client device 400 pursuant to the software instructions. In an exemplary embodiment, the processor 402 may include a mobile optimized processor such as optimized for power consumption and mobile applications. The I/O interfaces 404 can be used to receive data and user input and/or for providing system output. User input can be provided via a plurality of I/O interfaces 404, such as a keypad, a touch screen, a speaker 404A, a vibrator 404B, a camera, a microphone, a scroll ball, a scroll bar, buttons, barcode scanner, voice recognition, eye gesture, and the like. System output can be provided via a display screen such as a liquid crystal display (LCD), touch screen, and the like. The I/O interfaces 404 can also include, for example, a global positioning service (GPS) radio, a serial port, a parallel port, a small computer system interface (SCSI), an infrared (IR) interface, a radio frequency (RF) interface, a universal serial bus (USB) interface, and the like. The I/O interfaces 404 can include a graphical user interface (GUI) that enables a user to interact with the client device 400. Additionally, the I/O interfaces 404 may be used to output notifications to a user and can include a speaker 404A configured to emit audio notifications, a vibrational device or vibrator 404B configured to vibrate, shake, or produce any other series of rapid and repeated movements to produce haptic notifications, and/or a light emitting diode (LED) or other light emitting element which may be configured to illuminate to provide a visual notification. In some embodiments, the processor 402 may comprise a closed-loop control system 333 to regulate or control the output actuation when a neuromodulation system 300 is activated. (FIG. 17).

In some embodiments, the auricular ECG monitoring system 100 may comprise a network interface 53, 406, and the network interface 53, 406, may be configured to generate an electronic notification via an input/output (I/O) interface 404 of a client device 400 when the processing unit 50, 401, detects the presence or cessation of a serious cardiac arrhythmia or an impending serious cardiac arrhythmia of the wearer 900 of the auricular ECG monitoring system 100. In preferred embodiments, the auricular ECG monitoring system 100 may comprise one or more client devices 400 that may be configured to generate a notification when a processing unit 50, 401, detects the presence or cessation of a serious cardiac arrhythmia or an impending serious cardiac arrhythmia. For example, when a serious cardiac arrhythmia or an impending serious cardiac arrhythmia is detected by a processing unit 50, a network interface 53 of the processing unit 50 may send a notification to the client device 400 of the wearer 900 of the auricular ECG monitoring system 100 and/or may send a notification to the client device 400 of the wearer's healthcare provider 950 to take appropriate actions. When cessation of serious cardiac arrhythmia or cessation of the impending serious cardiac arrhythmia is detected by the processing unit 50 in the auricular housing (ECG recording housing) 11, the network interface 53 of the processing unit 50 may send a notification to the client device 400 of the wearer 900 of the auricular ECG monitoring system 100 and/or may send a notification to the client device 400 of the wearer's healthcare provider 950 to take appropriate actions. Similar functions may be provided by a processing unit 401 located on a client device 400. As an alternative example, when a serious cardiac arrhythmia or an impending serious cardiac arrhythmia is detected by a processing unit 401 of a client device 400 that is in electronic communication 19 with the ECG recording module 20 (via a network interface 53), the processing unit 401 of the client device 400 of the wearer 900 may generate a notification and/or a client device 400 of the wearer's healthcare provider 950 may generate a notification to enable the wearer's healthcare provider 950 to take appropriate actions. When cessation of serious cardiac arrhythmia or impending serious cardiac arrhythmia is detected by the processing unit 401 of the client device 400 that is in electronic communication 19 with the ECG recording module 20 (via a network interface 53), the processing unit 401 of the client device 400 of the wearer 900 of the ECG recording housing 11 may generate a notification and/or a client device 400 of the wearer's healthcare provider 950 may generate a notification to enable the wearer's healthcare provider 950 to take appropriate actions.

In some embodiments, the auricular ECG monitoring system 100 may comprise a data store 408. The data store 408 may be used to store data and is therefore a type of memory. The data store 408 may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 408 may incorporate electronic, magnetic, optical, and/or other types of storage media. The memory 410 may include any of volatile memory elements (e.g., random access memory (RAM, such as DRAM, SRAM, SDRAM, etc.)), and nonvolatile memory elements (e.g., ROM, hard drive, etc.). Moreover, the memory 410 may incorporate electronic, magnetic, optical, and/or other types of storage media. Note that the memory 410 may have a distributed architecture, where various components are situated remotely from one another, but can be accessed by the processor 402. The software in memory 410 can include software programs 420, which include an ordered listing of executable instructions for implementing logical functions.

The ECG recording module 20 may be configured to record ECG data of the wearer 900. The auricular ECG monitoring system 100 is in electronic communication with a processing unit 50, 401, that is configured to detect presence or cessation of serious cardiac arrhythmias and presence or cessation of impending serious cardiac arrhythmia, with the help of various ECG analysis algorithms (as known in the art). Recent advances in ECG analysis algorithms have helped detection of impending serious cardiac arrhythmia. Key predictive patterns of premature atrial contractions (PACs) have been found to help predict impending development of paroxysmal atrial fibrillation (AFib), including frequency of PACs (or PAC burden) (for example more than 3,000 PACs in a 24-hour period). Origin of PAC from pulmonary veins area is also a highly critical predictor. The pattern (clustering and timing) of PACs, including short rapid bursts or clusters of PACs, using clustering analysis algorithms and variable timing analysis algorithms can also help to predict paroxysmal atrial fibrillation. With artificial intelligence and machine learning, it has been shown that clustering PACs based on their P-wave morphology, timing, and coupling intervals can predict impending AFib. A high variability in the timing of PACs, especially in the coupling interval (the time between the premature beat and the preceding normal beat), has also been found to help predict impending AFib risk. Presence of these specific PACs patterns indicates atrial cardiomyopathy that make the patient vulnerable to atrial fibrillation.

Similarly, key predictive patterns of premature atrial contractions (PACs) have been found to help predict development of atrial flutter (AFL). These include frequency of PACs (PAC burden) (for example over 3,000 PACs in 24 hours). The temporal patterns of PACs are also correlated with risk of impending atrial flutter. With artificial intelligence and machine learning to analyze ECG data, distinct clusters (high-risk clusters) of PACs have been identified, including the PACs morphology, timing, and coupling interval and these have been developed into ECG analysis algorithms for prediction of impending AFL. Source of the PACs (from pulmonary vein area) also helps in prediction of impending atrial flutter.

Likewise, key predictive patterns of premature atrial contractions (PACs) have been found to help predict development of supraventricular tachycardia (SVT). Frequent PACs (for example: over 3,000 PACs in 24 hours) is again indicative of high risk for SVT. The PAC patterns can also help to predict SVT, including repetitive and organized PAC patterns significantly increase the risk of SVT as compared to simple, isolated PACs, including bigeminy and trigeminy. Occasional short run of non-sustained SVT can also predict impending sustained SVT. Early PACs that are blocked (not conducted to the ventricles) can also be a significant risk factor for impending more complex SVT.

There are ECG patterns that can help to predict impending ventricular tachycardia (VT). VT may occur with underlying cardiac diseases, for example Brugada syndrome with its specific ECG patterns (prominent, coved ST-segment elevation of at least 2 mm in leads V1 and V2 with an associated T-wave inversion.) or arrhythmogenic right ventricular cardiomyopathy (ECG showing T-wave inversion, Epsilon wave, prolong S-wave upstroke). Other ECG technics, like signal-averaged ECG and heart-rate variability may also help to predict impending ventricular tachycardia (VT). Frequent premature ventricular contractions (PVCs) or high burden of PVCs, especially with uniform morphology, can help to predict impending VT. Algorithms for differentiating VT from other wide-complex tachycardias are known in the art.

Similarly, there are ECG patterns and features that can help predict impending ventricular fibrillation (VF). These ECG markers may include T-wave alternans, prolonged T-peak to T-end interval, QT interval prolongation, early repolarization pattern, fragmented QRS, long QRS duration, Epsilon waves, frequent PVCs or episodes of non-sustained ventricular tachycardia, etc. and presence of these may indicate increased risks of impending ventricular fibrillation.

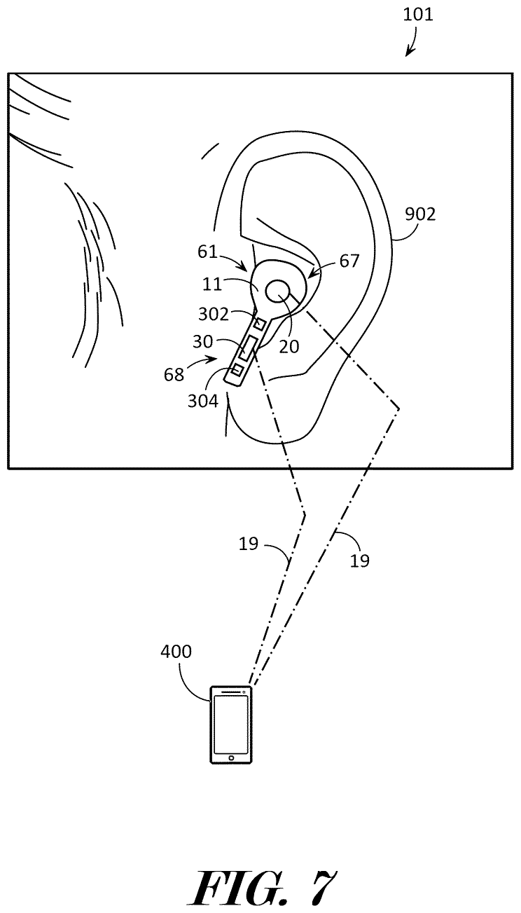

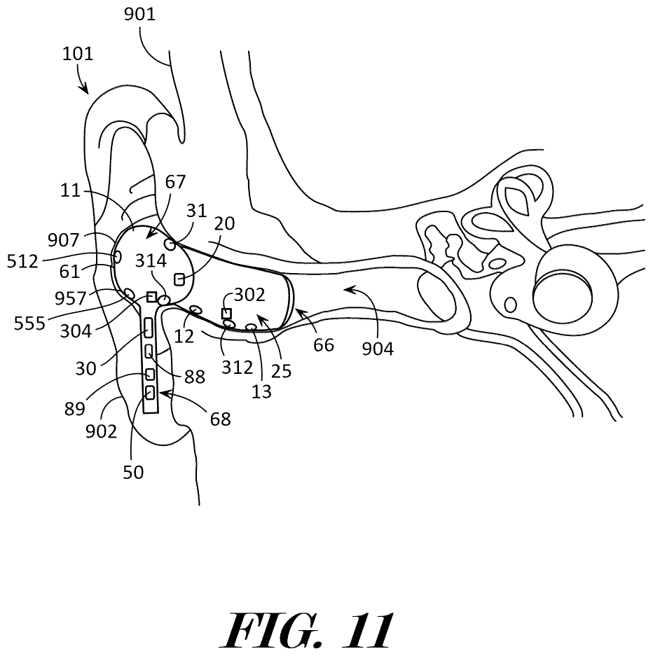

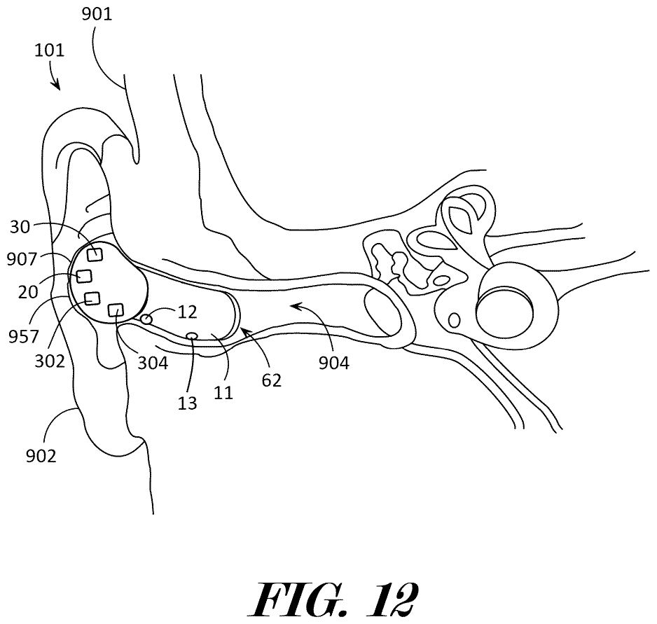

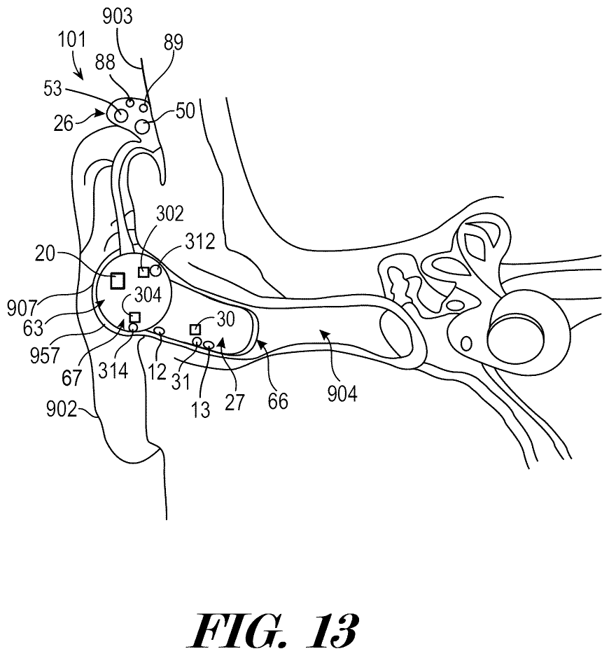

According to another aspect consistent with the principles of the present invention, an automatic detection-therapy system for cardiac arrhythmia 101 is disclosed (FIGS. 2, 7, 11, 12, 13). In some embodiments, an automatic detection-therapy system for cardiac arrhythmia 101 may comprise an auricular electrocardiogram (ECG) monitoring system 100 having an ECG recording module 20, such as described hereinbefore, that may be configured to record ECG data of the wearer 900. The auricular ECG monitoring system 100 is in electronic communication with a processing unit 50, 401, that is configured to detect presence or cessation of serious cardiac arrhythmias and presence or cessation of impending serious cardiac arrhythmias with the help of various ECG analysis algorithms (as known in the art). Serious cardiac arrhythmias may include atrial fibrillation (AFib), atrial flutter (AFL), supraventricular tachycardia (SVT), ventricular tachycardia (VT), and ventricular fibrillation (VF). The automatic detection-therapy system for cardiac arrhythmia 101 may further comprise a neuromodulation system 300. The neuromodulation system 300 may comprise at least one of the following components: a transcutaneous auricular vagus nerve stimulation (taVNS) unit 30, a supraorbital nerve stimulation unit 301, an auriculotemporal nerve stimulation unit 302, an occipital nerve stimulation unit 303, a greater auricular nerve stimulation unit 304, an infraorbital nerve stimulation unit 305 and a median nerve stimulation unit 306.

In some embodiments, an automatic detection-therapy system for cardiac arrhythmia 101 may have a neuromodulation system 300 that comprises a transcutaneous auricular vagus nerve stimulation unit (taVNS unit) 30. The taVNS unit 30 includes a miniature taVNS stimulating electrode 31 that is configured to contact or be attached to vagus nerve innervated auricular skin of the wearer 900. The vagus innervated auricular skin includes external ear canal 904, tragus 905, cymba concha 906, cavum concha 907 and small adjacent areas. The vagus innervated auricular skin that the stimulating electrode 31 is configured to contact may be selected from one of the following: external ear canal 904, tragus 905, cymba concha 906, and cavum concha 907 of the wearer's ear 902. When prompted (by signals from the processing unit 50, 401) the taVNS unit 30 is configured to generate electric stimulation to the vagus innervated auricular skin via the taVNS stimulating electrode 31. The ECG recording module 20 may be in wired or wireless electronic communication (e.g., through wire, Bluetooth, etc.) with a processing unit 50, 401. The taVNS unit 30 may also be in electronic communication with the processing unit 50, 401. The processing unit 50, 401, may be configured to analyze the ECG data recorded by the auricular ECG monitoring system 100 to detect the presence or cessation of ECG signals suggestive of serious cardiac arrhythmias or impending serious cardiac arrhythmia. When the presence of ECG signals suggestive of at least one serious cardiac arrhythmia is detected by the processing unit 50, 401, the processing unit 50, 401, may be configured to immediately send signals (arrhythmia-warning signals) to the taVNS unit 30. Upon receiving arrhythmia-warning signals from the processing unit 50, 401, the taVNS unit 30 is configured to automatically start sending pre-determined (or closed-loop controlled) neuromodulating electric stimulation to the vagus innervated auricular skin of the wearer's ear 902 to which the taVNS stimulating electrode 31 is in contact with. (Pre-determined stimulating parameters of taVNS 30 for serious cardiac arrhythmia as shown in Table 1). When cessation of ECG signals suggestive of the at least one serious cardiac arrhythmia is detected by the processing unit 50, 401, the processing unit 50, 401, may be further configured to immediately send signals (arrhythmia-cessation signals) to the taVNS unit 30. Upon receiving arrhythmia-cessation signals from the processing unit 50, 401, the taVNS unit 30 is configured to automatically stop sending electric stimulation to the vagus innervated auricular skin of the wearer's ear 902. When the presence of ECG signals suggestive of at least one impending serious cardiac arrhythmia is detected by the processing unit 50, 401, the processing unit 50, 401, may be configured to immediately send signals (impending-arrhythmia-warning signals) to the taVNS unit 30. Upon receiving impending-arrhythmia-warning signals from the processing unit 50, 401, the taVNS unit 30 is configured to automatically start sending pre-determined (or closed-loop controlled) neuromodulating electric stimulation to the vagus innervated auricular skin of the wearer's ear 902 to which the taVNS stimulating electrode 31 is in contact with. (Pre-determined stimulating parameters of taVNS 30 for impending serious cardiac arrhythmia as shown in Table 2). When cessation of ECG signals suggestive of the at least one impending serious cardiac arrhythmia is detected by the processing unit 50, 401, the processing unit 50, 401, may be further configured to immediately send signals (impending-arrhythmia-cessation signals) to the taVNS unit 30. Upon receiving impending-arrhythmia-cessation signals from the processing unit 50, 401, the taVNS unit 30 is configured to automatically stop sending electric stimulation to the vagus innervated auricular skin of the wearer's ear 902.

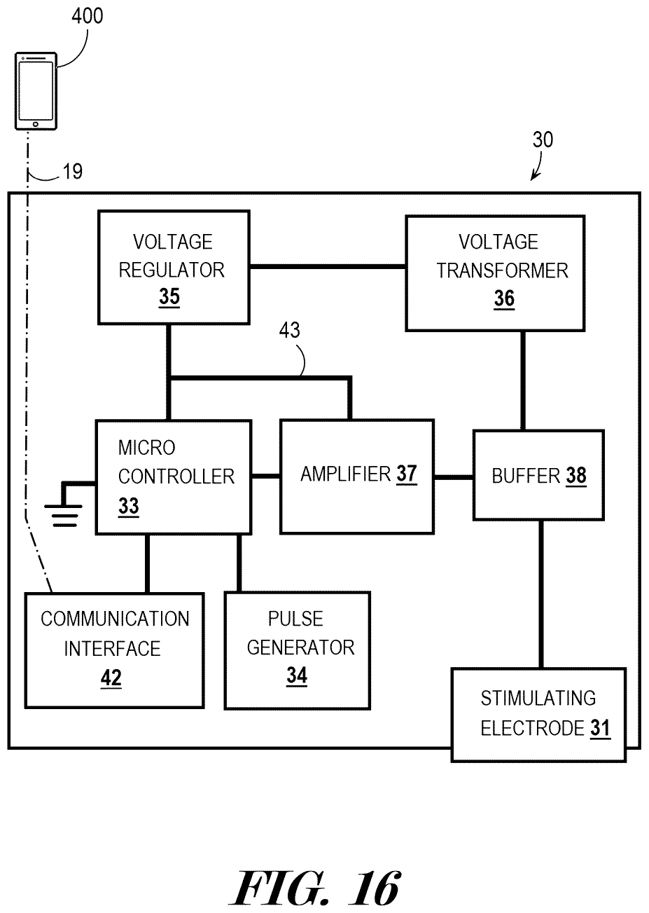

In preferred embodiments, an automatic detection-therapy system for cardiac arrhythmia 101 may comprise a transcutaneous auricular vagus nerve stimulation (taVNS) unit 30 and an auricular ECG monitoring system 100. The taVNS unit 30 may comprise any device that is able to provide transcutaneous auricular vagus nerve stimulation to a user's body. As an example, and referring to FIG. 16, a taVNS unit 30 may comprise a microcontroller 33 that may be in communication with a pulse generator 34, voltage regulator 35, voltage transformer 36, amplifier 37, and buffer 38, and that may be configured to generate taVNS electric stimuli that may be delivered to vagus nerve innervated auricular skin via a stimulating electrode 31. Optionally, a taVNS unit 30 may comprise a communication interface 42 which may enable electronic communication 19 (e.g., wired and/or wireless communication) between the taVNS unit 30 and another electronic device, such as a client device 400, an auricular ECG monitoring system 100, etc. Preferably, a communication interface 42 may comprise a radio that may operate via WiFi and/or Bluetooth communication standards. In some embodiments, a communication interface 42 may be configured as a network interface 53 described above so that it may operate on any wireless and/or wired electronic communication 19 protocol that a network interface 53 may use. In some embodiments, when ECG signals suggestive of a serious cardiac arrhythmia is detected, the processing unit 50, 401, may be configured to automatically send signals immediately to the taVNS unit 30 of the automatic detection-therapy system for cardiac arrhythmia 101 to actuate the vagus nerve stimulation via the taVNS stimulating electrode 31, utilizing pre-determined stimulation parameters, such as shown in Table 1.

TABLE 1
Example of taVNS unit 30 electric stimulation output
parameters for a serious cardiac arrhythmia:

| Output | Parameter |
|---|---|
| Power supply | Direct current 3-9 volts |
| Pulse width | 100-1000 microseconds, more preferably |
| | 0.25-1 milliseconds (ms) |
| Frequency | 0.5-200 Hz, more preferably 20-25 Hertz (Hz) |
| Modes | Continuous wave or sparse-dense wave |
| Intensity | 0.1-15 milliamperes (mA), more preferably |
| | 0.2-2 mA |

When ECG signals suggestive of an impending serious cardiac arrhythmia is detected, the processing unit 50, 401, may be configured to automatically send signals immediately to the taVNS units 30 of the automatic detection-therapy system for cardiac arrhythmia 101 to actuate the vagus nerve stimulation via the taVNS stimulating electrode 31, utilizing pre-determined stimulation parameters, such as shown in Table 2.

TABLE 2
Example of taVNS unit 30 electric stimulation output parameters
for an impending serious cardiac arrhythmia:

| Output | Parameter |
|---|---|
| Power supply | Direct current 3-9 volts |
| Pulse width | 100-800 microseconds, more preferably |
| | 0.2-0.7 millisecond (ms) |
| Frequency | 0.5-100 Hz, more preferably 10-20 Hertz (Hz) |
| Modes | Continuous wave or sparse-dense wave |
| Intensity | 0.1-15 milliamperes (mA), more preferably |
| | 0.15-1.5 mA |

It should be noted that, in some embodiments, the taVNS stimulation parameters may be adjusted by the closed-loop control system 333 in real-time in response to the real-time input data and feedback from at least one of: the auricular ECG monitoring system 100, the auricular SCOS unit 511, SPG unit 555 (or PPG unit 555A) and auricular EEG monitoring system 170.

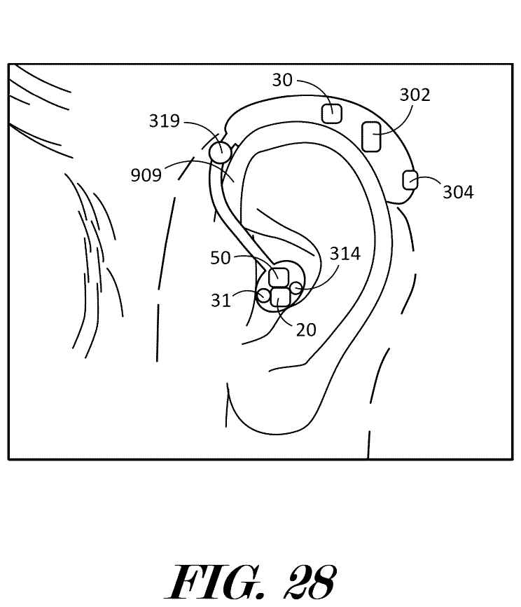

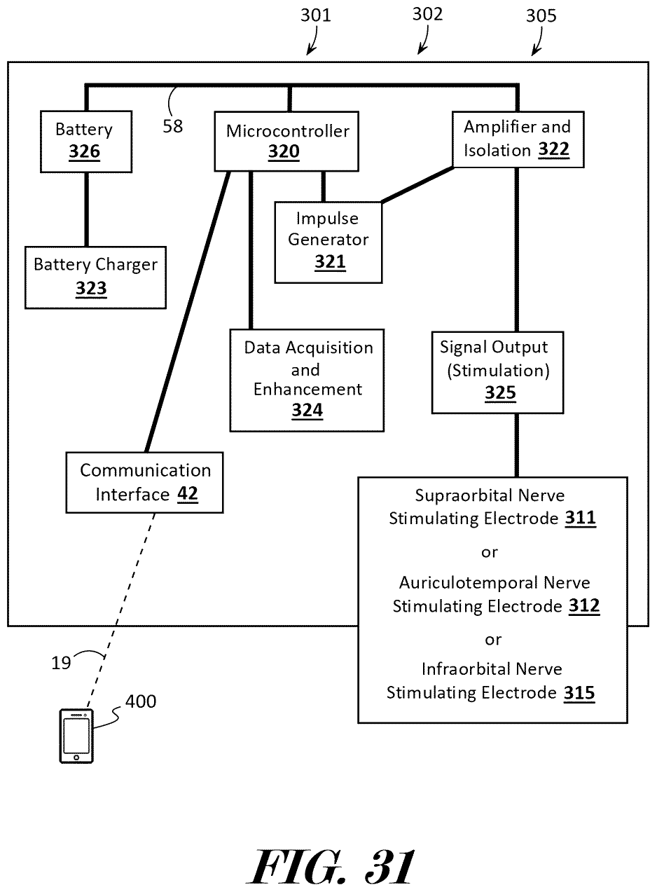

In some embodiments, an automatic detection-therapy system for cardiac arrhythmia 101 may include a neuromodulation system 300 that may comprise an auriculotemporal nerve (ATN) stimulation unit 302. The auriculotemporal nerve (ATN) is a branch of the mandibular division of the trigeminal nerve. The ATN stimulation unit 302 includes an ATN stimulating electrode 312 configured to contact the ATN innervated area of the wearer's ear 902. The ATN innervated area includes the anterior-outer part of tragus 905, upper anterior part of the helix 909, anterior portion of cavum concha 907, anterior and superior walls of external ear canal 904, outer surface of the tympanic membrane and area immediately in front of the tragus 905. The ATN innervated area is anatomically adjacent to or overlapping with the vagus-innervated auricular skin. Because of this, it is feasible for the ATN stimulation unit 902 and the taVNS unit 30 to share a housing device or even share a stimulating electrode. However, it is usually preferred to have separate devices (could be integrated into one housing) with separate stimulating electrodes and separate stimulating parameters because their optimal stimulating parameters are often different. The auriculotemporal nerve (ATN) stimulation unit 302 may comprise any device that is able to provide transcutaneous ATN stimulation to a user's body when activated. As an example, and referring to FIG. 31, an ATN stimulation unit 302 may comprise a microcontroller 320 that may be in communication with an impulse generator 321, amplifier and isolation 322, data acquisition and enhancement 324, signal output (stimuli) 325, and auriculotemporal nerve stimulating electrode 312, 319, to auriculotemporal nerve. The ATN stimulation unit 302 further comprises a battery 326, a battery charger 323 and a communication interface 42 for communication to a client device 400. The ATN stimulation unit 302 may be configured to generate electric stimulation that may be transmitted to ATN innervated auricular skin (such as external ear canal 904 or tragus-concha bowl 957) via the ATN stimulating electrode 312. Alternatively, the ATN stimulation unit 302 may be configured to generate electric stimulation that may be transmitted to ATN innervated auricular skin at anterior-superior helix 909 of the wearer's ear 902 via a clip electrode 319 for ATN stimulation. (FIG. 28). A processing unit 50, 401, is in electronic communication with the auricular ECG monitoring system 100 and the ATN stimulation unit 302. The processing unit 50, 401, is configured to analyze the ECG data recorded by the auricular ECG monitoring system 100 to detect the presence or cessation of ECG signals suggestive of serious cardiac arrhythmia. When the presence of ECG signals suggestive of serious cardiac arrhythmia is detected by the processing unit 50, 401, the processing unit is configured to immediately send signals to the ATN stimulation unit 302 to prompt it to automatically start sending closed-loop controlled (or pre-determined) neuromodulating electric stimulation to the wearer's ATN. When cessation of ECG signals suggestive of serious cardiac arrhythmia is detected by the processing unit 50, 401, the processing unit is further configured to immediately send signals to the ATN stimulation unit 302 to prompt it to automatically stop sending neuromodulating electric stimulation to the wearer's ATN. The processing unit 50, 401, is further configured to analyze the ECG data recorded by the ECG monitoring system 100 to detect presence or cessation of ECG signals suggestive of impending serious cardiac arrhythmia. When the presence of ECG signals suggestive of impending serious cardiac arrhythmia is detected by the processing unit 50, 401, the processing unit is configured to immediately send signals to the ATN stimulation unit 302 to prompt it to automatically start sending closed-loop controlled (or pre-determined) neuromodulating electric stimulation to the wearer's ATN. When cessation of ECG signals suggestive of impending serious cardiac arrhythmia is detected by the processing unit 50, 401, the processing unit is further configured to immediately send signals to the ATN stimulation unit 302 to prompt it to automatically stop sending neuromodulating electric stimulation to the wearer's auriculotemporal nerve (ATN).

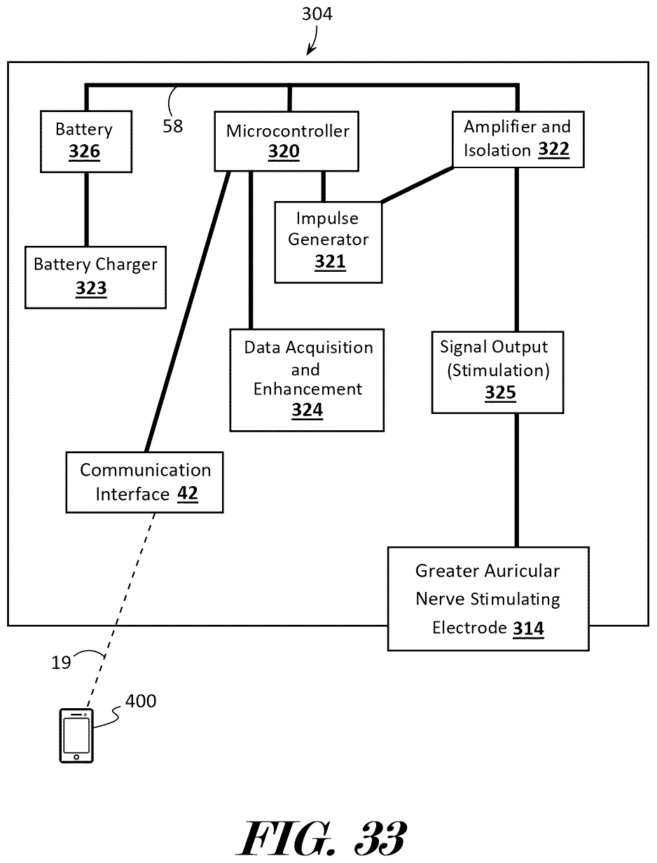

In some embodiments, an automatic detection-therapy system for cardiac arrhythmia 101 may include a neuromodulation system 300 that may comprise a greater auricular nerve (GAN) stimulation unit 304. The greater auricular nerve (GAN) is a pure sensory nerve originated from the cervical spinal cord (C2, C3) and provides sensory innervation to the skin over the parotid gland, the mastoid process, and the lower two-thirds of the outer ear (pinna). The GAN innervated auricular skin includes skin on both surfaces of the lower two-thirds of pinna (including the lobule) and cavum concha (inferior/lower part of concha). The posterior branch of greater auricular nerve provides sensory innervation to the skin of the lower part of the cavum concha on both the posterior (back) and anterior (front/lateral) surfaces. The GAN stimulation unit 304 includes a GAN stimulating electrode 314 configured to contact the GAN innervated area of the wearer's auricular skin. The GAN stimulation unit 304 may comprise any device that is able to provide GAN stimulation to a user's body when activated. As an example, and referring to FIG. 33, a GAN stimulation unit 304 may comprise a microcontroller 320 that may be in communication with an impulse generator 321, amplifier and isolation 322, data acquisition and enhancement 324, signal output (stimuli) 325, and greater auricular nerve (GAN) stimulating electrode 314. The GAN stimulation unit 304 further comprises a battery 326, a battery charger 323 and a communication interface 42 for communication with a client device 400. The GAN stimulation unit 304 may be configured to generate electric stimulation that may be transmitted to GAN innervated auricular skin via the GAN stimulating electrode 314. A processing unit 50, 401, is in electronic communication with the auricular ECG monitoring system 100 and the GAN stimulation unit 304. The processing unit 50, 401, is configured to analyze the ECG data recorded by the ECG monitoring system 100 to detect the presence or cessation of ECG signals suggestive of serious cardiac arrhythmia. When the presence of ECG signals suggestive of serious cardiac arrhythmia is detected by the processing unit 50, 401, the processing unit is configured to immediately send signals to the GAN stimulation unit 304 to prompt it to automatically start sending closed-loop controlled (or pre-determined) neuromodulating electric stimulation to the wearer's GAN. When cessation of ECG signals suggestive of serious cardiac arrhythmia is detected by the processing unit 50, 401, the processing unit is further configured to immediately send signals to the GAN stimulation unit 304 to prompt it to automatically stop sending neuromodulating electric stimulation to the wearer's GAN. The processing unit 50, 401, is further configured to analyze the ECG data recorded by the ECG monitoring system 100 to detect presence or cessation of ECG signals suggestive of impending serious cardiac arrhythmia. When the presence of ECG signals suggestive of impending serious cardiac arrhythmia is detected by the processing unit 50, 401, the processing unit is configured to immediately send signals to the GAN stimulation unit 304 to prompt it to automatically start sending closed-loop controlled (or pre-determined) neuromodulating electric stimulation to the wearer's GAN. When cessation of ECG signals suggestive of impending serious cardiac arrhythmia is detected by the processing unit 50, 401, the processing unit is further configured to immediately send signals to the GAN stimulation unit 304 to prompt it to automatically stop sending neuromodulating electric stimulation to the wearer's greater auricular nerve (GAN).

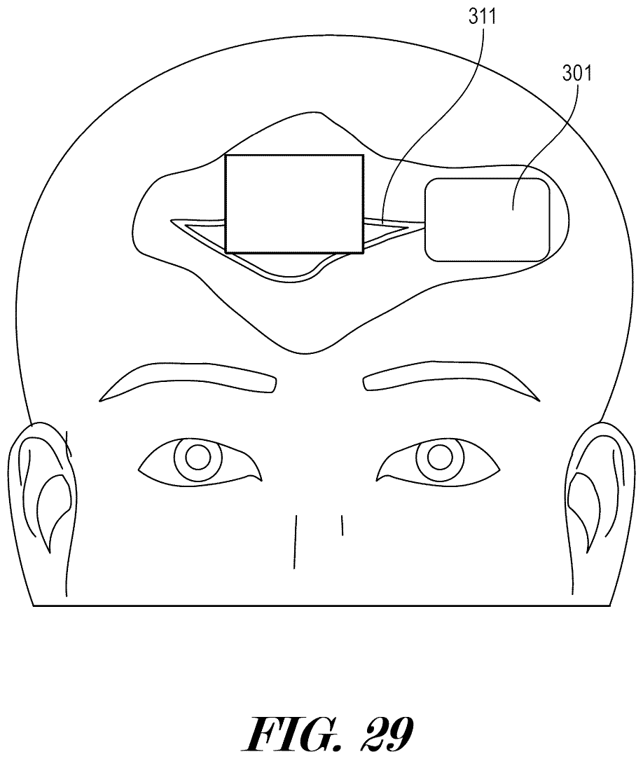

In some embodiments, an automatic detection-therapy system for cardiac arrhythmia 101 may include a neuromodulation system 300 that may comprise a supraorbital nerve stimulation unit 301. The supraorbital nerve stimulation unit 301 may comprise any device that is able to provide transcutaneous supraorbital nerve stimulation to a user's body when activated. As an example, and referring to FIGS. 29, 31, a supraorbital nerve stimulation unit 301 may comprise a microcontroller 320 that may be in communication with an impulse generator 321, amplifier and isolation 322, data acquisition and enhancement 324, signal output (stimuli) 325, and supraorbital nerve stimulating electrode 311 to supraorbital nerve(s). The supraorbital nerve stimulation unit 301 further comprises a battery 326, a battery charger 323 and a communication interface 42 for communication to a client device 400. The supraorbital nerve stimulation unit 301 may be configured to generate electric stimulation that may be transmitted to supraorbital nerve innervated mid-forehead skin via the supraorbital nerve stimulating electrode 311. The supraorbital nerve stimulation unit 301 includes a supraorbital nerve stimulating electrode 311 configured to contact the supraorbital nerve innervated area of the wearer's mid-forehead skin. The supraorbital nerve stimulating electrode 311 may contact unilateral supraorbital nerve or, more commonly, bilateral supraorbital nerves. A processing unit 50, 401, is in electronic communication with the auricular ECG monitoring system 100 and the supraorbital nerve stimulation unit 301. The processing unit is configured to analyze the ECG data recorded by the ECG monitoring system 100 to detect the presence or cessation of ECG signals suggestive of serious cardiac arrhythmia. When the presence of ECG signals suggestive of serious cardiac arrhythmia is detected by the processing unit 50, 401, the processing unit is configured to immediately send signals to the supraorbital nerve stimulation unit 301 to prompt it to automatically start sending closed-loop controlled (or pre-determined) neuromodulating electric stimulation to the wearer's supraorbital nerves. When cessation of ECG signals suggestive of serious cardiac arrhythmia is detected by the processing unit 50, 401, the processing unit is further configured to immediately send signals to the supraorbital nerve stimulation unit 301 to prompt it to automatically stop sending neuromodulating electric stimulation to the wearer's supraorbital nerves. The processing unit 50, 401, is configured to analyze the ECG data recorded by the ECG monitoring system 100 to detect presence or cessation of ECG signals suggestive of impending serious cardiac arrhythmia. When the presence of ECG signals suggestive of impending serious cardiac arrhythmia is detected by the processing unit 50, 401, the processing unit is configured to immediately send signals to the supraorbital nerve stimulation unit 301 to prompt it to automatically start sending closed-loop controlled (or pre-determined) neuromodulating electric stimulation to the supraorbital nerves. When cessation of ECG signals suggestive of impending serious cardiac arrhythmia is detected by the processing unit 50, 401, the processing unit is further configured to immediately send signals to the supraorbital nerve stimulation unit 301 to prompt it to automatically stop sending neuromodulating electric stimulation to the wearer's supraorbital nerves.

In some embodiments, an automatic detection-therapy system for cardiac arrhythmia 101 may include a neuromodulation system 300 that may comprise an infraorbital nerve stimulation unit 305. The infraorbital nerve stimulation unit 305 includes an infraorbital nerve stimulating electrode 315 configured to contact the infraorbital nerve innervated area of the wearer's mid-facial region. The infraorbital nerve stimulating electrode 315 may contact unilateral infraorbital nerve or, more commonly, bilateral infraorbital nerves. Infraorbital nerve is a branch from the second division of the trigeminal nerve. However, infraorbital nerve stimulation is more complex to set up and sometimes requires invasive procedure (although transcutaneous infraorbital nerve stimulator is available nowadays). The setups and functions of the infraorbital nerve stimulation unit 305 is essentially the same as those for the supraorbital nerve stimulation unit 301, as aforementioned description.

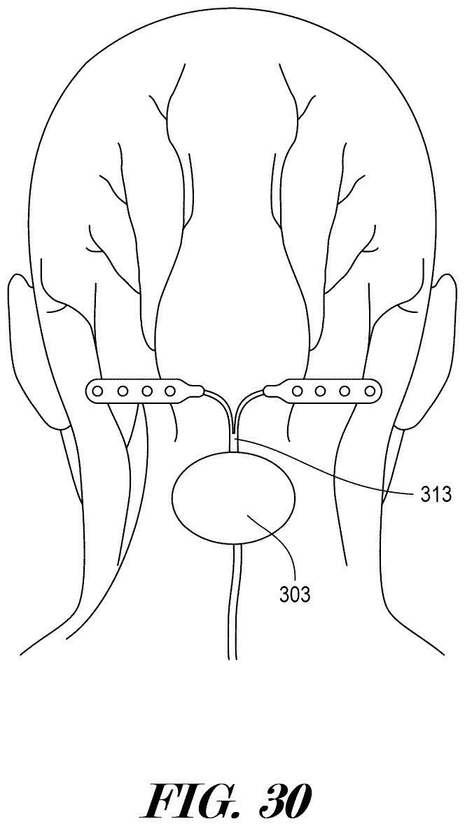

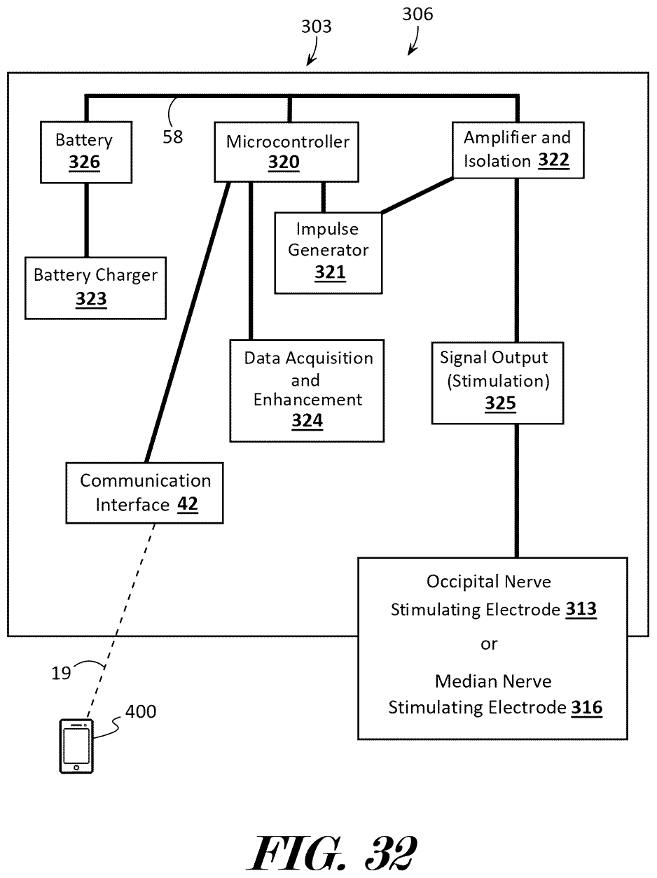

In some embodiments, an automatic detection-therapy system for cardiac arrhythmia 101 may include a neuromodulation system 300 that may comprise an occipital nerve stimulation unit 303. The occipital nerve stimulation unit 303 includes an occipital nerve stimulating electrode 313 configured to contact the occipital nerve innervated area of the wearer's mid-occipital region. The occipital nerve stimulating electrode 313 may contact unilateral occipital nerve or, more commonly, bilateral occipital nerves. The occipital nerve stimulation unit 303 may comprise any device that is able to provide occipital nerve stimulation to a user's body when activated. As an example, and referring to FIGS. 30, 32, an occipital nerve stimulation unit 303 may comprise a microcontroller 320 that may be in communication with an impulse generator 321, amplifier and isolation 322, data acquisition and enhancement 324, signal output (stimuli) 325, and occipital nerve stimulating electrode 313. The occipital nerve stimulation unit 303 further comprises a battery 326, a battery charger 323 and a communication interface 42 for communication with a client device 400. The occipital nerve stimulation unit 303 may be configured to generate electric stimulation that may be transmitted to occipital nerve innervated occipital region via the occipital nerve stimulating electrode 313. A processing unit 50, 401, is in electronic communication with the auricular ECG monitoring system 100 and the occipital nerve stimulation unit 303. The processing unit 50, 401, is configured to analyze the ECG data recorded by the ECG monitoring system 100 to detect the presence or cessation of ECG signals suggestive of serious cardiac arrhythmia. When the presence of ECG signals suggestive of serious cardiac arrhythmia is detected by the processing unit 50, 401, the processing unit is configured to immediately send signals to the occipital nerve stimulation unit 303 to prompt it to automatically start sending closed-loop controlled (or pre-determined) neuromodulating electric stimulation to the wearer's occipital nerves. When cessation of ECG signals suggestive of serious cardiac arrhythmia is detected by the processing unit 50, 401, the processing unit is further configured to immediately send signals to the occipital nerve stimulation unit 303 to prompt it to automatically stop sending neuromodulating electric stimulation to the wearer's occipital nerves. The processing unit 50, 401, is further configured to analyze the ECG data recorded by the ECG monitoring system 100 to detect presence or cessation of ECG signals suggestive of impending serious cardiac arrhythmia. When the presence of ECG signals suggestive of impending serious cardiac arrhythmia is detected by the processing unit 50, 401, the processing unit is configured to immediately send signals to the occipital nerve stimulation unit 303 to prompt it to automatically start sending closed-loop controlled (or pre-determined) neuromodulating electric stimulation to the wearer's occipital nerves. When cessation of ECG signals suggestive of impending serious cardiac arrhythmia is detected by the processing unit 50, 401, the processing unit is configured to immediately send signals to the occipital nerve stimulation unit 304 to prompt it to automatically stop sending neuromodulating electric stimulation to the wearer's occipital nerves.

In some embodiments, an automatic detection-therapy system for cardiac arrhythmia 101 may include a neuromodulation system 300 that may comprise a median nerve stimulation unit 306. The median nerve stimulation unit 306 includes a median nerve stimulating electrode 316 configured to contact the skin at Neiguan point for median nerve stimulation. The Neiguan point is located on the palmar side of distal forearm (on the palmar side or inner side of distal forearm), 1.3-2 inches (two to three finger-widths) proximal to the wrist crease, directly between two prominent central tendons (palmaris longus and flexor carpi radialis). The Neiguan point lies directly over the median nerve and corresponds anatomically to the median nerve pathway (the median nerve passes underneath). The Neiguan point is an easily identifiable location for median nerve stimulation. By placing the median nerve stimulating electrode 316 at the Neiguan point, the median nerve stimulation unit 306 is configured to give transcutaneous electric stimulation to the median nerve in a way similar to a Transcutaneous Electrical Nerve Stimulation (TENS). Median nerve stimulation may be given unilaterally or bilaterally. A processing unit 50, 401, is in electronic communication with the auricular ECG monitoring system 100 and the median nerve stimulation unit 306. The processing unit 50, 401, is configured to analyze the ECG data recorded by the ECG monitoring system 100 to detect the presence or cessation of ECG signals suggestive of serious cardiac arrhythmia. When the presence of ECG signals suggestive of serious cardiac arrhythmia is detected by the processing unit 50, 401, the processing unit is configured to immediately send signals to the median nerve stimulation unit 306 to prompt it to automatically start sending closed-loop controlled (or pre-determined) neuromodulating electric stimulation to the wearer's median nerve. When cessation of ECG signals suggestive of serious cardiac arrhythmia is detected by the processing unit 50, 401, the processing unit is further configured to immediately send signals to the median nerve stimulation unit 306 to prompt it to automatically stop sending neuromodulating electric stimulation to the wearer's median nerve. The processing unit 50, 401, is further configured to analyze the ECG data recorded by the ECG monitoring system 100 to detect presence or cessation of ECG signals suggestive of impending serious cardiac arrhythmia. When the presence of ECG signals suggestive of impending serious cardiac arrhythmia is detected by the processing unit 50, 401, the processing unit is configured to immediately send signals to the median nerve stimulation unit 306 to prompt it to automatically start sending closed-loop controlled (or pre-determined) neuromodulating electric stimulation to the wearer's median nerve. When cessation of ECG signals suggestive of impending serious cardiac arrhythmia is detected by the processing unit 50, 401, the processing unit is configured to immediately send signals to the median nerve stimulation unit 306 to prompt it to stop sending neuromodulating electric stimulation to the wearer's median nerve.

Besides using ECG to monitor cardiac arrhythmia, monitoring of cerebral blood flow, regional blood flow, blood pressure and electroencephalogram (EEG) may provide additional valuable information about the wearer's cardiac arrhythmia and hemodynamic status. Blood flow data, blood pressure data and qEEG data will also be very important to guide the neuromodulation therapy, using closed-loop control system 333 to enhance effectiveness and safety. In some embodiments, an automatic detection-therapy system for cardiac arrhythmia 101 may comprise a cardiovascular monitoring apparatus 600, a processing unit 50, 401, a neuromodulation system 300 and a network interface 53. The processing unit 50, 401, is in electronic communication with the cardiovascular monitoring apparatus 600, the neuromodulation system 300 and the network interface 53. The cardiovascular monitoring apparatus 600 comprises an auricular ECG monitoring system 100 and a cephalic blood flow monitoring system 530. The cephalic blood flow monitoring system 530 may comprise a laser speckle-based blood flow monitoring unit, such as a speckle contrast optical spectroscope unit (SCOS unit) 510 or an auricular speckle contrast optical spectroscopy unit (auricular SCOS unit) 511. The processing unit 50, 401, is in electronic communication with the auricular ECG monitoring system 100 and the auricular SCOS unit 511. In alternative embodiments, the cephalic blood flow monitoring system 530 may comprise at least one of: a speckle-plethysmography (SPG) unit 555 and a photoplethysmography (PPG) unit 555A. The at least one of: SPG unit 555 and PPG unit 555A may be in electronic communication with the processing unit 50, 401. (SPG and PPG are known in the art). The at least one of: SPG 555 unit and PPG unit 555A may be used to monitor regional blood flow and blood pressure. (Both SPG and PPG primarily monitor changes in blood volume and they are easy and convenient devices to monitor regional blood flow and systolic and diastolic blood pressure). In other embodiments, a cephalic blood flow monitoring system 530 may comprise a combination of both an auricular SCOS unit 511 and at least one of: a SPG unit 555 and a PPG unit 555A. The processing unit 50, 401, may be configured to analyze the data transmitted from the auricular SCOS unit 511 and the data transmitted from at least one of: the SPG unit 555 and the PPG unit 555A to assess the wearer's cerebral and extracranial blood flow data, regional blood flow data and systolic and diastolic blood pressure data. It is known that combination of SCOS and SPG (or PPG) may provide blood flow data and blood pressure data and may be more useful than using SCOS or SPG or PPG alone. A SPG unit 555 (or a PPG unit 555A) may be placed on a surface of the auricular housing 11 (in either the tubular-shaped structure 66 or the body-structure 67) to monitor blood pressure and regional blood flow of the wearer 900. (Ultrasound-doppler based blood flow monitoring device is another alternative for the cephalic blood flow monitoring system 530.) The auricular SCOS unit 511 comprises an auricular SCOS sensor 512 configured to contact the wearer's auricular skin when measuring the wearer's blood flow. The auricular SCOS sensor 512 is usually integrated with the CMOS camera 516.

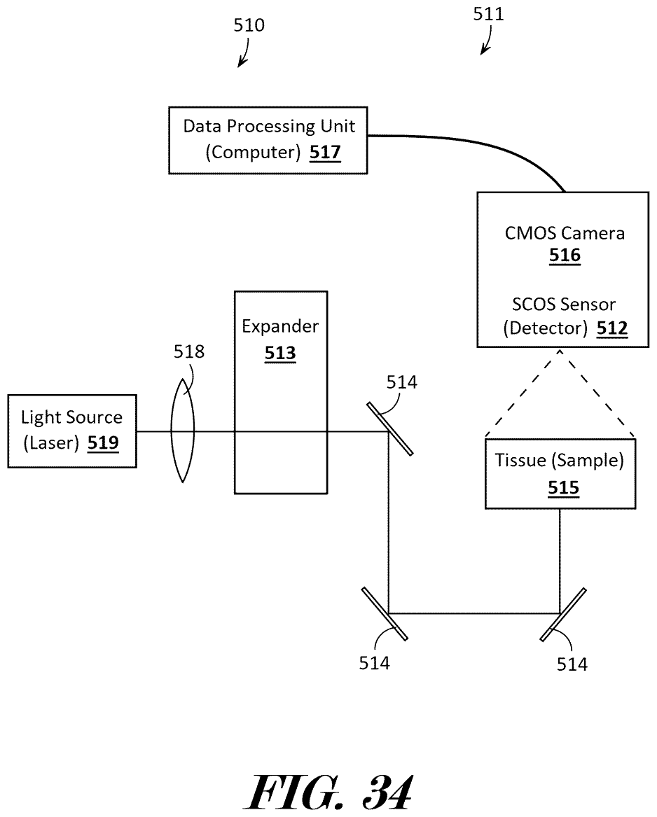

In some embodiments, an automatic detection-therapy system for cardiac arrhythmia 101 may comprise a cardiovascular monitoring apparatus 600 having an auricular ECG monitoring system 100 and a cephalic blood flow monitoring system 530. The cephalic blood flow monitoring system 530 may comprise an auricular speckle contrast optical spectroscopy (SCOS) unit 511. There are a few other laser speckle blood flow devices that can be used as alternatives. Speckle-plethysmography (SPG) or photoplethysmography (PPG) could be other alternative (SPG and PPG can monitor regional blood flow and blood pressure). In preferred embodiments, the cephalic blood flow monitoring system 530 may comprise an auricular Speckle Contrast Optical Spectroscope unit (auricular SCOS unit) 511 by placing a SCOS unit 510 in an auricular housing 11. The auricular SCOS sensor 512 is located on a surface of a wearer's tragus-concha bowl 957 or the wearer's external ear canal 904 when in use to collect the wearer's cerebral (intracranial) and extracranial blood flow data. The cardiovascular monitoring apparatus 600 is configured to simultaneously record the wearer's ECG data (via the auricular ECG monitoring system 100) and the wearer's cerebral and extracranial blood flow data (via the cephalic blood flow monitoring system 530). Referring to FIG. 34, a SCOS unit 510 or an auricular SCOS unit 511 comprises a light source (laser) 519, usually infrared laser diodes or near-infrared (NIR) laser diodes to ensure deep tissue penetration. They most commonly operating at wavelengths of 785 nm, 830 nm or 685 nm. These lasers often operate in continuous-wave (CW) mode with low power, focusing on detecting speckle pattern intensity fluctuations to measure blood flow. This type of laser light is effective because it can penetrate the skull and the brain, allowing for the measurement of blood flow and volume by analyzing the patterns of scattered light. The infrared light produces a back-scattered speckle pattern that varies with changes in blood flow and tissue oxygenation that passes through a focusing lens 518 and an expander 513 and mirrors 514 and then tissue (or sample) 515. The light is then collected by a SCOS sensor 512 that is integrated with a CMOS camera 516 and the data are transmitted to a data processing unit (computer) 517 for analysis and to get the blood flow data at different depths of the tissue. The auricular SCOS sensor 512 is configured to be located on a surface of a wearer's tragus-concha bowl 957 or the wearer's external ear canal 904 to collect the wearer's cerebral (intracranial) and extracranial blood flow data. There are two main types of SCOS designs: fiber-less (compact) design and fiber design. In fiber-less design, the CMOS camera sensor (or detector) is placed in direct contact with the user's skin (or scalp), whereas in fiber-based design, the sensor is remote and only the fiber optic bundle contacts the user's skin (or scalp). In some SCOS setups, there is usually an air gap (for example, about 6 mm) between the laser and the skin to achieve the desired illumination spot size and to maintain safety standards. The fiber-less design is usually quite compact and wearable as compared with the traditional fiber optic cables which are bulky. (While the camera-based approach uses an air gap, a wearable fiber-less optical sensor for deep tissue measurement may be designed for direct contact or very minimal space between the sensor and the skin.) Fiber-less SCOS unit 511, with or without a small air gap, may be preferred for this invention.

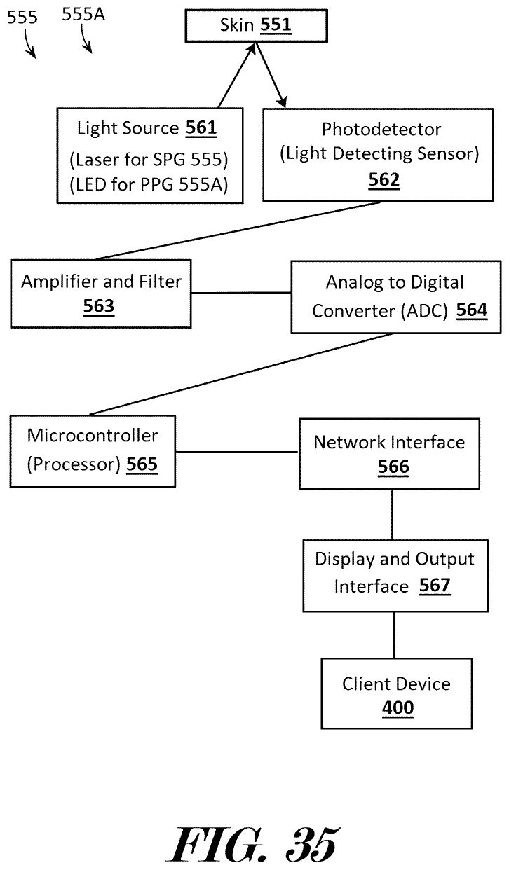

In some embodiments, a cephalic blood flow monitoring system 530 may include at least one of: a speckle-plethysmography (SPG) unit 555 and a photoplethysmography (PPG) unit 555A. Referring to FIG. 35, a SPG unit 555 (or a PPG unit 555A) typically includes the following components: a light source 561, a photodetector 562, amplifier and filter 563, analog-to-digital converter (ADC) 564, a microcontroller/processor 565, a network interface 566 and a display 567. For the SPG unit 555, the light source 561 is usually a laser light to illuminate the skin 551. When SPG 555 is used to monitor regional blood flow and systolic and diastolic blood pressure, the commonly used laser light may be near-infrared (NIR) (approximately 780 nm-1064 nm), particularly 808 nm-810 nm, for deep tissue penetration to monitor blood flow and blood pressure. For the PPG unit 555A, the light source 561 is usually a LED that emit lights at a specific wavelength, commonly around 940 nm, to illuminate the skin 551. The photodetector (or light detecting sensor) 562 can detect the light reflected from the skin 551 and can capture the changes in blood volume. The photodetector 562 then transmits the signals to an amplifier and filter 563 for conditioning of the signals to enhance the quality of the SPG (or PPG) signals. These signals are then transmitted to an analog-to-digital converter (ADC) 564. The ADC 564 can convert the analog signals from the photodetector 562 into digital form for processing. The ADC 564 is in electronic communication with a microcontroller (processor) 565 that can process the digital signals to extract relevant information (such as blood flow metrics, heart rate and blood pressure, etc.) The network interface 566 is in electronic communication with a display and output interface 567 which then generate a display to a user (such as a display on a client device 400).

In some embodiments, an automatic detection-therapy system for cardiac arrhythmia 101 may comprise a cardiovascular monitoring apparatus 600 having an auricular ECG monitoring system 100 and a cephalic blood flow monitoring system 530. The cephalic blood flow monitoring system 530 may further comprise an auricular electroencephalogram (EEG) monitoring system 170. (The EEG monitoring system 170 will also be a useful addition to provide input data for closed-loop control system 333). The auricular EEG monitoring system comprises an EEG recording module 71 in electronic communication with a plurality (a least two, but preferably more than 2) of EEG sensor electrodes 72, 73, 74, 75, 82, 83. The EEG sensor electrodes are configured to be located in an auricular housing 11, to be placed in an ear 902 of a wearer 900, such as in the external ear canal 904 or tragus-concha bowl 957 of the wearer's ear 902 or on the mastoid area of the wearer's peri-auricular area 903. The EEG recording module 71 is configured to record EEG data of the wearer 900. The EEG recording module 71 is in electronic communication with the processing unit 50, 401. The processing unit 50, 401, is configured to convert the raw EEG data into quantitative EEG (qEEG) data, using specialized software to apply mathematical algorithms to digitize, segment, and analyze the EEG data and transforms raw voltage-time signals into numerical data.

In some embodiments, the neuromodulation system 300 may be controlled or regulated by a closed-loop control system 333. A closed-loop control system 333 is located throughout the entire process it regulates. Generally, a closed-loop control system 333 consists of a controller, one or more sensors and one or more actuators, connected by a feedback loop to automatically adjust the operations. The controller of the closed-loop control system 333 is generally within the processor 51 of a processing unit 50 or a processor 402 of a processing unit 401 of a client device 400. The sensors consist of at least one of the following: an ECG monitoring system 100, an auricular SCOS unit 511, an EEG monitoring system 170 and at least one of: a SPG unit 555 and a PPG unit 555A. The actuators consist of at least one of the following: a taVNS unit, a supraorbital nerve stimulation unit 301, an auriculotemporal nerve stimulation unit 302, an occipital nerve stimulation unit 303, a greater auricular nerve stimulation unit 304, an infraorbital nerve stimulation unit 305 and a median nerve stimulation unit 306. The closed-loop control system 333 is built in the processing unit 50, 401. The closed-loop control system 333 and the processing unit 50, 401, are in electronic communication with each component of the cardiovascular monitoring apparatus 600 (comprising the auricular ECG monitoring system 100 and at least one of: the SCOS unit 511, the EEG monitoring system 170, the SPG unit 555 and the PPG unit 555A) and each component of the neuromodulation system 300 (comprising a taVNS unit 30, a supraorbital nerve stimulation unit 301, an auriculotemporal nerve stimulation unit 302, an occipital nerve stimulation unit 303, a greater auricular nerve stimulation unit 304, an infraorbital nerve stimulation unit 305 and a median nerve stimulation unit 306.) The closed-loop control system 333 continuously monitors cardiac status through pertinent input data and feedback data including: ECG data (from the auricular ECG monitoring system 100) and at least one of the following: cerebral blood flow data (from the auricular SCOS unit 511), regional blood flow data and blood pressure data (from at least one of: the SPG unit 555 and the PPG unit 555a) and EEG and qEEG data (from the auricular EEG monitoring system 170), using algorithms (known in the art) to automatically adjust the neuromodulation stimulation in real time based on the aforementioned input data and feedback data in real time. When a closed loop-control system 333 is utilized, the stimulation parameters will be automatically adjusted in response to the real-time data from the cardiovascular monitoring apparatus 600. Instead of using a particular set of pre-determined stimulation parameters (open-loop), in closed-loop control system 333, the stimulation parameters are constantly adjusted in response to the aforementioned data. As an example, when using the taVNS unit 30 for neuromodulation, the goal for the closed-loop control system 333 is to applying low-level stimulation to inhibit atrial fibrillation (AFib), ventricular arrhythmias and other types of cardiac arrhythmias, with the stimulation parameters adjusted to about 50% below the threshold for slowing the heart rate, and reducing arrhythmia inducibility through parasympathetic activation. The primary method to suppress atrial fibrillation (AFib) is low-level stimulation, which suppresses left stellate ganglion (LSG) activity, inhibits atrial ganglionated plexi (GPs), and reduces arrhythmia duration. The effective stimulation frequency commonly used is in the range of 1-30 Hz, often targeting 20 Hz to modulate neural activity. For the stimulation amplitude, to avoid unwanted bradycardia, taVNS intensity is often set just below the threshold required to slow the heart rate. The closed-loop control system 333 may use 12 second “on”/15 seconds “off” cycles in response to arrhythmic events. Both continuous (e.g., 30 s on/30 s off) and “burst” modes are used. A modern, effective approach for AFib is low-level stimulation at 1 Hz, 1 ms pulse width, at 1.5-2 mA, which has shown to reduce AFib burden. The stimulation intensity is adjusted by gradually increasing frequency (often 1-30 Hz) to increase neural activity or by using specific waveforms to manage heart rate. Vagus nerve stimulation by taVNS can increase parasympathetic activity, releasing acetylcholine to reduce heart rate and suppress irregular rhythms. However, in rare situations, when there is a need to reduce the parasympathetic activities (vagal activities), the closed-loop control system 333 can use High-Frequency Alternating Current (KHFAC) to block action potentials and reduce the vagal activities when necessary. R-wave synchronization can also be utilized to further improve the effectiveness and further decrease the side effects.

In some embodiments, an auriculotemporal nerve stimulation unit 302 of a neuromodulation system 300 may be in electronic communication with a closed-loop control system 333 and a processing unit 50, 401. The closed-loop control system 333 and the processing unit 50, 401, are also in electronic communication with the cardiovascular monitoring apparatus 600 and receives real-time input data, including ECG data (from the auricular ECG monitoring system 100) and at least one of: cerebral blood flow data (from the auricular SCOS unit 511), regional blood flow data, systolic blood pressure data and diastolic blood pressure data (from at least one of: the SPG unit 555 and the PPG unit 555A). Additionally, the cardiovascular monitoring apparatus 600 may comprise an auricular EEG monitoring system 170 and the closed-loop control system 333 may also receive input qEEG data. The closed-loop control system 333 and the processing unit 50, 301, are configured to analyze the real time ECG data, cerebral blood flow data, regional blood flow data, systolic and diastolic blood pressure data and EEG/qEEG data to automatically and immediately adjust the stimulation parameters (e.g. timing, intensity, frequency, waveform, cycle, duration etc.). This closed-loop control system 333 is an improvement over the open-loop, fixed (or pre-determined) stimulation parameters because it is responsive and adaptive to the feedbacks and, thus, can achieve better therapeutic effects while reducing the potential side effects. R-wave synchronization may also be utilized to further improve the effectiveness and further decrease the side effect of neuromodulation from auriculotemporal nerve stimulation unit 302. The closed-loop control system 333 and R-wave synchronization can be similarly applied to other components of the neuromodulation system 300, including a supraorbital nerve stimulation unit 301, an occipital nerve stimulation unit 303, a greater auricular nerve stimulation unit 304, an infraorbital nerve stimulation unit 305 and a median nerve stimulation unit 306.

In some embodiments, an automatic detection-therapy system for cardiac arrhythmia 101 may comprise a trigeminal nerve stimulation unit (including one of: a supraorbital nerve stimulation unit 301, an auriculotemporal nerve stimulation unit 302 and an infraorbital nerve stimulation unit 305) under the regulation or control by a closed-loop control system 333 (frequency, intensity, timing, duration, waveform etc. are regulated by the closed-loop control system 333) to shift the autonomic balance towards parasympathetic dominance (increasing vagal tone) without inducing significant side effects or bradycardia. The goal is to apply low-level stimulation that acts through the brainstem to modulate cardiac autonomic nervous system activity, often targeting to reduce arrhythmia burden, such as in atrial fibrillation (AFib). The stimulation intensity may be tailored to the individual's comfort, typically starting at 0 mA and increasing until the patient reports a mild tingling sensation, then reducing it slightly to avoid pain. The intensity is often set slightly below the threshold of sensory discomfort, commonly in the range of 2-16 mA, depending on the device. Lower frequencies (1-20 Hz) are generally used to induce parasympathetic, calming effects, such as increasing heart rate variability (HRV). Studies have shown that high frequencies (120-200 Hz) can induce the most pronounced, though sometimes temporary, decrease in heart rate, while 20 Hz stimulation may produce more rapid adaptation (habituation). In trigeminal nerve stimulation, higher frequencies (e.g., 200 Hz) stimulation often produce more pronounced reductions in heart rate and blood pressure compared to low frequencies (e.g., 2 Hz) stimulation, with 200 Hz triggering a greater autonomic response. To avoid habituation (where the body stops responding to the stimulation), continuous stimulation should be avoided. A commonly used protocol is 30 seconds “on”/30 seconds “off”. The closed-loop control system 333 is particularly important to avoid the risk of trigeminal cardiac reflex (TCR). TCR is a brainstem reflex that can cause abrupt bradycardia or even asystole. If this occurs, the closed-loop control system 333 is configured to automatically reduce the stimulation intensity immediately. For acute arrhythmia suppression, lower-intensity, low-level stimulation is preferred to avoid the pro-arrhythmic effects of high-intensity nerve stimulation.

In some embodiments, an automatic detection-therapy system for cardiac arrhythmia 101 may comprise one of: an occipital nerve stimulation unit 303, a greater auricular nerve stimulation unit 305 and a median nerve stimulation unit 306, and under the regulation or control by a closed-loop control system 333. The setups of the closed-loop control system 333 for occipital nerve stimulation unit 303 or greater auricular nerve stimulation unit 304 or median nerve stimulation unit 306 are similar to the aforementioned descriptions for the auriculotemporal nerve stimulation unit 302. With constant feedback inputs, the closed-loop control system 333 will automatically adjust the stimulation parameters until the auricular ECG monitoring system 100 showing the heart rate and heart rhythm reach target range.

R-wave synchronization is essential for electric cardioversion of atrial fibrillation (electric cardioversion means: using a synchronized, low-energy electrical stimulation to reset an irregular heart rhythm, e.g. atrial fibrillation, back to a normal cardiac rhythm). During R-wave synchronization, the electric stimulation is delivered during the R-wave on the ECG. For taVNS unit 30, R-wave synchronization has been shown to be more effective and more precise than continuous non-gated stimulation. The purpose of R-wave synchronization is to avoid giving electric stimulation during T-wave. (T-wave is “vulnerable period” because stimulation during T-wave might induce dangerous ventricular fibrillation if the stimulation is given directly to the heart during cardioversion.) R-wave synchronization may also be used in trigeminal nerve stimulation and other neuromodulation (e.g. occipital nerve stimulation unit 303 or greater auricular nerve stimulation unit 304 or median nerve stimulation unit 306). Synchronization to other physiological markers, like respiration (respiratory-gated) may also enhance effectiveness.

R-wave synchronization is a type of closed-loop control that aligns stimulation with the cardiac cycle to improve AFib management. R-wave gating allows stimulation during specific phases of the cardiac cycle (e.g., during the ventricular systole or diastole) to ensure maximum impact on atrial electrical remodeling. Systole-gated taVNS, in particular, has been shown to produce more profound changes in RR interval modulation (a proxy for vagal activity) compared to non-synchronized approaches. Low-level stimulation synchronized with specific phases of the heartbeat has been found to lengthen the atrial effective refractory period, which helps prevent the induction of AFib. R-wave synchronization increases the effectiveness of taVNS for atrial fibrillation. Studies using auricular vagus nerve stimulation synchronized with the R-wave have shown a significant reduction in the ventricular rate (up to 40% increase in ventricular interval) during AFib. Synchronized stimulation allows for precise delivery of the stimulation right after the R-wave, maximizing the impact on the atrioventricular node and inhibiting excessive ventricular conduction. Synchronization also improves safety. Synchronization significantly reduces the risk of inducing ventricular fibrillation by ensuring stimulation does not occur during the relative refractory period. Synchronized stimulation allows for precise delivery of the stimulation right after the R-wave, maximizing the impact on the atrioventricular node and inhibiting excessive ventricular conduction. In some embodiments, an automatic detection-therapy system for cardiac arrhythmia 101 comprises a processing unit 50, 401, that is configured to incorporate R-wave synchronization into the closed-loop control system 333 to precisely time the actuation of neuromodulating electric stimulation by the neuromodulation system 300. Please note that closed-loop control system 333 and R-wave synchronization are automatically and instantly available for neuromodulating therapy in this invention due to the novel integration of the auricular ECG monitoring system 100, blood flow and blood pressure monitors (via at least one of: SCOS 511, SPG 555 and PPG 555A) and the neuromodulation system 300 into a single integrated system (system 101).

In some embodiments, an auricular ECG monitoring system 100 may comprise an auricular housing 11 selected from one of the followings: a modified earbud housing 61, a modified in-the-ear housing 62, a behind-the-ear-hearing-aid-style housing 63 and a tubular-shaped structure 66. As used herein, the term “auricular housing 11” refers to housing structures in or around the ear. As used herein, the term “tragus-concha bowl” 957 refers to an area surrounding the opening of the external ear canal 904 and this area is bounded by inner tragus 905 and concha 906, 907, forming a bowl-shaped cavity to funnel sound into the external ear canal 904. This bowl-shaped cavity is called tragus-concha bowl 957 herein (official name is concha auriculae and is sometimes referred to as the concha bowl or concha auricularis).

In preferred embodiments, an automatic detection-therapy system for cardiac arrhythmia 101 may have an auricular housing 11 that may be configured as a modified earbud housing 61. The modified earbud housing 61 includes a tubular-body portion 25 and a stem portion 68. (FIGS. 1, 2). The stem portion 68 is equivalent to a “stem” (or a “stalk”) of an earbud. The tubular-body portion 25 includes a tubular-shaped structure 66 (to be placed inside a wearer's external ear canal 904 when in use) and a body-structure 67 (to be placed immediately at the opening of the wearer's external ear canal 904 and be placed inside the tragus-concha bowl 957 when in use). The tubular-shaped structure 66 is equivalent to a modified elongated version of an “ear-tip” and a “nozzle” of an earbud. The body-structure 67 is equivalent to a “body” (or a “shell”) of an earbud. Similarly, a modified in-the-ear housing 62 (modified from an in-the-ear hearing aid or ITE, modified by elongating its “ear-tip” and “nozzle”) also includes a tubular-shaped structure 66 and a body-structure 67. The tubular-shaped structure 66 is configured to be inserted into a wearer's external ear canal 904 when in use, while the body-structure 67 is configured to be placed at immediate opening of the wearer's external ear canal 904 and be placed inside the tragus-concha bowl 957 of the wearer's ear 902 when in use. Likewise, a behind-the-ear-hearing-aid-style housing 63 includes an in-the-ear portion 27 and a behind-the-ear portion 26. The in-the-ear portion 27 (of a behind-the-ear-hearing-aid-style housing 63) also includes a tubular-shaped structure 66 (to be placed inside the wearer's external ear canal 904 when in use) and a body-structure 67 (to be placed at the opening of the wearer's external ear canal 904 and be placed inside the tragus-concha bowl 905 of the wearer's ear 902 when in use.)

From the above description, it is obvious that these auricular housings 11 (such as the modified earbud housing 61, the modified in-the-ear housing 62 and the behind-the-ear-hearing-aid style housing 63) all include a tubular-shaped structure 66 and a body-structure 67. In some embodiments for an automatic detection-therapy system for cardiac arrhythmia 101, the ECG recording module 20, the auricular SCOS unit 511, at least one of: the SPG unit 555 and the PPG unit 555A, the taVNS unit 30, the auriculotemporal nerve stimulation unit 302, the greater auricular nerve stimulation unit 304 and the processing unit 50, 401, may be housed together as a single structure and be placed in the external ear canal 904, tragus 905, concha 906, 907, and/or peri-auricular area 903. Alternatively, the taVNS unit 30, the auricular SCOS unit 511, the SPG unit 555 (or the PPG unit 555A), the auriculotemporal nerve stimulation unit 302, the greater auricular nerve stimulation unit 304, and the ECG recording module 20 may be in separate housings and may be located adjacent to each other with each of them attached to one or more of the following: the external ear canal 904, the tragus-concha bowl 957, and the peri-auricular area 903. The taVNS stimulating electrode 31, the auriculotemporal nerve stimulating electrode 312 and the greater auricular nerve stimulating electrode 314 may be housed in a same auricular housing 11 due to proximity of their respective innervation areas in the ear 902. (For comparison: The vagus-innervated auricular skin includes: inner posterior portion of tragus 905, cymba-concha 906, cavum-concha 907, posterior and inferior walls of the external ear canal 904 and small adjacent regions of the external ear. The auriculotemporal nerve innervated auricular skin includes: anterior outer part of tragus 905, anterior-superior part of cavum concha 907, anterior and superior walls of the external ear canal 904 and anterior and superior part of pinna including anterior-superior helix 909. The greater auricular nerve innervated auricular skin includes lower part of cavum concha 907, including the inferior/lower portion of cavum concha on both the posterior (outside) and anterior (inside) surfaces, the lobule, posterior helix 909, posterior surface of the auricle, with branches extending to the tail of the helix 909 and antitragus.)

In some embodiments, all of the ECG sensor electrodes 12, 13, the auricular SCOS sensor 512, at least one of: the SPG unit 555 and the PPG unit 555A, the taVNS stimulating electrode 31, the auriculotemporal nerve stimulating electrode 312 and the greater auricular nerve stimulating electrode 314 may be all housed together sharing a same auricular housing 11. The auricular housing 11 may be selected from one of the following: a modified earbud housing 61, a modified in-the-ear housing 62, and a behind-the-ear-hearing-aid-style housing 63, as described hereinbefore. All of these housings 61, 62, 63, include a tubular-shaped structure 66 (configured to be inserted into an external ear canal 904 of a wearer when in use) and a body-structure 67 (configured to be placed at the opening of the external ear canal 904 and to sit or be placed inside a tragus-concha bowl 957 of the wearer's ear when in use). At least one of the ECG sensor electrodes 12, 13, (for example the first ECG sensor electrode 12) may be configured to be located on a surface 91 of the tubular-shaped structure 66, while the auricular SCOS sensor 512, at least one of: the SPG unit 555 and the PPG unit 555A, the taVNS stimulating electrode 31 and the auriculotemporal nerve stimulating electrode 312 are configured to be located on a surface of one of: the tubular-shaped structure 66 and the body-structure 67. The greater auricular nerve (GAN) stimulating electrode 314 and another ECG sensor electrode (for example: the second ECG sensor electrode 13) are configured to be located at a surface of the body-structure 67. The first ECG sensor electrodes 12 is configured to be partially embedded in the surface with slight protrusion at the surface of the tubular-shaped structure 66. The auricular SCOS sensor 512, at least one of: the SPG unit 555 and the PPG unit 555A, the taVNS stimulating electrode 31, the auriculotemporal nerve stimulating electrode 312 are configured to be partially embedded in the surface with slight protrusion at the surface of one of: the tubular-shaped structure 66 and the body-structure 67. (Alternatively, the auricular SCOS sensor 512 may be placed slightly below the surface of the tubular-shaped structure 66 or the body-structure 67 so that there is a small air gap between the SCOS sensor 512 and the wearer's skin when the tubular-shaped structure 66 or the body-structure 67 are placed in the wearer's ear 902.) The greater auricular nerve (GAN) stimulating electrode 514 and the second ECG sensor electrode 13 are configured to be partially embedded in the surface with slight protrusion at the surface of the body-structure 67. The tubular-shaped structure 66 and the body-structure 67 may be configured to comprise a flexible elastic and adaptable material (such as soft foam or soft silicone-type material) and the flexible elastic and adaptable material is configured to have appropriate flexibility, elasticity and adaptability so that the tubular-shaped structure 66 will naturally adapt to the contour of the external ear canal 904 and snugly fill the interior of the wearer's external ear canal 904 when the tubular-shaped structure 66 is inserted into the external ear canal 904 of the wearer's ear 902 and, meanwhile, the body-structure 67 will naturally adapt to the contour of the tragus-concha bowl 957 of the wearer's ear 902 and snugly fill the interior of the tragus-concha bowl 957 of the wearer's ear 902 when the body-structure 67 is placed inside the tragus-concha bowl 957 of the wearer's ear 902, so that the first ECG sensor electrodes 12 is naturally snugly in contact with the skin of the external ear canal 904 of the wearer's ear 902, and, at the same time, the auricular SCOS sensor 512, at least one of: the SPG unit 555 and the PPG unit 555A, the taVNS stimulating electrode 31 and the auriculotemporal nerve stimulating electrode 312 are naturally and snugly in contact with the skin of the tragus-concha bowl 957 of the wearer's ear 902 or the skin of the external ear canal 904 of the wearer's ear 902, while the GAN stimulating electrode 314 and the second ECG sensor electrode 13 are naturally snugly in contact with the skin of the wearer's tragus-concha bowl 957, and so that the taVNS stimulating electrode 31 is naturally and snugly in contact with its target skin of vagus-innervated auricular skin of the wearer's first ear 902, provided by carefully selecting a location for the taVNS stimulating electrode 31 on the tubular-shaped structure 66 or the body-structure 67 to match one of the innervation locations of the vagus innervated skin on one of: the external ear canal 904 and the tragus-concha bowl 957, and, at the same time, the auriculotemporal nerves stimulating electrode 312 will be naturally in close contact with its target skin of auriculotemporal nerve innervated auricular skin, provided by carefully selecting a location for the auriculotemporal nerve stimulating electrode 312 on the tubular-shaped structure 66 or the body-structure 67 to match one of the innervation locations of the auriculotemporal nerve innervated skin on the external ear canal 904 or the tragus-concha bowl 957, and, at the same time, the greater auricular nerve stimulating electrode 314 will be naturally in close contact with greater auricular nerve innervated auricular skin, provided by carefully selecting a location for the greater auricular nerve stimulating electrode 314 on the body-structure 67 to match one of the innervation locations of the greater auricular nerve innervated skin on tragus-concha bowl 957. As described hereinbefore, by comparing the innervation patterns, it is obvious that the tragus-concha 957 of a wearer's ear 902 receives mixed and overlapped innervation from the auricular branch of vagus nerve, the auriculotemporal nerve and the greater auricular nerve. The external ear canal 904 receives mixed innervation from the auricular branch of vagus nerve and the auriculotemporal nerve.

Generally, an elastic flexible and adaptable material may be flexible to allow slight deformation and optionally resilient so as to return to its original shape after deformation. In preferred embodiments, all or portions of a modified earbud housing 61 (e.g., a tubular-body portion 25), all or portions of a modified in-the-ear housing 62, all or portions of a behind-the-ear-hearing-aid-style housing 63 (e.g., in-the-ear portion 27), all or portions of the tubular-shaped structure 66 may be made from or comprise an elastic flexible and adaptable material such as natural and/or synthetic rubber material such as latex rubber, silicone foam, silicone rubber or polysiloxanes, rubber foam, urethane foam, plastic foam, neoprene foam, latex foam rubber, polyurethane foam rubber, forms of the organic compound isoprene, Polyacrylate Rubber, Ethylene-acrylate Rubber, Polyester Urethane, flexible plastics, such as high-density polyethylene (HDPE), polyvinyl chloride (PVC), polypropylene (PP), Polycarbonate (PC), low density polyethylene (LDPE), Polystyrene (PS), etc. including combinations of materials.

It should be noted that the at least two ECG sensor electrodes 12, 13, may both be located in the tubular-shaped structure 66 or both be located in the body-structure 67. However, it would be better for the at least two ECG sensor electrodes 12, 13, to be more separated from each other (for example the first ECG sensor electrode 12 be placed on the tubular-shaped structure 66 and to be in the external ear canal 904 when in use while the second ECG sensor electrode 13 to be placed in the body-structure 67 and to be in the tragus-concha bowl 957 when in use.) More separation between the first and the second ECG sensor electrodes will produce better and stronger ECG signals.

In some embodiments, a neuromodulation system 300 may include 3 components: a taVNS unit 30 having a taVNS stimulating electrode 31, an auriculotemporal nerve (ATN) stimulation unit 302 having an ATN stimulating electrode 312 and a greater auricular nerve (GAN) stimulation unit 304 having a GAN stimulating electrode 314 and all of these electrodes (31, 312, 314) may be located on the body-structure 67. By carefully selecting the location for the taVNS stimulating electrode 31 on the body-structure 67 to match the innervation locations of the vagus innervated skin on the tragus-concha bowl 957, the taVNS stimulating electrode 31 will automatically get in close contact with its target skin on the tragus-concha bowl 957 when the body-structure 67 is placed in the tragus-concha bowl 957. By carefully selecting the location for the ATN stimulating electrode 312 on the body-structure 67 to match the innervation locations of the ATN on the tragus-concha bowl 957, the ATN stimulating electrode 312 will automatically get in close contact with its target skin on the tragus-concha bowl 957 when the body-structure 67 is placed in the tragus-concha bowl 957. Similarly, by carefully selecting the location for the GAN stimulating electrode 314, on the body-structure 67 to match the innervation locations of the GAN innervated skin on the tragus-concha bowl 957, the GAN stimulating electrode 314 will automatically get in close contact with its target skin on the tragus-concha bowl 957 when the body-structure 67 is placed in the tragus-concha bowl 957. Alternatively, the taVNS stimulating electrode 31 and the ATN stimulating electrode 312 may be located on the tubular-shaped structure 66. By carefully selecting the location for the taVNS stimulating electrode 31 on the tubular-shaped structure 66 to match its target skin locations on the external ear canal 904, the taVNS stimulating electrode 31 will be automatically get in close contact with its target skin in the external ear canal 904. Similarly, by carefully selecting the location for the ATN stimulating electrode 312 on the tubular-shaped structure 66 to match its target skin locations on the external ear canal 904, the ATN stimulating electrode 312 will be automatically in close contact with its target skin in the external ear canal 904.

In some embodiments of the auricular ECG monitoring system 100 and the automatic detection-therapy system for cardiac arrhythmia 101, the first ECG sensor electrode 12 and the second ECG sensor electrode 13 may be configured as wireless ECG electrodes and the ECG recording module 20 may be configured to comprise a wireless ECG amplifier 21 to allow the first wireless ECG sensor electrode 12 and the second wireless ECG sensor electrode 13 to be housed in the first auricular housing 11 while the ECG recording module 20 and the processing unit 50, 401 may be housed in one of the following: a wearable watch-type client device 400 and a portable smart-phone-type client device 400. The automatic detection-therapy system for cardiac arrhythmia 101 may further comprise a network interface 53, 406, and at least one of the following: a speaker 15, 404A, and a vibrator 17, 404B, on a client device 400 of the wearer 900. The network interface 53, 406, is in electronic communication with the processing unit 50, 401, the speaker 15, 404A and the vibrator 17, 404B, on a client device 400 of the wearer 900. The processing unit 50, 401, is in electronic communication with the auricular ECG monitoring system 100, the cephalic blood flow monitoring system 530 (including the auricular SCOS unit 511 and at least one of: the SPG unit 555 and the PPG unit 555A). The network interface 53, 406 is configured to generate a visible notification to a display screen on a client device 400 of the wearer 900 and/or a client device 400 of the wearer's healthcare provider 950 describing the wearer's electrocardiographic profile and the cerebral blood flow and blood pressure status. The network interface 53, 406, is further configured to generate an audible notification to the speaker 15, 404A, when the processing unit 50, 401, detects at least one of: presence of a serious cardiac arrhythmia, presence of an impending serious cardiac arrhythmia, presence of a decrease of cerebral blood flow more than a pre-determined percentage (e.g. a decrease of 30% or more), presence of a decrease of regional blood flow more than a predetermined percentage (e.g. a decrease of 30% or more), presence of a decrease of systolic blood pressure more than a pre-determined amount (e.g. a decrease of 20 mmHg or more) and presence of a decrease of diastolic blood pressure more than a predetermined amount (e.g. a decrease of 20 mmHg or more). The network interface 53, 406, is also configured to generate a tactile notification to the vibrator 17, 404B, when the processing unit 50, 401, detects at least one of the following: presence of a serious cardiac arrhythmia, presence of an impending serious cardiac arrhythmia, presence of a decrease of cerebral blood flow more than a predetermined percentage (e.g. a decrease of 30% or more), presence of a decrease of regional blood flow more than a predetermined percentage (e.g. a decrease of 30% or more), presence of a decrease of systolic blood pressure more than a predetermined amount (e.g. a decrease of 20 mmHg or more), and presence of a decrease of diastolic blood pressure more than a predetermined amount (e.g. a decrease of 20 mmHg or more).

In some embodiments, an automatic detection-therapy system for cardiac arrhythmia 101 may comprise an auricular ECG monitoring system 100 having a first ECG sensor electrode located in a first ear 902 of a wearer 900 or a periauricular area 903 around the first ear, and a second ECG sensor electrode 13 located in a second ear 902 of the wearer 900 or in a peri-auricular area 903 around the wearer's second ear 902. The first ECG sensor electrode 12, the auricular SCOS unit 511, at least one of: the SPG unit 555 and the PPG unit 555A, and each component of the neuromodulation unit 300 may be housed in a first auricular housing 11 to be placed in the wearer's first ear, while the second ECG sensor electrode 13 may be housed in a second auricular housing 11 to be placed in the wearer's second ear. The setups and the material for the second auricular housing 11 are essentially the same as the aforementioned descriptions for the first auricular housing 11. In some embodiments, an automatic detection-therapy system for cardiac arrhythmia 101 may comprise two neuromodulation systems 300. The first neuromodulation system 300 may include at least one of the following components: a first taVNS unit 30, a first auriculotemporal nerve stimulation unit 302 and a first greater auricular nerve stimulation unit 304 and these are configured to be housed in a first auricular housing to be placed in the wearer's first ear. The first neuromodulation system 300 may further include at least one of: a supraorbital nerve stimulation unit 301, an occipital nerve stimulation unit 303, an infraorbital nerve stimulation unit 305 and a median nerve stimulation unit 306. The second neuromodulation system 300 may include at least one of the following: a second taVNS unit 30, a second auriculotemporal nerve stimulation unit 302 and a second greater auricular nerve stimulation unit 304 and these are configured to be housed in a second auricular housing to be placed in the wearer's second ear. The setups and the functions of the second neuromodulation system 300 are essentially the same as the first neuromodulation system 300 as described hereinbefore. It has been shown that stimulation of more than one component of the neuromodulation system 300 has stronger effect due to the synergistic effect. It has also been shown that bilateral neuromodulation stimulation to a component of the neuromodulation unit 300 is more effective than unilateral stimulation.

In alternative embodiments, an automatic detection-therapy system for cardiac arrhythmia 101 may further comprise an auricular electroencephalogram (EEG) monitoring system 170 comprising an EEG recording module 71 and a plurality (at least two, but preferably more than two) of EEG sensor electrodes 72, 73, 74, 75, 82, 83. The EEG sensor electrodes 72, 73, 74, 75, 82, 83, are configured to be located on a surface 91 of an auricular housing 11. Each of the EEG sensor electrode 72, 73, 74, 75, 82, 83, is configured to be in electronic communication with the EEG recording module 71. The EEG recording module 71 is configured to record EEG data of the wearer 900. The EEG recording module 71 is in electronic communication with a processing unit 50, 401. The processing unit 50, 401, is configured to analyze the EEG data recorded by the EEG recording module 71 to assess the wearer's EEG status and to detect any sudden slowing of the wearer's EEG (sudden increase of theta and delta waves). The processing unit 50, 401, is further configured to convert the raw EEG data recorded by the EEG recording module 71 into quantitative EEG (qEEG) data, including relative delta power, delta/alpha ratio, cordance z-score and other quantitative EEG metrics.

In alternative embodiments, an automatic detection-therapy system for cardiac arrhythmia 101, may comprise a cardiovascular monitoring apparatus 600, a neuromodulation system 300, a network interface 53 and a processing unit 50, 401. The cardiovascular monitoring apparatus 600 may comprise an auricular ECG monitoring system 100, an auricular EEG monitoring system 170 and a cephalic blood flow monitoring system 530. The auricular ECG monitoring system 100 includes an ECG recording module 20 having at least two wired or wireless ECG sensor electrodes 12, 13 (such as a first ECG sensor electrode 12 and a second ECG sensor electrode 13). The auricular EEG monitoring system 170 includes an EEG recording module 71 having a plurality of wired or wireless EEG sensor electrodes 72, 73, 74, 75, 82, 83. The cephalic blood flow monitoring system 530 may include an auricular speckle contrast optical spectroscopy unit (auricular SCOS unit) 511 and at least one of: a speckle-plethysmography (SPG) unit 555 and a photoplethysmography (PPG) unit 555A. The auricular SCOS unit 511 comprises an auricular SCOS sensor 512. The neuromodulation system 300 may include at least one of the following: a taVNS unit 30 having a taVNS stimulating electrode 31, an auriculotemporal nerve (ATN) stimulation unit 302 having an ATN stimulating electrode 312 and a greater auricular nerve (GAN) stimulation unit 304 having a GAN stimulating electrode 314. In some embodiments, all of the above electrodes and sensors, including the ECG sensor electrodes 12, 13, the EEG sensor electrodes 72, 73, 74, 75, 82, 83, at least one of: the SPG unit 555 and the PPG unit 555A, the auricular SCOS sensor 512, the taVNS stimulating electrode 31, the ATN stimulating electrode 312 and the GAN stimulating electrode 413 may be configured to be housed together in an auricular housing 11 (such as in a modified earbud housing 61 or a modified in-the-ear housing 62 or a behind-the-ear-hearing-aid-style housing 63). The auricular housing 11 may include a tubular-shaped structure 66 (to be inserted into a wearer's external ear canal 904 when in use) and a body-structure 67 (to be placed at immediate opening of the wearer's external ear canal 904 and be placed inside the wearer's tragus-concha bowl 957 when in use.) The tubular-shaped structure 66 may be configured to be made with elastic flexible and adaptable material (such as soft silicone or soft foam), in which the material for the tubular-shaped structure 66 is configured to have appropriate elasticity, flexibility and adaptability so that the tubular-shaped structure 66 will naturally adapt to the contour of the wearer's external ear canal 904 and will naturally fill the interior of the external ear canal 904 when it is inserted into the wearer's external ear canal 904. All of the EEG sensor electrodes 72, 73, 74, 75, 82, 83, and the first ECG sensor electrode 12 may be placed on a surface 91 of the tubular-shaped structure 66. All of the EEG sensor electrodes 72, 73, 74, 75, 82, 83 and the first ECG sensor electrode 12 may be partially embedded in the surface 91 with slight protrusion at the surface 91 of the tubular-shaped structure 66 so that all of the EEG sensor electrodes 72, 73, 74, 75, 82, 83, and the first ECG sensor electrode 12 may be naturally in close contact with the skin of the wearer's external ear canal 904 when the tubular-shaped structure 66 is inserted into the wearer's external ear canal 904. Likewise, the body-structure 67 may be configured to be made with elastic flexible and adaptable material (such as soft silicone or soft foam), in which the material for the body-structure 67 is configured to have appropriate elasticity, flexibility and adaptability so that the body-structure 67 will naturally adapt to the contour of the wearer's tragus-concha bowl 957 and will naturally fill the interior of the tragus-concha bowl 957 when it is placed inside the wearer's tragus-concha bowl 957. The second ECG sensor electrode 13 and the GAN stimulating electrode 314 may be placed on a surface 91 and partially embedded in the surface 91 with slight protrusion at the surface 91 of the body-structure 67 so that the second ECG sensor electrode 13 and the GAN stimulating electrode 314 may be naturally in close contact with the skin of the wearer's tragus-concha bowl 957 when the body-structure 67 is placed inside the wearer's tragus-concha bowl 957. The auricular SCOS sensor 512, at least one of: the SPG unit 555 and the PPG unit 555A, the taVNS stimulating electrode 31 and the auriculotemporal nerve (ATN) stimulating electrode 312 may be configured to be placed on a surface 91 (and partially embedded in the surface with slight protrusion at the surface) of one of: the tubular-shaped structure 66 and the body-structure 67 so that when the tubular-shaped structure 66 is inserted into the wearer's external ear canal 904 and the body-structure 67 is placed inside the tragus-concha bowl 957, the auricular SCOS sensor 512, at least one of: the SPG unit 555 and the PPG unit 555A, the taVNS stimulating electrode 31 and the ATN stimulating electrode 312 will all be naturally in close contact with the skin of the wearer's external ear canal 904 or the skin of the wearer's tragus-concha bowl 957. (Alternatively, the auricular SCOS sensor 512 may be placed slightly below the surface of the body-structure 67 or tubular-shaped structure 66 so that there is a small air gap between the SCOS sensor 512 and the wearer's skin when the body-structure 67 and the tubular-shaped structure 66 are placed in the wearer's ear 902). At the same time, the taVNS stimulating electrode 31 will be naturally in close contact with vagus innervated auricular skin since external ear canal 904 and the tragus-concha bowl 957 are part of vagus innervated auricular skin. Meanwhile, the ATN stimulating electrode 312 will also be naturally in close contact with ATN innervated auricular skin since external ear canal 904 and the tragus-concha bowl 957 are also part of ATN innervated auricular skin. Further, the greater auricular nerve (GAN) stimulating electrode 314 will be naturally in close contact with the GAN innervated auricular skin since cavum concha (part of tragus-concha bowl 957) is part of the GAN innervated auricular skin. Thus, the taVNS stimulating electrode 31 will be naturally in close contact with the vagus innervated auricular skin, provided by carefully selecting a location for the taVNS stimulating electrode 31 on the tubular-shaped structure 66 or the body-structure 67 to match one of the innervation locations of the vagus innervated skin on the external ear canal 904 or the tragus-concha bowl 957, and, at the same time, the ATN stimulating electrode 312 will be naturally in close contact with ATN innervated auricular skin, provided by carefully selecting a location for the ATN stimulating electrode 312 on the tubular-shaped structure 66 or the body-structure 67 to match one of the innervation locations of the ATN innervated skin on the external ear canal 904 or the tragus-concha bowl 957, and, at the same time, the GAN stimulating electrode 314 will be naturally in close contact with GAN innervated auricular skin, provided by carefully selecting a location for the GAN stimulating electrode 314 on the body-structure 67 to match one of the innervation locations of the GAN innervated skin on tragus-concha bowl 957. (This is feasible due to the proximity of the innervation patterns of the auricular branch of vagus nerve, the auriculotemporal nerve and the greater auricular nerve in the auricular area, and by careful selection of the locations for 31, 312, 314, to match the locations of their respective target skin on the wearer's ear, as described hereinbefore.)

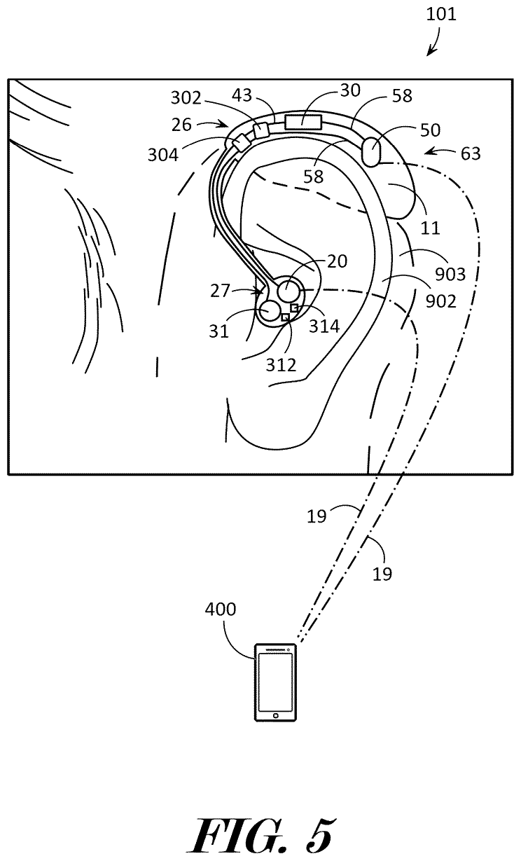

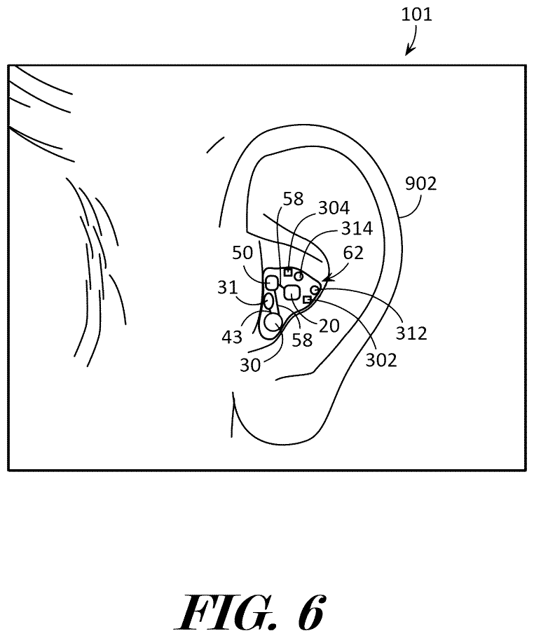

For a wearer 900, installing and removing all of these electrodes 12, 13, 72, 73, 74, 75, 82, 83, 31, 312, 314, 512 and at least one of: SPG unit 555 and PPG unit 555A will be as easy as placing and removing the tubular-shaped structure 66 and the body-structure 67 from the wearer's ear 902. There will be no need for a certified technologist to install all of these electrodes 12, 13, 72, 73, 74, 75, 82, 83, 31, 312, 314, 512 and at least one of: SPG unit 555 and PPG unit 555A. Applying adhesive material to secure these electrodes 12, 13, 72, 73, 74, 75, 82, 83, 31, 312, 314, 512 and at least one of: SPG unit and PPG unit 555A will also be unneeded. Thus, this automatic detection-therapy system for cardiac arrhythmia 101 is fully wearable, user-installable, user-removable, ambulatory and very convenient for wearers (users). The auricular housing 11 may be selected from one of the following: a modified earbud housing 61, a modified in-the-ear housing 62, a behind-the-ear-hearing-aid-style housing 63 and a tubular-shaped structure 66. These auricular housings 11 are inherently stable and suitable for long-term attachment. They can be easily removed temporarily to charge the power sources 16 (batteries). They can also be easily put back into place. Since cardiac arrhythmia is usually a long-lasting or permanent problem, a long-term (months or years) way to securely attach a cardiovascular monitoring apparatus 600 (including an auricular ECG monitoring system 100, and at least one of: a SCOS unit 511, a SPG unit 555, a PPG unit 555A, and an auricular EEG monitoring system 170) and a neuromodulation system 300 to a wearer 900 is greatly needed. No surgery would be needed. Nowadays, earphones, air-pods and earbuds have become quite popular. They are nice-looking and well accepted by most people. (FIGS. 2, 5, 6, 7, 11, 12, 13). These housing structures enable convenient long-term monitoring and automatic therapeutic intervention of the cardiac arrhythmias when cardiac arrhythmia is detected. The auricular housing 11 design will make all of the following: ECG sensor electrodes 12, 13, EEG sensor electrodes 72, 73, 74, 75, 82, 83, auricular SCOS sensor 512, at least one of: SPG unit 555 and PPG unit 555A, taVNS stimulating electrode 31, auriculotemporal nerve stimulating electrode 312 and greater auricular nerve stimulating electrode 314 be user-installable, user-removable and the automatic detection-therapy system for cardiac arrhythmia 101 is fully wearable and freely ambulatory for long-term use.

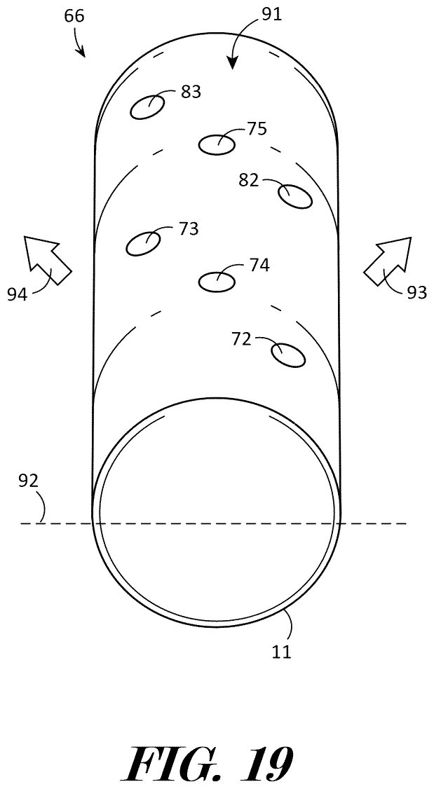

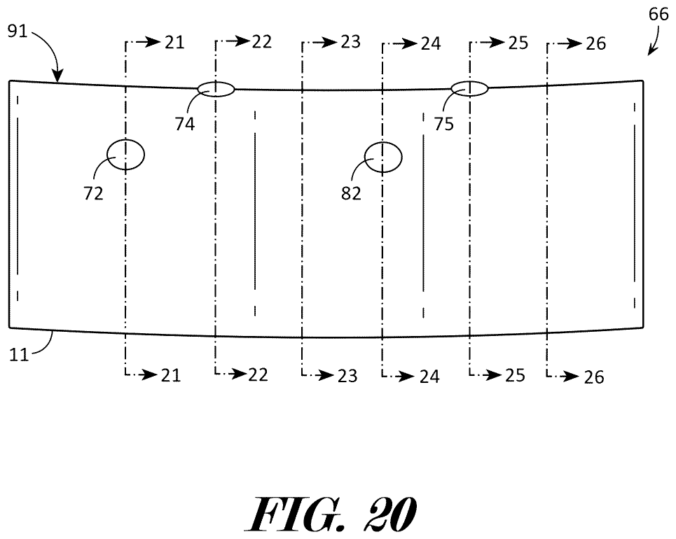

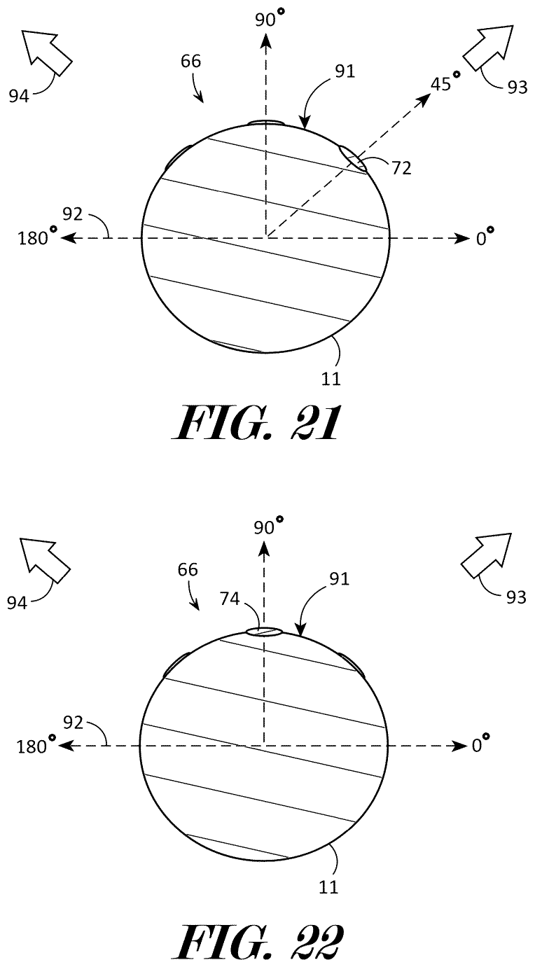

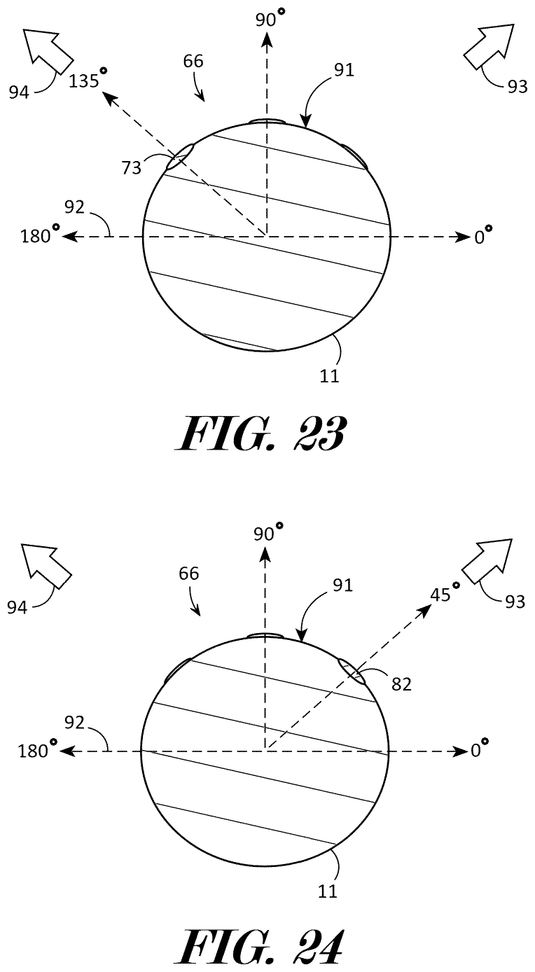

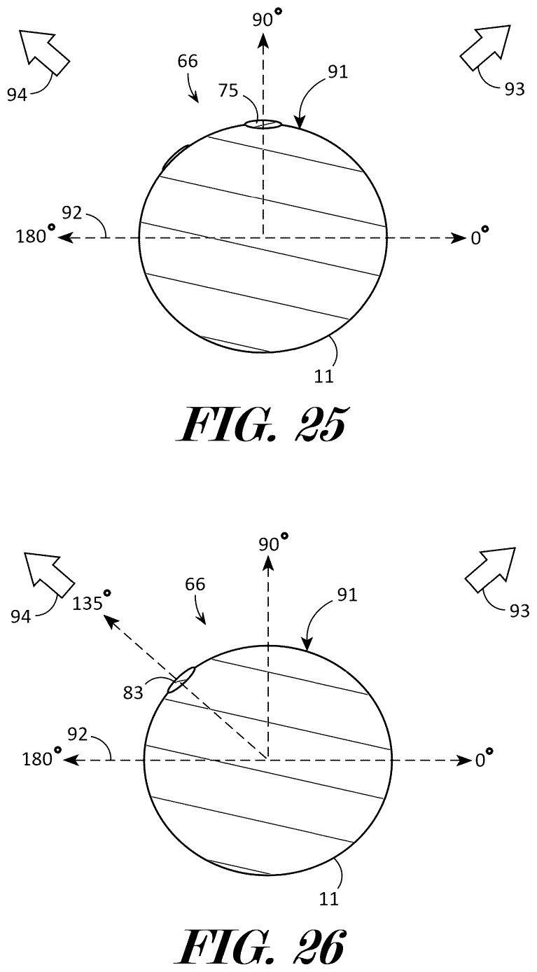

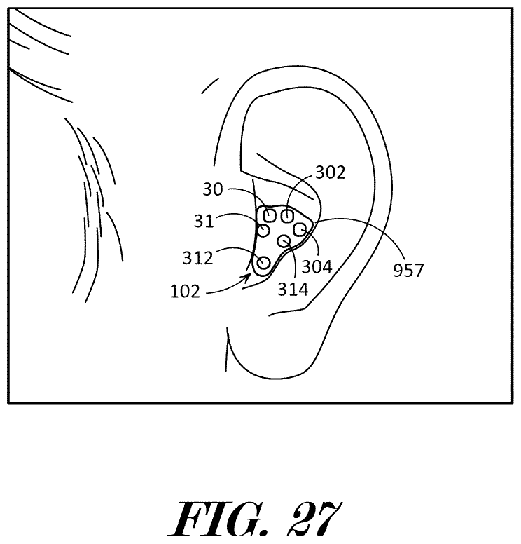

The auricular housing 11 (including all of these 3 types of housing, i.e. a modified earbud housing 61, a behind-the-ear-hearing-aid-style housing 63 and a modified in-the-ear housing 62) comprises a tubular-shaped structure 66. In preferred embodiments for an automatic detection-therapy system for cardiac arrhythmia 101 that comprises an auricular EEG monitoring system 170, all of the EEG sensor electrodes 72, 73, 74, 75, 82, 83, may be housed at the surface 91 of the tubular-shaped structure 66 and partially embedded with slight protrusion at the surface 91 of the tubular-shaped structure 66. One or more of the EEG sensor electrodes 72, 73, 74, 75, 82, 83, is/are configured to be located at the upper surface 91 (upper surface at approximately 90 degrees above horizontal level 92, as shown by electrodes 74, 75, in FIGS. 19, 22, 25) of the tubular-shaped structure 66. In preferred embodiments, one or more of the EEG sensor electrodes 72, 73, 74, 75, 82, 83, is/are configured to be located at between 0 and 90 degrees, and more preferably at approximately 45 degrees (plus or minus fifteen degrees or 30-60 degrees) above the horizontal level 92 of the tubular-shaped structure 66 and is/are configured to face forward-upward direction 93 (e.g., as shown by electrodes 72, 82, in FIGS. 19, 21, 24). In preferred embodiments, one or more of the EEG sensor electrodes is/are configured to be located at between 90 and 180 degrees, and more preferably at approximately 135 degrees (plus or minus fifteen degrees or 120-150 degrees) above the horizontal level 92 of the tubular-shaped structure 66 and is/are configured to face backward-upward direction 94 (e.g., as shown by electrodes 73, 83, in FIGS. 19, 23, 26). These arrangements will enable the EEG sensor electrodes 72, 73, 74, 75, 82, 83, to have one of the best locations and directions from the external ear canal 904 to record the wearer's EEG activities. (Upper surface, horizontal level, forward, backward, and upward all refer to directions relative to the head 901 of the wearer 900 with the wearer in an upright position after the tubular-shaped structure 66 has been inserted into a wearer's external ear canal 904.)

In some embodiments, an automatic detection-therapy system for cardiac arrhythmia 101 may comprises a switch 88 and a timer 89 to enable the wearer 900 to turn on the neuromodulation system 300 (of the system 101) manually and setting a duration, an interval or a schedule, etc. Preferably, the switch 88 may be configured as a multi-mode switch 88 and the timer 89 may be configured as a multi-mode timer 89. In some embodiments, a multi-mode switch 88 may be configured to enable the wearer to set the system 101 at an automatic mode or a manual mode. The functions and setups of the automatic mode for system 101 are as described hereinbefore. In some embodiments for an automatic detection-therapy system for cardiac arrhythmia 101, the multi-mode switch 88 may be configured to allow the wearer 900 to choose at least one of the following switch selections: turning on or off an auricular ECG monitoring system 100, turning on or off an auricular SCOS unit 511, turning on or off a SPG unit 555, (or turning on or off a PPG unit 555A), turning on or off a taVNS unit 30, turning on or off an auriculotemporal nerve (ATN) stimulation unit 302, turning on or off a greater auricular nerve (GAN) stimulation unit 304, turning on or off a supraorbital nerve stimulation unit 301, turning on or off an occipital nerve stimulation unit 303, turning on or off an infraorbital nerve stimulation unit 305, turning on or off a median nerve stimulation unit 306, or a combination thereof. The multi-mode timer 89 may be configured to function together with the multi-mode switch 88 to set each switch selection at a desirable timing course such as duration, interval, countdown, daily/weekly schedule, 10 minute/30 minutes/60 minutes (or other duration) selection, continuous operation, manual turning on and manual shutdown etc. The multi-mode switch 88 and multi-mode timer 89 may also be configured as a multifunctional switch-timer or as a programmable multifunctional timer-switch.

In some embodiments, a programmable multifunctional timer-switch 88, 89, may be incorporated in a client device 400 of a wearer 900 to allow the wearer flexibility and ease in choosing manual or automatic control and choosing various time courses and various switch selections (selection of various combinations of the 7 components of the neuromodulation unit, including the taVNS unit 30, the supraorbital nerve stimulation unit 301, the auriculotemporal nerve stimulation unit 302, the occipital nerve stimulation unit 303, the greater auricular nerve stimulation unit 304, the infraorbital nerve stimulation unit 305 and the median nerve stimulation unit 306.)

In some embodiments, the multi-mode switch 88 functions and the multi-mode timer 89 functions may be configured to be incorporated into a wearer's client device 400 and can be easily set up as programmable. In the manual mode, the wearer 900 can use the taVNS unit 30 or the ATN stimulation unit 302 or the GAN stimulation unit 304, or supraorbital nerve stimulation unit 301, or occipital nerve stimulation unit 303, or infraorbital nerve stimulation unit 305, or median nerve stimulation unit 306 or various combinations thereof for prophylactic purpose against cardiac arrhythmia or other illnesses or for health maintenance purpose. Studies have shown that the taVNS unit 30 is effective not only for therapy of cardiac arrhythmia and impending cardiac arrhythmia, but also for prophylaxis of cardiac arrhythmia. Studies have also shown that neuromodulating electric stimulation from the taVNS unit 30, the supraorbital nerve stimulation unit 301, the ATN stimulation unit 302, the occipital nerve stimulation unit 303, the GAN stimulation unit 304, the infraorbital nerve stimulation unit 305 and the median nerve stimulation unit 306 (and combination thereof) are effective not only for therapy of cardiac arrhythmia, impending cardiac arrhythmia, and cardiac arrhythmia prophylaxis but also for neuropsychiatric diseases and for health maintenance. The aforementioned programmable set up for the system 101 will enable the system 101 to be used for prophylactic purpose and health maintenance purpose. The multi-mode switch 88, multi-mode timer 89 and the programmable design may enable the wearer the option to choose from one of the following: neuromodulation from one neuromodulation component (single neuromodulation), simultaneous neuromodulation from two neuromodulation components (double neuromodulation) and simultaneous neuromodulation from three neuromodulation components (triple neuromodulation). Thus, when prompted or activated, the neuromodulation unit 300 is configured to generate neuromodulating electric stimulation to a wearer 900 according to the choice of the stimulation mode selected by the wearer 900.

In some embodiments, a separate client device 400 may be used for housing of one or more of the components of an automatic detection-therapy system for cardiac arrhythmia 101. For example, an ECG recording module 20, an EEG recording module 170 and the processing unit 50, 401, may be housed remotely in a client device 400, such as a wearable smart watch-type structure 400A or a portable smart phone type device 400B. The ECG sensor electrodes 12, 13, the EEG sensor electrodes 72, 73, 74, 75, 82, 83, the taVNS stimulating electrode 31, the ATN stimulating electrode 312 and the GAN stimulating electrode 314 may be housed in an auricular housing structure 11 (such as a modified earbud housing 61, a modified in-the-ear housing 61, a behind-the-ear-hearing-aid-style housing 63 or a tubular-shaped structure 66.) Wireless ECG sensor electrodes may be used for all of the ECG electrodes 12, 13. Wireless EEG sensor electrodes may also be used for all of the EEG sensor electrodes 72, 73, 74, 75, 82, 83. Wireless dry electrodes are known in the art, such as that described by Ryan Kaveh et al. in Nature Communications on Aug. 2, 2024. Wireless dry ECG electrodes are available from various companies, such as iWork System, BIOPAC and PASCO Scientific that offer various wireless ECG monitoring systems, including wireless ECG electrodes. There are well-known wired or wireless ECG amplifiers available, such as ECG Electrocardiogram Smart Amplifier (Part #ECG100D), or BioNomadix 2Ch Wireless ECG Amplifier (Part #BN-ECG2). Both are also available from BIOPAC Systems, Inc. (Goleta, California). Wireless dry electrodes for in-ear EEG such as that described by Ryan Kaveh et al. in Nature Communications on Aug. 2, 2024. Wireless dry EEG electrodes are also available from Zeto, Inc. headquarter in Santa Clara, California. There are well-known wired or wireless EEG amplifiers available, such as EEG Electroencephalogram Smart Amplifier (Part #: EEG100D), or preferably BioNomadix 2Ch Wireless EEG Amplifier (Part #: BN-EEG2), both being made by the same company BIOPAC Systems, Inc. (Goleta, California). By using wireless ECG sensor electrodes 12, 13, wireless EEG sensor electrodes 72, 73, 74, 75, 82, 83, wireless ECG amplifier 21 and wireless EEG amplifier, the ECG recording module 20, the EEG recording module 71, together with the processing unit 401, may be housed remotely in a wearable client device 400 (such as a watch-type device or a smart phone type device) and communicate wirelessly with their respective ECG sensor electrodes 12, 13 and the EEG sensor electrodes 72, 73, 74, 75, 82, 83. By using wireless ECG electrodes, wireless EEG electrodes, wireless ECG amplifier and wireless EEG amplifier, the ECG recording module 20 and the EEG recording module 170 together with the processing unit 401, may be housed remotely in a client device 400 (such as a wearable watch-type client device 400A or a portable smart phone type client device 400B) and communicate wirelessly with all of the ECG sensor electrodes 12, 13, and EEG sensor electrodes 72, 73, 74, 75, 82, 83.

In some embodiments, an auricular ECG monitoring system 100 and/or an automatic detection-therapy system for cardiac arrhythmia 101 may comprise a network interface 53, 406. The network interface 53, 406, is in electronic communication with the processing unit 50, 401. The network interface 53, 406, may also be in electronic communication with at least one of the following: a speaker 15, 404A, and a vibrator 17, 404B, on a client device 400. When the processing unit 50, 401 detects presence of serious cardiac arrhythmia or presence of impending serious cardiac arrhythmia, the network interface 53, 406, is configured to send signals to at least one of: the speaker 15, 404A, and the vibrator 17, 404B. The speaker 15, 404A may be configured to generate an audible notification to a wearer 900 when serious cardiac arrhythmia or impending serious cardiac arrhythmia is detected by the processing unit 50, 401. The vibrator 17, 404B, is configured to generate a tactile notification to the wearer 900 when serious cardiac arrhythmia or impending serious cardiac arrhythmia is detected by the processing unit 50, 401. When the processing unit 50, 401, detects cessation of the serious cardiac arrhythmia or impending serious cardiac arrhythmia, the network interface 53, 406, is further configured to send signals to the speaker 15, 404A, and the vibrator 17, 404B, to stop the notification of serious cardiac arrhythmia or impending serious cardiac arrhythmia.

In some embodiments, an automatic detection-therapy system for cardiac arrhythmia 101 may comprise a speaker 15, 404A or a vibrator 17, 404B, which may be configured to generate an audible or tactile notification when presence of a serious cardiac arrhythmia or presence of an impending serious cardiac arrhythmia is detected by a processing unit 50, 401. The speaker 15, 404A, may comprise a buzzer, a piezoelectric sound producing device, a dielectric elastomer sound producing device, a buzzer, a moving coil loudspeaker, an electrostatic loudspeaker, an isodynamic loudspeaker, a piezo-electric loudspeaker, or any other device capable of producing one or more sounds. The vibrator 17, 404B, may comprise a weight that may be rapidly moved by a long life brushless (BLDC) vibration motor, a coin or pancake vibration motor, an encapsulated vibration motor, an enclosed vibration motor, a pager motor, an eccentric rotating mass (ERM) motor, a linear resonant actuator (LRA), a printed circuit board (PCB) mounted vibration motor, or any other electrical device capable of producing a series of rapid and repeated movements.

In some embodiments, an automatic detection-therapy system for cardiac arrhythmia 101 may comprise a neuromodulation system 300 having a taVNS unit 30. Preferably, the taVNS unit 30 will be placed in or around the left external ear 902. However, if one-sided (left side) vagus nerve stimulation is not enough to achieve the desired therapeutic effect for the cardiac arrhythmia, the automatic detection-therapy system for cardiac arrhythmia 101 may utilize or comprise two taVNS units 30, with a first stimulating electrode 31 of a first taVNS unit 30 placed in the first external ear 902 and a second stimulating electrode 31 of a second taVNS unit 30 placed in the second external ear 902. It has been reported that bilateral vagal stimulation could be more effective than unilateral vagal stimulation.

Similarly, the neuromodulation system 300 may include two auriculotemporal nerve (ATN) stimulation unit 302, with one ATN stimulation unit located in the wearer's first ear 902 while the second ATN stimulation unit 302 located in the wearer's second ear 902. Likewise, the neuromodulation unit 300 may include two greater auricular nerve (GAN) stimulation unit 304 with one GAN stimulation unit 304 located in the wearer's first ear 902 and the second GAN stimulation unit 304 located in the wearer's second ear 902.

In some embodiments, an automatic detection-therapy system for cardiac arrhythmia 101 may comprise two neuromodulation systems 300. A first neuromodulation system 300 may be configured to include at least one of the following: a first transcutaneous auricular vagus nerve stimulation unit (taVNS unit) 30, a supraorbital nerve stimulation unit 301, a first auriculotemporal nerve (ATN) stimulation unit 302, an occipital nerve stimulation unit 303, a first greater auricular nerve (GAN) stimulation unit 304, an infraorbital nerve stimulation unit 305, and a median nerve stimulation unit, as aforementioned description. The first taVNS unit 30, the first ATN stimulation unit 302 and the first GAN stimulation unit 304 may be configured to be located at the wearer's first ear 902. The supraorbital nerve stimulation unit 301 may be located at midforehead and be configured to stimulate unilateral or bilateral supraorbital nerves. The occipital nerve stimulation unit 303 may be located at mid-occipital region and be configured to stimulate unilateral or bilateral occipital nerves. The infraorbital nerve stimulation unit 305 may be located at mid-face and be configured to stimulate unilateral or bilateral infraorbital nerves. The median nerve stimulation unit 306 may be located on the wearer's skin at a Neiguan point which is on the palmar side of distal forearm about 1.5 to 2 inches (or 2 to 3 finger widths) proximal to the wrist crease. The median nerve stimulation unit 306 is configured to give neuromodulating electric stimulation to the wearer's median nerve when prompted or activated. Optionally, a second neuromodulation system 300 may include a second taVNS unit 30, a second ATN stimulation unit 302 and a second GAN stimulation unit 304, configured to be located at the wearer's second ear 902, and a second median nerve stimulation unit 306 configured to be located at a Neiguan point on the wearer's second forearm. The setups and functions of the second taVNS unit 30, the second ATN stimulation unit 302, the second GAN stimulation unit 304 and the second median nerve stimulation unit 306 are essentially the same as the first taVNS unit 30, the first ATN stimulation unit 302, the first GAN stimulation unit 304 and the first median nerve stimulation unit 306 respectively. Studies have shown that bilateral neuromodulation is more effective than unilateral neuromodulation. Separately, studies have shown that simultaneous neuromodulation of two or three nerves on the same side is more effective than single nerve neuromodulation due to their synergistic effects.

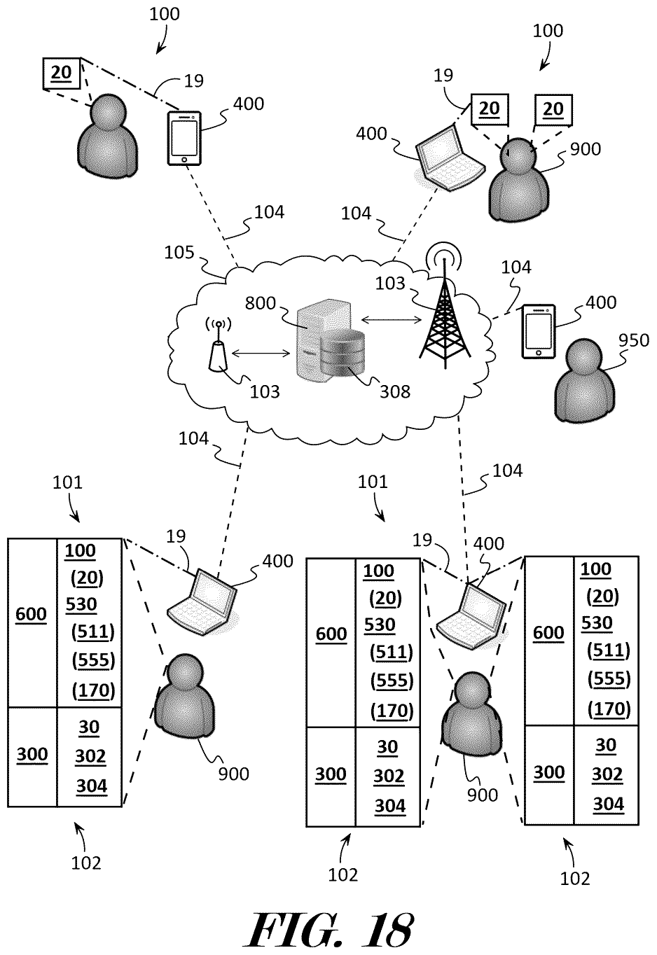

As perhaps best shown by FIG. 18, an illustrative example of some of the physical components which may be used with an auricular ECG monitoring system 100, an automatic detection-therapy system for cardiac arrhythmia 101, a cardiovascular monitoring apparatus 600, a cephalic blood flow monitoring system 530, including an auricular SCOS unit 511, a SPG unit 555 (or a PPG unit 555A) and an auricular EEG monitoring system 170, and a closed-loop neuromodulation system 102 (including various components of a neuromodulation unit 300, such as a taVNS unit 30, an auriculotemporal nerve stimulation unit 302 and a greater auricular nerve stimulation unit 304), according to some embodiments are presented. A system 100 and a system 101 are configured to facilitate the transfer of data and information between one or more access points 103, client devices 400, and servers 800 over a data network 105. Client devices 400 and servers 800 may send data to and receive data from the data network 105 through a network connection 104 with an access point 103. A data store 308 accessible by the server 800 may contain one or more databases. The data may comprise any data recorded and generated by a system 100 and a system 101.

In this example of FIG. 18, an auricular ECG monitoring system 100 and an automatic detection-therapy system for cardiac arrhythmia 101 may be in communication with at least one client device 400 configured to be operated by one or more users 900, 950. Client devices 400 may include mobile devices, such as laptops, tablet computers, personal digital assistants, smart phones, and the like, that are equipped with a wireless network interface capable of sending data to one or more servers 800 with access to one or more data stores 308 over a network 105, such as a wireless local area network (WLAN). Additionally, client devices 400 may include fixed devices, such as desktops, workstations, and the like, that are equipped with a wireless or wired network interface capable of sending data to one or more servers 800 with access to one or more data stores 308 over a wireless or wired local area network 105. The present invention may be implemented on at least one computing device, such as a client device 400 and/or server 800, programmed to perform the steps described herein. In some embodiments, more than one client device 400 and server 800 may be used, with each being programmed to carry out one or more steps of a process described herein.

In another aspect consistent with the principles of this invention, in some embodiments, a cardiovascular monitoring apparatus 600 may comprise an auricular ECG monitoring system 100, a cephalic blood flow monitoring system 530, a network interface and a processing unit 50, 401. The cephalic blood flow monitoring system 530 may comprise an auricular speckle contract optical spectroscopy unit (auricular SCOS unit) 511. Alternatively, the cephalic blood flow monitoring system may comprise an auricular SCOS unit 511 and at least one of: a speckle-plethysmography (SPG) unit 555 and a photoplethysmography (PPG) unit 555A. There is evidence that combining SCOS and SPG (or PPG) together can enhance the accuracy of blood flow assessments compared to using either method alone, as SCOS captures high-frequency changes in blood flow while SPG (or PPG) provides volume data, thus, complementary effects. SPG (or PPG) also provide valuable estimate of systolic and diastolic blood pressure.

In some embodiments, a cardiovascular monitoring apparatus 600 may comprise an auricular ECG monitoring system 100, an auricular SCOS unit 511, at least one of: a SPG unit 555 and a PPG unit 555A, an auricular EEG monitoring system 170 and a network interface 53. The auricular ECG monitoring system 100, the auricular SCOS unit 511, at least one of: the SPG unit 555 and the PPG unit 555A, and the auricular EEG monitoring system 170 may be configured to be housed in an auricular housing 11 to be placed in an ear 902 of the wearer 900 when in use, similar to descriptions hereinbefore. The auricular SCOS unit 511 is configured to collect the cerebral and extracranial blood flow data of a wearer 900. The at least one of: SPG unit 555 and PPG unit 555A is configured to collect the wearer's regional blood flow data, systolic blood pressure data and diastolic blood pressure data. The processing unit 50, 401, is in electronic communication with the auricular ECG monitoring system 100, the auricular SCOS unit 511, at least one of: the SPG unit 555 and the PPG unit 555A, the auricular EEG monitoring system 170 and the network interface 53. The processing unit 50, 401, is configured to convert the raw EEG data recorded by the auricular EEG monitoring system 170 into quantitative EEG (qEEG) data. The processing unit 50, 401, is configured to analyze the ECG data recorded by the auricular ECG monitoring system 100 to detect presence or cessation of serious cardiac arrhythmia and presence or cessation of impending serious cardiac arrhythmia. The processing unit is configured to analyze the data recorded by the auricular SCOS unit 511 to detect presence of a decrease of cerebral blood flow more than a predetermined percentage (e.g. a decrease of 30% or more).

The processing unit is configured to analyze the data recorded by at least one of: the SPG unit 555 and the PPG unit 555A to detect presence of at least one of: a decrease of systolic blood pressure more than a predetermined amount (e.g. a decrease of 20 mmHg or more), a decrease of diastolic blood pressure more than a predetermined amount (e.g. a decrease of 20 mmHg or more) and a decrease of regional blood flow more than a predetermined percentage (e.g. a decrease of 30% or more). The processing unit 50, 401, is also configured to analyze the EEG and qEEG data recorded by the auricular EEG monitoring system 170 to detect presence of at least one of: an increase of relative delta power more than a predetermined percentage (e.g. an increase of 30% or more), an increase of delta/alpha ratio more than a predetermined percentage (e.g. an increase of 30% or more), and a decrease of cordance z-score more than a predetermined amount (e.g. a decrease of 1 SD or more) (or significant changes of other metrics or parameters from qEEG). When the processing unit 50, 401, detects at least one of: presence of serious cardiac arrhythmia and presence of impending serious cardiac arrhythmia, the processing unit 50, 401, is configured to send signals to the network interface 53 to prompt the network interface 53 to generate a notification to at least one of: a client device 400 of the wearer and a client device 400 of a healthcare provider 950 of the wearer. When presence of serious cardiac arrhythmia co-exists with significant hemodynamic changes, the processing unit 50, 401, is configured to send signals to the network interface 53 to generate another and more urgent notification to at least one of: a client device 400 of the wearer 900 and a client device 400 of a healthcare provider 950 of the wearer. As used herein, significant hemodynamic change comprises at least one of the following: presence of a decrease of cerebral blood flow more than a predetermined percentage (e.g. a decrease of 30% of more), presence of a decrease of systolic blood pressure more than a predetermined amount (e.g. a decrease of 20 mmHg or more), presence of a decrease of diastolic blood pressure more than a predetermined amount (e.g. a decrease of 20 mmHg or more), presence of a decrease of regional blood flow more than a predetermined percentage (e.g. a decrease of 30% or more) and presence of at least one of: an increase of relative delta power from qEEG of more than a predetermined percentage (e.g. an increase of 30% or more), an increase of delta/alpha ratio from qEEG more than a predetermined percentage (e.g. an increase of 30% or more) and a decrease of cordance z-score more than a predetermined amount (e.g. a decrease of 1 SD or more) (or significant changes of other metrics data from qEEG).

In some embodiments, an automatic detection-therapy system for cardiac arrhythmia 101 may comprise an auricular ECG monitoring system 100, a cephalic blood flow monitoring system 530, a neuromodulation system 300 and a processing unit 50, 401. The auricular ECG monitoring system 100 is configured to record the wearer's ECG data and to detect presence of serious cardiac arrhythmia and presence of impending serious cardiac arrhythmia, as described hereinbefore. The cephalic blood flow monitoring system 530 may comprise at least one of: an auricular SCOS unit 511, a SPG unit 555, a PPG unit 555A, and an auricular EEG monitoring system 170, each of these (511, 555 or 555A, 170) as described hereinbefore. The auricular SCOS unit 511 is configured to record the wearer's cerebral and extracranial blood flow data. The at least one of: SPG unit 555 and PPG unit 555A is configured to record the wearer's regional blood flow data, systolic blood pressure and diastolic blood pressure data. The auricular EEG monitoring system 170 is configured to record the wearer's EEG data. The processing unit 50, 401, is in electronic communication with the auricular ECG monitoring system 100, the auricular SCOS unit 511, at least one of: the SPG unit 555 and the PPG unit 555A, and the auricular EEG monitoring system 170. The processing unit 50, 401, is configured to convert the raw EEG data recorded by the auricular EEG monitoring system 170 into quantitative EEG (qEEG) data. The neuromodulation system 300 comprises at least one of the following components: a taVNS unit 30, a supraorbital nerve stimulation unit 301, an auriculotemporal nerve stimulation unit 302, an occipital nerve stimulation unit 303, a greater auricular nerve stimulation unit 304, an infraorbital nerve stimulation unit 305, and a median nerve stimulation unit 306, and each of these components (30, 301, 302, 303, 304, 305, 306) is configured to give neuromodulating electric stimulation to the wearer when activated or prompted, as described hereinbefore. The cephalic blood flow monitoring system 530 is configured to monitor the wearer's hemodynamic status and the hemodynamic status is very important supplemental information when assessing the wearer's cardiac arrhythmia. The hemodynamic status is also very important to provide input data for the closed-loop control system 333 when delivering therapeutic neuromodulating electric stimulation to the wearer 900 for the cardiac arrhythmia. When the processing unit 50, 401, detects presence of a serious cardiac arrhythmia and (co-exists with) presence of significant hemodynamic change, the processing unit 50, 401, is configured to send signals to the neuromodulation system 300 to prompt the neuromodulation system 300 to start sending more potent neuromodulating electric stimulation from at least one of the following components: the taVNS unit 30, the supraorbital nerve stimulation unit 301, the auriculotemporal nerve stimulation unit 302, the occipital nerve stimulation unit 303, the greater auricular nerve stimulation unit 304, the infraorbital nerve stimulation unit 305 and the median nerve stimulation unit 306. (As used herein, more potent neuromodulating electric stimulation refers to at least one of: higher frequency, higher strength, higher duration of the neuromodulating electric stimulation, and recruiting more components of the neuromodulation system 300.) As used herein, the term “significant hemodynamic change” refers to (comprises) at least one of the following: a decrease of cerebral blood flow by more than a predetermined percentage (e.g. a decrease of 30% or more), a decrease of systolic blood pressure more than a predetermined amount (e.g. a decrease of 20 mmHg or more), a decrease of diastolic blood pressure more than a predetermined amount (e.g. a decrease of 20 mmHg or more), an increase of relative delta power of the qEEG by more than a predetermined percentage (e.g. an increase of 30% or more), an increase of delta/alpha ratio by more than a predetermined percentage (e.g. an increase of 30% or more), a decrease of cordance z-score more than a predetermined amount (e.g. a decrease of 1 SD or more) (or significant changes of other metrics data from the qEEG). The processing unit 50, 401 further comprises a closed-loop control system 333. When giving the neuromodulating electric stimulation, the stimulation parameters, the duration, interval and other timing course and the selection of how many components of the neuromodulation system 300 is guided or regulated by the closed-loop control system 333, using input data from the auricular ECG monitoring system 100 and input hemodynamic data from at least one of: the auricular SCOS unit 511, the SPG unit 555, the PPG unit 555A, and the auricular EEG monitoring system 170, as described hereinbefore

In some embodiments, each component of a neuromodulation system 300 of an automatic detection-therapy system for cardiac arrhythmia 101 may comprise a communication interface 42. Optionally, a taVNS unit 30 may comprise a communication interface 42 which may enable electronic communication 19 (e.g., wired and/or wireless communication) between the taVNS unit 30 and another electronic device, such as a client device 400, an auricular ECG monitoring system 100, etc. Optionally, an auriculotemporal nerve stimulation unit 302 may comprise a communication interface 42 which may enable electronic communication 19 (e.g., wired and/or wireless communication) with another electronic device, such as a client device 400, an auricular ECG monitoring system 100, etc. Similarly, a median nerve stimulation unit 306 may comprise a communication interface 42 which may enable electronic communication 19 (e.g., wired and/or wireless communication) with another electronic device, such as a client device 400, an auricular ECG monitoring system 100, etc. Likewise, a greater auricular nerve stimulation unit 304 (or a supraorbital nerve stimulation unit 301, or an occipital nerve stimulation unit 303 or an infraorbital nerve stimulation unit 305) may comprise a communication interface 42 which may enable electronic communication 19 (e.g., wired and/or wireless communication) with another electronic device, such as a client device 400, an auricular ECG monitoring system 100, etc. Preferably, a communication interface 42 may comprise a radio that may operate via WiFi and/or Bluetooth communication standards. In further embodiments, a communication interface 42 may be configured as a network interface 53 described above so that it may operate on any wireless and/or wired electronic communication 19 protocol that a network interface 53 may use.

It should be noted that although both cerebral hypoperfusion and sleep or drowsiness may produce increase of slow waves (theta waves and delta waves) in EEG and qEEG, there are distinct features that the processing unit 50, 401, can use to differentiate between them. Studies have shown that quantitative EEG (qEEG) is a highly sensitive, though not perfectly specific, tool for detecting cerebral hypoperfusion, with studies indicating a sensitivity greater than 80% and specificity of roughly 70% between qEEG changes and actual blood flow reductions. It can detect ischemia before clinical symptoms or imaging (CT/MRI) reveal damage. There are features that the processing unit 50, 401, can use to differentiate between hypoperfusion-induced slow waves and sleep-induced slow waves, such as analyzing topography (location or where they appear) and coherence (how regions communicate), as sleep slow waves are generally global, while hypoperfusion-related slow waves are often focal or regional. They also differ in other electrophysiological signatures. When the qEEG data are combined with data from auricular SCOS unit 511 and data from at least one of: SPG unit 555 and PPG unit 555A, the sensitivity and specificity for qEEG in detecting cerebral hypoperfusion are very high. Cordance is an especially valuable tool from qEEG to differentiate hypoperfusion from sleep or drowsiness induced EEG slowing. Cordance is a qEEG method developed to measure cortical deafferentation, combining absolute and relative power to provide a surrogate marker for regional cerebral perfusion. It is particularly effective in distinguishing pathological states from normal physiologic changes like sleep. Positive cordance values (concordance) indicate normally functioning brain. Even if sleep causes high relative theta power, the absolute power remains high enough to keep cordance positive, and this helps to differentiate it from the discordance caused by hypoperfusion. Quantitative EEG can detect cerebral ischemia and hypoperfusion before clinical symptoms and CT or MRI imaging.

### A First Preferred Embodiment

According to one aspect consistent with the principles of the invention, a novel auricular electrocardiogram (ECG) monitoring system 100 is disclosed. An auricular electrocardiogram (ECG) monitoring system 100 that is wearable, convenient, suitable for long-term monitoring and with much less muscle artifacts and movement artifacts is disclosed. Referring to FIG. 1, in some embodiments, an auricular ECG monitoring system 100 may comprise a miniature ECG recording module 20. The ECG recording module 20 may comprise at least two wired or wireless ECG electrodes (ECG sensor electrodes) 12, 13, in electronic communication with the ECG recording module 20. At least one of the auricular ECG sensor electrodes 12, 13, (for example a first ECG sensor electrode 12) may be configured to be located in a wearer's first ear 902 or a peri-auricular area 903 around the wearer's first ear 902. The first ECG sensor electrode 12 is positioned in contact with the skin on an area of the wearer, the area selected from at least one of the following: external ear 902 of the wearer's first ear, external ear canal 904 of the wearer's first ear 902, and the peri-auricular area 903 around the wearer's first ear 902. In some embodiments, the second ECG sensor electrode 13 may also be configured to be located in the wearer's first ear 902 or the periauricular area 903 around the wearer's first ear 902. These locations that the auricular ECG sensor electrodes 12, 13, are attached to may be adequately separated (with different angles or directions) relative to the location of the heart of the wearer so that they can pick up some differences of the cardiac action potentials in order to enable the ECG recording module 20 to record an ECG. The ECG recording module 20 may be configured to record ECG data of the wearer via the ECG sensor electrodes 12, 13, and the ECG recording module 20 may be in electronic communication with a processing unit 50, 40. With the help of ECG analysis algorithms, the processing unit 50, 401, may be configured to record and analyze the ECG data from the ECG recording module 20 to assess the wearer's ECG profile. The processing unit 50, 401, may be further configured to analyze ECG data recorded by the ECG recording module 20 to detect presence or cessation of a serious cardiac arrhythmia of the wearer 900 and presence or cessation of impending serious cardiac arrhythmia. The serious cardiac arrhythmia may include atrial fibrillation (AFib), atrial flutter (AFL), supraventricular tachycardia (SVT), ventricular tachycardia (VT), and ventricular fibrillation (VF).

In alternative embodiments for the auricular ECG monitoring system 100, the first ECG sensor electrode 12 may be configured to be located in an ear 902 of the wearer, as aforementioned. The second ECG sensor electrode 13 may be remotely located in one of the following: a second ear 902 of the wearer, a wrist of the wearer, a finger of the wearer, an ankle of the wearer and a toe of the wearer. When the first ECG sensor electrode 12 and the second ECG electrode 13 are located in a same ear, they should be separated enough in order to increase the strength of the ECG signals. When the second ECG electrode 13 is located more away from the first ECG sensor electrode 12 (for example, in a second ear, on a wrist, a finger, an ankle or a toe), the ECG signals will be stronger although it will be less convenient for the wearer (user). The first and the second ECG sensor electrodes 12, 13, may be configured as wireless ECG sensor electrodes and in electronic communication wirelessly with the ECG recording module 20. The ECG recording module 20 may comprise a wireless amplifier 21. The ECG recording module 20 is configured to record ECG data of the wearer. The ECG recording module 20 may be in electronic communication with a processing unit 50, 401. With the help of ECG analysis algorithms, the processing unit 50, 401, may be configured to record and analyze the ECG data recorded by the ECG recording module 20 to assess the wearer's ECG profile. The processing unit 50, 401, may be further configured to analyze ECG data recorded by the ECG recording module 20 to detect presence or cessation of a serious cardiac arrhythmia of the wearer and presence or cessation of an impending serious cardiac arrhythmia of the wearer. The serious cardiac arrhythmia may include atrial fibrillation (AFib), atrial flutter (AFL), supraventricular tachycardia (SVT), ventricular tachycardia (VT), and ventricular fibrillation (VF).

As used herein, a peri-auricular area 903 refers to a portion of the head around the auricle (pinna) and this portion of the head 901 is typically hairless. The peri-auricular area 903 includes a portion of the head in front of the auricle (pre-auricular area) and a portion of the head 901 above and behind the auricle (post-auricular area). The pre-auricular area is small, about one inch wide and two inches long and curved along the anterior edge of the auricle. The post-auricular area is also small, about one inch wide and about three inches long and curved along the superior and posterior edges of the auricle (pinna). The post-auricular area is where a behind-the-ear hearing aid is usually located. The small pre-auricular area and post-auricular area together will be called “peri-auricle area” 903 herein. (Anterior, posterior, superior, in front of and behind etc. all refer to the directions relative to the wearer's head 901 when the wearer 900 is in an upright position.) As referred to herein, the external ear canal 904 refers to the part of the ear 902 that connects the visible outer ear (pinna) to the middle ear, essentially the tube that carries sound waves to the eardrum.

The ECG recording module 20 may be in electronic communication with a processing unit 50, 401, so that the ECG data recorded by the ECG recording module 20 may be communicated to the processing unit 50, 401. With the help of ECG analysis algorithms, the processing unit 50, 401, is configured to analyze the ECG data to detect presence or cessation of any serious cardiac arrhythmia and presence or cessation of any impending serious cardiac arrhythmia, as aforementioned. This auricular ECG monitoring system 100 may further comprise a network interface 53, 406, in electronic communication with the processing unit 50, 401, and at least one of the following: a speaker 15, 404A, and a vibrator 17, 404B. The network interface 53, 406, may be configured to generate a notification, such as a text message, email, or other electronic user readable message to the client device 400 of the wearer 900 of this auricular ECG monitoring system 100 and/or to the client device 400 of the wearer's 900 healthcare provider 950 to take appropriate actions when serious cardiac arrhythmia or impending serious cardiac arrhythmia is detected by the processing unit 50, 401. When cessation of serious cardiac arrhythmia or cessation of impending serious cardiac arrhythmia is detected, the processing unit 50, 401, may be further configured to send a notification to the client device 400 of the wearer 900 of this ECG monitoring system 100 and/or to the client device 400 of the wearer's 900 healthcare provider 950. In some embodiments, the network interface 53, 406, may be configured to send signals to a speaker 15, 404A, to generate an audible notification when serious cardiac arrhythmia or impending serious cardiac arrhythmia is detected. In some embodiments, the network interface 53, 406, may be configured to send signals to a vibrator 17, 404B, to generate a tactile notification when serious cardiac arrhythmia or impending serious cardiac arrhythmia is detected. Additionally, the network interface 53, 406, may be further configured to send signals to the speaker, 15, 404A, or vibrator 17, 404B, to stop the notification (or send a cessation notification) when cessation of serious cardiac arrhythmia or cessation of impending serious cardiac arrhythmia is detected.

In some embodiments, an auricular ECG monitoring system 100 may comprise an ECG recording module 20 having at least two ECG sensor electrodes 12, 13, including a first ECG sensor electrode 12 and a second ECG sensor electrode 13. The first ECG sensor electrode 12 may be configured to contact a skin area of a first ear 902 of a wearer 900 and the skin area that the first ECG sensor electrode 12 is configured to contact may be selected from one of the following: an external ear 902 of the first ear of the wearer 900, an external ear canal 904 of the first ear of the wearer and a periauricular area 903 around the first ear of the wearer. The second ECG sensor electrode 13 may be configured to contact a separate skin area of the first ear 902 of the wearer, and the skin area that the second ECG sensor electrode 13 is configured to contact may be selected from one of the following: the external ear 902 of the first ear of the wearer, the external ear canal 904 of the first ear of the wearer and the periauricular area 903 around the first ear of the wearer 900. The skin area selected for the first ECG sensor electrode 12 to contact is different from the skin area selected for the second ECG sensor electrode 13 to contact. The ECG recording module 20 is configured to record ECG data of the wearer via the first ECG sensor electrode 12 and the second ECG sensor electrode 13.

In alternative embodiments, the auricular ECG monitoring system 100 may comprise an ECG recording module 20 having at least two ECG sensor electrodes 12, 13, including a first ECG sensor electrode 12 and a second ECG sensor electrode 13. The first ECG sensor electrode 12 may be configured to contact a skin area of a first ear 902 of the wearer and the skin area that the first ECG sensor electrode 12 is configured to contact is selected from one of the following: an external ear 902 of the first ear of the wearer, an external ear canal 904 of the first ear of the wearer and a periauricular area 903 around the first ear of the wearer. The second ECG sensor electrode 13 may be configured to contact a skin area of a second ear 902 of the wearer and the skin area that the second ECG sensor electrode 13 is configured to contact may be selected from one of the following: an external ear 902 of the second ear of the wearer, an external ear canal 904 of the second ear of the wearer and a periauricular area 903 around the second ear of the wearer. The ECG sensor electrodes 12, 13, may be configured as wireless ECG sensor electrodes in wireless electronic communication with the ECG recording module 20. The ECG recording module 20 is configured to record ECG data of the wearer via the first ECG sensor electrode 12 and the second ECG sensor electrode 13. Having the first and the second ECG sensor electrodes contacting different ears has the advantage that it will significantly improve the strength and quality of the ECG signals.

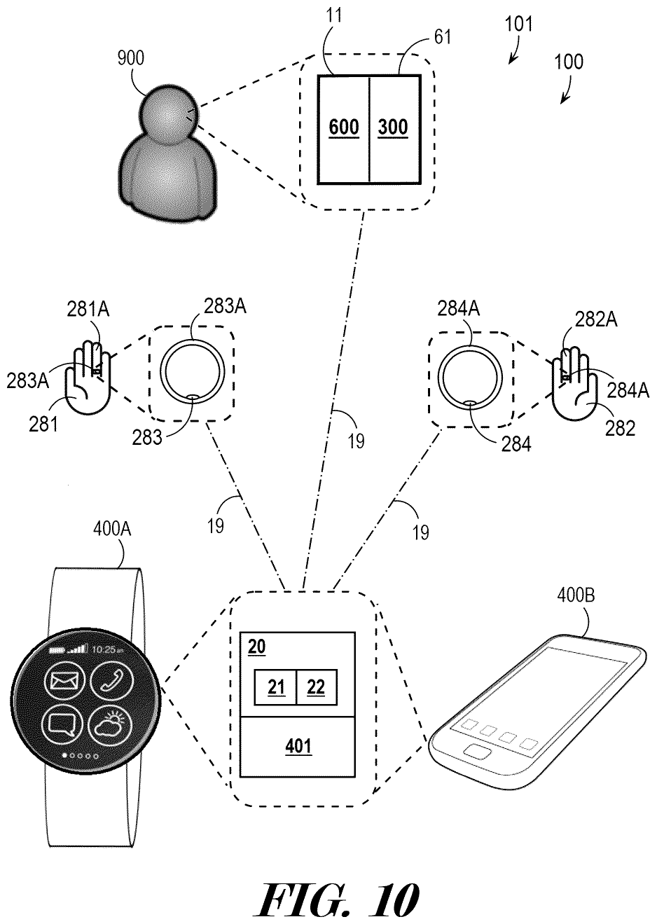

In some other alternative embodiments, an auricular ECG monitoring system 100 may comprise an ECG recording module 20 having at least two ECG sensor electrodes 12, 13, including a first ECG sensor electrode 12 and a second ECG sensor electrode 13. The first ECG sensor electrode 12 may be configured to contact the skin area of a first ear 902 of a wearer 900 and the skin area that the first ECG sensor electrode 12 is configured to contact may be selected from one of the following: an external ear 902 of the first ear of the wearer 900, an external ear canal 904 of the first ear of the wearer and a periauricular area 903 around the first ear of the wearer. The second ECG sensor electrode 13 may be configured to contact a skin area selected from one of the following: a finger 281A of the wearer via a finger ring 283A, an ankle 174 of the wearer via an ankle band 171, a toe 271A of the wearer via a toe ring 273A and a wrist of the wearer via one of: a wrist band 150, a smart watch 400A and a health tracker. The ECG sensor electrodes 12, 13, may be configured as wireless ECG sensor electrodes in wireless electronic communication with the ECG recording module 20. The ECG recording module 20 is configured to record ECG data of the wearer via the first ECG sensor electrode 12 and the second ECG sensor electrode 13. Having the first and the second ECG sensor electrodes contacting different parts of the human body has the advantage that it will significantly improve the strength and quality of the ECG signals. Referring to FIG. 3, a second ECG sensor electrode 13 may be configured as a wrist electrode 151 located on an inner surface of a wrist band 150 (alternatively a watch electrode on an undersurface of a smart watch 400A). Referring to FIG. 8, a second ECG sensor electrode 13 may be configured as a second finger ring electrode 283 that is configured to be located on the inside surface of a second finger ring 283A to be worn on a Y-finger 281A of the wearer's Y-hand 281. Referring to FIG. 9, a second ECG sensor electrode 13 may be configured as a toe-ring electrode 273 that is configured to be located on the inside surface 277 of a toe ring 273A to be worn on a toe 271A of the wearer's foot 271. Alternatively, the second ECG sensor electrode 13 may be configured as an ankle electrode 173 that may be configured to be located on an ankle band 171 to be worn on an ankle 174 of the wearer. In yet a modified embodiment, and referring to FIG. 10, a first ECG sensor electrode 12 may be configured as a first finger ring electrode 284 that is configured to be located on the inside surface of a first finger ring 284A to be worn on an X-finger 282A of the wearer's X-hand 282, while a second ECG sensor electrode 13 may be configured as the second ring electrode 283 configured to be located on the inside surface of the second finger ring 283A to be worn on the Y-finger 281A of the wearer's Y-hand 281. Having the ECG sensor electrodes located in an ear or on a surface of a finger-ring, a toe-ring, an ankle band, a wrist band, a smart phone or a health tracker, etc. will all have the advantage that they can be easily user-installable, user-removable and the wearer can ambulate freely.

### A Second Preferred Embodiment

According to another aspect consistent with the principles of the invention, an automatic detection-therapy system for cardiac arrhythmia 101 is disclosed. In some embodiments, an automatic detection-therapy system for cardiac arrhythmia 101 may comprise a cardiovascular monitoring apparatus 600, a neuromodulation system 300 and a processing unit 50, 401. The cardiovascular monitoring apparatus 600 may comprise at least an auricular ECG monitoring system 100 that have been described above and in the First Preferred Embodiment. The cardiovascular monitoring apparatus 600 may further comprise a cephalic blood flow monitoring system 530. The cephalic blood flow monitoring system 530 may comprise at least one of: an auricular speckle contrast optical spectroscopy (SCOS) unit 511, a speckle-plethysmography (SPG) unit 555, a photoplethysmography (PPG) unit 555A, and an auricular electroencephalogram (EEG) monitoring system 170, for monitoring of the wearer's hemodynamic status. The auricular SCOS unit 511 includes an auricular SCOS sensor 512 that is housed in an auricular housing 11. The auricular SCOS unit 511 is configured to record the wearer's cerebral and extracranial blood flow data, as described hereinbefore. The SPG unit 555 and the PPG unit 555A are configured to be housed in the auricular housing 11 and to be placed in an ear 902 of a wearer 900. The SPG unit 555 is configured to record the wearer's regional blood flow data (regional blood flow at the location where the SPG unit 555 contacts the wearer's auricular skin) and systolic blood pressure data and diastolic blood pressure data, as described hereinbefore. The SPG unit 555 may be substituted by the PPG unit 555A. The PPG unit 555A is also configured to record the wearer's regional blood flow data (regional blood flow at the location where the PPG unit 555A contacts the auricular skin) and systolic blood pressure data and diastolic blood pressure data, as described hereinbefore. The auricular EEG monitoring system 170 may comprise an EEG recording module 71 in electronic communication with a plurality (at least two, but preferably more than two) of wired or wireless EEG sensor electrodes 72, 73, 74, 75, 82, 84. The EEG sensor electrodes 72, 73, 74, 75, 82, 83, are configured to be located in the auricular housing 11, as described hereinbefore. The EEG recording module 71 is configured to record EEG data of the wearer. The processing unit 50, 401, is in electronic communication with the auricular ECG monitoring system 100, the auricular SCOS unit 511, at least one of: the SPG unit 555 and the PPG unit 555A, and the auricular EEG monitoring system 170. The processing unit 50, 401, is configured to convert the raw EEG data recorded by the EEG recording module 71 into quantitative EEG (qEEG) data.

The cardiovascular monitoring apparatus 600 is configured to record the wearer's ECG data and to detect presence of significant hemodynamic change. As used herein, significant hemodynamic change comprises (or refers to) at least one of the following: a decrease of cerebral blood flow more than a predetermined percentage (e.g. a decrease of 30% or more from the baseline) as recorded by the auricular SCOS unit 511, a decrease of systolic blood pressure more than a predetermined amount (e.g. a decrease of 20 mmHg or more from the baseline) (or a decrease of systolic blood pressure below a predetermined level, e.g. 90 mmHg) as recorded by at least one of: the SPG unit 555 and the PPG unit 555A, a decrease of diastolic blood pressure more than a predetermined amount (e.g. a decrease of 20 mmHg or more from the baseline) (or a decrease of diastolic blood pressure below a predetermined level, e.g. 55 mmHg) as recorded by at least one of: the SPG unit 555 and the PPG unit 555A, an increase of relative delta power more than a predetermined percentage (e.g. an increase of 30% of more from the baseline) as revealed by qEEG data, an increase of delta/alpha ratio more than a predetermined percentage (e.g. an increase of 30% or more from the baseline) as revealed by qEEG data, and a decrease of cordance z-score more than a predetermined amount (e.g. a decrease of 1 standard deviation or more) as revealed by qEEG data.

The neuromodulation system 300 comprises at least one of the following components: a transcutaneous auricular vagus nerve stimulation unit (taVNS unit) 30, a supraorbital nerve stimulation unit 301, an auriculotemporal nerve stimulation unit 302, an occipital nerve stimulation unit 303, a greater auricular nerve stimulation unit 304, an infraorbital nerve stimulation unit 305 and a median nerve stimulation unit 306. The taVNS unit 30 may include a taVNS stimulating electrode 31 configured to contact vagus innervated auricular skin of the wearer 900. The vagus innervated auricular skin includes external ear canal 904, tragus 905, cymba-concha 906, cavum-concha 907 and small adjacent areas. More specifically, the vagus innervated auricular skin includes: inner posterior portion of tragus 905, cymba-concha 906, cavum-concha 907, posterior and inferior walls of the external ear canal 904 and small adjacent regions of the external ear. The supraorbital nerve stimulation unit 301 may include a supraorbital nerve stimulating electrode 311, configured to contact supraorbital nerve innervated mid-forehead skin of the wearer 900. The auriculotemporal nerve (ATN) stimulation unit 302 may include an ATN stimulating electrode 312 configured to contact ATN innervated auricular skin of the wearer. The ATN innervated auricular skin includes: anterior outer part of tragus 905, anterior-superior part of cavum concha 907, anterior and superior walls of the external ear canal 904 and, anterior and superior part of pinna including anterior-superior helix 909. The occipital nerve stimulation unit 303 may include an occipital nerve stimulating electrode 313 configured to contact occipital nerve innervated mid-occipital region of the wearer. The greater auricular nerve (GAN) stimulation unit 304 may include a GAN stimulating electrode 314 configured to contact GAN innervated auricular skin. The GAN innervated auricular skin includes: the cavum concha 907, including the lower/inferior portion of cavum concha, the inferior part of auricle (both anterior and posterior surfaces), the skin over the mastoid process, the lobule, and the lower portion of the antihelix. The infraorbital nerve stimulation unit 305 may include an infraorbital nerve stimulating electrode 315 configured to contact infraorbital nerve innervated mid-facial skin of the wearer. The median nerve stimulation unit 306 may include a median nerve stimulating electrode 316 configured to contact the wearer's skin at Neiguan point on the palmar surface of distal forearm, directly over the median nerve pathway. (The Neiguan point is located directly over the median nerve pathway on the inner forearm about 1.3-2 inches proximal to the wrist crease. The median nerve passes underneath the Neiguan point.) Each component (30, 301, 302, 303, 304, 305, 306) of the neuromodulation unit 300 is configured to give neuromodulating electric stimulation to the wearer when activated or prompted.

In some embodiments, an automatic detection-therapy system for cardiac arrhythmia 101 may comprise an auricular ECG monitoring system 100, a neuromodulation system 300 and a processing unit 50, 401. The neuromodulation system 300 may comprise a transcutaneous auricular vagus nerve stimulation unit (taVNS unit) 30 having a taVNS stimulating electrode 31, configured to contact vagus innervated auricular skin when in use. The vagus innervated auricular skin that the stimulating electrode 31 is configured to contact may be selected from at least one of the following: external ear canal 904, tragus 905, cymba-concha 906, and cavum-concha 907 of the wearer's ear 902. The ECG recording module 20 may be in wired or wireless electronic communication (e.g., through wire, Bluetooth, etc.) with the processing unit 50, 401. The taVNS unit 30 may also be in electronic communication with the processing unit 50, 401. The processing unit 50, 401, may be configured to analyze ECG data recorded by the auricular ECG monitoring system 100 to detect the presence or cessation of ECG signals (ECG data) suggestive of serious cardiac arrhythmias or impending serious cardiac arrhythmias. When the presence of ECG data suggestive of at least one serious cardiac arrhythmia is detected by the processing unit 50, 401, the processing unit 50, 401, may be configured to immediately send arrhythmia-warning-signals to the taVNS unit 30 and upon receiving the arrhythmia-warning-signals from the processing unit 50, 401, the taVNS unit 30 is configured to automatically start sending pre-determined (or closed-loop controlled) electric stimulation to the vagus innervated auricular skin of the wearer's ear 902 to which the taVNS stimulating electrode 31 is in contact with (examples of stimulating parameters as in Table 1). When the presence of ECG signals suggestive of at least one impending serious cardiac arrhythmia is detected by the processing unit 50, 401, the processing unit 50, 401, may be configured to immediately send impending-arrhythmia-warning-signals to the taVNS unit 30 and upon receiving the impending-arrhythmia-warning-signals from the processing unit 50, 401, the taVNS unit 30 is configured to automatically start sending pre-determined (or closed-loop controlled) electric stimulation to the vagus innervated auricular skin of the wearer's ear 902 to which the taVNS stimulating electrode 31 is in contact with (examples of stimulating parameters as in Table 2). When cessation of ECG signals suggestive of the at least one serious cardiac arrhythmias is detected by the processing unit 50, 401, the processing unit 50, 401, may be further configured to immediately send arrhythmia-cessation-signals to the taVNS unit 30 and upon receiving arrhythmia-cessation-signals from the processing unit the taVNS unit 30 is configured to automatically stop sending electric stimulation to the vagus innervated auricular skin of the wearer's ear 902. When cessation of ECG signals suggestive of the at least one impending serious cardiac arrhythmias is detected by the processing unit 50, 401, the processing unit 50, 401, may be further configured to immediately send impending-arrhythmia-cessation signals to the taVNS unit 30 and upon receiving the impending-arrhythmia-cessation signals from the processing unit the taVNS unit 30 is configured to automatically stop sending electric stimulation to the vagus innervated auricular skin of the wearer's ear 902.

In some embodiments, an automatic detection-therapy system for cardiac arrhythmia 101 may comprise one or two transcutaneous auricular vagus nerve stimulation (taVNS) units 30 such as shown in FIG. 18. Optionally, the automatic detection-therapy system for cardiac arrhythmia 101 may comprise a first taVNS unit 30 and a second taVNS unit 30. Preferably, the first taVNS unit 30 comprises a first taVNS stimulating electrode 31 that is configured to contact vagus innervated auricular skin of the wearer's first ear 902, while the second taVNS unit 30 comprises a second taVNS stimulating electrode 31 that is configured to contact vagus innervated auricular skin of the wearer's second ear 902. (It had been reported that bilateral vagus nerve stimulation with bilateral taVNS units may be more effective than unilateral vagus nerve stimulation). A processing unit 50, 401, may be in electronic communication with the first and the second taVNS units 30. When the presence of ECG signals suggestive of a serious cardiac arrhythmia is detected by the processing unit 50, 401, the processing unit 50, 401, may be configured to immediately send arrhythmia-warning-signals to the first and the second taVNS units 30 to cause the first and the second taVNS units 30 to automatically start sending pre-determined (or closed-loop controlled) electric stimulation to the vagus innervated auricular skin of the wearer's first and second ears 902 (examples of stimulation parameters as in Table 1). When the presence of ECG signals suggestive of an impending serious cardiac arrhythmia is detected by the processing unit 50, 401, the processing unit 50, 401, may be configured to immediately send impending-arrhythmia-warning-signals to the first and the second taVNS units 30 to cause the first and the second taVNS units 30 to automatically start sending pre-determined (or closed-loop controlled) electric stimulation to the vagus innervated auricular skin of the wearer's first and second ears 902. (examples of stimulation parameters as shown in Table 2). When cessation of ECG signals suggestive of serious cardiac arrhythmia or impending cardiac arrhythmia is detected by the processing unit 50, 401, the processing unit 50, 401, may be further configured to immediately send arrhythmia-cessation-signals or impending-arrhythmia-cessation signals to the first and the second taVNS units 30 to cause the first and the second taVNS units 30 to automatically stop or modify sending electric stimulation to the vagus innervated auricular skin of the wearer's first and second ears 902.

The ECG recording module 20 and the taVNS unit 30 may both be in electronic communication 19 with a processing unit 50, 401, through Bluetooth, a wired connection, or other electronic connecting means. The ECG data recorded by the ECG recording module 20 are transmitted or otherwise communicated to the processing unit 50, 401. The processing unit 50, 401, may be configured to analyze the ECG data to detect presence or cessation of any serious cardiac arrhythmia and presence or cessation of any impending serious cardiac arrhythmia, including atrial fibrillation (AFib), atrial flutter (AFL), supraventricular tachycardia (SVT), ventricular tachycardia (VT), ventricular fibrillation (VF) or other cardiac arrhythmia, using ECG analysis algorithms (ECG analysis algorithms as known in the art). Upon detection of presence of a serious cardiac arrhythmia, the processing unit 50, 401, may be configured to automatically send arrhythmia-warning-signals to actuate the taVNS unit 30 to immediately start sending closed-loop controlled (or pre-determined) electric stimulation to the auricular branch of vagus nerve via the stimulating electrode 31 (examples of stimulating parameters as shown in Table 1). Upon detection of presence of an impending serious cardiac arrhythmia, the processing unit 50, 401, may be configured to automatically send impending-arrhythmia-warning-signals to actuate the taVNS unit 30 to immediately start sending closed-loop controlled (or pre-determined) electric stimulation to the auricular branch of vagus nerve via the stimulating electrode 31 (examples of stimulating parameters as shown in Table 2). When the processing unit 50, 401, detects cessation of the serious cardiac arrhythmia or cessation of the impending serious cardiac arrhythmia, the processing unit 50, 401, may be configured to send arrhythmia-cessation-signals or impending-arrhythmia-cessation signals to the taVNS unit 30 to modify or stop the electric stimulation to the vagus nerve. This automatic detection-therapy system for cardiac arrhythmia 101 may automatically initiate therapeutic intervention instantly when a serious cardiac arrhythmia or an impending serious cardiac arrhythmia is detected. This can avoid the inevitable delay associated with the traditional set-up where the ECG data being sent to a monitoring center and the monitoring center notifying healthcare provider 950 via their client device 400 to arrange for therapeutic intervention.

In preferred embodiments, an automatic detection-therapy system for cardiac arrhythmia 101 may comprise a cardiovascular monitoring apparatus 600, a neuromodulation system 300, and a processing unit 50, 401. The processing unit 50, 401, may be in electronic communication with the cardiovascular monitoring apparatus 600 and the neuromodulation system 300. The cardiovascular monitoring apparatus 600 may comprise an auricular ECG monitoring system 100. The auricular ECG monitoring system 100 includes an ECG recording module 20 and at least two ECG sensor electrodes 12, 13, in electronic communication with the ECG recording module 20. At least one of the ECG sensor electrodes 12, 13, (for example a first ECG sensor electrode 12) may be configured to be housed in an auricular housing 11 and the auricular housing 11 is configured to be placed in a first ear 902 of a wearer. The first ECG sensor electrode 12 is configured to be located on the skin of a first ear 902 of a wearer, selected from at least one of the following: an external ear 902 of the wearer's first ear, an external ear canal 904 of the wearer's first ear and a peri-auricular area 903 around the first ear of the wearer. At lease another ECG sensor electrode (for example a second ECG sensor electrode 13) may be also configured to be located in the first ear 902 of the wearer, at a separate location selected from one of the following: the external ear 902 of the wearer's first ear, the external ear canal 904 of the wearer's first ear and the peri-auricular area 903 around the wearer's first ear. Alternatively, the second ECG sensor electrode 13 may be configured to be located on one of the following: a second ear 902 of the wearer, a wrist of the wearer (such as in a wrist band 150 or a wrist watch 400A or a health tracker), a finger 281A of the wearer, an ankle 174 of the wearer and a toe 271A of the wearer. The first ECG sensor electrode 12 and the second ECG sensor electrode 13 are in electronic communication with the processing unit 50, 401. The ECG recording module 20 is configured to record ECG data of the wearer. The neuromodulation system 300 comprises at least a neuromodulation unit selected from at least one of the following components: a taVNS unit 30, a supraorbital nerve stimulation unit 301, an auriculotemporal nerve stimulation unit 302, an occipital nerve stimulation unit 303, a greater auricular nerve stimulation unit 304, an infraorbital nerve stimulation unit 305 and a median nerve stimulation unit 306. Each component of the neuromodulation system 300 is configured to give neuromodulating electric stimulation to the wearer 900 when activated or prompted. The processing unit 50, 401, may comprise a closed-loop control system 333 and R-wave synchronization mode. The processing unit 50, 401, may be configured to analyze the ECG data recorded by the ECG recording module 20 (transmitted from the auricular ECG monitoring system 100) to detect presence or cessation of ECG signals suggestive of serious cardiac arrhythmia. The processing unit 50, 401 is also configured to analyze the ECG data recorded by the ECG recording module 20 to detect presence or cessation of ECG signals suggestive of impending serious cardiac arrhythmia. When serious cardiac arrhythmia is detected by the processing unit 50, 401, the processing unit 50, 401, is configured to send signals to the neuromodulation system 300 to prompt the neuromodulation system 300 to start sending neuromodulating electric stimulation to the wearer 900 using the R-wave synchronization mode under the guidance of the closed-loop control system 333 utilizing continuous input data from the cardiovascular monitoring apparatus 600. When impending serious cardiac arrhythmia is detected by the processing unit 50, 401, the processing unit is further configured to send signals to the neuromodulation system 300 to prompt the neuromodulation system 300 to start sending neuromodulating electric stimulation to the wearer using the R-wave synchronization mode under the guidance of the closed-loop control system utilizing continuous input data from the cardiovascular monitoring apparatus 600.

In some embodiments, an automatic detection-therapy system for cardiac arrhythmia 101 may comprise a cardiovascular monitoring apparatus 600 that comprises an auricular ECG monitoring system 100 and an auricular speckle contrast optical spectroscopy (SCOS) unit 511. The auricular ECG monitoring system 100 is as described hereinbefore. The auricular SCOS unit 511 may be configured to be located at an area of a first ear 902 of the wearer. The area that the auricular SCOS unit 511 is configured to be located is selected from one of the following: an external ear 902 of the first ear of the wearer 900, an external ear canal 904 of the first ear of the wearer and a periauricular area 903 around the first ear of the wearer 900. The auricular SCOS unit 511 may be configured to record the cerebral and extracranial blood flow data of the wearer 900. The processing unit 50, 401, is in electronic communication with the auricular SCOS unit 511 and the auricular ECG monitoring system 100. The processing unit 50, 401, is configured to analyze the cerebral and extracranial blood flow data recorded by the auricular SCOS unit 511 to assess the wearer's cerebral and extracranial blood flow. The processing unit 50, 401, comprises a closed-loop control system 333 that includes a R-wave synchronization mode. When presence of serious cardiac arrhythmia or impending serious cardiac arrhythmia is detected by the processing unit 50, 401, the processing unit 50, 401, and the closed-loop control system 333 are configured to utilize continuous input data of the wearer's ECG from the auricular ECG monitoring system 100 and continuous input data of the wearer's cerebral blood flow from the auricular SCOS unit 511 to guide the neuromodulating electric stimulation to the wearer 900, including controlling the stimulation parameters, duration, timing, interval and selection of which component(s) of the neuromodulation system 300, etc. The closed-loop control system 333 is further configured to use the R-wave synchronization mode to guide the neuromodulating electric stimulation to the wearer.

In some embodiments, an automatic detection-therapy system for cardiac arrhythmia 101, may comprise a cardiovascular monitoring apparatus 600 that may comprise an auricular ECG monitoring system 100 and at least one of: a speckle-plethysmography unit (SPG unit) 555 and a photoplethysmography (PPG) unit 555A. The auricular ECG monitoring system 100 is as described hereinbefore. The SPG unit 555 may be configured to contact a skin area located at the wearer's first ear 902 and the skin area that the SPG unit 555 is configured to contact may be selected from one of the following: the external ear 902 of the first ear of the wearer, the external ear canal 904 of the first ear of the wearer and the periauricular area 903 around the first ear of the wearer. The SPG unit 555 may be configured to record the wearer's systolic blood pressure data, diastolic blood pressure data and regional blood flow data at the location of the skin area where the SPG unit 555 contacts the wearer's ear. The SPG unit 555 and the auricular ECG monitoring system 100 may be in electronic communication with the processing unit 50, 401. The processing unit 50, 401, may be configured to analyze the regional blood flow data, the systolic blood pressure data and the diastolic blood pressure data recorded by the SPG unit 555 to assess the wearer's regional blood flow status and blood pressure status. When presence of serious cardiac arrhythmia or impending serious cardiac arrhythmia is detected by the processing unit 50, 401, the processing unit 50, 401, and the closed-loop control system 333 may be configured to utilize continuous input data of the wearer's ECG data from the auricular ECG monitoring system 100 and continuous input data of the wearer's regional blood flow data and systolic blood pressure data and diastolic blood pressure data from the SPG unit 555 to guide the neuromodulating electric stimulation to the wearer, including controlling the stimulation parameters, duration, timing, interval and selection of which component(s) of the neuromodulation system 300, etc. The closed-loop control system 333 is further configured to use the R-wave synchronization mode to guide the neuromodulating electric stimulation to the wearer. Likewise, the PPG unit 555A may be configured to contact a skin area located at the wearer's first ear 902 and the skin area that the PPG unit 555A is configured to contact may be selected from one of the following: the external ear 902 of the first ear of the wearer, the external ear canal 904 of the first ear of the wearer and the periauricular area 903 around the first ear of the wearer. The PPG unit 555A may be configured to record the wearer's systolic blood pressure data, diastolic blood pressure data and regional blood flow data at the location of the skin area where the PPG unit 555A contacts the wearer's ear. The PPG unit 555A and the auricular ECG monitoring system 100 may be in electronic communication with the processing unit 50, 401. The processing unit 50, 401, may be configured to analyze the regional blood flow data, the systolic blood pressure data and the diastolic blood pressure data recorded by the PPG unit 555A to assess the wearer's regional blood flow status and blood pressure status. When presence of serious cardiac arrhythmia or impending serious cardiac arrhythmia is detected by the processing unit 50, 401, the processing unit 50, 401, and the closed-loop control system 333 may be configured to utilize continuous input data of the wearer's ECG data from the auricular ECG monitoring system 100 and continuous input data of the wearer's regional blood flow data and systolic blood pressure data and diastolic blood pressure data from the PPG unit 555A to guide the neuromodulating electric stimulation to the wearer, including controlling the stimulation parameters, duration, timing, interval and selection of which component(s) of the neuromodulation system 300, etc. The closed-loop control system 333 is further configured to use the R-wave synchronization mode to guide the neuromodulating electric stimulation to the wearer.

In some embodiments, an automatic detection-therapy system for cardiac arrhythmia 101, may comprise a cardiovascular monitoring apparatus 600 that may comprise an auricular ECG monitoring system 100 and an auricular electroencephalogram (EEG) monitoring system 170. The auricular electroencephalogram (EEG) monitoring system (auricular EEG system) 170 may comprise an EEG recording module 71 having a plurality (at least two, but preferably more than two) of EEG sensor electrodes 72, 73, 74, 75, 82, 83, and each EEG sensor electrode 72, 73, 74, 75, 82, 83, is in electronic communication with the EEG recording module 71. Each EEG sensor electrode 72, 73, 74, 75, 82, 83, may be configured to contact a skin area of a first ear 902 of the wearer 900. The skin area that each EEG sensor electrode 72, 73, 74, 75, 82, 83, may be configured to contact is selected from one of the following: an external ear 902 of the first ear of the wearer, an external ear canal 904 of the first ear of the wearer and a periauricular area 903 around the first ear of the wearer. Each EEG sensor electrode 72, 73, 74, 75, 82, 83, may be configured to contact different skin area of the wearer's first ear 902. The EEG recording module 20 may be configured to record EEG data of the wearer 900. The auricular EEG monitoring system 100 is in electronic communication with a processing unit 50, 401. The processing unit 50, 401, may be configured to convert the EEG data into quantitative EEG (qEEG) data. The processing unit 50, 401, may be configured to analyze the EEG data and qEEG data recorded by the EEG recording module 71 to assess the wearer's relative delta power, delta/alpha ratio and cordance z-score (or other relevant parameters or metrics available from qEEG), etc. The processing unit 50, 401, may be configured to analyzed the EEG data and qEEG data recorded by the EEG recording module 20 to assess the wearer's cerebral blood perfusion data and to detect presence of a significant decrease of cerebral blood perfusion. As used herein, significant decrease of cerebral blood perfusion as detected by qEEG comprises at least one of the following: an increase of relative delta power more than a predetermined percentage (e.g. an increase of 30% or more), an increase of delta/alpha ratio more than a predetermined percentage (e.g. an increase of 30% or more), and a decrease of cordance z-score more than a predetermined amount (e.g. a decrease of 1 SD or more) (these are suggestive of decrease of cerebral blood perfusion below a certain level). (Assessing cerebral blood perfusion via qEEG metrics is one of the factors in assessing hemodynamic changes.) When presence of serious cardiac arrhythmia or impending serious cardiac arrhythmia is detected by the processing unit 50, 401, the processing unit 50, 401, and the closed-loop control system 333 may be configured to utilize continuous input data of the wearer's ECG data from the auricular ECG monitoring system 100 and continuous input data of at least one of: the relative delta power per the qEEG, the delta/alpha ratio per the qEEG and the cordance z-score per the qEEG from the auricular EEG monitoring system 170 to guide the neuromodulating electric stimulation to the wearer, including controlling the stimulation parameters, duration, timing, interval and selection of at least one component of the neuromodulation system 300, etc. The closed-loop control system 333 and the processing unit 50, 401, are further configured to use the R-wave synchronization mode to guide the neuromodulating electric stimulation to the wearer 900.

In some embodiments, an automatic detection-therapy system for cardiac arrhythmia 101, may comprise a cardiovascular monitoring apparatus 600 that may comprise an auricular ECG monitoring system 100, an auricular speckle contrast optical spectroscopy unit (auricular SCOS unit) 511, and at least one of: a speckle-plethysmography (SPG) unit 555 and a photoplethysmography unit (PPG unit) 555A. The auricular ECG monitoring system 100, the auricular SCOS unit 511, the SPG unit 555 and the PPG unit 555A are as described hereinbefore. The processing unit 50, 401, may be in electronic communication with the auricular ECG monitoring system 100, the auricular SCOS unit 511 and at least one of: the SPG unit 555 and the PPG unit 555A. The processing unit 50, 401, may be configured to analyze the cerebral and extracranial blood flow data recorded by the auricular SCOS unit 511 and the systolic blood pressure data, diastolic blood pressure data and regional blood flow data recorded by at least one of: the SPG unit 555 and the PPG unit 555A, to assess the wearer's cerebral hemodynamic data. The closed-loop control system 333 and the processing unit 50, 401, may be configured to utilize the wearer's ECG data from the auricular ECG monitoring system 100 and the cerebral hemodynamic data from at least one of: the auricular SCOS unit 511, the SPG unit 555 and the PPG unit 555A, to guide the neuromodulating electric stimulation to the wearer 900. The closed-loop control system 333 and the processing unit 50, 401, may be further configured to use the R-wave synchronization mode to guide the neuromodulating electric stimulation to the wearer 900. When presence of serious cardiac arrhythmia or impending serious cardiac arrhythmia is detected by the processing unit 50, 401, the processing unit 50, 401, and the closed-loop control system 333 may be configured to utilize the continuous input data of the wearer's ECG data from the auricular ECG monitoring system 100, the continuous input data of the wearer's cerebral blood flow from the auricular SCOS unit 511, and the continuous input data of the wearer's regional blood flow data and systolic and diastolic blood pressure data from at least one of: the SPG unit 555 and the PPG unit 555A to guide the neuromodulating electric stimulation to the wearer 900, including controlling the stimulation parameters, duration, timing, interval and selection of at least one component of the neuromodulation system 300, etc. The closed-loop control system 333 and the processing unit 50, 401, are further configured to use the R-wave synchronization mode to guide the neuromodulating electric stimulation to the wearer 900.

In some embodiments, an automatic detection-therapy system for cardiac arrhythmia 101, may comprise a cardiovascular monitoring apparatus 600 that may comprise an auricular ECG monitoring system 100, an auricular speckle contrast optical spectroscopy unit (auricular SCOS unit) 511, at least one of: a speckle-plethysmography unit (SPG unit) 555 and a photoplethysmography (PPG) unit 555A, and an auricular electroencephalogram (EEG) monitoring system 170. The auricular ECG monitoring system 100, the auricular SCOS unit 511, the SPG unit 555 and the PPG unit 555A are as described hereinbefore. The auricular electroencephalogram (EEG) monitoring system (auricular EEG system) 170 may comprise an EEG recording module 71 having a plurality (at least two, but preferably more than two) of EEG sensor electrodes 72, 73, 74, 75, 82, 83 and each EEG sensor electrode 72, 73, 74, 75, 82, 83, is in electronic communication with the EEG recording module 71. Each EEG sensor electrode 72, 73, 74, 75, 82, 83, may be configured to contact a skin area of a first ear 902 of the wearer 900. The skin area that each EEG sensor electrode 72, 73, 74, 75, 82, 83, may be configured to contact is selected from one of the following: an external ear 902 of the first ear of the wearer, an external ear canal 904 of the first ear of the wearer and a periauricular area 903 around the first ear of the wearer. Each EEG sensor electrode 72, 73, 74, 75, 82, 83, may be configured to contact different skin area of the wearer's first ear 902. The EEG recording module 20 may be configured to record EEG data of the wearer 900. The auricular EEG monitoring system 170 is in electronic communication with a processing unit 50, 401. The processing unit 50, 401, may be configured to convert the EEG data into quantitative EEG (qEEG) data. The processing unit 50, 401, is configured to analyze the EEG data and qEEG data recorded by the auricular EEG monitoring system to assess the wearer's relative delta power, delta/alpha ratio and cordance z-score (or other relevant parameters or metrics available from qEEG), etc. The processing unit 50, 401, may be further configured to analyzed the EEG data and qEEG data recorded by the EEG recording module 71 to assess (indirectly) the wearer's cerebral blood perfusion data and to detect presence of a significant decrease of cerebral blood perfusion. (It is known that qEEG assesses cerebral blood perfusion indirectly by measuring the electrical activity of the brain, which is closely related to metabolic activity and blood flow. While not a direct measure of blood flow itself, qEEG (specifically cordance) strongly correlates with perfusion, allowing detection of ischemia, reduced blood flow, and metabolic changes.) As used herein, significant decrease of cerebral blood perfusion as detected by qEEG comprises at least one of the following: an increase of relative delta power more than a predetermined percentage (e.g. an increase of 30% or more), an increase of delta/alpha ratio more than a predetermined percentage (e.g. an increase of 30% or more), and a decrease of cordance z-score more than a predetermined amount (e.g. a decrease of 1 SD or more). The processing unit 50, 401, is also configured to analyze the cerebral and extracranial blood flow data recorded by the auricular SCOS unit 511, the systolic blood pressure data, diastolic blood pressure data and regional blood flow data recorded by at least one of: the SPG unit 555 and the PPG unit 555A, and the cerebral blood perfusion data recorded by the auricular EEG monitoring system 170 to guide the neuromodulating electric stimulation. When presence of serious cardiac arrhythmia or impending serious cardiac arrhythmia is detected by the processing unit 50, 401, the processing unit 50, 401, and the closed-loop control system 333 are configured to utilize continuous input data of the wearer's ECG from the auricular ECG monitoring system 100, continuous input data of the cerebral blood flow from the auricular SCOS unit 511, continuous input data of the systolic blood pressure diastolic blood pressure and regional blood flow from at least one of: the SPG unit 555 and the PPG unit 555A, and continuous input data of the cerebral blood perfusion from the auricular EEG monitoring system 170 to guide the neuromodulating electric stimulation to the wearer 900, including controlling the stimulation parameters, duration, timing, interval and selection of at least one component of the neuromodulation system 300. The closed-loop control system 333 and the processing unit 50, 401, are further configured to use the R-wave synchronization mode to guide the neuromodulating electric stimulation to the wearer 900. Please note that relative delta power, delta/alpha ratio and cordance z-score as obtained from qEEG may be used to assess the wearer's cerebral blood perfusion, cerebral blood flow and metabolism, etc. There are other qEEG parameters and metrics, for example: cordance (combining absolute and relative power), spectral edge frequency/median frequency, absolute delta power, alpha+beta/theta+delta ratio, spectral power, coherence, event-related potentials, phase-locking values and brain symmetry index, etc. that are also useful to assess the wearer's cerebral blood perfusion, cerebral blood flow and metabolism. Usage of the other parameters and metrics from the qEEG for cardiac arrhythmia assessment and for guidance of the closed-loop control system 333 is within the scope of this invention.

In some embodiments, an automatic detection-therapy system for cardiac arrhythmia 101 may comprise a neuromodulation system 300 that comprises at least one of the following components: a first transcutaneous auricular vagus nerve stimulation unit (taVNS unit) 30, a supraorbital nerve stimulation unit 301, a first auriculotemporal nerve stimulation unit 302, an occipital nerve stimulation unit 303, a first greater auricular nerve stimulation unit 304, an infraorbital nerve stimulation unit 305 and a median nerve stimulation unit 306. Each component of the neuromodulation system 300 is configured to give neuromodulating electric stimulation to the wearer 900 when prompted or activated. The first taVNS unit 30 may comprise a first taVNS stimulating electrode 31 configured to contact vagus innervated auricular skin of the wearer's first ear 902 when in use. The supraorbital nerve stimulation unit 301 may comprise a supraorbital nerve stimulating electrode 311 configured to contact supraorbital nerve innervated midforehead skin of the wearer 900 when in use. The first auriculotemporal nerve stimulating unit 302 may comprise a first auriculotemporal nerve stimulating electrode 312 configured to contact auriculotemporal nerve innervated auricular skin of the wearer's first ear 902 when in use. The occipital nerve stimulation unit 303 may comprise an occipital nerve stimulating electrode 313 configured to contact the wearer's mid-occipital region when in use. The first greater auricular nerve stimulation unit 304 comprises a first greater auricular nerve stimulating electrode 314 configured to contact greater auricular nerve innervated auricular skin of the wearer's first ear 902 when in use. The infraorbital nerve stimulation unit 305 may comprise an infraorbital nerve stimulating electrode 315 that is configured to contact infraorbital nerve innervated mid-facial skin of the wearer 900 when in use. The median nerve stimulation unit 306 may comprise a median nerve stimulating electrode 316 that is configured to contact the wearer's Neiguan point on the palmar side of the wearer's distal forearm. (The Neiguan point, also known as PC6 or Pericardium 6, is located on the inner forearm directly over the area where the median nerve passes. It is specifically positioned between two large, central tendons—the palmaris longus and the flexor carpi radialis—about two to three finger-widths proximal to the wrist crease. The Neiguan point is the primary target for transcutaneous stimulation of the median nerve.)

In some embodiments, an automatic detection-therapy system for cardiac arrhythmia 101 may comprise a neuromodulation system 300 that comprises at least two of the following components: a first transcutaneous auricular vagus nerve stimulation unit (taVNS unit) 30, a supraorbital nerve stimulation unit 301, a first auriculotemporal nerve stimulation unit 303, an occipital nerve stimulation unit 303, a first greater auricular nerve stimulation unit 304, an infraorbital nerve stimulation unit 305 and a median nerve stimulation unit 306. Studies have shown that simultaneous stimulation of two different neuromodulation units is more effective than stimulation of each neuromodulation unit alone, due to the synergistic effects. Each component of the neuromodulation system 300 is configured to give neuromodulating electric stimulation to the wearer 900 when activated or prompted. The first taVNS unit 30 comprises a first taVNS stimulating electrode 31 configured to contact vagus innervated auricular skin of the wearer's first ear 902 when in use. The supraorbital nerve stimulation unit 301 may comprise a supraorbital nerve stimulating electrode 311 configured to contact supraorbital nerve innervated midforehead skin of the wearer 900 when in use. The first auriculotemporal nerve stimulating unit 302 may comprise a first auriculotemporal nerve stimulating electrode 312 configured to contact auriculotemporal nerve innervated auricular skin of the wearer's first ear 902 when in use. The occipital nerve stimulation unit 303 may comprise an occipital nerve stimulating electrode 313 configured to contact the wearer's mid-occipital region when in use. The first greater auricular nerve stimulation unit 304 may comprise a first greater auricular nerve stimulating electrode 314 configured to contact greater auricular nerve innervated auricular skin of the wearer's first ear when in use. The infraorbital nerve stimulation unit 305 may comprise an infraorbital nerve stimulating electrode 315 that is configured to contact infraorbital nerve innervated mid-facial skin of the wearer 900 when in use. The median nerve stimulation unit 306 may comprise a median nerve stimulating electrode 316 that is configured to contact the Neiguan point (where the median nerve passes underneath) of the wearer's distal anterior forearm.

In some embodiments, an automatic detection-therapy system for cardiac arrhythmia 101 may have a neuromodulation system 300 that includes a first and a second neuromodulation systems 300. The first neuromodulation system 300 comprises at least one of the following components: a first transcutaneous auricular vagus nerve stimulation unit (taVNS unit) 30, a supraorbital nerve stimulation unit 301, a first auriculotemporal nerve stimulation unit 302, an occipital nerve stimulation unit 303, a first greater auricular nerve stimulation unit 304, an infraorbital nerve stimulation unit 305 and a median nerve stimulation unit 306. The first taVNS unit 30, the first auriculotemporal nerve stimulation unit 302 and first greater auricular nerve stimulation unit 304 are configured to be located in a first ear 902 of the wearer 900. The supraorbital nerve stimulation unit 301 may be configured to be located at midforehead area. The occipital nerve stimulation unit 303 may be configured to be located at mid-occipital region, while the infraorbital nerve stimulation unit 305 may be configured to be located at mid-facial area. The median nerve stimulation unit 306 may be configured to be located at a first distal anterior forearm. The second neuromodulation system 300 may be configured to be located in a second ear 902 of the wearer. Studies have shown that bilateral neuromodulating electric stimulation is more effective than unilateral neuromodulating electric stimulation. The second neuromodulation system 300 may comprise at least one of the following components: a second taVNS unit 30, a second auriculotemporal nerve stimulation unit 302 and a second greater auricular nerve stimulation unit 304. Each component of the first and the second neuromodulation system 300 is in electronic communication with the processing unit 50, 401. Each component of the second neuromodulation system 300 is configured to give neuromodulating electric stimulation to the wearer 900 when prompted or activated. The second taVNS unit 30 comprises a second taVNS stimulating electrode 31 configured to contact vagus innervated auricular skin of the wearer's second ear 902. The second auriculotemporal nerve stimulation unit 302 comprises a second auriculotemporal nerve stimulating electrode 312 configured to contact auriculotemporal nerve innervated auricular skin of the wearer's second ear 902; while the second greater auricular nerve stimulation unit 304 may comprise a second greater auricular nerve stimulating electrode 314 configured to contact greater auricular nerve innervated auricular skin of the wearer's second ear 902. Optionally, the second neuromodulation unit 300 may further comprise a second median nerve stimulation unit 306 to be located at a second distal anterior forearm.

In some embodiments, an automatic detection-therapy system for cardiac arrhythmia 101 may comprise a network interface 53, 406, and at least one of: a speaker 15, 404A, and a vibrator 17, 404B for notification. The processing unit 50, 401, is in electronic communication with the network interface 53, 406, the speaker 15, 404A and the vibrator 17, 404B. When the processing unit 50, 401, detects presence of ECG data suggestive of serious cardiac arrhythmia, the processing unit 50, 401, is configured to generate signals to the network interface 53, 406, and the network interface 53, 406, may be configured to send notification to at least one of: a clint device 400 of the wearer 900 and a client device 400 of a healthcare provider 950 of the wearer. When the processing unit 50, 401, detects presence of ECG data suggestive of serious cardiac arrhythmia, the processing unit 50, 401, is also configured to generate signals to at least one of: the speaker 15, 404A, to generate an audible notification to the client device 400 of the wearer and the vibrator 17, 404B, to generate a tactile notification to the client device 400 of the wearer. When the processing unit 50, 401, detects presence of ECG data suggestive of impending serious cardiac arrhythmia, the processing unit 50, 401, may be configured to generate signals to the network interface 53, 406, and the network interface 53, 406, may be configured to send notification to at least one of: the clint device 400 of the wearer and the client device 400 of the healthcare provider 950 of the wearer. When the processing unit 50, 401, detects presence of ECG data suggestive of impending serious cardiac arrhythmia the processing unit 50, 401, may be also configured to generate signals to at least one of: the speaker 15, 404A, to generate an audible notification to the client device 400 of the wearer and the vibrator 17, 404B, to generate a tactile notification to the client device 400 of the wearer. When the processing unit 50, 401, detects both of the following: cessation of ECG data suggestive of serious cardiac arrhythmia and cessation of ECG data suggestive of impending serious cardiac arrhythmia, the processing unit 50, 401, is further configured to generate signals to the network interface 53, 406, and the network interface 53, 406, is configured to send cessation notification (notice of cessation notification) to one of: the client device 400 of the wearer and the client device 400 of the healthcare provider 950 of the wearer. When the processing unit 50, 401, detect both of the following: cessation of ECG data suggestive of serious cardiac arrhythmia and cessation of ECG data suggestive of impending serious cardiac arrhythmia, the processing unit 50, 401, may be further configured to generate signals to at least one of: the speaker 15, 404A, to generate an audible cessation notification to the client device 400 of the wearer and the vibrator 17, 404B, to generate a tactile cessation notification to the client device 400 of the wearer.

In some embodiments, an automatic detection-therapy system for cardiac arrhythmia 101 may comprise an auricular ECG monitoring system 100, an auricular SCOS unit 511, at least one of: a SPG unit 555 and a PPG unit 555A, an auricular EEG monitoring system 170, a taVNS unit 30, an auriculotemporal nerve (ATN) stimulation unit 302 and a greater auricular nerve (GAN) stimulation unit 304. The auricular ECG monitoring system 100 comprises at least two ECG sensor electrodes, including a first ECG sensor electrode 12 and a second ECG sensor electrode 13. The auricular EEG monitoring system includes a plurality of EEG sensor electrodes 72, 72, 74, 75, 82, 83. The auricular SCOS unit 511 includes a SCOS sensor 512. The taVNS unit 30 includes a taVNS stimulating electrode 31. The ATN stimulation unit 302 includes an ATN stimulating electrode 312 and the GAN stimulation unit 304 includes a GAN stimulating electrode 314. All of the ECG sensor electrodes 12, 13, all of the EEG sensor electrode 72, 73, 74, 75, 82, 83, the auricular SCOS sensor 512, at least one of: the SPG unit 555 and the PPG unit 555A, the taVNS stimulating electrode 31, the ATN stimulating electrode 312 and the GAN stimulating electrode 314 may be all housed together sharing a same auricular housing 11. The auricular housing 11 may be selected from one of the following: a modified earbud housing 61, a modified in-the-ear housing 62, and a behind-the-ear-hearing-aid-style housing 63. All of these housings 61, 62, 63, include a tubular-shaped structure 66 (configured to be inserted into an external ear canal 904 of a wearer when in use) and a body-structure 67 (configured to be placed at the opening of the external ear canal 904 and to sit or be placed inside a tragus-concha bowl 957 of the wearer's ear when in use). All of the EEG sensor electrodes 72, 73, 74, 75, 82, 83 and the first ECG sensor electrode 12 may be configured to be located on a surface 91 of the tubular-shaped structure 66, while the auricular SCOS sensor 512, at least one of: the SPG unit 555 and the PPG unit 555A, the taVNS stimulating electrode 31 and the ATN stimulating electrode 312 are configured to be located on a surface of one of: the tubular-shaped structure 66 and the body-structure 67. The GAN stimulating electrode 314 and the second ECG sensor electrode 13 are configured to be located on a surface of the body-structure 67. The first ECG sensor electrodes 12 and all of the EEG sensor electrode 72, 73, 74, 75, 82, 83, are configured to be partially embedded in the surface with slight protrusion at the surface of the tubular-shaped structure 66. The auricular SCOS sensor 512, at least one of: the SPG unit 555 and the PPG unit 555A, the taVNS stimulating electrode 31, the auriculotemporal nerve (ATN) stimulating electrode 312 are configured to be partially embedded in the surface with slight protrusion at the surface of one of: the tubular-shaped structure 66 and the body-structure 67. (Alternatively, the auricular SCOS sensor 512 may be placed slightly below the surface of the tubular-shaped structure 66 or the body-structure 67 so that there is a small air gap between the SCOS sensor 512 and the wearer's skin when the tubular-shaped structure 66 or the body-structure 67 are placed in the wearer's ear 902.) The GAN stimulating electrode 314 and the second ECG sensor electrode 13 are configured to be partially embedded in the surface with slight protrusion at the surface of the body-structure 67. The tubular-shaped structure 66 and the body-structure 67 may be configured to comprise a flexible elastic and adaptable material (such as soft foam or soft silicone-type material) and the flexible elastic and adaptable material is configured to have appropriate flexibility, elasticity and adaptability so that the tubular-shaped structure 66 will naturally adapt to the contour of the external ear canal 904 and snugly fill the interior of the wearer's external ear canal 904 when the tubular-shaped structure 66 is inserted into the external ear canal 904 of the wearer's ear 902 and, at the same time, the body-structure 67 will naturally adapt to the contour of the tragus-concha bowl 957 of the wearer's ear 902 and snugly fill the interior of the tragus-concha bowl 957 of the wearer's ear 902 when the body-structure 67 is placed inside the tragus-concha bowl 957 of the wearer's ear 902, so that the first ECG sensor electrodes 12 and all of the EEG sensor electrodes 72, 73, 74, 75, 82, 83, are naturally snugly in contact with the skin of the external ear canal 904 of the wearer's ear 902, and, at the same time, the auricular SCOS sensor 512, at least one of: the SPG unit 555 and the PPG unit 555A, the taVNS stimulating electrode 31 and the ATN stimulating electrode 312 are naturally and snugly in contact with the skin of the tragus-concha bowl 957 of the wearer's ear 902 or the skin of the external ear canal 904 of the wearer's ear 902, while the GAN stimulating electrode 314 and the second ECG sensor electrode 13 are naturally snugly in contact with the skin of the wearer's tragus-concha bowl 957, and so that the taVNS stimulating electrode 31 is naturally and snugly in contact with its target skin of vagus-innervated auricular skin of the wearer's first ear 902, provided by carefully selecting a location for the taVNS stimulating electrode 31 on the tubular-shaped structure 66 or the body-structure 67 to match one of the innervation locations of the vagus innervated skin on one of: the external ear canal 904 and the tragus-concha bowl 957, and, at the same time, the ATN stimulating electrode 312 will be naturally in close contact with its target skin of ATN innervated auricular skin, provided by carefully selecting a location for the ATN stimulating electrode 312 on the tubular-shaped structure 66 or the body-structure 67 to match one of the innervation locations of the ATN innervated skin on the external ear canal 904 or the tragus-concha bowl 957, and, at the same time, the GAN stimulating electrode 314 will be naturally in close contact with GAN innervated auricular skin, provided by carefully selecting a location for the GAN stimulating electrode 314 on the body-structure 67 to match one of the innervation locations of the GAN innervated skin on tragus-concha bowl 957. As described hereinbefore, by comparing the innervation patterns, it is obvious that the tragus-concha 957 of a wearer's ear 902 receives mixed and overlapped innervation from the auricular branch of vagus nerve, the ATN and the GAN. The external ear canal 904 receives mixed innervation from the auricular branch of vagus nerve and the auriculotemporal nerve (ATN).

Please note that the external ear canal 904 and tragus-concha bowl 957 of the wearer's ear received mixed and sometimes overlapping innervation from the auricular branch of vagus nerve, the auriculotemporal nerve (ATN) and greater auricular nerve (GAN). For comparison: The vagus-innervated auricular skin includes: inner posterior portion of tragus 905, cymba-concha 906, cavum-concha 907, posterior and inferior walls of the external ear canal 904 and small adjacent regions of the external ear. The auriculotemporal nerve (ATN) innervated auricular skin includes: anterior outer part of tragus 905, anterior-superior part of cavum concha 907, anterior and superior walls of the external ear canal 904 and, anterior and superior part of pinna including anterior-superior helix 909. The greater auricular nerve (GAN) innervated auricular skin includes the cavum concha (the lower or inferior portion of cavum concha of both anterior and posterior surfaces), the lower or inferior part of auricle including both anterior and posterior surfaces, lobule, lower part of antihelix and the mastoid process.

It should be noted that the designs, setups and the material for the auricular housing 11 as described hereinbefore enable all of the pertinent electrodes and sensors, including the ECG sensor electrodes 12, 13, the EEG sensor electrodes 72, 73, 74, 75, 82, 83, the SCOS sensor 512, the SPG unit 555 (or the PPG unit 555A), the taVNS stimulating electrode 31, the auriculotemporal nerve (ATN) stimulating electrode 312 and the greater auricular nerve (GAN) stimulating electrode 314, to be self-installable (user-installable) and self-removable (user-removable). The designs, setups and the material for the auricular housing 11 as described hereinbefore also enable the taVNS stimulating electrode 31 to automatically contact the vagus innervated auricular skin, the ATN stimulating electrode 312 to automatically contact the ATN innervated auricular skin and the GAN stimulating electrode 314 to automatically contact the GAN innervated auricular skin when the auricular housing 11 is placed in the wearer's ear 902 (including the external ear canal 904 and the tragus-concha bowl 957 of the wearer's ear 902). In some embodiments, an automatic detection-therapy system for cardiac arrhythmia 101 may comprise a neuromodulation system 300 that comprises the following 3 components: a transcutaneous auricular vagus nerve stimulation unit (taVNS unit) 30, an auriculotemporal nerve (ATN) stimulation unit 302, and a greater auricular nerve (GAN) stimulation unit 304. Each component of the neuromodulation system 300 may be configured to give neuromodulating electric stimulation to the wearer when activated or prompted. The taVNS unit 30 may include a taVNS stimulating electrode 31, the ATN stimulation unit 302 may include an ATN stimulating electrode 312, and the GAN stimulation unit 304 may include a GAN stimulating electrode 314. The taVNS stimulating electrode 31, the ATN stimulating electrode 312 and the GAN stimulating electrode 314 may be configured to be housed together in an auricular housing 11 and are further configured to automatically contact their respective innervated auricular skin when the auricular housing 11 is placed in the wearer's ear 902, similar to the aforementioned descriptions.

Similarly, in some embodiments, a closed-loop neuromodulation apparatus 300 (to be further described hereinafter) may comprise a neuromodulation system 300 that comprises the following 3 components: a transcutaneous auricular vagus nerve stimulation unit (taVNS unit) 30, an auriculotemporal nerve stimulation unit 302, and a greater auricular nerve stimulation unit 304. Each component of the neuromodulation system 300 may be configured to give neuromodulating electric stimulation to the wearer when activated or prompted. The taVNS unit 30 may include a taVNS stimulating electrode 31, the auriculotemporal nerve stimulation unit 302 may include an auriculotemporal nerve stimulating electrode 312, and the greater auricular nerve stimulation unit 304 may include a greater auricular nerve stimulating electrode 314. The taVNS stimulating electrode 31, the auriculotemporal nerve stimulating electrode 312 and the greater auricular nerve stimulating electrode 314 may be configured to be housed together in an auricular housing 11 and are further configured to automatically contact their respective innervated auricular skin when the auricular housing 11 is placed in the wearer's ear 902, similar to the aforementioned descriptions.

In some embodiments, an automatic detection-therapy system for cardiac arrhythmia 101 may comprise a cardiovascular monitoring apparatus 600 and a neuromodulation system 300. The cardiovascular monitoring apparatus 600 may comprise an auricular ECG monitoring system 100, and at least one of: an auricular SCOS unit 511, a SPG unit 555, a PPG unit 555A, and an auricular EEG monitoring system 170. The neuromodulation system 300 may comprise a taVNS stimulation unit 30, an auriculotemporal nerve stimulation unit 302 and a greater auricular nerve stimulation unit 304. All of the ECG sensor electrodes 12, 13, all of the EEG sensor electrode 72, 73, 74, 75, 82, 83, (if incorporated), the auricular SCOS sensor 512 (if incorporated), the SPG unit 555 (if incorporated), the PPG unit 555A (if incorporated), the taVNS stimulating electrode 31, the auriculotemporal nerve stimulating electrode 312 and the greater auricular nerve stimulating electrode 314 may be all housed together sharing a same auricular housing 11. The aforementioned designs, setups and material for the auricular housing 11 may be similarly utilized so that all of the incorporated sensors and electrodes are self-installable (user-installable) and self-removable (user-removable) and all of the neuromodulating electrodes will automatically contact their respective innervated auricular skin when the auricular housing 11 is placed in the wearer's ear 902 (including the external ear canal 904 and the tragus-concha bowl 957 of the wearer's ear 902).

In some embodiments, an automatic detection-therapy system for cardiac arrhythmia 101 may comprise a cardiovascular monitoring apparatus 600 and a neuromodulation system 300. The cardiovascular monitoring apparatus 600 may comprise an auricular ECG monitoring system 100, an auricular SCOS unit 511, at least one of: a SPG unit 555 and a PPG unit 555A, and an auricular EEG monitoring system 170. The neuromodulation system 300 may comprise at least one of the following: a taVNS stimulation unit 30, an auriculotemporal nerve stimulation unit 302 and a greater auricular nerve stimulation unit 304. All of the ECG sensor electrodes 12, 13, all of the EEG sensor electrode 72, 73, 74, 75, 82, 83, the auricular SCOS sensor 512, at least one of: the SPG unit 555 and the PPG unit 555A, the taVNS stimulating electrode 31 (if incorporated), the auriculotemporal nerve stimulating electrode 312 (if incorporated) and the greater auricular nerve stimulating electrode 314 (if incorporated) may be all housed together sharing a same auricular housing 11. The aforementioned designs, setups and material for the auricular housing 11 may be similarly utilized so that all of the incorporated sensors and electrodes are self-installable (user-installable) and self-removable (user-removable) and all of the neuromodulating electrodes will automatically contact their respective innervated auricular skin when the auricular housing 11 is placed in the wearer's ear 902 (including the external ear canal 904 and the tragus-concha bowl 957 of the wearer's ear 902). In alternative embodiments, an automatic detection-therapy system for cardiac arrhythmia 101 may comprise a cardiovascular monitoring apparatus 600 having different combination of the components and a neuromodulation system 300 having different combination of the components and all of these alternative embodiments may have the similar features and advantages of the designs, setups and material of the auricular housing 11, as described hereinbefore.

In some embodiments, an automatic detection-therapy system for cardiac arrhythmia 101, may comprise a neuromodulation system 300, a cardiovascular monitoring apparatus 600 and a processing unit 50, 401. The neuromodulation system 300 may comprise at least one of the following components: a taVNS unit 30, a supraorbital nerve stimulation unit 301, an auriculotemporal nerve stimulation unit 302, an occipital nerve stimulation unit 303, a greater auricular nerve stimulation unit 304, an infraorbital nerve stimulation unit 305 and a median nerve stimulation unit 306. Each component of the neuromodulation system 300 may be configured to give neuromodulating electric stimulation to the wearer 900 when prompted or activated. The cardiovascular monitoring system 600 may comprise an auricular ECG monitoring system 100, an auricular SCOS unit 511, at least one of: a SPG unit 555 and a PPG unit 555A, and an auricular EEG monitoring system 170, as described hereinbefore. The processing unit 50, 401, comprises a closed-loop control system 333 and a R-wave synchronization mode. The closed-loop control system 333 and the processing unit 50, 401, are in electronic communication with the cardiovascular monitoring apparatus 600 and each component of the neuromodulation system 300. The closed-loop control system 333 may be configured to receive real-time continuous input data from the cardiovascular monitoring apparatus 600, including the ECG data and the cerebral hemodynamic data. The cerebral hemodynamic data comprises at least one of the following: cerebral and extracranial blood flow data from the auricular SCOS unit 511, systolic blood pressure data diastolic blood pressure data and regional blood flow data from at least one of: the SPG unit 555 and the PPG unit 555A, relative delta power data, delta/alpha ratio data and cordance z-score data from the auricular EEG monitoring system 170. The closed-loop control system 333 and the processing unit 50, 401, may be configured to analyze the real-time continuous ECG data and cerebral hemodynamic data, using controlling algorithms, to turn on or turn off at least a component of the neuromodulation system 300 and to continuously adjust the stimulating parameters of the neuromodulation system 300 during the time when the neuromodulation system 300 is turned on. In addition, the closed-loop control system 333 and the processing unit 50, 401, are further configured to use the R-wave synchronization mode to guide the neuromodulating electric stimulation to the wearer 900.

In some embodiments, an automatic detection-therapy system for cardiac arrhythmia 101 may comprise a cardiovascular monitoring apparatus 600 that comprises an auricular electroencephalogram (EEG) monitoring system 170. The auricular EEG monitoring system 170 may comprise a first EEG recording module 71 having a plurality of EEG sensor electrodes 72, 73, 74, 75, 82, 83. The first EEG recording module 71 may be in electronic communication with each EEG sensor electrode 72, 73, 74, 75, 82, 83, of the first EEG recording module 71. The first EEG recording module 71 may be configured to record EEG data of the wearer 900. All of the EEG sensor electrodes 72, 73, 74, 75, 82, 83, of the first EEG recording module 71 may be configured to be housed in a first auricular housing 11. The first auricular housing 11 includes a first tubular-shaped structure 66. The first tubular-shaped structure 66 may be configured to be inserted into an external ear canal 904 of the wearer's first ear 902 when in use. All of the EEG sensor electrodes 72, 73, 74, 75, 82, 83, of the first EEG recording module 71 may be configured to be located on a surface 91 and partially embedded in the surface 91 with protrusion at the surface of the first tubular-shaped structure 66. One or more EEG sensor electrode(s) 72, 73, 74, 75, 82, 83, may be located at the upper surface of the first tubular-shaped structure 66. One or more EEG sensor electrode(s) 72, 73, 74, 75, 82, 83, may be located above the horizontal level of the first tubular-shaped structure 66 and are facing forward-upward (at approximately 45 degrees plus/minus 15 degrees), while one or more EEG sensor electrode(s) 72, 73, 74, 75, 82, 83, may be located above the horizontal level of the first tubular-shaped structure 66 and are facing backward-upward (at approximately 135 degrees plus/minus 15 degrees). The first tubular-shaped structure 66 may comprise an elastic flexible and adaptable material, and the elastic flexible and adaptable material of the first tubular-shaped structure 66 may be configured to have appropriate elasticity flexibility and adaptability so that when the first tubular-shaped structure 66 is inserted into the external ear canal 904 of the wearer's first ear 902 the first tubular-shaped structure 66 will naturally adapt to the contour of the external ear canal 904 of the wearer's first ear 902 and snugly fill the interior of the external ear canal 904 of the wearer's first ear, so that all of the EEG sensor electrodes 72, 73, 74, 75, 82, 83, of the first EEG recording module 71 are naturally in close contact with the skin of the external ear canal 904 of the wearer's first ear 902 when the first tubular-shaped structure 66 is inserted into the external ear canal of the wearer's first ear 902. This setup and design will enable the EEG sensor electrodes to have one of the best locations and directions in the external ear canal 904 to record the wearer's 900 EEG.

In some embodiments, an automatic detection-therapy system for cardiac arrhythmia 101 may further comprise a network interface 53 and at least one of: a speaker 15, 404A, on a client device 400 of the wearer 900 and a vibrator 17, 404B, on the client device 400 of the wearer. The processing unit 50, 401, may be in electronic communication with the network interface 53, the speaker 15, 404A, and the vibrator 17, 404B. The processing unit 50, 401, may be configured to analyze the cerebral blood flow data recorded by the auricular SCOS unit 511, and the regional blood flow data and the systolic and diastolic blood pressure data recorded by at least one of: the SPG unit 555 and the PPG unit 555A to detect presence of significant hemodynamic change of the wearer 900. As used herein, significant hemodynamic change of the wearer 900 comprises (refers to) presence of at least one of the following: a decrease of cerebral blood flow more than a predetermined percentage (e.g. a decrease of 30% or more), a decrease of regional blood flow more than a predetermined percentage (e.g. a decrease of 30% or more), a decrease of systolic blood pressure more than a predetermined amount (e.g. a decrease of 20 mmHg or more), and a decrease of diastolic blood pressure more than a predetermined amount (e.g. a decrease of 20 mmHg or more). When the processing unit 50, 401, detects presence of ECG data suggestive of serious cardiac arrhythmia without co-occurrence of presence of significant hemodynamic change, the processing unit 50, 401, may be configured to send signals to the neuromodulation system 300 to prompt the neuromodulation system 300 to start sending neuromodulating electric stimulation to the wearer 900 under the guidance of the closed-loop control system 333 using the continuous input data from the auricular ECG monitoring system 100, the auricular SCOS unit 511 and at least one of: the SPG unit 555 and the PPG unit 555A and using R-wave synchronization mode when delivering the neuromodulating electric stimulation. When the processing unit 50, 401, detects co-occurrence of both of the following: presence of ECG data suggestive of serious cardiac arrhythmia and presence of significant hemodynamic change, the processing unit 50, 401, may be configured to send signals to the neuromodulation system 300 to prompt the neuromodulation system 300 to do at least one of: increasing potency of neuromodulating electric stimulation to the wearer 900 and recruiting more components of the neuromodulation system 300 under the guidance of the closed-loop control system 333 using the continuous input data from the auricular ECG monitoring system 100, the auricular SCOS unit 511 and at least one of: the SPG unit 555 and the PPG unit 555A, and using R-wave synchronization mode when delivering the neuromodulating electric stimulation. Since co-occurrence of serious cardiac arrhythmia and significant hemodynamic change represents an urgent condition, the closed-loop control system 333 may be configured to guide the neuromodulation system 300 to give more potent neuromodulating electric stimulation (more potent stimulation such as higher frequency, higher strength, longer duration, etc. and recruiting more components of the neuromodulation system 300.) When the processing unit 50, 401, detects co-occurrence of both of the following: presence of a serious cardiac arrhythmia and presence of significant hemodynamic change, the processing unit 50, 401, may be further configured to send signals to the network interface 53 to prompt the network interface 53 to generate an urgent notification to at least one of: a client device 400 of the wearer 900 and a client device 400 of a healthcare provider 950 of the wearer. When the processing unit 50, 401, detects co-occurrence of both of the following: presence of a serious cardiac arrhythmia and presence of significant hemodynamic change, the processing unit 50, 401, is also configured to send signals to the network interface 53 to prompt the network interface 53 to generate an urgent notification to at least one of: the speaker 15, 404A on the client device 400 of the wearer to generate an urgent audible notification and the vibrator 17, 404B on the client device 400 of the wearer to generate an urgent tactile notification.

In some embodiments, an automatic detection-therapy system for cardiac arrhythmia 101 may comprise a cardiovascular monitoring apparatus 600 and a neuromodulation system 300. The neuromodulation system 300 may comprise a median nerve stimulation unit 306 having a median nerve stimulating electrode 316. The median nerve stimulating electrode 316 may be configured to contact the skin at the Neiguan point on the palmar side of the wearer's distal forearm (where the median nerve passes underneath). The median nerve stimulation unit 306 may be configured to give transcutaneous neuromodulating electric stimulation to the wearer's median nerve when prompted. The median nerve stimulation unit 306 may be in electronic communication with the processing unit 50, 401. When serious cardiac arrhythmia is detected by the processing unit 50, 401, the processing unit is configured to send signals to the median nerve stimulation unit 306 to prompt the median nerve stimulation unit 306 to start sending neuromodulating electric stimulation to the wearer 900 using the R-wave synchronization mode under the guidance of the closed-loop control system 333 utilizing continuous input data from the auricular ECG monitoring system 100. When impending serious cardiac arrhythmia is detected by the processing unit 50, 401, the processing unit is further configured to send signals to the median nerve stimulation unit 306 to prompt the median nerve stimulation unit 306 to start sending neuromodulating electric stimulation to the wearer 900 using the R-wave synchronization mode under the guidance of the closed-loop control system 333 utilizing continuous input data from the cardiovascular monitoring apparatus 600. Median nerve stimulation is an emerging, non-invasive neuromodulation technique effective at reducing cardiac arrhythmia, particularly atrial fibrillation and ventricular arrhythmias, by restoring sympatho-vagal balance. Studies have shown that it can decrease premature atrial contractions (PAC) and atrial fibrillation burden, and protecting against ventricular arrhythmia by inhibiting sympathetic activity. In some embodiments, an auricular ECG monitoring system 100 may comprise an ECG recording module 20 having a first ECG sensor electrode 12 located in one ear and a second ECG sensor electrode 13 located on a wrist band 150. The median nerve stimulation unit 306 and the median nerve stimulating electrode 316 may also be configured to be located in the same wrist band 150. (FIG. 3). The median nerve is a mixed nerve containing both motor fibers and sensory fibers. A median nerve stimulator may be configured to stimulate both the sensory and motor fibers of the median nerve, depending on the device's intensity and purpose. High-intensity stimulation typically induces motor contraction, while lower-intensity, therapeutic stimulation (such as for neuromodulation) often targets sensory afferent fibers. The median nerve stimulation unit 306 may be configured to deliver low-intensity stimulation to stimulate only the sensory fiber for neuromodulation. The median nerve stimulation unit 306 may be configured to deliver transcutaneous electric stimulation via surface electrode to the skin at wrist area, similar to transcutaneous electric nerve stimulator (TENS).

In some preferred embodiments, an automatic detection-therapy system for cardiac arrhythmia 101 may comprise an auricular housing 11 and a wrist housing 150 (such as a wrist band 150). The ECG recording module 20 may comprise at least two ECG sensor electrodes 12, 13, including a first ECG sensor electrode 12 and a second ECG sensor electrode 13. The neuromodulation system 300 may comprise a median nerve stimulation unit 306 and at least one of the following: a transcutaneous auricular vagus nerve stimulation (taVNS) unit 30, an auriculotemporal nerve (ATN) stimulation unit 302 and a greater auricular nerve (GAN) stimulation unit 304. The first ECG sensor electrode 12 and at least one of the following: the taVNS unit 30, the ATN stimulation unit 302 and the GAN stimulation unit 304 may be configured to be housed in the auricular housing 11, while the second ECG sensor electrode 13 and the median nerve stimulation unit 306 may be configured to be housed in the wrist housing 150 (wrist band 150). (FIG. 3). The taVNS stimulation unit 30 includes a taVNS stimulating electrode 31; the ATN stimulation unit 302 includes an ATN stimulating electrode 312; the GAN stimulation unit 304 includes a GAN stimulating electrode 314; and the median nerve stimulation unit 306 includes a median nerve stimulating electrode 316. The first ECG sensor electrode 12, the taVNS stimulating electrode 31, the ATN stimulating electrode 312 and the GAN stimulating electrode 314 may be configured to be located on a surface and partially embedded in the surface with protrusion at the surface of the auricular housing 11. The auricular housing 11 may comprise a flexible elastic and adaptable material. The material for the auricular housing 11 may be configured to have appropriate flexibility elasticity and adaptability so that when the auricular housing 11 is placed in the wearer's ear 902 the first ECG sensor electrode 12, the taVNS stimulating electrode 31, the ATN stimulating electrode 312 and the GAN stimulating electrode 314 are naturally in close contact with the skin of the wearer's ear 902 and the taVNS stimulating electrode 31, the ATN stimulating electrode 312 and the GAN stimulating electrode 314 are automatically contacting their respective innervated auricular skin, similar to descriptions hereinbefore. The second ECG sensor electrode 13 and the median nerve stimulating electrode 316 are configured to be located on the surface of the wrist housing 150 (wrist band 150) with the median nerve stimulating electrode 316 configured to be located at the Neiguan point on the palmar-side of the distal forearm over where the median nerve is located (passes underneath) when the wrist housing 150 is worn by the wearer 900, so that when the wrist housing 150 is worn by the wearer 900 the second ECG sensor electrode 13 is in close contact with the wearer's forearm skin and the median nerve stimulating electrode 316 is in close contact with the wearer's forearm skin at the Neiguan point where the median nerve passes underneath. The wrist housing 150 may be configured as a wrist band 150 that snugly fit the wearer's wrist area. Alternatively, the wrist housing 150 may be configured to be incorporated into a smart watch or a health tracker. Combination of neuromodulation from the taVNS unit 30 and the median nerve stimulation unit 306 has synergistic effect, particularly in reducing sympathetic arousal and enhance regulation of autonomic nervous system. This will help to control the cardiac arrhythmia.

In some embodiments, an automatic detection-therapy system for cardiac arrhythmia 101, may comprise an auricular ECG monitoring system 100, a SCOS unit 511, a SPG unit 555, an auricular EEG monitoring system 170 and a neuromodulation system 300 having at least two neuromodulation units (includes at least two of: a taVNS unit 30, a supraorbital nerve stimulation unit 301, an auriculotemporal nerve stimulation unit 302, an occipital nerve stimulation unit 303, a greater auricular nerve unit 304, an infraorbital nerve stimulation unit 305, and a median nerve stimulation unit 306, as described hereinbefore). The processing unit 50, 401, may be configured to analyze the ECG data recorded by the auricular ECG monitoring system 100 to detect presence of serious cardiac arrhythmia. The processing unit 50, 401, may be configured to analyze the data recorded by the auricular SCOS unit 511 to detect presence of a decrease of cerebral blood flow more than a predetermined percentage (e.g. a decrease of 30% or more). The processing unit 50, 401, may be configured to analyze the data recorded by the SPG unit 555 to detect presence of at least one of: a decrease of systolic blood pressure more than a predetermined amount (e.g. a decrease of 20 mmHg or more), a decrease of diastolic blood pressure more than a predetermined amount (e.g. a decrease of 20 mmHg or more) and a decrease of regional blood flow more than a predetermined percentage (e.g. a decrease of 30% or more). The processing unit 50, 401, may be configured to analyze the qEEG data recorded by the auricular EEG monitoring system 170 to detect presence of at least one of: an increase of relative delta power more than a predetermined percentage (e.g. an increase of 30% or more), an increase of delta/alpha ratio more than a predetermined percentage (e.g. an increase of 30% or more) and a decrease of cordance z-score more than a predetermined amount (e.g. a decrease of one standard deviation or more). When the processing unit 50, 401, detects presence of serious cardiac arrhythmia that co-occurs with at least one of the following: a decrease of cerebral blood flow more than the predetermined percentage, a decrease of systolic blood pressure more than the predetermined amount, a decrease of diastolic blood pressure more than the predetermined amount, a decrease of regional blood flow more than the predetermined percentage, an increase of relative delta power more than the predetermined percentage, an increase of delta/alpha ratio more than the predetermined percentage and a decrease of cordance z-score more than the predetermined amount, the processing unit 50, 401, may be further configured to send signals to the neuromodulation system 300 to prompt the neuromodulation system 300 to do at least one of the following: to increase the potency of the neuromodulating electric stimulation (for example: increase the stimulation frequency, more strength or longer duration, etc.) and to recruit more neuromodulating units to start neuromodulating electric stimulation. The processing unit 50, 401, and the closed-loop control system 333 are configured to utilized R-wave synchronization mode and continuous input data from the auricular ECG monitoring system 100, the SCOS unit 511, the SPG unit 555 and the auricular EEG monitoring system 170 to regulate and guide the neuromodulation (adjusting stimulation parameters and time courses, etc.)

In another aspect consistent with the principle of this invention, in preferred embodiments, a cardiovascular monitoring apparatus 600 is disclosed. The cardiovascular monitoring apparatus 600 comprises an auricular electrocardiogram (ECG) monitoring system 100, an auricular speckle contrast optical spectroscopy (auricular SCOS) unit 511, a network interface 53, and a processing unit 50, 401. The auricular ECG monitoring system 100 comprises an ECG recording module 20 and at least two ECG sensor electrodes 12, 13, including a first ECG sensor electrode 12 and a second ECG sensor electrode 13. The first ECG sensor electrode 12 and the second ECG sensor electrode 13 may be configured to be in contact with separate areas on a first ear 902 of a wearer 900. The first ECG sensor electrode 12 and the second ECG sensor electrode 13 are in electronic communication with the ECG recording module 20. The ECG recording module 20 may be configured to record ECG data of the wearer 900. The auricular speckle contrast optical spectroscopy (SCOS) unit 511 may be configured to contact the skin of the first ear 902 of the wearer 900. The auricular SCOS unit 511 may be configured to record the wearer's cerebral and extracranial blood flow data. The processing unit 50, 401, may be configured to analyze the SCOS data recorded by the SCOS unit 511 to detect presence or cessation of a decrease of cerebral blood flow more than a predetermined percentage. The processing unit 50, 401, is in electronic communication with the auricular ECG monitoring system 101, the SCOS unit 511 and the network interface 53. The processing unit 50, 401, may be configured to analyze the ECG data recorded by the auricular ECG monitoring system 100 to detect presence or cessation of serious cardiac arrhythmia and presence or cessation of impending serious cardiac arrhythmia. The processing unit 50, 401, may be also configured to analyze the SCOS data recorded by the SCOS unit 511 to detect presence or cessation of a decrease of cerebral blood flow more than a predetermined percentage. When the processing unit 50, 401, detects presence of ECG data suggestive of serious cardiac arrhythmia that does not co-occur with presence of cerebral blood flow data showing a decrease of cerebral blood flow more than the predetermined percentage, the processing unit 50, 401, may be configured to send signals to the network interface 53 to prompt the network interface to send a notification to at least one of: a client device 400 of the wearer 900 and a client device 400 of the wearer's healthcare provider 950. When the processing unit 50, 401, detects co-occurrence of both of the following: presence of ECG data suggestive of serious cardiac arrhythmia and presence of cerebral blood flow data showing a decrease of cerebral blood flow more than the predetermined percentage, the processing unit 50, 401, may be configured to send signals to the network interface 53 to prompt the network interface to send an urgent notification to at least one of: a client device 400 of the wearer and a client device 400 of the wearer's healthcare provider 950. When the processing unit 50, 401, detects presence of ECG data suggestive of impending serious cardiac arrhythmia, the processing unit 50, 401, may be configured to send signals to the network interface 53 to prompt the network interface 53 to send a notification to at least one of: the client device 400 of the wearer 900 and the client device 400 of the healthcare provider 950 of the wearer. When the processing unit 50, 401, detects all of the following: cessation of ECG signals suggestive of serious cardiac arrhythmia, cessation of ECG signals suggestive of impending serious cardiac arrhythmia, cessation of a decrease of cerebral blood flow more than the predetermined percentage, the processing unit 50, 401, may be further configured to send signals to the network interface 53, to prompt the network interface 53 to send a cessation notification to at least one of: the client device 400 of the wearer 900 and the client device 400 of the healthcare provider 950 of the wearer.

In some embodiments, a cardiovascular monitoring apparatus 600 may comprise an auricular electrocardiogram (ECG) monitoring system 100, an auricular speckle contrast optical spectroscopy (auricular SCOS) unit 511, a speckle-plethysmography (SPG) unit 555, a network interface 53, and a processing unit 50, 401. The auricular ECG monitoring system 100 and the auricular SCOS unit 511 are as described hereinbefore. The SPG unit 555 is configured to contact the skin of the first ear 902 of the wearer. The SPG unit 555 may be configured to record the wearer's regional blood flow data at the area where the SPG unit 555 contacts the wearer's first ear 902. The SPG unit 555 may be also configured to record the wearer's systolic blood pressure data and diastolic blood pressure data. The processing unit 50, 401, may be in electronic communication with the SPG unit 555, the auricular ECG monitoring system 100 and the auricular SCOS unit 511. The processing unit 50, 401, may be configured to analyze the ECG data recorded by the auricular ECG monitoring system 100 to detect presence or cessation of serious cardiac arrhythmia and presence or cessation of impending serious cardiac arrhythmia. The processing unit 50, 401, may be configured to analyze the cerebral blood flow data recorded by the SCOS unit 511 to detect presence or cessation of a decrease of cerebral blood flow more than a predetermined percentage (e.g. a decrease of 30% or more). The processing unit 50, 401, may be also configured to analyze the regional blood flow data, systolic blood pressure data and diastolic blood pressure data recorded by the SPG unit 555 to detect presence or cessation of a decrease of regional blood flow more than a predetermined percentage (e.g. a decrease of 30% or more), presence or cessation of a decrease of systolic blood pressure more than a predetermined amount (e.g. a decrease of 20 mmHg or more) and presence or cessation of a decrease of diastolic blood pressure more than a predetermined amount (e.g. a decrease of 20 mmHg or more). When the processing unit 50, 401, detects presence of ECG data suggestive of serious cardiac arrhythmia that does not co-occur with any of the following: presence of a decrease of cerebral blood flow more than the predetermined percentage, presence of a decrease of regional blood flow more than the predetermined percentage, presence of a decrease of systolic blood pressure more than the predetermined amount, and presence of a decrease of diastolic blood pressure more than the predetermined amount, the processing unit 50, 401, is configured to send signals to the network interface 53 to prompt the network interface to send a notification to at least one of: the client device 400 of the wearer 900 and the client device 400 of the healthcare provider 950 of the wearer. When the processing unit 50, 401, detects presence of ECG data suggestive of serious cardiac arrhythmia that co-occurs with any of the following: presence of a decrease of cerebral blood flow more than the predetermined percentage, presence of a decrease of regional blood flow more than the predetermined percentage, presence of a decrease of systolic blood pressure more than the predetermined amount, and presence of a decrease of diastolic blood pressure more than the predetermined amount, the processing unit 50, 401, is configured to send signals to the network interface 53 to prompt the network interface to send an urgent notification to at least one of: the client device 400 of the wearer 900 and the client device 400 of the healthcare provider 950 of the wearer. When the processing unit 50, 401, detects presence of ECG data suggestive of impending serious cardiac arrhythmia, the processing unit 50, 401, may be configured to send signals to the network interface 53 to prompt the network interface to send a notification to at least one of: the client device 400 of the wearer 900 and the client device 400 of the healthcare provider 950 of the wearer. When the processing unit 50, 401, detects all of the following: cessation of ECG data suggestive of serious cardiac arrhythmia, cessation of ECG data suggestive of impending serious cardiac arrhythmia, cessation of a decrease of cerebral blood flow more than the predetermined percentage, cessation of a decrease of regional blood flow more than the predetermined percentage, cessation of a decrease of systolic blood pressure more than the pre-determined amount and cessation of a decrease of diastolic blood pressure more than the pre-determined amount, the processing unit 50, 401, may be further configured to send signals to the network interface 53 to prompt the network interface to send a cessation notification to at least one of: the client device 400 of the wearer 900 and the client device 400 of the healthcare provider 950 of the wearer.

In some embodiments, a cardiovascular monitoring apparatus 600 may comprise an auricular electrocardiogram (ECG) monitoring system 100, an auricular electroencephalogram (EEG) monitoring system 170, an auricular speckle contrast optical spectroscopy (SCOS) unit 511, a network interface 53 and a processing unit 50, 401. The auricular ECG monitoring system 100 and the auricular EEG monitoring system 170 are as described hereinbefore. The auricular speckle contrast optical spectroscopy (SCOS) unit 511 may be configured to contact the skin of a first ear 902 of a wearer. The auricular SCOS unit 511 may be configured to record the wearer's cerebral and extracranial blood flow data. The SCOS unit 511, the auricular ECG monitoring system 100 and the auricular EEG monitoring system 170 may be in electronic communication with the processing unit 50, 401. The processing unit 50, 401, may be configured to analyze the ECG data recorded by the auricular ECG monitoring system 100 to detect presence or cessation of serious cardiac arrhythmia and presence or cessation of impending serious cardiac arrhythmia. The processing unit 50, 401, is configured to convert the EEG data recorded by the auricular EEG monitoring system 170 into quantitative EEG (qEEG) data. The processing unit 50, 401, may be configured to analyze the EEG data and qEEG data recorded by the auricular EEG monitoring system 170 to detect presence of at least one of: an increase of relative delta power more than a predetermined percentage (e.g. an increase of 30% or more), an increase of delta/alpha ratio more than a predetermined percentage (e.g. an increase of 30% or more) and a decrease of cordance z-score more than a predetermined amount (e.g. a decrease of 1 standard deviation or more). The processing unit 50, 401, may be also configured to analyze the SCOS data recorded by the SCOS unit 511 to detect presence or cessation of a decrease of cerebral blood flow more than a predetermined percentage (e.g. a decrease of 30% or more). When the processing unit 50, 401, detects presence of ECG data suggestive of serious cardiac arrhythmia that does not co-occur with any of the following: presence of SCOS data showing a decrease of cerebral blood flow more than the predetermined percentage, presence of EEG data and qEEG data showing at least one of: an increase of relative delta power more than the predetermined percentage, an increase of delta/alpha ratio more than the predetermined percentage and a decrease of cordance z-score more than the predetermined amount, the processing unit 50, 401, is configured to send signals to the network interface 53 to prompt the network interface to send a notification to at least one of: a client device 400 of the wearer and a client device 400 of the healthcare provider 950 of the wearer. When the processing unit 50, 401, detects presence of ECG data suggestive of serious cardiac arrhythmia that co-occurs with at least one of the following: presence of SCOS data showing a decrease of cerebral blood flow more than the predetermined percentage, presence of EEG data and qEEG data showing at least one of: an increase of relative delta power more than the predetermined percentage, an increase of delta/alpha ratio more than the predetermined percentage and a decrease of cordance z-score more than the predetermined amount, the processing unit 50, 401, may be configured to send signals to the network interface 53 to prompt the network interface 53 to send an urgent notification to at least one of: a client device 400 of the wearer and a client device 400 of the healthcare provider 950 of the wearer. When the processing unit 50, 401, detects presence of ECG data suggestive of impending serious cardiac arrhythmia, the processing unit 50, 401, may be configured to send signals to the network interface 53 to prompt the network interface 53 to send a notification to at least one of: the client device 400 of the wearer and the client device 400 of the healthcare provider 950 of the wearer. When the processing unit 50, 401, detects all of the following: cessation of ECG data suggestive of serious cardiac arrhythmia, cessation of ECG data suggestive of impending serious cardiac arrhythmia, cessation of a decrease of cerebral blood flow more than the predetermined percentage, cessation an increase of relative delta power more than the predetermined percentage, cessation of an increase of delta/alpha ratio more than the predetermined percentage and cessation of a decrease of cordance z-score more than the predetermined amount, the processing unit 50, 401, may be further configured to send signals to the network interface 53 to prompt the network interface 53 to send a cessation notification to at least one of: the client device 400 of the wearer and the client device 400 of the healthcare provider 950 of the wearer.

In alternative embodiments, a cardiovascular monitoring apparatus 600 may comprise an auricular electrocardiogram (ECG) monitoring system 100, an auricular speckle contrast optical spectroscopy (auricular SCOS) unit 511, a photoplethysmography (PPG) unit 555A, a network interface 53, and a processing unit 50, 401. The auricular ECG monitoring system 100 and the auricular SCOS unit 511 are as described hereinbefore. The PPG unit 555A is configured to contact the skin of the first ear 902 of the wearer. The PPG unit 555A may be configured to record the wearer's regional blood flow data at the area where the PPG unit 555A contacts the wearer's first ear 902. The PPG unit 555A may be also configured to record the wearer's systolic blood pressure data and diastolic blood pressure data. The processing unit 50, 401, may be in electronic communication with the PPG unit 555A. The processing unit 50, 401, may be configured to analyze the ECG data recorded by the auricular ECG monitoring system 100 to detect presence or cessation of serious cardiac arrhythmia and presence or cessation of impending serious cardiac arrhythmia. The processing unit 50, 401, may be configured to analyze the cerebral blood flow data recorded by the SCOS unit 511 to detect presence or cessation of a decrease of cerebral blood flow more than a predetermined percentage (e.g. a decrease of 30% or more). The processing unit 50, 401, may be also configured to analyze the regional blood flow data, systolic blood pressure data and diastolic blood pressure data recorded by the PPG unit 555A to detect presence or cessation of a decrease of regional blood flow more than a predetermined percentage (e.g. a decrease of 30% or more), presence or cessation of a decrease of systolic blood pressure more than a predetermined amount (e.g. a decrease of 20 mmHg or more) and presence or cessation of a decrease of diastolic blood pressure more than a predetermined amount (e.g. a decrease of 20 mmHg or more). When the processing unit 50, 401, detects presence of ECG data suggestive of serious cardiac arrhythmia co-occurts with at least one of the following: presence of a decrease of cerebral blood flow more than the predetermined percentage, presence a decrease of regional blood flow more than the predetermined percentage, presence of a decrease of systolic blood pressure more than the predetermined amount and presence of a decrease of diastolic blood pressure more than the predetermined amount, the processing unit 50, 401, may be configured to send signals to the network interface 53 to prompt the network interface 53 to send an urgent notification to at least one of: a client device 400 of the wearer 900 and a client device 400 of the healthcare provider 950 of the wearer. When the processing unit 50, 401, detects presence of ECG data suggestive of impending serious cardiac arrhythmia, the processing unit 50, 401, may be configured to send signals to the network interface 53 to prompt the network interface to send a notification to at least one of: the client device 400 of the wearer and the client device 400 of the healthcare provider 950 of the wearer. When the processing unit 50, 401, detects all of the following: cessation of ECG data suggestive of serious cardiac arrhythmia, cessation of ECG data suggestive of impending serious cardiac arrhythmia, cessation of a decrease of cerebral blood flow more than the predetermined percentage, cessation of a decrease of regional blood flow more than the predetermined percentage, cessation of a decrease of systolic blood pressure more than the predetermined amount and cessation of a decrease of diastolic blood pressure more than the predetermined amount, the processing unit 50, 401, may be further configured to send signals to the network interface 53 to prompt the network interface to send a cessation notification to at least one of: the client device 400 of the wearer 900 and the client device 400 of the healthcare provider 950 of the wearer.

In some embodiments, an auricular EEG monitoring system 100 and an automatic detection-therapy system for cardiac arrhythmia 101 may comprises a switch 88 and a timer 89 to enable the wearer 900 to turn on the system 100 or system 101 manually and setting a duration, an interval or a schedule etc. Preferably, the switch 88 may be configured as a multi-mode switch 88 and the timer 89 may be configured as a multi-mode timer 89. In some embodiments, a multi-mode switch 88 may be configured to enable the wearer to set the system 100 and system 101 at an automatic mode or a manual mode. The functions and setups of the automatic mode for system 101 are as described hereinbefore. In some embodiments for an automatic detection-therapy system for cardiac arrhythmia 101, the multi-mode switch 88 may be configured to allow the wearer 900 to choose at least one of the following switch selections: turning on or off an auricular ECG monitoring system 100, turning on or off an auricular SCOS unit 511, turning on or off at least one of: a SPG unit 555 and a PPG unit 555A, turning on or off an auricular EEG monitoring system 170, turning on or off a taVNS unit 30, turning on or off a supraorbital nerve stimulation unit 301, turning on or off an auriculotemporal nerve stimulation unit 302, turning on or off an occipital nerve stimulation unit 303, turning on or off a greater auricular nerve stimulation unit 304, turning on or off an infraorbital nerve stimulation unit 305, turning on or off a median nerve stimulation unit 306, or various combinations thereof. The multi-mode timer 89 may be configured to function together with the multi-mode switch 88 to set each switch selection at desirable timing courses such as duration, interval, countdown, daily/weekly schedule, 10 minute/30 minutes/60 minutes (or other duration) selection, continuous operation, manual turning on and manual shutdown etc. Furthermore, the multi-mode switch 88 and multi-mode timer 89 may be configured as a multifunctional switch-timer or as a programmable multifunctional timer-switch. In some embodiments, the programmable multifunctional timer-switch may be incorporated in a client device 400 of a wearer 900 to allow the wearer flexibility and ease in choosing manual or automatic control and choosing various time courses and various switch selections (selection of various combinations of the 7 components of the neuromodulation system 300). In some embodiments, the multi-mode switch 88 function and the multi-mode timer 89 function may be configured to be incorporated into a wearer's client device 400 and can be easily set up as programmable (programmable setup as known in the art). In the manual mode, the wearer 900 can use the neuromodulation system 300 (including taVNS unit 30, the unit 301, unit 302, unit 303, unit 304, unit 305, unit 306 or various combinations thereof) for prophylactic purpose against cardiac arrhythmia and neuropsychiatric disorders or for health maintenance purpose. Studies have shown that various components of the neuromodulation system 300 are effective not only for therapy, but also for prophylaxis, for cardiac arrhythmia and various neuropsychiatric disorders. The aforementioned programmable set up for the automatic detection-therapy system for cardiac arrhythmia 101 will enable the system 101 to be used for prophylactic purpose and health maintenance purpose. The multi-mode switch 88, multi-mode timer 89 and the programmable design may enable the wearer the option to choose from one of the following stimulation modes: neuromodulation from one neuromodulation component (single neuromodulation), simultaneous neuromodulation from two neuromodulation components (double neuromodulation) and simultaneous neuromodulation from three neuromodulation components (triple neuromodulation). Thus, when prompted, the neuromodulation system 300 is configured to generate neuromodulating electric stimulation to a wearer 900 according to the choice of the stimulation mode selected by the wearer 900.

In yet another aspect consistent with the principle of this invention, in preferred embodiments, a closed-loop neuromodulation apparatus 102 is disclosed. The closed-loop neuromodulation apparatus 102 may comprise a cardiovascular monitoring apparatus 600, a neuromodulation system 300, a multi-functional switch-timer 88, 89, and a processing unit 50, 401. The closed-loop neuromodulation apparatus 102 may comprise a cardiovascular monitoring system 600. The cardiovascular monitoring system 600 comprises an auricular electrocardiogram (ECG) monitoring system 100 and at least one of: a speckle contrast optical spectroscopy (SCOS) unit 511, a speckle-plethysmography (SPG) unit 555 and a photoplethysmography (PPG) unit 555A. The auricular electrocardiogram (ECG) monitoring system 100 may be configured to record ECG data of a wearer 900. The auricular ECG monitoring system 100 may be configured to be housed in an auricular housing 11. The auricular housing 11 may be configured to be placed in a first ear 902 of a wearer when in use. The auricular speckle contrast optical spectroscopy (SCOS) unit 511 may be configured to be housed in the auricular housing 11. The auricular SCOS unit 511 is configured to record the wearer's cerebral and extracranial blood flow data. The SPG unit 555 is configured to be housed in the auricular housing 11 and configured to contact a location of the skin of the wearer's first ear 902 when in use. The SPG unit 555 may be configured to record the wearer's regional blood flow data at the location where the SPG unit 555 contacts the wearer's first ear 902. The SPG unit 555 may be also configured to record systolic blood pressure data and diastolic blood pressure data of the wearer 900. Similarly, the PPG unit 555A is configured to record the wearer's regional blood flow data at the location where the PPG unit 555A contacts the wearer's first ear 902. The PPG unit 555A is also configured to record systolic blood pressure data and diastolic blood pressure data of the wearer 900. The neuromodulation system 300 may comprise at least one of the following components: a transcutaneous auricular vagus nerve stimulation unit (taVNS unit) 30, an auriculotemporal nerve stimulation unit 302, and a greater auricular nerve stimulation unit 304. Each component of the neuromodulation system 300 may be configured to give neuromodulating electric stimulation to the wearer 900 when activated or prompted. The taVNS unit 30 includes a taVNS stimulating electrode 31; the auriculotemporal nerve stimulation unit 302 includes an auriculotemporal nerve stimulating electrode 312; and the greater auricular nerve stimulation unit 304 includes a greater auricular nerve stimulating electrode 314. The taVNS stimulating electrode 31, the auriculotemporal nerve stimulating electrode 312 and the greater auricular nerve stimulating electrode 314 may be configured to be housed together in the same auricular housing 11 and are further configured to automatically contact their respective innervated auricular skin when the auricular housing 11 is placed in the wearer's ear 902 (per the setups, designs and the material for the auricular housing 11, as described hereinbefore). The multi-functional switch-timer 88, 89, is configured to be in electronic communication with each component of the neuromodulation system 300. The multi-functional switch-timer 88, 89, is configured to enable the wearer to turn on or turn off each component of the neuromodulation system 300, to select at least one component of the neuromodulation system 300 and to select timing course for each component of the neuromodulation system 300. The processing unit 50, 401, is configured to be in electronic communication with the auricular ECG monitoring system 100 and at least one of: the SCOS unit 511, the SPG unit 555, and the PPG unit 555A. The processing unit 55, 401, is also in electronic communication with the multi-functional switch-timer 88, 89, and each component of the neuromodulation system 300. The processing unit 50, 401, comprises a closed-loop control system 333 and R-wave synchronization mode. When at least one component of the neuromodulation system 300 is turned on by the wearer 900 via the multifunctional switch-timer 88, 89, the processing unit 50, 401, and the closed-loop control system 333 are configured to regulate the stimulation parameters and the timing course using the continuous input ECG data recorded by the auricular ECG monitoring system 100 and at least one of the following continuous input data: the wearer's cerebral and extracranial blood flow data recorded by the auricular SCOS unit 511, the wearer's regional blood flow data, systolic blood pressure data and diastolic blood pressure data recorded by the SPG unit 555 and the wearer's regional blood flow data, systolic blood pressure data and diastolic blood pressure data recorded the PPG unit 555A. The closed-loop control system 333 and the processing unit 50, 401, are further configured to use the R-wave synchronization mode to guide the neuromodulating electric stimulation to the wearer 900. Optionally, an auricular EEG monitoring system 170 may be incorporated into the closed-loop neuromodulation apparatus 102, as described hereinbefore.

In some embodiments, a closed-loop neuromodulation apparatus 102 may comprise an auricular housing 11. The auricular housing 11 may be selected from one of the following: a modified earbud housing 61, a modified in-the-ear housing 62 and a behind-the-ear-hearing-aid style housing 63. All of these housings 61, 62, 63, include a tubular-shaped structure 66 (configured to be inserted into an external ear canal 904 of a wearer when in use) and a body-structure 67 (configured to be placed at the opening of the external ear canal 904 and to sit or be placed inside a tragus-concha bowl 957 of the wearer's ear when in use). The auricular ECG monitoring system 100 comprises at least two ECG sensor electrodes 12, 13, including a first ECG sensor electrode 12 and a second ECG sensor electrode 13. The auricular SCOS unit 511 comprises a SCOS sensor 512. The taVNS stimulation unit 30 includes a taVNS stimulating electrode 31. The auriculotemporal nerve (ATN) stimulation unit 302 includes an ATN stimulating electrode 312. The greater auricular nerve (GAN) stimulation unit 304 includes a GAN stimulating electrode 314. All of the ECG sensor electrodes 12, 13, may be configured to be located on a surface 91 of the tubular-shaped structure 66. Preferably, the first ECG sensor electrode 12, may be configured to be located on the surface 91 of the tubular-shaped structure 66, while the second ECG sensor electrode 13 may be configured to be located on a surface of the body-structure 67. The auricular SCOS sensor 512, at least one of: the SPG unit 555 and the PPG unit 555A, the taVNS stimulating electrode 31 and the ATN stimulating electrode 312 are configured to be housed on a surface of one of: the tubular-shaped structure 66 and the body-structure 67. The GAN stimulating electrode 314 and the second ECG sensor electrode 13 are configured to be located on a surface of the body-structure 67. All of the ECG sensor electrodes 12, 13, the auricular SCOS sensor 512, the at least one of: the SPG unit 555 and the PPG unit 555A, the taVNS stimulating electrode 31, the ATN stimulating electrode 312 and the GAN stimulating electrode 314 are configured to be partially embedded in the surface with slight protrusion at the surface of one of: the tubular-shaped structure 66 and the body-structure 67. (Alternatively, the auricular SCOS sensor 512 may be placed slightly below the surface of the tubular-shaped structure 66 or the body-structure 67 so that there is a small air gap between the SCOS sensor 512 and the wearer's skin when the tubular-shaped structure 66 or the body-structure 67 are placed in the wearer's ear 902.) The tubular-shaped structure 66 and the body-structure 67 may be configured to comprise a flexible elastic and adaptable material and the flexible elastic and adaptable material is configured to have appropriate flexibility, elasticity and adaptability so that the tubular-shaped structure 66 will naturally adapt to the contour of the external ear canal 904 and snugly fill the interior of the wearer's external ear canal 904 when the tubular-shaped structure 66 is inserted into the external ear canal 904 of the wearer's ear 902 and, meanwhile, the body-structure 67 will naturally adapt to the contour of the tragus-concha bowl 957 of the wearer's ear 902 and snugly fill the interior of the tragus-concha bowl 957 of the wearer's ear 902 when the body-structure 67 is placed inside the tragus-concha bowl 957 of the wearer's ear 902, so that the first ECG sensor electrode 12 will be naturally snugly in contact with the skin of the external ear canal 904 of the wearer's ear 902, and, at the same time, the auricular SCOS sensor 512, the at least one of: the SPG unit 555 and the PPG unit 555A, the taVNS stimulating electrode 31 and the ATN stimulating electrode 312 are naturally and snugly in contact with the skin of the tragus-concha bowl 957 of the wearer's ear 902 or the skin of the external ear canal 904 of the wearer's ear 902, while the GAN stimulating electrode 314 and the second ECG sensor electrode 13 are naturally snugly in contact with the skin of the wearer's tragus-concha bowl 957, and so that the taVNS stimulating electrode 31 is naturally and snugly in contact with its target skin of vagus-innervated auricular skin of the wearer's first ear 902, provided by carefully selecting a location for the taVNS stimulating electrode 31 on the tubular-shaped structure 66 or the body-structure 67 to match one of the innervation locations of the vagus innervated skin on one of: the external ear canal 904 and the tragus-concha bowl 957, and, at the same time, the ATN stimulating electrode 312 will be naturally in close contact with its target skin of ATN innervated auricular skin, provided by carefully selecting a location for the ATN stimulating electrode 312 on the tubular-shaped structure 66 or the body-structure 67 to match one of the innervation locations of the ATN innervated skin on the external ear canal 904 or the tragus-concha bowl 957, and, at the same time, the GAN stimulating electrode 314 will be naturally in close contact with GAN innervated auricular skin, provided by carefully selecting a location for the GAN stimulating electrode 314 on the body-structure 67 to match one of the innervation locations of the GAN innervated skin on tragus-concha bowl 957.

While some exemplary shapes and sizes have been provided for elements of the auricular EEG monitoring system 100 and the automatic detection-therapy system for cardiac arrhythmia 101, it should be understood to one of ordinary skill in the art that the elements described herein may be configured in a plurality of sizes and shapes including “T” shaped, “X” shaped, square shaped, rectangular shaped, cylinder shaped, cuboid shaped, hexagonal prism shaped, triangular prism shaped, or any other geometric or non-geometric shape, including combinations of shapes. It is not intended herein to mention all the possible alternatives, equivalent forms or ramifications of the invention. It is understood that the terms and proposed shapes used herein are merely descriptive, rather than limiting, and that various changes, such as to size and shape, may be made without departing from the spirit or scope of the invention.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. An automatic detection-therapy system for cardiac arrhythmia, comprising:
a cardiovascular monitoring apparatus comprising an auricular electrocardiogram (ECG) monitoring system, wherein the auricular ECG monitoring system includes an ECG recording module and at least two ECG sensor electrodes, wherein at least one of the ECG sensor electrodes is configured to be located at a first ear of a wearer, wherein the at least two ECG sensor electrodes are in electronic communication with the ECG recording module, wherein the ECG recording module is configured to record ECG data of the wearer;
a neuromodulation system comprising at least a neuromodulation unit configured to give neuromodulating electric stimulation to the wearer when prompted; and
a processing unit in electronic communication with the cardiovascular monitoring apparatus and the neuromodulation system, wherein the processing unit comprises a closed-loop control system and R-wave synchronization mode, wherein the processing unit is configured to analyze the ECG data recorded by the auricular ECG monitoring system to detect presence or cessation of ECG signals suggestive of serious cardiac arrhythmia, wherein the processing unit is also configured to analyze the ECG data recorded by the auricular ECG monitoring system to detect presence or cessation of ECG signals suggestive of impending serious cardiac arrhythmia, wherein when serious cardiac arrhythmia is detected by the processing unit, the processing unit is configured to send signals to the neuromodulation system to prompt the neuromodulation system to start sending neuromodulating electric stimulation to the wearer using the R-wave synchronization mode under the guidance of the closed-loop control system utilizing continuous input data from the cardiovascular monitoring apparatus, and wherein when impending serious cardiac arrhythmia is detected by the processing unit, the processing unit is further configured to send signals to the neuromodulation system to prompt the neuromodulation system to start sending neuromodulating electric stimulation to the wearer using the R-wave synchronization mode under the guidance of the closed-loop control system utilizing continuous input data from the cardiovascular monitoring apparatus.

2. The automatic detection-therapy system for cardiac arrhythmia of claim 1, wherein the ECG recording module comprises at least two ECG sensor electrodes including a first ECG sensor electrode and a second ECG sensor electrode, wherein the first ECG sensor electrode is configured to contact a skin area of the first ear of the wearer, wherein the skin area that the first ECG sensor electrode is configured to contact is selected from one of the following: an external ear of the first ear of the wearer, an external ear canal of the first ear of the wearer and a periauricular area around the first ear of the wearer, wherein the second ECG sensor electrode is configured to contact a separate skin area of the first ear of the wearer, wherein the skin area that the second ECG sensor electrode is configured to contact is selected from one of the following: the external ear of the first ear of the wearer, the external ear canal of the first ear of the wearer and the periauricular area around the first ear of the wearer, wherein the skin area selected for the first ECG sensor electrode to contact is different from the skin area selected for the second ECG sensor electrode to contact, and wherein the ECG recording module is configured to record ECG data of the wearer via the first and the second ECG sensor electrodes.

3. The automatic detection-therapy system for cardiac arrhythmia of claim 1, wherein the ECG recording module comprises at least two ECG sensor electrodes including a first ECG sensor electrode and a second ECG sensor electrode, wherein the first ECG sensor electrode is configured to contact a skin area of the first ear of the wearer, wherein the skin area that the first ECG sensor electrode is configured to contact is selected from one of the following: an external ear of the first ear of the wearer, an external ear canal of the first ear of the wearer and a periauricular area around the first ear of the wearer, wherein the second ECG sensor electrode is configured to contact a skin area of a second ear of the wearer, wherein the skin area that the second ECG sensor electrode is configured to contact is selected from one of the following: an external ear of the second ear of the wearer, an external ear canal of the second ear of the wearer and a periauricular area around the second ear of the wearer, wherein the first and the second ECG sensor electrodes are configured as wireless ECG sensor electrodes and are in wireless electronic communication with the ECG recording module, and wherein the ECG recording module is configured to record ECG data of the wearer via the first and the second ECG sensor electrodes.

4. The automatic detection-therapy system for cardiac arrhythmia of claim 1, wherein the ECG recording module comprises at least two ECG sensor electrodes including a first ECG sensor electrode and a second ECG sensor electrode, wherein the first ECG sensor electrode is configured to contact a skin area of the first ear of the wearer, wherein the skin area that the first ECG sensor electrode is configured to contact is selected from one of the following: an external ear of the first ear of the wearer, an external ear canal of the first ear of the wearer and a periauricular area around the first ear of the wearer, wherein the second ECG sensor electrode is configured to contact a skin area selected from one of the following: a finger of the wearer via a finger ring, an ankle of the wearer via an ankle band, a toe of the wearer via a toe ring and a wrist of the wearer via one of: a wrist band, a smart watch and a health tracker, wherein the first and the second ECG sensor electrodes are configured as wireless ECG sensor electrodes and are in wireless electronic communication with the ECG recording module, and wherein the ECG recording module is configured to record ECG data of the wearer via the first and the second ECG sensor electrodes.

5. The automatic detection-therapy system for cardiac arrhythmia of claim 1, wherein the cardiovascular monitoring apparatus further comprises at least one of the following: an auricular speckle contrast optical spectroscopy unit (auricular SCOS unit) and a photoplethysmography unit (PPG unit), wherein the auricular SCOS unit is configured to be located at an area of a first ear of the wearer, wherein the area that the auricular SCOS unit is configured to be located is selected from one of the following: an external ear of the first ear of the wearer, an external ear canal of the first ear of the wearer and a periauricular area around the first ear of the wearer, wherein the auricular SCOS unit is configured to record the wearer's cerebral and extracranial blood flow data, wherein the PPG unit is configured to contact a skin area located at the wearer's first ear, wherein the skin area that the PPG unit is configured to contact is selected from one of the following: the external ear of the first ear of the wearer, the external ear canal of the first ear of the wearer and the periauricular area around the first ear of the wearer, wherein the PPG unit is configured to record the wearer's systolic blood pressure data and diastolic blood pressure data and regional blood flow data at the location of the skin area where the PPG unit contacts the wearer's ear, wherein the processing unit is in electronic communication with at least one of: the auricular SCOS unit and the PPG unit, wherein the processing unit is configured to analyze at least one of: the cerebral and extracranial blood flow data recorded by the auricular SCOS unit and the systolic blood pressure, diastolic blood pressure and regional blood flow recorded by the PPG unit to assess the wearer's cerebral hemodynamic data, wherein when presence of serious cardiac arrhythmia is detected by the processing unit, the processing unit and the closed-loop control system are configured to utilize continuous input data of the wearer's ECG from the auricular ECG monitoring system, continuous input data of the cerebral hemodynamic data from at least one of: the auricular SCOS unit and the PPG unit to guide the neuromodulating electric stimulation to the wearer, including controlling the stimulation parameters, duration, timing, interval and selection of at least one component of the neuromodulation system, wherein the closed-loop control system and the processing unit are further configured to use the R-wave synchronization mode to guide the neuromodulating electric stimulation to the wearer, wherein when presence of impending serious cardiac arrhythmia is detected by the processing unit, the processing unit and the closed-loop control system are configured to utilize continuous input data of the wearer's ECG from the auricular ECG monitoring system, continuous input data of the cerebral hemodynamic data from at least one of: the auricular SCOS unit and the PPG unit to guide the neuromodulating electric stimulation to the wearer, including controlling the stimulation parameters, duration, timing, interval and selection of at least one component of the neuromodulation system, and wherein the closed-loop control system and the processing unit are further configured to use the R-wave synchronization mode to guide the neuromodulating electric stimulation to the wearer.

6. The automatic detection-therapy system for cardiac arrhythmia of claim 1, wherein the cardiovascular monitoring apparatus further comprises at least one of the following: an auricular speckle contrast optical spectroscopy unit (auricular SCOS unit) and a speckle-plethysmography unit (SPG unit), wherein the auricular SCOS unit is configured to be located at an area of a first ear of the wearer, wherein the area that the auricular SCOS unit is configured to be located is selected from one of the following: an external ear of the first ear of the wearer, an external ear canal of the first ear of the wearer and a periauricular area around the first ear of the wearer, wherein the auricular SCOS unit is configured to record the wearer's cerebral and extracranial blood flow data, wherein the SPG unit is configured to contact a skin area located at the wearer's first ear, wherein the skin area that the SPG unit is configured to contact is selected from one of the following: the external ear of the first ear of the wearer, the external ear canal of the first ear of the wearer and the periauricular area around the first ear of the wearer, wherein the SPG unit is configured to record the wearer's systolic blood pressure data and diastolic blood pressure data and regional blood flow data at the location of the skin area where the SPG unit contacts the wearer's ear, wherein the processing unit is in electronic communication with at least one of: the auricular SCOS unit and the SPG unit, wherein the processing unit is configured to analyze at least one of: the cerebral and extracranial blood flow data recorded by the auricular SCOS unit and the systolic blood pressure, diastolic blood pressure and regional blood flow recorded by the SPG unit to assess the wearer's cerebral hemodynamic data, wherein when presence of serious cardiac arrhythmia is detected by the processing unit, the processing unit and the closed-loop control system are configured to utilize continuous input data of the wearer's ECG from the auricular ECG monitoring system, continuous input data of the cerebral hemodynamic data from at least one of: the auricular SCOS unit and the SPG unit to guide the neuromodulating electric stimulation to the wearer, including controlling the stimulation parameters, duration, timing, interval and selection of at least one component of the neuromodulation system, wherein the closed-loop control system and the processing unit are further configured to use the R-wave synchronization mode to guide the neuromodulating electric stimulation to the wearer, wherein when presence of impending serious cardiac arrhythmia is detected by the processing unit, the processing unit and the closed-loop control system are configured to utilize continuous input data of the wearer's ECG from the auricular ECG monitoring system, continuous input data of the cerebral hemodynamic data from at least one of: the auricular SCOS unit and the SPG unit to guide the neuromodulating electric stimulation to the wearer, including controlling the stimulation parameters, duration, timing, interval and selection of at least one component of the neuromodulation system, and wherein the closed-loop control system and the processing unit are further configured to use the R-wave synchronization mode to guide the neuromodulating electric stimulation to the wearer.

7. The automatic detection-therapy system for cardiac arrhythmia of claim 6, wherein the cardiovascular monitoring apparatus further comprises an auricular electroencephalogram (EEG) monitoring system, wherein the auricular EEG monitoring system comprises an EEG recording module having a plurality of EEG sensor electrodes, wherein each EEG sensor electrode is in electronic communication with the EEG recording module, wherein each EEG sensor electrode is configured to contact a skin area of the first ear of the wearer, wherein the skin area that each EEG sensor electrode is configured to contact is selected from one of the following: an external ear of the first ear of the wearer, an external ear canal of the first ear of the wearer and a periauricular area around the first ear of the wearer, wherein each EEG sensor electrode is configured to contact different skin area of the wearer's first ear 902, wherein the EEG recording module is configured to record EEG data of the wearer, wherein the auricular EEG monitoring system is in electronic communication with the processing unit, wherein the processing unit is configured to convert the EEG data into quantitative EEG (qEEG) data, wherein the processing unit is configured to analyzed the EEG data and qEEG data recorded by the EEG recording module to detect at least one of the following: an increase of relative delta power more than a predetermined percentage, an increase of delta/alpha ratio more than a predetermined percentage, and a decrease of cordance z-score more than a predetermined amount, wherein when presence of serious cardiac arrhythmia is detected by the processing unit, the processing unit and the closed-loop control system are configured to utilize continuous input data of the wearer's ECG from the auricular ECG monitoring system, continuous input data of the cerebral hemodynamic data from at least one of: the auricular SCOS unit and the SPG unit, and continuous input data of the relative delta power, delta/alpha ratio and cordance z-score from the auricular EEG monitoring system to guide the neuromodulating electric stimulation to the wearer, including controlling the stimulation parameters, duration, timing, interval and selection of at least one component of the neuromodulation system, wherein the closed-loop control system and the processing unit are further configured to use the R-wave synchronization mode to guide the neuromodulating electric stimulation to the wearer, wherein when presence of impending serious cardiac arrhythmia is detected by the processing unit, the processing unit and the closed-loop control system are configured to utilize continuous input data of the wearer's ECG from the auricular ECG monitoring system, continuous input data of the cerebral hemodynamic data from at least one of: the auricular SCOS unit and the SPG unit, and continuous input data of the relative delta power, delta/alpha ratio and cordance z-score from the auricular EEG monitoring system to guide the neuromodulating electric stimulation to the wearer, including controlling the stimulation parameters, duration, timing, interval and selection of at least one component of the neuromodulation system, and wherein the closed-loop control system and the processing unit are further configured to use the R-wave synchronization mode to guide the neuromodulating electric stimulation to the wearer.

8. The automatic detection-therapy system for cardiac arrhythmia of claim 1, wherein the neuromodulation system comprises at least one of the following components: a first transcutaneous auricular vagus nerve stimulation unit (taVNS unit), a supraorbital nerve stimulation unit, a first auriculotemporal nerve stimulation unit, an occipital nerve stimulation unit, a first greater auricular nerve stimulation unit, an infraorbital nerve stimulation unit and a median nerve stimulation unit, and wherein each component of the neuromodulation system is configured to give neuromodulating electric stimulation to the wearer when prompted.

9. The automatic detection-therapy system for cardiac arrhythmia of claim 7, wherein the neuromodulation system comprises at least two of the following components: a first transcutaneous auricular vagus nerve stimulation unit (taVNS unit), a supraorbital nerve stimulation unit, a first auriculotemporal nerve stimulation unit, an occipital nerve stimulation unit, a first greater auricular nerve stimulation unit, an infraorbital nerve stimulation unit and a median nerve stimulation unit, wherein each component of the neuromodulation system is configured to give neuromodulating electric stimulation to the wearer when prompted, wherein the first taVNS unit comprises a first taVNS stimulating electrode configured to contact vagus innervated auricular skin of the wearer's first ear when in use, wherein the supraorbital nerve stimulation unit comprises a supraorbital nerve stimulating electrode configured to contact supraorbital nerve innervated midforehead skin of the wearer when in use, wherein the first auriculotemporal nerve stimulating unit comprises a first auriculotemporal nerve stimulating electrode configured to contact auriculotemporal nerve innervated auricular skin of the wearer's first ear when in use, wherein the occipital nerve stimulation unit comprises an occipital nerve stimulating electrode configured to contact the wearer's mid-occipital region when in use, wherein the first greater auricular nerve stimulation unit comprises a first greater auricular nerve stimulating electrode configured to contact greater auricular nerve innervated auricular skin of the wearer's first ear when in use, wherein the infraorbital nerve stimulation unit comprises an infraorbital nerve stimulating electrode configured to contact infraorbital nerve innervated mid-facial skin of the wearer when in use, and wherein the median nerve stimulation unit comprises a median nerve stimulating electrode configured to contact a Neiguan point on the wearer's palmar side of distal forearm where the median nerve passes underneath.

10. The automatic detection-therapy system for cardiac arrhythmia of claim 8, further comprising a second neuromodulation system, wherein the second neuromodulation system includes at least one of the following components: a second taVNS unit, a second auriculotemporal nerve stimulation unit and a second greater auricular nerve stimulation unit, wherein each component of the second neuromodulation system is in electronic communication with the processing unit, wherein each component of the second neuromodulation system is configured to give neuromodulating electric stimulation to the wearer when prompted, wherein the second taVNS unit comprises a second taVNS stimulating electrode configured to contact vagus innervated auricular skin of the wearer's second ear, wherein the second auriculotemporal nerve stimulation unit comprises a second auriculotemporal nerve stimulating electrode configured to contact auriculotemporal nerve innervated auricular skin of the wearer's second ear, and wherein the second greater auricular nerve stimulation unit comprises a second greater auricular nerve stimulating electrode configured to contact greater auricular nerve innervated auricular skin of the wearer's second ear.

11. The automatic detection-therapy system for cardiac arrhythmia of claim 1, further comprising a network interface and at least one of: a speaker and a vibrator, wherein the processing unit is in electronic communication with the network interface, the speaker and the vibrator, wherein when the processing unit detect presence of ECG data suggestive of serious cardiac arrhythmia, the processing unit is configured to generate signals to the network interface and the network interface is configured to send notification to at least one of: a clint device of the wearer and a client device of a healthcare provider of the wearer, wherein when the processing unit detect presence of ECG data suggestive of serious cardiac arrhythmia the processing unit is also configured to generate signals to at least one of: the speaker to generate an audible notification to the client device of the wearer and the vibrator to generate a tactile notification to the client device of the wearer, wherein when the processing unit detect presence of ECG data suggestive of impending serious cardiac arrhythmia, the processing unit is configured to generate signals to the network interface and the network interface is configured to send notification to at least one of: the clint device of the wearer and the client device of the healthcare provider of the wearer, wherein when the processing unit detects presence of ECG data suggestive of impending serious cardiac arrhythmia the processing unit is also configured to generate signals to at least one of: the speaker to generate an audible notification to the client device of the wearer and the vibrator to generate a tactile notification to the client device of the wearer, wherein when the processing unit detect both of: cessation of ECG data suggestive of serious cardiac arrhythmia and cessation of ECG data suggestive of impending serious cardiac arrhythmia, the processing unit is further configured to generate signals to the network interface and the network interface is configured to send cessation notification to one of: the clint device of the wearer and the client device of the healthcare provider of the wearer, and wherein when the processing unit detect both of: cessation of ECG data suggestive of serious cardiac arrhythmia and cessation of ECG data suggestive of impending serious cardiac arrhythmia the processing unit is further configured to generate signals to at least one of: the speaker to generate an audible cessation notification to the client device of the wearer and the vibrator to generate a tactile cessation notification to the client device of the wearer.

12. The automatic detection-therapy system for cardiac arrhythmia of claim 8, further comprising at least one of the following: a multifunctional timer-switch and a programmable multifunctional timer-switch to enable the wearer to do at least one of the following: to select manual or automatic control, to select various time courses, and to select at least one of the following components of the neuromodulation system: the first taVNS unit, the supraorbital nerve stimulation unit, the infraorbital nerve stimulation unit, the first auriculotemporal nerve stimulation unit, the occipital nerve stimulation unit, the first greater auricular nerve stimulation unit and the median nerve stimulation unit including various combinations thereof, wherein when prompted the neuromodulation system is configured to generate neuromodulating electric stimulation to the wearer according to the selection of the stimulation mode selected by the wearer, and wherein the selections of the stimulation mode include the following: single neuromodulation by one component of the neuromodulation system, double neuromodulation by various combinations of two components of the neuromodulation system, and triple neuromodulation by various combinations of three components of the neuromodulation system.

13. The automatic detection-therapy system for cardiac arrhythmia of claim 7, wherein the neuromodulation system comprises at least one of the following components: a taVNS unit, a supraorbital nerve stimulation unit, an auriculotemporal nerve stimulation unit, an occipital nerve stimulation unit, a greater auricular nerve stimulation unit, an infraorbital nerve stimulation unit and a median nerve stimulation unit, wherein each component of the neuromodulation system is configured to give neuromodulating electric stimulation to the wearer when prompted, wherein the closed-loop control system is in electronic communication with the cardiovascular monitoring apparatus and each component of the neuromodulation system, wherein the closed-loop control system receives real-time continuous input data from the cardiovascular monitoring apparatus including the ECG data and the cerebral hemodynamic data, wherein the cerebral hemodynamic data comprises at least one of the following: cerebral and extracranial blood flow data from the auricular SCOS unit, systolic blood pressure data diastolic blood pressure data and regional blood flow data from the SPG unit, relative delta power data delta/alpha ratio data and accordance z-score data from the auricular EEG monitoring system, wherein the closed-loop control system and the processing unit are configured to analyze the real-time continuous ECG data and cerebral hemodynamic data, using controlling algorithms, to turn on or turn off at least a component of the neuromodulation system and to continuously adjust the stimulating parameters of the neuromodulation system during the time when the neuromodulation system is turned on, and wherein the closed-loop control system and the processing unit are further configured to use the R-wave synchronization mode to guide the neuromodulating electric stimulation to the wearer.

14. The automatic detection-therapy system for cardiac arrhythmia of claim 9, wherein the auricular ECG monitoring system includes a first ECG sensor electrode and a second ECG sensor electrode, wherein the auricular SCOS unit includes a SCOS sensor, wherein the neuromodulation system comprises: a first taVNS unit having a first taVNS stimulating electrode, a first auriculotemporal nerve stimulation unit having a first auriculotemporal nerve stimulating electrode and a first greater auricular nerve stimulation unit having a first greater auricular nerve stimulating electrode, wherein the first ECG sensor electrode, the second ECG sensor electrode, all of the EEG sensor electrodes, the SCOS sensor, the SPG unit, the first taVNS stimulating electrode, the first auriculotemporal nerve stimulating electrode and the first greater auricular nerve stimulating electrode are configured to be housed in a first auricular housing, wherein the first auricular housing includes a first tubular-shaped structure and a first body-structure, wherein the first tubular-shaped structure is configured to be inserted into an external ear canal of the wearer's first ear when in use, wherein the first body-structure is configured to be placed at immediate opening of the external ear canal of the wearer's first ear and be placed inside a tragus-concha bowl of the wearer's first ear when in use, wherein the first ECG sensor electrode and all of the EEG sensor electrodes are configured to be located on a surface and partially embedded in the surface with protrusion at the surface of the first tubular-shaped structure, wherein the second ECG sensor electrode and the first greater auricular nerve stimulating electrode are configured to be located on a surface and partially embedded in the surface with protrusion at the surface of the first body-structure, wherein the SCOS sensor, the SPG unit, the first taVNS stimulating electrode and the first auriculotemporal nerve stimulating electrode are configured to be located on the surface and partially embedded in the surface with protrusion at the surface of one of: the first tubular-shaped structure and the first body-structure, wherein the first tubular-shaped structure is configured to be made of elastic flexible adaptable material and the material is configured to have appropriate elasticity flexibility and adaptability so that the first tubular-shaped structure will naturally adapt to the contour of the external ear canal of the wearer's first ear and snugly fill the interior of the external ear canal of the wearer's first ear when the first tubular-shaped structure is inserted into the external ear canal of the wearer's first ear and so that the first ECG sensor electrode and all of the EEG sensor electrodes will be naturally in close contact with the skin of the external ear canal of the wearer's first ear; wherein the first body-structure is configured to be made of elastic flexible adaptable material and the material is configured to have appropriate elasticity flexibility and adaptability so that the first body-structure will naturally adapt to the contour of the tragus-concha bowl of the wearer's first ear and snugly fill the interior of the tragus-concha bowl of the wearer's first ear when the first body-structure is placed inside the tragus-concha bowl of the wearer's first ear and so that the second ECG electrode and the first greater auricular nerve stimulating electrode will be naturally in close contact with the skin of the tragus-concha bowl of the wearer's first ear, and, at the same time, the first taVNS stimulating electrode, the first auriculotemporal nerve stimulating electrode, the SPG unit and the SCOS sensor will be naturally in close contact with one of: the skin of the tragus-concha bowl of the wearer's first ear and the skin of the external ear canal of the wearer's first ear, and so that the first taVNS stimulating electrode will be naturally in close contact with the vagus innervated auricular skin of the wearer's first ear, provided by carefully selecting a location for the first taVNS stimulating electrode on the first tubular-shaped structure or the first body-structure to match one of the innervation locations of the vagus innervated auricular skin on the external ear canal of the wearer's first ear or the tragus-concha bowl of the wearer's first ear, and, at the same time, the first auriculotemporal nerves stimulating electrode will be naturally in close contact with auriculotemporal nerve innervated auricular skin of the wearer's first ear, provided by carefully selecting a location for the first auriculotemporal nerve stimulating electrode on the first tubular-shaped structure or the first body-structure to match one of the innervation locations of the auriculotemporal nerve innervated skin on the external ear canal of the wearer's first ear or the tragus-concha bowl of the wearer's first ear, and, at the same time, the first greater auricular nerve stimulating electrode will be naturally in close contact with greater auricular nerve innervated auricular skin of the wearer's first ear, provided by carefully selecting a location for the first greater auricular nerve stimulating electrode on the first body-structure to match one of the innervation locations of the greater auricular nerve innervated skin on tragus-concha bowl of the wearer's first ear.

15. The automatic detection-therapy system for cardiac arrhythmia of claim 7, wherein the auricular electroencephalogram (EEG) monitoring system comprises a first EEG recording module having a plurality of EEG sensor electrodes, wherein the first EEG recording module is in electronic communication with each EEG sensor electrode of the first EEG recording module, wherein the first EEG recording module is configured to record EEG data of the wearer, wherein all of the EEG sensor electrodes of the first EEG recording module are configured to be housed in a first auricular housing having a first tubular-shaped structure, wherein the first tubular-shaped structure is configured to be inserted into an external ear canal of the wearer's first ear when in use, wherein all of the EEG sensor electrodes of the first EEG recording module are configured to be located on a surface and partially embedded in the surface with protrusion at the surface of the first tubular-shaped structure, wherein one or more EEG sensor electrode(s) are located at the upper surface of the first tubular-shaped structure, wherein one or more EEG sensor electrode(s) are located above the horizontal level of the first tubular-shaped structure and are facing forward-upward, wherein one or more EEG sensor electrode(s) are located above the horizontal level of the first tubular-shaped structure and are facing backward-upward, wherein the first tubular-shaped structure comprises an elastic flexible and adaptable material, and wherein the elastic flexible and adaptable material of the first tubular-shaped structure is configured to have appropriate elasticity flexibility and adaptability so that when the first tubular-shaped structure is inserted into the external ear canal of the wearer's first ear the first tubular-shaped structure will naturally adapt to the contour of the external ear canal of the wearer's first ear and snugly fill the interior of the external ear canal of the wearer's first ear, so that all of the EEG sensor electrodes of the first EEG recording module are naturally in close contact with the skin of the external ear canal of the wearer's first ear when the first tubular-shaped structure is inserted into the external ear canal of the wearer's first ear.

16. The automatic detection-therapy system for cardiac arrhythmia of claim 6, further comprising a network interface and at least one of: a speaker on a client device of the wearer and a vibrator on the client device of the wearer, wherein the processing unit is in electronic communication with the network interface, the speaker and the vibrator, wherein the processing unit is configured to analyze the cerebral blood flow data recorded by the auricular SCOS unit, and the systolic and diastolic blood pressure data and regional blood flow data recorded by the SPG unit to detect presence of significant hemodynamic change of the wearer, wherein significant hemodynamic change of the wearer comprises presence of at least one of the following: a decrease of cerebral blood flow more than a predetermined percentage, a decrease of systolic blood pressure more than a predetermined amount, a decrease of diastolic blood pressure more than a predetermined amount and a decrease of regional blood flow more than a predetermined percentage, wherein when the processing unit detects presence of ECG data suggestive of serious cardiac arrhythmia without co-occurrence of presence of significant hemodynamic change, the processing unit is configured to send signals to the neuromodulation system to prompt the neuromodulation system to start sending neuromodulating electric stimulation to the wearer under the guidance of the closed-loop control system using the continuous input data from the auricular ECG monitoring system, the auricular SCOS unit and the SPG unit and using R-wave synchronization mode, wherein when the processing unit detects co-occurrence of both of the following: presence of ECG data suggestive of serious cardiac arrhythmia and presence of significant hemodynamic change, the processing unit is configured to send signals to the neuromodulation system to prompt the neuromodulation system to do at least one of: increasing potency of neuromodulating electric stimulation to the wearer and recruiting more components of the neuromodulation system, under the guidance of the closed-loop control system using the continuous input data from the auricular ECG monitoring system, the auricular SCOS unit and the SPG unit and using R-wave synchronization mode, wherein when the processing unit detects co-occurrence of both of the following: presence of a serious cardiac arrhythmia and presence of significant hemodynamic change, the processing unit is further configured to send signals to the network interface to prompt the network interface to generate an urgent notification to at least one of: a client device of the wearer and a client device of a healthcare provider of the wearer, and wherein when the processing unit detects co-occurrence of both of the following: presence of a serious cardiac arrhythmia and presence of significant hemodynamic change, the processing unit is further configured to send signals to the network interface to prompt the network interface to generate an urgent notification to at least one of: the speaker on the client device of the wearer to generate an urgent audible notification and the vibrator on the client device of the wearer to generate an urgent tactile notification.

17. The automatic detection-therapy system for cardiac arrhythmia of claim 1, wherein the neuromodulation system comprises the following components: a transcutaneous auricular vagus nerve stimulation unit (taVNS unit), an auriculotemporal nerve stimulation unit, and a greater auricular nerve stimulation unit, wherein each component of the neuromodulation system is configured to give neuromodulating electric stimulation to the wearer when activated, wherein the taVNS unit includes a taVNS stimulating electrode, wherein the auriculotemporal nerve stimulation unit includes an auriculotemporal nerve stimulating electrode, wherein the greater auricular nerve stimulation unit includes a greater auricular nerve stimulating electrode, wherein the taVNS stimulating electrode, the auriculotemporal nerve stimulating electrode and the greater auricular nerve stimulating electrode are configured to be housed together in an auricular housing and are further configured to automatically contact their respective innervated auricular skin when the auricular housing is placed in the wearer's ear.

18. The automatic detection-therapy system for cardiac arrhythmia of claim 1, wherein the neuromodulation system comprises a median nerve stimulation unit having a median nerve stimulating electrode, wherein the median nerve stimulating electrode is configured to contact the skin at a Neiguan point on the palmar side of the wearer's distal forearm, wherein the median nerve stimulation unit is configured to give neuromodulating electric stimulation to the wearer's median nerve when prompted, wherein the median nerve stimulation unit is in electronic communication with the processing unit, wherein when serious cardiac arrhythmia is detected by the processing unit, the processing unit is configured to send signals to the median nerve stimulation unit to prompt the median nerve stimulation unit to start sending neuromodulating electric stimulation to the wearer using the R-wave synchronization mode under the guidance of the closed-loop control system utilizing continuous input data from the cardiovascular monitoring apparatus, and wherein when impending serious cardiac arrhythmia is detected by the processing unit, the processing unit is further configured to send signals to the median nerve stimulation unit to prompt the median nerve stimulation unit to start sending neuromodulating electric stimulation to the wearer using the R-wave synchronization mode under the guidance of the closed-loop control system utilizing continuous input data from the cardiovascular monitoring apparatus.

19. The automatic detection-therapy system for cardiac arrhythmia of claim 1, further comprising an auricular housing and a wrist housing, wherein the ECG recording module comprises at least two ECG sensor electrodes including a first ECG sensor electrode and a second ECG sensor electrode, wherein the neuromodulation system comprises a median nerve stimulation unit and at least one of the following: a transcutaneous auricular vagus nerve stimulation (taVNS) unit, an auriculotemporal nerve stimulation unit and a greater auricular nerve stimulation unit, wherein the first ECG sensor electrode and at least one of the following: the taVNS unit, the auriculotemporal nerve stimulation unit and the greater auricular nerve stimulation unit are configured to be housed in the auricular housing, wherein the second ECG sensor electrode and the median nerve stimulation unit are configured to be housed in the wrist housing, wherein the taVNS stimulation unit comprises a taVNS stimulating electrode, wherein the auriculotemporal nerve stimulation unit comprises an auriculotemporal nerve stimulating electrode, wherein the greater auricular nerve stimulation unit comprises a greater auricular nerve stimulating electrode, wherein the median nerve stimulation unit comprises a median nerve stimulating electrode, wherein the first ECG sensor electrode, the taVNS stimulating electrode, the auriculotemporal nerve stimulating electrode and the greater auricular nerve stimulating electrode are configured to be located on a surface and partially embedded in the surface with protrusion at the surface of the auricular housing, wherein the auricular housing is configured to comprise a flexible elastic and adaptable material, wherein the material for the auricular housing is configured to have appropriate flexibility elasticity and adaptability so that when the auricular housing is placed in the wearer's ear the first ECG sensor electrode, the taVNS stimulating electrode, the auriculotemporal nerve stimulating electrode and the greater auricular nerve stimulating electrode are naturally in close contact with the skin of the wearer's ear and so that the taVNS stimulating electrode, the auriculotemporal nerve stimulating electrode and the greater auricular nerve stimulating electrode are automatically contacting their respective innervated auricular skin, and wherein the second ECG sensor electrode and the median nerve stimulating electrode are configured to be located on a surface of the wrist housing with the median nerve stimulating electrode configured to be located at the Neiguan point where the median nerve passes underneath when the wrist housing is worn by the wearer so that when the wrist housing is worn by the wearer the second ECG sensor electrode is in close contact with the wearer's forearm skin and the median nerve stimulating electrode is in close contact with the wearer's forearm skin at the Neiguan point where the median nerve passes underneath.

20. The automatic detection-therapy system for cardiac arrhythmia of claim 9, wherein the processing unit is configured to analyze the ECG data recorded by the auricular ECG monitoring system to detect presence of serious cardiac arrhythmia, wherein the processing unit is configured to analyze the data recorded by the auricular SCOS unit to detect presence of a decrease of cerebral blood flow more than a predetermined percentage, wherein the processing unit is configured to analyze the data recorded by the SPG unit to detect presence of at least one of: a decrease of systolic blood pressure more than a predetermined amount, a decrease of diastolic blood pressure more than a predetermined amount and a decrease of regional blood flow more than a predetermined percentage, wherein the processing unit is configured to analyze the qEEG data recorded by the auricular EEG monitoring system to detect presence of at least one of: an increase of relative delta power more than a predetermined percentage, an increase of delta/alpha ratio more than a predetermined percentage and a decrease of cordance z-score more than a predetermined amount, wherein when the processing unit detects presence of serious cardiac arrhythmia that co-occurs with at least one of the following: a decrease of cerebral blood flow more than the predetermined percentage, a decrease of systolic blood pressure more than the predetermined amount, a decrease of diastolic blood pressure more than the predetermined amount, a decrease of regional blood flow more than the predetermined percentage, an increase of relative delta power more than the predetermined percentage, an increase of delta/alpha ratio more than the predetermined percentage and a decrease of cordance z-score more than the predetermined amount, the processing unit is further configured to send signals to the neuromodulation system to prompt the neuromodulation system to do at least one of the following: to increase the potency of the neuromodulating electric stimulation and to recruit more neuromodulating units to start neuromodulating electric stimulation, and wherein the processing unit and the closed-loop control system are configured to utilized R-wave synchronization mode and continuous input data from the auricular ECG monitoring system, the SCOS unit, the SPG unit and the auricular EEG monitoring system to regulate and guide the neuromodulation.

21. A cardiovascular monitoring apparatus, comprising:
an auricular electrocardiogram (ECG) monitoring system comprising an ECG recording module and at least two ECG sensor electrodes including a first ECG sensor electrode and a second ECG sensor electrode, wherein the first ECG sensor electrode and the second ECG sensor electrode are configured to contact separate areas on a first ear of a wearer, wherein the first and the second ECG sensor electrodes are in electronic communication with the ECG recording module, wherein the ECG recording module is configured to record the wearer's ECG data;
an auricular speckle contrast optical spectroscopy (SCOS) unit configured to contact the skin of the first ear of the wearer, wherein the auricular SCOS unit is configured to record the wearer's cerebral blood flow data;
a network interface; and
a processing unit in electronic communication with the auricular ECG monitoring system, the auricular SCOS unit and the network interface, wherein the processing unit is configured to analyze the ECG data recorded by the auricular ECG monitoring system to detect presence or cessation of serious cardiac arrhythmia and presence or cessation of impending serious cardiac arrhythmia, wherein the processing unit is also configured to analyze the wearer's cerebral blood flow data recorded by the auricular SCOS unit to detect presence or cessation of a decrease of cerebral blood flow more than a predetermined percentage,
wherein when the processing unit detects presence of ECG data suggestive of serious cardiac arrhythmia that does not co-occur with presence of cerebral blood flow data showing a decrease of cerebral blood flow more than the predetermined percentage, the processing unit is configured to send signals to the network interface to prompt the network interface to send a notification to at least one of: a client device of the wearer and a client device of the wearer's healthcare provider;
wherein when the processing unit detects co-occurrence of both of the following: presence of ECG data suggestive of serious cardiac arrhythmia and presence of cerebral blood flow data showing a decrease of cerebral blood flow more than the predetermined percentage, the processing unit is configured to send signals to the network interface to prompt the network interface to send an urgent notification to at least one of: a client device of the wearer and a client device of the wearer's healthcare provider,
wherein when the processing unit detects presence of ECG data suggestive of impending serious cardiac arrhythmia, the processing unit is configured to send signals to the network interface to prompt the network interface to send a notification to at least one of: the client device of the wearer and the client device of the healthcare provider of the wearer, and
wherein when the processing unit detects all of the following: cessation of ECG signals suggestive of serious cardiac arrhythmia, cessation of ECG signals suggestive of impending serious cardiac arrhythmia, cessation of a decrease of cerebral blood flow more than the predetermined percentage, the processing unit is further configured to send signals to the network interface to prompt the network interface to send a cessation notification to at least one of: the client device of the wearer and the client device of the healthcare provider of the wearer.

22. The cardiovascular monitoring apparatus of claim 21, further comprising a speckle-plethysmography (SPG) unit, wherein the SPG unit is configured to contact the skin of the first ear of the wearer, wherein the SPG unit is configured to record the wearer's regional blood flow data at the area where the SPG unit contacts the wearer's first ear, wherein the SPG unit is also configured to record the wearer's systolic blood pressure data and diastolic blood pressure data, wherein the processing unit is in electronic communication with the SPG unit, wherein the processing unit is configured to analyze the ECG data recorded by the auricular ECG monitoring system to detect presence or cessation of serious cardiac arrhythmia and presence or cessation of impending serious cardiac arrhythmia, wherein the processing unit is configured to analyze the cerebral blood flow data recorded by the SCOS unit to detect presence or cessation of a decrease of cerebral blood flow more than a predetermined percentage, wherein the processing unit is also configured to analyze the regional blood flow data, systolic blood pressure data and diastolic blood pressure data recorded by the SPG unit to detect: presence or cessation of a decrease of regional blood flow more than a predetermined percentage, presence or cessation of a decrease of systolic blood pressure more than a predetermined amount, and presence or cessation of a decrease of diastolic blood pressure more than a predetermined amount,
Wherein when the processing unit detects presence of ECG data suggestive of serious cardiac arrhythmia that does not co-occur with any of the following: presence of a decrease of cerebral blood flow more than the predetermined percentage, presence of a decrease of regional blood flow more than the predetermined percentage, presence of a decrease of systolic blood pressure more than the predetermined amount, and presence of a decrease of diastolic blood pressure more than the predetermined amount, the processing unit is configured to send signals to the network interface to prompt the network interface to send a notification to at least one of: the client device of the wearer and the client device of the healthcare provider of the wearer;
wherein when the processing unit detects presence of ECG data suggestive of serious cardiac arrhythmia that co-occurs with any of the following: presence of a decrease of cerebral blood flow more than the predetermined percentage, presence of a decrease of regional blood flow more than the predetermined percentage, presence of a decrease of systolic blood pressure more than the predetermined amount, and presence of a decrease of diastolic blood pressure more than the predetermined amount, the processing unit is configured to send signals to the network interface to prompt the network interface to send an urgent notification to at least one of: the client device of the wearer and the client device of the healthcare provider of the wearer;
wherein when the processing unit detects presence of ECG data suggestive of impending serious cardiac arrhythmia, the processing unit is configured to send signals to the network interface to prompt the network interface to send a notification to at least one of: the client device of the wearer and the client device of the healthcare provider of the wearer, and
wherein when the processing unit detects all of the following: cessation of ECG data suggestive of serious cardiac arrhythmia, cessation of ECG data suggestive of impending serious cardiac arrhythmia, cessation of a decrease of cerebral blood flow more than the predetermined percentage, cessation a decrease of regional blood flow more than the predetermined percentage, cessation of a decrease of systolic blood pressure more than the pre-determined amount and cessation of a decrease of diastolic blood pressure more than the pre-determined amount, the processing unit is further configured to send signals to the network interface to prompt the network interface to send a cessation notification to at least one of: the client device of the wearer and the client device of the healthcare provider of the wearer.

23. The cardiovascular monitoring apparatus of claim 21, further comprising an auricular electroencephalogram (EEG) monitoring system, wherein the auricular EEG monitoring system comprises an EEG recording module and a plurality of EEG sensor electrodes configured to contact separate areas on a first ear of a wearer, wherein each EEG sensor electrodes is in electronic communication with the EEG recording module, wherein the EEG recording module is configured to record EEG data of the wearer, wherein the SCOS unit, the auricular ECG monitoring system and the auricular EEG monitoring system are in electronic communication with the processing unit, wherein the processing unit is configured to convert the EEG data into quantitative EEG (qEEG) data, wherein the processing unit is configured to analyze the ECG data recorded by the auricular ECG monitoring system to detect presence or cessation of serious cardiac arrhythmia and presence or cessation of impending serious cardiac arrhythmia, wherein the processing unit is configured to analyze the EEG data and qEEG data recorded by the auricular EEG monitoring system to detect presence of at least one of: an increase of relative delta power more than a predetermined percentage, an increase of delta/alpha ratio more than a predetermined percentage and a decrease of cordance z-score more than a predetermined amount, wherein the processing unit is also configured to analyze the SCOS data recorded by the SCOS unit to detect presence or cessation of a decrease of cerebral blood flow more than a predetermined percentage, wherein when the processing unit detects presence of ECG data suggestive of serious cardiac arrhythmia that does not co-occur with any of the following: presence of SCOS data showing a decrease of cerebral blood flow more than the predetermined percentage, presence of EEG data and qEEG data showing at least one of: an increase of relative delta power more than the predetermined percentage, an increase of delta/alpha ratio more than the predetermined percentage and a decrease of cordance z-score more than the predetermined amount, the processing unit is configured to send signals to the network interface to prompt the network interface to send a notification to at least one of: a client device of the wearer and a client device of the healthcare provider of the wearer, wherein when the processing unit detects presence of ECG data suggestive of serious cardiac arrhythmia co-occurs with at least one of the following: presence of SCOS data showing a decrease of cerebral blood flow more than the predetermined percentage, presence of EEG data and qEEG data showing at least one of: an increase of relative delta power more than the predetermined percentage, an increase of delta/alpha ratio more than the predetermined percentage and a decrease of cordance z-score more than the predetermined amount, the processing unit is configured to send signals to the network interface to prompt the network interface to send an urgent notification to at least one of: a client device of the wearer and a client device of the healthcare provider of the wearer, wherein when the processing unit detects presence of ECG data suggestive of impending serious cardiac arrhythmia, the processing unit may be configured to send signals to the network interface to prompt the network interface to send a notification to at least one of: the client device of the wearer and the client device of the healthcare provider of the wearer, and wherein when the processing unit detects all of the following: cessation of ECG data suggestive of serious cardiac arrhythmia, cessation of ECG data suggestive of impending serious cardiac arrhythmia, cessation of a decrease of cerebral blood flow more than the predetermined percentage, cessation of an increase of relative delta power more than the predetermined percentage, cessation of an increase of delta/alpha ratio more than the predetermined percentage and cessation of a decrease of cordance z-score more than the predetermined amount, the processing unit is further configured to send signals to the network interface to prompt the network interface to send a cessation notification to at least one of: the client device of the wearer and the client device of the healthcare provider of the wearer.

24. The cardiovascular monitoring apparatus of claim 21, further comprising a photoplethysmography (PPG) unit, wherein the PPG unit is configured to contact the skin of the first ear of the wearer, wherein the PPG unit is configured to record the wearer's regional blood flow data at the area where the PPG unit contacts the wearer's first ear, wherein the PPG unit is also configured to record the wearer's systolic blood pressure data and diastolic blood pressure data, wherein the processing unit is in electronic communication with the PPG unit, wherein the processing unit is configured to analyze the ECG data recorded by the auricular ECG monitoring system to detect presence or cessation of serious cardiac arrhythmia and presence or cessation of impending serious cardiac arrhythmia, wherein the processing unit is configured to analyze the cerebral blood flow data recorded by the SCOS unit to detect presence or cessation of a decrease of cerebral blood flow more than a predetermined percentage, wherein the processing unit is also configured to analyze the regional blood flow data, systolic blood pressure data and diastolic blood pressure data recorded by the PPG unit to detect: presence or cessation of a decrease of regional blood flow more than a predetermined percentage, presence or cessation of a decrease of systolic blood pressure more than a predetermined amount, and presence or cessation of a decrease of diastolic blood pressure more than a predetermined amount, wherein when the processing unit detects presence of ECG data suggestive of serious cardiac arrhythmia co-occurs with at least one of the following: presence of a decrease of cerebral blood flow more than the predetermined percentage, presence a decrease of regional blood flow more than the predetermined percentage, presence of a decrease of systolic blood pressure more than the predetermined amount and presence of a decrease of diastolic blood pressure more than the predetermined amount, the processing unit is configured to send signals to the network interface to prompt the network interface to send an urgent notification to at least one of: a client device of the wearer and a client device of the healthcare provider of the wearer, wherein when the processing unit detects presence of ECG data suggestive of impending serious cardiac arrhythmia, the processing unit may be configured to send signals to the network interface to prompt the network interface to send a notification to at least one of: the client device of the wearer and the client device of the healthcare provider of the wearer, and wherein when the processing unit detects all of the following: cessation of ECG data suggestive of serious cardiac arrhythmia, cessation of ECG data suggestive of impending serious cardiac arrhythmia, cessation of a decrease of cerebral blood flow more than the predetermined percentage, cessation of a decrease of regional blood flow more than the predetermined percentage, cessation of a decrease of systolic blood pressure more than the predetermined amount and cessation of a decrease of diastolic blood pressure more than the predetermined amount, the processing unit is further configured to send signals to the network interface to prompt the network interface to send a cessation notification to at least one of: the client device of the wearer and the client device of the healthcare provider of the wearer.

25. A closed-loop neuromodulation apparatus, comprising
a cardiovascular monitoring system, comprising an auricular electrocardiogram (ECG) monitoring system, and at least one of: a speckle contrast optical spectroscopy (SCOS) unit, a speckle-plethysmography (SPG) unit and a photoplethysmography (PPG) unit, wherein the auricular electrocardiogram (ECG) monitoring system is configured to record ECG data of a wearer, wherein the auricular ECG monitoring system is configured to be housed in an auricular housing, wherein the auricular housing is configured to be placed in a first ear of a wearer when in use, wherein the auricular speckle contrast optical spectroscopy (SCOS) unit is configured to be housed in the auricular housing, wherein the auricular SCOS unit is configured to record the wearer's cerebral and extracranial blood flow data, wherein the SPG unit is configured to be housed in the auricular housing and configured to contact a location of the skin of the wearer's first ear when in use, wherein the SPG unit is configured to record the wearer's regional blood flow data at the location where the SPG unit contacts the wearer's first ear, wherein the SPG unit is also configured to record systolic blood pressure data and diastolic blood pressure data of the wearer, and wherein the PPG unit is configured to record the wearer's regional blood flow data at the location where the PPG unit contacts the wearer's first ear, and wherein the PPG unit is also configured to record systolic blood pressure data and diastolic blood pressure data of the wearer;
a neuromodulation system, comprising at least one of the following components: a transcutaneous auricular vagus nerve stimulation unit (taVNS unit), an auriculotemporal nerve stimulation unit, and a greater auricular nerve stimulation unit, wherein each component of the neuromodulation system is configured to give neuromodulating electric stimulation to the wearer when activated, wherein the taVNS unit includes a taVNS stimulating electrode, wherein the auriculotemporal nerve stimulation unit includes an auriculotemporal nerve stimulating electrode, wherein the greater auricular nerve stimulation unit includes a greater auricular nerve stimulating electrode, and wherein the taVNS stimulating electrode, the auriculotemporal nerve stimulating electrode and the greater auricular nerve stimulating electrode are configured to be housed together in the auricular housing and are further configured to automatically contact their respective innervated auricular skin when the auricular housing is placed in the wearer's ear;
a multi-functional switch-timer in electronic communication with each component of the neuromodulation system, wherein the multi-functional switch-timer is configured to enable the wearer to turn on or turn off each component of the neuromodulation system, to select at least one component of the neuromodulation system and to select timing course for each component of the neuromodulation system, and
a processing unit in electronic communication with the auricular ECG monitoring system and at least one of: the SCOS unit the SPG unit and the PPG unit, wherein the processing unit is also in electronic communication with the multi-functional switch-timer and each component of the neuromodulation system, wherein the processing unit comprises a closed-loop control system and R-wave synchronization mode, wherein when at least one component of the neuromodulation system is turned on by the wearer via the multifunctional switch-timer, the processing unit and the closed-loop control system are configured to regulate the stimulation parameters and the timing course using the continuous input ECG data recorded by the auricular ECG monitoring system and at least one of the following continuous input data: the wearer's cerebral and extracranial blood flow data recorded by the auricular SCOS unit, the wearer's regional blood flow data, systolic blood pressure data and diastolic blood pressure data recorded by the SPG unit and the wearer's regional blood flow data, systolic blood pressure data and diastolic blood pressure data recorded the PPG unit, and wherein the closed-loop control system and the processing unit are further configured to use the R-wave synchronization mode to guide the neuromodulating electric stimulation to the wearer.
